(19) United States
(12) Patent Application Publication
(10) Pub. No.: US 2024/0190854 A1
(43) Pub. Date: Jun. 13, 2024

(54) PYRIMIDINE OR PYRIDINE DERIVATIVES USEFUL AS HCN2 MODULATORS

(71) Applicant: King's College London, London (GB)

(72) Inventors: Peter A. McNaughton, London (GB); Karen Williams, High Peak (GB); Sue Cramp, Harlow (GB); Alan Naylor, Cambridge (GB)

(73) Assignee: King's College London, London (GB)

(21) Appl. No.: 18/279,815

(22) PCT Filed: Mar. 2, 2022
(86) PCT No.: PCT/GB2022/050552

§ 371 Date: Aug. 31, 2023

(30) Foreign Application Priority Data
Mar. 3, 2021 (GB) 2103008.5

(51) Int. Cl. C07D 413/10; A61K 31/4439; A61K 31/506; A61K 31/5377; A61P 25/02; C07D 401/10; C07D 413/14

(52) U.S. Cl. CPC C07D 413/10; A61K 31/4439; A61K 31/506; A61K 31/5377; A61P 25/02; C07D 401/10; C07D 413/14

(57) ABSTRACT

Compounds of the formula (I) and pharmaceutically acceptable salts thereof: (I) wherein the substituents are defined in the specification. The compounds are hyperpolarisation activated cyclic-nucleotide modulated ion channel 2 (HCN2) inhibitors. Also disclosed are pharmaceutical compositions comprising the compounds, and the use of the compounds for the treatment or prevention of medical conditions mediated by HCN2, including neuropathic pain.

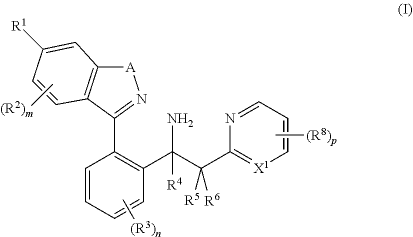

### COMPOUNDS

This invention relates to benzisoxazole and indazole compounds, to pharmaceutical compositions comprising the compounds, and to the use of the compounds for the treatment of medical conditions mediated by hyperpolarisation activated cyclic-nucleotide modulated ion channel 2 (HCN2), for example for the treatment of pain, particularly the treatment of inflammatory and/or neuropathic pain.

### BACKGROUND

Nociception is the ability to detect potentially harmful stimuli to the body resulting from the internal or external stimuli, such as extreme temperatures or tissue injury, and is generated by the activation of nociceptors. The nociceptors transmit information to the brain where the perception of acute pain is generated. Nociception is an important sense that warns an individual against present or imminent damage resulting in an acute pain signal. However, in patients with chronic pain, this warning signal persists in the absence of any genuine threat and can impose major limitations on lifestyle and working patterns. Pain results in around 40 million physician visits per year, approximately 4 billion lost working days, and a dramatic reduction in the quality of life for many patients.

Inflammatory pain (IP) results from the increased excitability of peripheral nociceptive sensory fibres produced by the action of inflammatory mediators released from injured, inflamed or stressed tissues on nociceptive (pain-sensing) nerve terminals. IP may be chronic or acute. Acute IP is associated with the immediate inflammatory response following tissue damage or injury and includes, for example, post-operative pain, dental pain and injury such as sprains or muscle tears. Generally acute IP resolves as the injury heals. However, IP can also be chronic. Chronic IP is a feature of many medical conditions, for example infection, injury, osteoarthritis and rheumatoid arthritis.

IP is typically treated with non-steroidal anti-inflammatory drugs (NSAIDs) or in more severe cases with opioids, both of which are effective but have major side effects. Undesirable side-effects associated with NSAIDs include gastric and renal complications, together with an increased incidence of myocardial infarction. Side effects associated with opioids include constipation and CNS side effects, for example cognitive impairment, sedation and addiction. Additionally, even at normal doses opiates promote respiratory depression and are the cause of many premature deaths

Neuropathic pain (NP), a form of chronic pain caused by damage and/or dysfunction of sensory nerves of the peripheral or sympathetic nervous system, for example a lesion or disease of the somatosensory system, including peripheral fibres (Aβ, Aδ and C fibres) and central neurons. The damage to the somatosensory system results in disordered transmission of sensory signals to the brain resulting in the generation of pain. Symptoms of neuropathic pain include abnormal sensation of painful and other stimuli, known as dysesthesia (e.g. hyperesthesia, hyperalgesia, allodynia (pain due to a non-noxious stimulus), and hyperpathia) and/or ongoing pain, typically sensed as deep and aching pain. NP is often long-lasting and typically persists after apparent resolution of the primary cause.

An estimated 50 million patients world-wide suffer from chronic non-malignant pain, defined as pain of greater than 3 months' duration that is not related to cancer. Neuropathic pain affects about 8% of people in the Western World at some point in their life.

Painful diabetic neuropathy (PDN), the pain resulting from nerve damage caused by Type 2 diabetes, is a major patient burden which is rapidly growing with the increasing incidence of obesity and has no highly efficacious treatment options at this stage. Postherpetic neuralgia (PHN), a long-lasting pain following a Herpes zoster (shingles) eruption, is also a significant problem, particularly amongst the elderly. Pain caused either by cancer or by the chemotherapeutic agents used to treat it (chemotherapy-induced peripheral neuropathy, CIPN) imposes an additional patient burden, and the ability of patients to tolerate the neuropathic pain induced by chemotherapy is often a limiting factor in treatment. Post-operative neuropathic pain sometimes occurs following surgical procedures causing patients chronic pain which may persist long after the surgical wound has healed. In addition to these major patient groups there are many rarer but excruciating neuropathic pain conditions such as trigeminal neuralgia, complex regional pain syndrome (CRPS) and pudendal neuralgia. In addition, many clinicians believe, on the basis that drugs used to treat neuropathic pain have some efficacy in these conditions, that there is a neuropathic pain component in many common conditions involving nerve damage or compression, such as lower back pain, nerve damage following traumatic injury (e.g. whiplash injury in car crash), fibromyalgia and carpal tunnel syndrome.

Existing therapies for NP, such as gabapentinoids, serotonin, noradrenaline-selective reuptake inhibitors (SNRIs) and tricyclic antidepressants, have poor efficacy, with as many as 70% of patients reporting limited or no relief and with number needed to treat to obtain 50% relief in a single patient (NNT) typically in the range 7-10 (Finnerup, N. B. et al., 2015, Lancet Neurol 14, 162-173). There are also numerous side effects associated with existing therapies for NP. For example, gabapentin, the current first-line therapy for NP, causes sedation, while amitriptyline (a tricyclic antidepressant) has psychotropic effects such as sedation, nightmares, impotence and confusion together with numerous drug-drug interactions.

There remains a need for new treatments for pain, particularly IP and NP.

The Hyperpolarization activated, Cyclic-Nucleotide modulated (HCN) ion channels comprise four isoforms, HCN 1, 2, 3 and 4, which carry an inward current called Iₕ (also known as I_{q} or I_{f}) activated by hyperpolarization in the range of membrane potentials between −60 and −90 mV (Kaupp & Seifert (2001) “Molecular diversity of pacemaker ion channels.” Annu. Rev. Physiol 63: 235-257; Biel et al., (2002) “Cardiac HCN channels: structure, function, and modulation.” Trends Cardiovasc. Med. 12(5): 206-212).

The HCN isoforms perform an important pacemaker function in both cardiac and nervous tissue.

HCN4 is the major regulator of cardiac rhythmicity. Inducible deletion of cardiac HCN4 causes a progressive decrease in heart rate which is fatal in mice after a few days (Baruscotti et al, “Deep bradycardia and heart block caused by inducible cardiac-specific knockout of the pacemaker channel gene HCN4”; Proc. Natl. Acad. Sci. USA 108, 2011, 1705-1710). HCN2 is expressed in atrial and ventricular cardiac tissue but appears to be largely excluded from the pacemaker region, the sino-atrial node, in both animals and humans (Herrmann S, Layh B & Ludwig A. “Novel insights into the distribution of cardiac HCN channels: an expression study in the mouse heart”. J Mol Cell Cardiol 51, 997-1006, 2011; Herrmann S, Hofmann F, Stieber J & Ludwig A. “HCN channels in the heart: lessons from mouse mutants”. BrJPharmacol 166, 501-509, 2012; Chandler, N. J., et al. “Molecular architecture of the human sinus node: insights into the function of the cardiac pacemaker.” Circulation 119(12): 1562-1575, 2009). The role of HCN2 is also thought to be less critical than HCN4, because the cardiac function of both an HCN2 global knockout mice and a human HCN2 deletion mutant is relatively normal suggesting that HCN2-selective blockers will not cause bradycardia (Ludwig et al. “Absence epilepsy and sinus dysrhythmia in mice lacking the pacemaker channel HCN2”, EMBO J 22, 2003, 216-224; and DiFrancesco et al, “Recessive loss-of-function mutation in the pacemaker HCN2 channel causing increased neuronal excitability in a patient with idiopathic generalized epilepsy”; J Neurosci 31, 2011, 17327-17337).

HCN1 and HCN2 are the predominant isoforms expressed in both brain and somatosensory neurons (Ludwig et al 2003, ibid).

NP has traditionally been attributed to sensitisation and/or remodelling of the CNS. However, in more recent work it has been shown by the use of peripherally restricted blockers of HCN ion channels and by recordings of activity in single nociceptors (pain-sensitive nerve fibres) that pain continues to have its origin in repetitive firing of peripheral nociceptors even long after the initial injury has apparently resolved. These findings suggest that peripherally restricted blockers of HCN ion channels would provide a new class of analgesics. (Young et al, “Inflammatory and neuropathic pain are rapidly suppressed by peripheral block of hyperpolarisation-activated cyclic nucleotide-gated ion channels”; Pain. 155; 2014, 1708-19; Noh, S., et al. (2014). “The heart-rate-reducing agent, ivabradine, reduces mechanical allodynia in a rodent model of neuropathic pain.” Eur J Pain 18(8): 1139-1147; Serra, J., et al. (2012), “Microneurographic identification of spontaneous activity in C-nociceptors in neuropathic pain states in humans and rats.” Pain 153(1): 42-55; reviewed in Tsantoulas, C., et al. (2016). “HCN2 ion channels: basic science opens up possibilities for therapeutic intervention in neuropathic pain.” Biochem J 473(18): 2717-2736).

The negative range of activation of HCN ion channels means that they are hardly activated at the resting membrane potential of nerve fibres, which seldom exceeds −60 mV. However, many inflammatory mediators, amongst them the potent pro-inflammatory agents PGE2 and bradykinin, bind to Gs-coupled GPCRs which thus activate adenylate cyclase and so cause an increase in cAMP (cyclic adenosine monophosphate), which in turn binds directly to a site in the C-terminal domain of HCN ion channels. The voltage range of activation of the HCN2 and HCN4 isoforms, but not HCN1 and HCN3, is shifted in the positive direction by increased intracellular cAMP. The inward current passing through activated HCN2 ion channels in nociceptive nerve fibres therefore triggers repetitive firing, resulting in a sensation of pain in vivo (Emery et al, “HCN2 ion channels play a central role in inflammatory and neuropathic pain”; Science 333, 2011, 1462-1466).

A number of studies have shown increased HCN2 channel expression and/or Iₕ current in nociceptors following neuronal damage or inflammation, though other studies have failed to find a change in expression or even found a decrease (reviewed in Tsantoulas, C., et al. (2016), ibid). Increased inward Iₕ current is expected to shift the membrane potential to more depolarized values, and so lower the activation threshold. Upregulation of HCN2 has been demonstrated in cell bodies and terminals of nociceptive neurons in preclinical models of inflammatory pain, in line with an increase in Iₕ current and hyperexcitability of the neurons. The same is not true for neuropathic pain models, where there are reports showing no change, or a reduction in HCN ion channel expression (Chaplan S R, Guo H Q, Lee D H, Luo L, Liu C, Kuei C, Velumian A A, Butler M P, Brown S M & Dubin A E., 2003, Neuronal hyperpolarization-activated pacemaker channels drive neuropathic pain. J. Neurosci 23, 1169-1178; Tsantoulas et al, 2017, ibid). However, there are alternative routes to enhanced Iₕ current than channel overexpression, such as increases in intracellular cAMP, as outlined above (reviewed in Tsantoulas et al, 2016, ibid).

It has been shown that HCN2 is expressed in nociceptive (pain-sensitive) neurons, and that modulation of the voltage-dependence of HCN2 by inflammatory mediators such as PGE2 is a major contributor to IP. It has also been shown in mouse models for inflammatory pain (including pain elicited by injection of PGE2, carrageenan and formalin) that blockage and/or targeted genetic deletion of HCN2 provides analgesia (Emery et al. 2011, ibid).

A study in a chronic constriction injury (CCI) mouse model of NP in which HCN2 had been genetically deleted from nociceptors, the mice showed no sign of NP following a nerve lesion (Emery et al, 2011 ibid.). Subsequent studies have shown that ivabradine, a non-selective blocker of HCN ion channels, is an effective analgesic in a variety of mouse models of neuropathic pain, including nerve injury, cancer chemotherapy and diabetic neuropathy models (Young et al, 2014, ibid). Further evidence for the central role of HCN2 ion channels in animal pain models is set out in Emery et al, “HCN2 ion channels: an emerging role as the pacemakers of pain” Trends Pharmacol. Sci. 33(8): 2012, 456-463; and Tsantoulas et al., Hyperpolarization-activated cyclic nucleotide-gated 2 (HCN2) ion channels drive pain in mouse models of diabetic neuropathy. Sci Transl. Med 9, 2017, eaam6072. This work suggests that HCN2-selective blockers will provide effective treatments for NP and IP.

The analgesia observed in these mouse models was achieved by blocking or genetically deleting HCN2 ion channels in peripheral nociceptive neurons alone, because the blockers used were peripherally restricted and the targeted genetic deletion was restricted to peripheral nociceptive neurons. In mouse models global genetic deletion of all HCN2, in contrast, caused epilepsy, failure to gain weight and early death (Ludwig et al. Int. J. Mol. Sci. 2015 January; 16(1): 1429-1447). Thus, a peripherally restricted HCN2 blocker is expected to provide an effective analgesic for NP and IP whilst also avoiding CNS mediated side effects which may be associated with blocking HCN2 channels in the brain. The avoidance or minimisation of CNS side-effects would also address a major problem with other analgesics such as opioids and gabapentinoids. Selective HCN2 blockers may also avoid some or all of the undesirable gastric, renal and cardiac side effects associated with NSAIDs or the constipation caused by opiates.

Experiments (Tsantoulas C et al. 2017 ibid) have shown that ivabradine and nociceptor-targeted genetic deletion of HCN2 both give complete analgesia in a mouse model of diabetic neuropathy, which closely mimics the human condition. These experiments demonstrate that neuropathic pain is primarily peripheral in origin, because in each case the intervention was peripheral, as ivabradine is peripherally restricted and the HCN2 genetic deletion was targeted to peripheral nociceptors. The view that peripheral HCN2 block will provide effective analgesia contrasts with the prevailing belief that NP, in particular, is a CNS phenomenon which would require a CNS-penetrant therapy to treat NP.

Several non-selective HCN ion channel blockers are known including ZD7288, zatebradine, cilobradine, KW-3407, YM758 and ivabradine. These compounds were developed primarily as bradycardic agents (Romanelli et al. Current Topics in Medicinal Chemistry, 16:1764-1791 and Postea et al. Nature Reviews Drug Discovery 10, 2011, 903-914).

The non-selective and peripherally restricted HCN blocker, ivabradine, has been approved by the FDA to treat symptoms associated with stable angina and heart failure. HCN4 and HCN1 channels, the targets of ivabradine in these conditions, are critical for the regulation of heart rate, and the mode of action of ivabradine is to cause bradycardia by blocking HCN4 and HCN1, and thereby to reduce the oxygen demand of the heart. Thus, although the studies described above have shown that ivabradine provides an analgesic effect on NP, the compound is not suitable as an analgesic in the clinic, because of its effects on cardiac pacemaking associated with HCN4 and/or HCN1 inhibition. Accordingly, preferred analgesics targeting HCN2 ion channels for the treatment of, for example, pain should not interact to any significant extent with HCN4 and/or HCN1 to avoid or minimise cardiac side-effects such as bradycardia.

WO02/100408 discloses a method for treating neuropathic pain using a compound that decreases the current mediated by an HCN pacemaker channel in a sensory cell. This document focuses on modulation of HCN1 and HCN3 and discloses ZD7288, ZM-227189, Zatebradine, DK-AH268, alinidine, and ivabradine as possible analgesic agents.

WO97/40027 discloses certain benzisoxazole and benzimidazole compounds which are stated to be useful in the treatment of various psychotic conditions.

WO99/18941 claims the use of Iₕ modulators for the treatment of psychiatric disorders.

WO2011/003895 discloses certain benzisoxazole compounds which are substituted by a carboxamide group at the 5, 6, or 7-position on the benzisoaxzole ring. The compounds are stated to be Iₕ channel blockers that may be useful in the treatment of neuropathic pain or inflammatory pain. This reference states that compounds disclosed the earlier filed WO97/40027 and WO99/18941 have a high CNS penetration resulting in undesirable side effects compared to the carboxamide substituted compounds claimed in WO2011/003895.

WO2011/000915 discloses certain zatebradine derivatives which are stated to selectively inhibit one or more HCN isoforms.

WO2011/019747 discloses certain propofol derivatives stated to be useful as HCN channel modulators for the treatment of chronic pain.

There remains a need for HCN channel inhibitors, particularly compounds which selectively inhibit HCN2 channels.

Tinnitus is the conscious perception of sound heard in the absence of physical sound sources external to the body. Tinnitus commonly manifests itself as ringing, buzzing, whistling or hissing sounds in the ear. Tinnitus is estimated to occur in 25.3% of American adults with 7.9% experiencing it frequently (Shargorodsky et al., Prevalence and characteristics of tinnitus among US adults. Am. J. Med. 2010 August; 123(8):711-8). Tinnitus can severely affect quality of life, by, for example, affecting sleep and the ability to concentrate and perform intellectual tasks. It can also lead to anxiety, depression and in extreme cases, suicide.

Tinnitus can be triggered by a number of factors including exposure to loud noise, presbyacusis, ear or head injuries, ear infections, tumours which impact on auditory nerves and certain diseases of the ear (e.g. Meniere's disease). Tinnitus is also a known side-effect of certain drugs, for example, salicylates (e.g. mesalamine or aspirin, particularly when taken in high doses), quinine anti-malarial agents, aminoglycoside antibiotics, certain chemotherapies, particularly platinum cytotoxic agents (e.g. cisplatin, carboplatin and oxaliplatin) and loop diuretics (e.g. furosemide, ethacrynic acid and torsemide). Tinnitus is also associated with auditory dysfunctions such as hyperacusis, distortion of sounds, misophonia, phonophobia and central auditory processing disorders.

There are no drug therapies currently approved by the FDA for the treatment of tinnitus and there is, therefore, an unmet medical need for an effective treatment of the condition.

The inventors have demonstrated for the first time that HCN2 inhibitors, including the novel compounds disclosed herein, are effective in the treatment of tinnitus using animal models for the condition. Tinnitus is generally considered to be a CNS phenomenon originating in the brain and resulting in referred noise in the ear (Henry et al. Underlying Mechanisms of Tinnitus: Review and Clinical Implications; J. Am. Acad. Audiol. 2014 January; 25(1): 5-126). It was therefore expected that a CNS-penetrant therapy would be required to treat tinnitus. Contrary to this expectation, the Examples herein show that the peripherally restricted HCN blocker, ivabradine and peripherally restricted HCN2 inhibitors of the present invention, provide an effective treatment for tinnitus in an in-vivo model for the condition. These results suggest that a peripherally restricted HCN2 inhibitor may provide an effective treatment of tinnitus and related conditions such as Meniere's disease with the additional benefit of a reduced risk of CNS related side-effects resulting from HCN2 inhibition in the brain.

### BRIEF SUMMARY OF THE DISCLOSURE

In accordance with the present inventions there is provided a compound of the formula (I), or a pharmaceutically acceptable salt thereof:

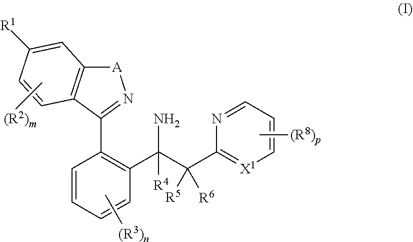

wherein
R¹ is selected from: H, halo, —CN, C₁₋₆ alkyl, C₁₋₆ haloalkyl, —OR^{B1}, C₂₋₆ alkenyl, C₂₋₆ alkynyl, C₃₋₆ cycloalkyl and C₃₋₆ cycloalkyl-C₁₋₆ alkyl-, and wherein any alkyl, alkenyl, alkynyl or cycloalkyl group in R¹ is optionally substituted with 1 to 4 substituents independently selected from halo, C₁₋₄ alkyl, C₁₋₄ haloalkyl and —OR^{B2};
R² is independently at each occurrence selected from: halo, C₁₋₆ alkyl and C₁₋₆ haloalkyl;
A is O or NR⁹;
R⁹ is selected from H, C₁₋₆ alkyl, C₃₋₆ cycloalkyl and C₃₋₆ cycloalkyl-C₁₋₆ alkyl-;
R³ is independently at each occurrence selected from: halo, —CN, C₁₋₆ alkyl, C₁₋₆ haloalkyl, —NR^{A3}R^{A3} and —OR^{B3};
R⁴, R⁵ and R⁶ are each independently selected from: H and C₁₋₄alkyl, or R⁵ and R⁶ together with the carbon atom to which they are attached form a C₃₋₆ cycloalkyl;
X¹ is N or CR⁷;
R⁷ is selected from: H, halo, —CN, C₁₋₆ alkyl, C₁₋₆ haloalkyl, C₃₋₆ cycloalkyl, —C(O)NR^{A4}R^{A4}, —N(R^{A4})C(O)R^{B4}, —C(O)R^{B4} and —S(O)XR^{B4};
R⁸ is independently at each occurrence selected from: halo, —CN, nitro, C₁₋₆ alkyl, C₁₋₆ haloalkyl, C₂₋₆alkenyl, C₂₋₆ alkynyl, —OR¹⁰, —NR¹⁰R¹¹, —S(O)ₓR¹⁰, —C(O)R¹⁰, —OC(O)R¹⁰, —C(O)OR^{10A}, —C(O)NR¹⁰R¹¹, —N(R¹¹)C(O)R¹⁰, —N(R¹¹)C(O)NR¹⁰R¹¹, —N(R¹¹)C(O)OR¹⁰, —N(R¹¹) SO₂R¹⁰, —SO₂NR¹⁰R¹¹, C₃₋₆ cycloalkyl, 3 to 7 membered heterocyclyl, phenyl and 5 or 6 membered heteroaryl; wherein said alkyl, alkenyl, alkynyl, cycloalkyl, or heterocyclyl group is optionally substituted with from 1 to 4 R¹² groups, and said phenyl or heteroaryl group is optionally substituted with from 1 to 4 R¹³ groups;
R¹⁰ is independently at each occurrence selected from: H, C₁₋₆ alkyl, C₁₋₆ haloalkyl and C₃₋₆ cycloalkyl; wherein said alkyl or cycloalkyl group is optionally substituted with from 1 to 4 R¹⁴ groups;
R^{10A} is selected from: C₁₋₆ alkyl, C₁₋₆ haloalkyl and C₃₋₆ cycloalkyl; wherein said alkyl or cycloalkyl group is optionally substituted with from 1 to 4 R¹⁴ groups;
R¹¹ is independently at each occurrence selected from: H and C₁₋₆ alkyl;
   or R¹⁰ and R¹¹ together with the nitrogen to which they are attached form a 4 to 7 membered heterocyclyl, wherein said heterocyclyl is optionally substituted with 1 or 2 substituents selected from halo, ═O, C₁₋₄ alkyl, C₁₋₄ haloalkyl and —OR^{B7}
R¹² and R¹⁴ are each independently at each occurrence selected from: halo, ═O, —CN, nitro, C₁₋₄ alkyl, C₁₋₄ haloalkyl, C₃₋₆ cycloalkyl, —OR^{B5}, —NR^{A5}R^{A5}, —S(O)ₓR^{B5}, —C(O)R^{B5}, —NR^{A5}C(O)R^{B5}, —C(O)NR^{A5}R^{A5}, —NR^{A5}SO₂R^{B5} and —SO₂NR^{A5}R^{A5};
R¹³ is independently at each occurrence selected from: halo, —CN, nitro, C₁₋₄ alkyl, C₁₋₄ haloalkyl, C₃₋₆ cycloalkyl, —OR^{B6}, —NR^{A6}R^{A6}, —S(O)ₓR^{B6}, —C(O)R^{A6}, —NR^{A6}C(O)R^{B6}, —C(O)NR^{A6}R^{A6}, —NR^{A6}SO₂R^{B6}, —SO₂NR^{A6}R^{A6};
R^{B1} and R^{B3} are independently at each occurrence selected from: H, C₁₋₆ alkyl and C₁₋₆ haloalkyl;
R^{B2}, R^{B4}, R^{B5}, R^{B6} and R^{B7} are independently at each occurrence selected from: H, C₁₋₄ alkyl and C₁₋₄ haloalkyl;
R^{A3}, R^{A4}, R^{A5} and R^{A6} are independently at each occurrence selected from H and C₁₋₄ alkyl;
m and p are each independently an integer selected from: 0, 1, 2 and 3;
n is an integer selected from: 0, 1, 2, 3 and 4; and
x is independently at each occurrence an integer selected from 0, 1, 2 and 3;
with the proviso that the compound is not a compound of the formula (A) or formula (B):

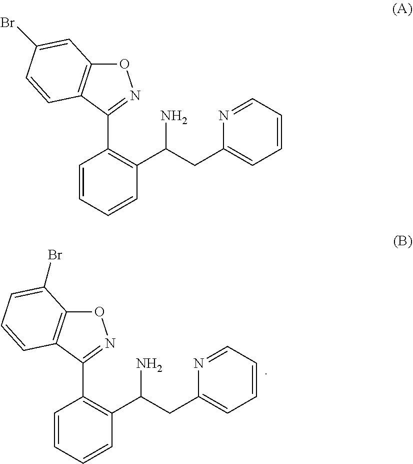 [SMILES: NC(Cc1ccccn1)c1ccccc1-c1noc2cc(Br)ccc12 ; NC(Cc1ccccn1)c1ccccc1-c1noc2c(Br)cccc12]

Also provided is a pharmaceutical composition comprising a compound of the invention, except the compounds of the formulae (A) and (B) are not excluded, and a pharmaceutically acceptable excipient.

Also provided is a compound of the invention, or a pharmaceutically acceptable salt thereof, or a pharmaceutical composition of the invention, for use as a medicament. In some embodiments there is provided a compound of the invention, is for use in the treatment of a disease or medical condition mediated by HCN2.

Also provided is a method of treating a disease or medical condition mediated by HCN2 in a subject, the method comprising administering to the subject an effective amount of a compound of the invention or a pharmaceutical composition of the invention.

In some embodiments a compound of the invention is for use in treatment of pain, including neuropathic pain and/or inflammatory pain. In some embodiments a compound of the invention is for use in the treatment of neuropathic pain, particularly chronic neuropathic pain. In some embodiments a compound of the invention is for use in the treatment of peripheral neuropathic pain, particularly chronic peripheral neuropathic pain. In some embodiments a compound of the invention is for use in the treatment of inflammatory pain, particularly chronic inflammatory pain.

A further aspect provides an HCN2 inhibitor for use in the treatment of tinnitus or a related condition. In some embodiments of this aspect, the HCN2 inhibitor is a peripherally restricted HCN2 inhibitor, for example ivabradine. In some embodiments the HCN2 inhibitor in this aspect is a compound of the invention, except the compounds of the formulae (A) and (B) are not excluded. Preferably the HCN2 inhibitor is a peripherally restricted compound of the invention. Accordingly, also provided is a compound of the invention for use in the treatment or prevention of tinnitus or a related condition (e.g. Meniere's disease or hyperacusis).

### BRIEF DESCRIPTION OF FIGURES

FIG. 1A illustrates the HCN1 and HCN2 voltage step protocol used in Example 126. FIG. 1B illustrates the HCN4 voltage step protocol used in Example 126.

FIG. 2 illustrates the HCN current amplitudes in accordance with Example 126.

FIG. 3 illustrates the voltage protocol used in the measurement of hERG signal in accordance with Example 127.

FIG. 4 illustrates the voltage protocol used in the measurement of hNay1.5 signal in accordance with Example 128.

FIG. 5 shows the effect on tinnitus by pharmacological block of HCN2 ion channels using the gap induced inhibition of the acoustic startle (GPIAS) test of Example 130.

FIG. 6 illustrates the effect of HCN ion channel block on behavioural signs of tinnitus in a short-term (salicylate) model in accordance with Example 130.

FIG. 7 illustrates the effect of HCN ion channel block on behavioural signs of tinnitus in a noise-exposure model in accordance with Example 130 using the compound of Example 5 compared with ivabradine.

FIG. 8 illustrates the effect of genetic deletion of HCN2 on auditory brainstem response (ABR) thresholds to tone pulses in accordance with Example 132. The open circle data points in FIG. 8 are from the auditory-targeted HCN2 deletion mice. The shaded data points are from the WT mice.

FIG. 9 illustrates the mechanical analgesic effect of the compound of Example 5 in a mouse neuropathic pain model tested using a von Frey filament. The compound of Example 5 showed full analgesia at an i.p. dose of 0.5 mg/kg and partial analgesia at an i.p. dose of 0.1 mg/kg. The effects are shown relative to vehicle (Veh”) and ivabradine (“IVA) dosed at 5 mg/kg. Significance over vehicle injection shown in the figure (*, p<0.05). The mechanical pain threshold on the y-axis is shown relative to baseline prior to partial sciatic nerve ligation (PSNL), which was carried out 5 days prior to testing the compounds in the model.

FIG. 10 shows the analgesic effects of the compound of Example 5 in a mouse model of neuropathic pain tested with paw pressure (Randall Sellito test). The compound of Example 5 (“HCN2 Blocker”) was tested at i.p. doses of 0.1 and 0.5 mg/kg and was compared with vehicle control (phosphate buffered saline (PBS) and ivabradine at an i.p. dose of 0.5 mg/kg. Measurements were taken at baseline and 7 and 10 days after partial sciatic nerve ligation (Op) and prior to administration of the test compounds. Pain threshold was then measured at 10 minutes, 90 minutes and 1 day after i.p. administration of each test compound.

FIG. 11 shows the thermal analgesia effects of the compound of Example 116 in a mouse neuropathic pain model. The compound of Example 116 was tested at an i.p. dose of 1.5 mg/kg (shaded circles) and is compared to ivabradine (open circles) at 5 mg/kg i.p. and vehicle (bold circles). The y-axis shows thermal withdrawal latency in seconds measured using infrared stimulus. The basal latency was measured prior to partial sciatic nerve ligation (PSNL), which was carried out 5 days prior to testing the compounds in the model.

FIG. 12 shows the effect of ivabradine dose on heart rate (left axis, solid circles) and inflammatory pain (right axis, open circles) in a formalin model in Black6 mice.

FIG. 13 shows the effect of the compound of Example 5 on bradycardia following i.p. injection in Black6 mice at doses from 0.1 mg/kg to 40 mg/kg compared to vehicle (“Veh”).

FIG. 14 compares the % change in pain (solid circles) and heart rate (open circles) from baseline following i.p. administration of various doses of the compound of Example 5.

FIG. 15 shows the analgesic effect of subthreshold i.p. doses of compound of Example 5 (solid circles) in a mouse neuropathic pain model tested using a von Frey filament compared to i.p. administered vehicle (open circles). The y-axis shows the mechanical pain threshold relative to baseline prior to partial sciatic nerve ligation (marked “surgery” in the figure).

FIG. 16 shows the analgesic effect of administering the compound of Example 5 (solid circles) twice per day at 8 hour intervals at a dose of 0.5 mg/kg i.p. in a mouse neuropathic pain model tested using a von Frey filament 4 hours after the first dose, compared to i.p. administered vehicle (“Veh”, open circles). The y-axis shows the mechanical pain threshold relative to baseline prior to partial sciatic nerve ligation (marked “surgery” in the figure).

### DETAILED DESCRIPTION

### Definitions

Unless otherwise stated, the following terms used in the specification and claims have the following meanings set out below.

As used herein “HCN2” designates the “hyperpolarization activated cyclic nucleotide gated potassium and sodium channel 2”. A reference sequence of full-length human HCN2 mRNA transcript is available from the GenBank database under accession number NM_001194, version NM_001194.3.

The terms “a compound of the invention”, “HCN2 inhibitor of the invention”, “HCN2 blocker of the invention” or the like refers to a compound of the Formulae (I), (II), (Ill), (IV), (V), (VI), (VII), (VIII) or (IX) or a pharmaceutically acceptable salt, solvate, or salt of a solvate thereof, including any of the Examples listed herein.

The terms “treating” or “treatment” refers to any indicia of success in the treatment or amelioration of a disease, pathology or condition, including any objective or subjective parameter such as abatement; remission; diminishing of symptoms or making the pathology or condition more tolerable to the patient; slowing in the rate of degeneration or decline; making the final point of degeneration less debilitating; improving a patient's physical or mental well-being. For example, certain methods herein treat pain, particularly inflammatory pain and/or neuropathic pain by decreasing a symptom of the pain. The term “treating” and conjugations thereof, include prevention of a pathology, condition, or disease (e.g. preventing the development of one or more symptoms of inflammatory pain or neuropathic pain.

The term “associated” or “associated with” in the context of a substance or substance activity or function associated with a disease of condition means that the disease or condition is caused by (in whole or in part), or a symptom of the disease or condition is caused by (in whole or in part) the substance or substance activity or function. For example, a symptom of a disease or condition associated with HCN2 channel activity may be a symptom that results (entirely or partially) from an increase in the level of activity of HCN2 channels or an increase in the expression of the channels. As used herein, what is described as being associated with a disease, if a causative agent, could be a target for treatment of the disease. For example, a disease associated with an increase in the level of activity of a HCN2 channel, may be treated with an agent (e.g. compound as described herein) effective for decreasing the level of activity of HCN2 channels.

As defined herein, the term “inhibition”, “inhibit”, “inhibiting”, “block” or “blocking” and the like in reference to an inhibitor of HCN2 means negatively affecting (e.g. decreasing) the level of activity or function of the HCN2 channel (e.g. a component of the HCN2 channel relative to the level of activity or function of channel in the absence of the inhibitor). In some embodiments inhibition refers to reduction of a disease or symptoms of disease (e.g. pain associated with an increased level of activity of HCN2). In some embodiments, inhibition refers to a reduction in the level of channel current. For example, a compound of the invention may bind to the HCN2 channel to block or prevent current flow through the channel or to produce an allosteric effect which acts to inhibit the action of the channel. Thus, inhibition may include, at least in part, partially or totally blocking stimulation, decreasing, preventing, or delaying activation, or inactivating, desensitizing, or down-regulating channel activity or the amount of a channel protein.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

The term “halo” or “halogen” refers to one of the halogens, group 17 of the periodic table. In particular, the term refers to fluorine, chlorine, bromine and iodine. Preferably, the term refers to fluorine, chlorine or bromine.

The term Cₘ₋ₙ refers to a group with m to n carbon atoms.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. “C₁₋₄ alkyl” similarly refers to such groups containing up to 4 carbon atoms. Alkylene groups are divalent alkyl groups and may likewise be linear or branched and have two points of attachment to the remainder of the molecule. Furthermore, an alkylene group may, for example, correspond to one of those alkyl groups listed in this paragraph. The alkyl and alkylene groups may be unsubstituted or substituted by one or more substituents. Possible substituents are described below. Substituents for the alkyl group may be halogen, e.g. fluorine, chlorine, bromine and iodine, OH, C₁-C₄ alkoxy. Other substituents for the alkyl group may alternatively be used.

The term “C₁₋₆ haloalkyl”, e.g. “C₁₋₄ haloalkyl”, refers to a hydrocarbon chain substituted with at least one halogen atom independently chosen at each occurrence, for example fluorine, chlorine, bromine and iodine. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoroethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be a fluoroalkyl group, i.e. a hydrocarbon chain substituted with at least one fluorine atom.

The term “C₂₋₆ alkenyl” includes a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl.

The term “C₂₋₆ alkynyl” includes a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl.

The term “C₃₋₆ cycloalkyl” includes a saturated hydrocarbon ring system containing 3, 4, 5 or 6 carbon atoms. For example, the “C₃-C₆ cycloalkyl” may be cyclopropyl, cyclobutyl, cyclopentyl, cyclohexyl, bicyclo[2.1.1]hexane or bicycle[1.1.1]pentane.

The term “heterocyclyl”, “heterocyclic” or “heterocycle” includes a 3- to 7-membered non-aromatic monocyclic or bicyclic saturated or partially saturated group comprising 1, 2 or 3 heteroatoms independently selected from O, S and N in the ring system (in other words 1, 2 or 3 of the atoms forming the ring system are selected from 0, S and N). By partially saturated it is meant that the ring may comprise one or two double bonds. This applies particularly to monocyclic rings with from 5 to 7 members. The double bond will typically be between two carbon atoms but may be between a carbon atom and a nitrogen atom. Bicyclic systems may be spiro-fused, i.e. where the rings are linked to each other through a single carbon atom; vicinally fused, i.e. where the rings are linked to each other through two adjacent carbon or nitrogen atoms; or they may be share a bridgehead, i.e. the rings are linked to each other through two non-adjacent carbon or nitrogen atoms. Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyridinyl, homopiperidinyl, homopiperazinyl, 2,5-diaza-bicyclo[2.2.1]heptanyl and the like. Typical sulfur containing heterocycles include tetrahydrothienyl, dihydro-1,3-dithiolane, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. For example, the terms “piperidinyl” or “morpholinyl” includes a piperidin-1-yl or morpholin-4-yl ring that is linked via the ring nitrogen (i.e. a piperidino or morpholino ring), the term also includes carbon linked rings (e.g. piperidin-4-yl or morpholin-3-yl).

The term “bridged ring systems” includes ring systems in which two rings share more than two atoms, see for example Advanced Organic Chemistry, by Jerry March, 4th Edition, Wiley Interscience, pages 131-133, 1992.

The term “spiro bi-cyclic ring systems” includes ring systems in which two ring systems share one common spiro carbon atom, i.e. the heterocyclic ring is linked to a further carbocyclic or heterocyclic ring through a single common spiro carbon atom.

“Heterocyclyl-Cₘ₋ₙ alkyl” includes a heterocyclyl group covalently attached to a Cₘ₋ₙ alkylene group, both of which are defined herein; and wherein the Heterocyclyl-Cₘ₋ₙ alkyl group is linked to the remainder of the molecule via a carbon atom in the alkylene group. The groups “aryl-Cₘ₋ₙ alkyl” “heteroaryl-Cₘ₋ₙ alkyl” are defined in the same way.

“—Cₘ₋ₙ alkyl substituted by —NRR” and “Cₘ₋ₙ alkyl substituted by —OR” similarly refer to an —NRR or —OR group covalently attached to a Cₘ₋ₙ alkylene group and wherein the group is linked to the remainder of the molecule via a carbon atom in the alkylene group.

Reference to “R¹⁰ and R¹¹ together with the nitrogen to which they are attached form a 4 to 7 membered heterocyclyl” refers to R¹⁰ and R¹¹ being attached to the same nitrogen atom and forming a nitrogen-linked heterocyclyl. By way of example, the group —NR¹⁰R¹¹ may form e.g. a pyrrolidn-1-yl, piperidin-1yl, piperazin-1yl or morpholin-4yl group.

The term “aromatic” when applied to a substituent as a whole includes a single ring or polycyclic ring system with 4n+2 electrons in a conjugated π system within the ring or ring system where all atoms contributing to the conjugated π system are in the same plane.

The term “aryl” includes an aromatic hydrocarbon ring system. The ring system has 4n+2 electrons in a conjugated π system within a ring where all atoms contributing to the conjugated π system are in the same plane. For example, the “aryl” may be phenyl and naphthyl. The aryl system itself may be substituted with other groups.

The term “heteroaryl” includes an aromatic mono- or bicyclic ring incorporating one or more (for example 1-4, particularly 1, 2 or 3) heteroatoms selected from nitrogen, oxygen or sulfur. The ring or ring system has 4n+2 electrons in a conjugated π system where all atoms contributing to the conjugated π system are in the same plane.

The heteroaryl group can be, for example, a 5- or 6-membered monocyclic ring. The ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

The term “optionally substituted” includes either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”.

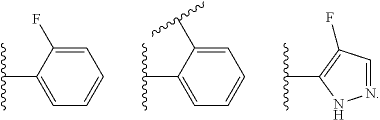

“Meta” substitution is a substitution pattern where two substituents are on carbons one carbon removed from each other, i.e. with a single carbon atom between the substituted carbons. In other words, there is a substituent on the second atom away from the atom with another substituent. For example, the groups below are meta substituted.

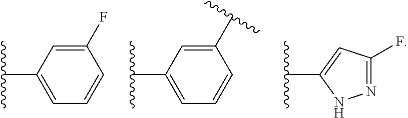

“Para” substitution is a substitution pattern where two substituents are on carbons two carbons removed from each other, i.e. with two carbon atoms between the substituted carbons. In other words, there is a substituent on the third atom away from the atom with another substituent. For example, the groups below are para substituted.

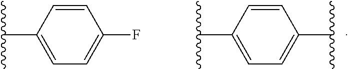 [SMILES: *c1ccc(F)cc1 ; *c1ccc(*)cc1]

A bond terminating in a “ [symbol] ” or “*” represents that the bond is connected to another atom that is not shown in the structure. A bond terminating inside a cyclic structure and not terminating at an atom of the ring structure represents that the bond may be connected to any of the atoms in the ring structure where allowed by valency.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or less than 550. More preferably, the molecular weight is less than 525 and, for example, is 500 or less.

Suitable or preferred features of any compounds of the present invention may also be suitable features of any other aspect.

The invention contemplates pharmaceutically acceptable salts of the compounds of the invention. These may include the acid addition and base salts of the compounds. These may be acid addition and base salts of the compounds.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of the invention may be prepared by for example, one or more of the following methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereo centres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%

The compounds of this invention may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess HCN2 inhibitory activity.

Z/E (e.g. cis/trans) isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC) or chiral supercritical fluid chromatography (SFC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Alternatively, when chiral SFC is employed a supercritical fluid, generally CO₂, is used as the mobile phase. The properties of the supercritical fluid may be modified by the inclusion of one or more co-solvents, e.g. an alcohol such as methanol, ethanol or isopropanol, acetonitrile or ethylacetate. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person. An enantiomer of a compound may also be prepared using a chiral auxiliary during the synthesis of the compound, in which a suitable chiral intermediate is reacted with an intermediate of the compound followed by one or more diastereoselective transformations. The resulting diastereomers are then separated using conventional methods, such as those described above, followed by removal of the chiral auxiliary to provide the desired enantiomer.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium), ¹¹C ¹³C, ¹⁴C, ¹⁵O, ¹⁷O, ¹⁸O ¹³N, ¹⁵N, ¹⁸F, ³⁶Cl, ¹²³I, ²⁵I, ³²P ³⁵S and the like. The radionuclide that is used will depend on the specific application of that radio-labelled derivative. For example, for in vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

Isotopically-labelled compounds can generally be prepared by conventional techniques known to those skilled in the art or by processes analogous to those described using an appropriate isotopically-labelled reagent in place of the non-labelled reagent previously employed.

The selective replacement of hydrogen with deuterium in a compound may modulate the metabolism of the compound, the PK/PD properties of the compound and/or the toxicity of the compound. For example, deuteration may increase the half-life or reduce the clearance of the compound in-vivo. Deuteration may also inhibit the formation of toxic metabolites, thereby improving safety and tolerability. It is to be understood that the invention encompasses deuterated derivatives of compounds of formula (I). As used herein, the term deuterated derivative refers to compounds of the invention where in a particular position at least one hydrogen atom is replaced by deuterium. For example, one or more hydrogen atoms in a C₁₋₄-alkyl group may be replaced by deuterium to form a deuterated C₁₋₄-alkyl group, for example CD₃.

Certain compounds of the invention may exist in solvated as well as unsolvated forms such as, for example, hydrated forms. It is to be understood that the invention encompasses all such solvated forms or pharmaceutically acceptable salts thereof that possess HCN2 inhibitory activity.

It is also to be understood that certain compounds of the invention may exhibit polymorphism, and that the invention encompasses all such forms that possess HCN2 inhibitory activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

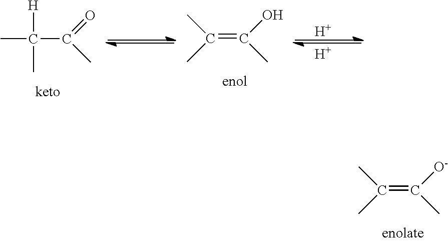

The in vivo effects of a compound of the invention may be exerted in part by one or more metabolites that are formed within the human or animal body after administration of a compound of the invention.

It is further to be understood that a suitable pharmaceutically-acceptable pro-drug of a compound of the formula (I) also forms an aspect of the present invention. Accordingly the compounds of the invention encompass pro-drug forms of the compounds and the compounds of the invention may be administered in the form of a pro-drug, that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the invention and in-vivo cleavable amide derivatives that may be formed at a carboxy group or an amino group in a compound of the invention.

Accordingly, the present invention includes those compounds of the invention as defined herein when made available by organic synthesis and when made available within the human or animal body by way of cleavage of a pro-drug thereof. Accordingly, the present invention includes those compounds of the formula (I) that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically-acceptable pro-drug of a compound of the invention is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

### Compounds

In some embodiments the compound of the formula (I) is a compound of the formula (II), or a pharmaceutically acceptable salt thereof:

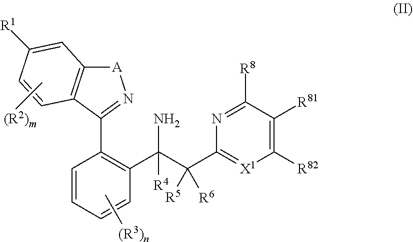

wherein R⁸¹ and R⁸² are independently selected from: H, halo, —CN, C₁₋₄alkyl, C₁₋₄ haloalkyl, —OH and —OC₁₋₄ alkyl.

In some embodiments the compound of the formula (I) is a compound of the formula (III), or a pharmaceutically acceptable salt thereof:

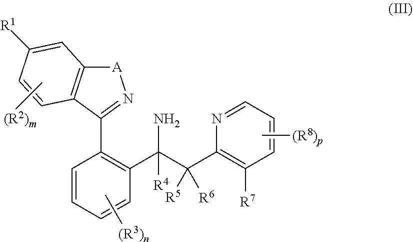

wherein R⁷ is not H.

In some embodiments the compound of the formula (I) is a compound of the formula (IV), or a pharmaceutically acceptable salt thereof:

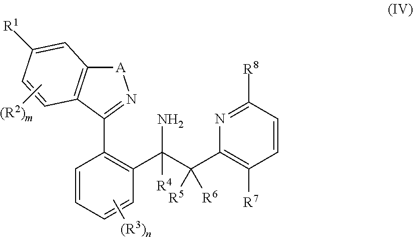

wherein R⁷ is not H.

In some embodiments the compound of the formula (I) is a compound of the formula (V), or a pharmaceutically acceptable salt thereof:

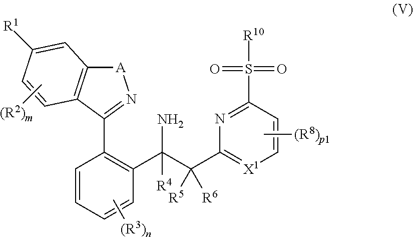

Wherein R¹⁰ is not H; and
   p1 is an integer selected from 0, 1 and 2.

In some embodiments the compound of the formula (I) is a compound of the formula (VI), or a pharmaceutically acceptable salt thereof:

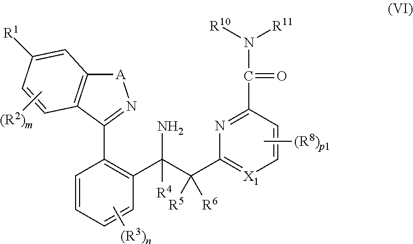

wherein p1 is an integer selected from 0, 1 and 2.

In some embodiments the compound of the formula (I) is a compound of the formula (VII), or a pharmaceutically acceptable salt thereof:

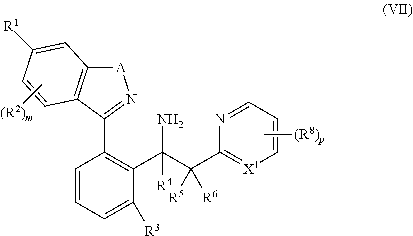

In some embodiments the compound of the formula (I) is a compound of the formula (VIII), or a pharmaceutically acceptable salt thereof:

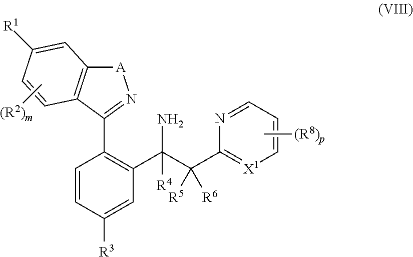

In some embodiments the compound of the formula (I) is a compound of the formula (IX), or a pharmaceutically acceptable salt thereof:

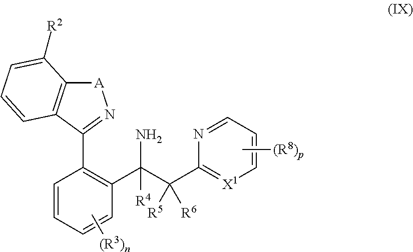

Particular compounds of the invention include, for example, compounds of formulae (I), (II), (Ill), (IV), (V), (VI), (VII), (VIII) or (IX), or a pharmaceutically acceptable salt thereof, wherein, unless otherwise stated, each of R¹, R², R³, R⁴, R⁵, R⁶, R⁸, A, X¹, n, m and p has any of the meanings defined hereinbefore or in any one or more of paragraphs (1) to (132) hereinafter:
1. R¹ is selected from: H, halo, —CN, C₁₋₄ alkyl, C₁₋₄ haloalkyl, C₂₋₄ alkenyl, C₂₋₄ alkynyl, C₃₋₆ cycloalkyl and C₃₋₆ cycloalkyl-C₁₋₆ alkyl-, and wherein said alkyl, alkenyl, alkynyl or a cycloalkyl group in R¹ is optionally substituted with 1 to 4 substituents independently selected from halo, C₁₋₄ alkyl, C₁₋₄ haloalkyl and —OR^{B2}.
2. R¹ is selected from: H, halo, —CN, C₁₋₄ alkyl, C₁₋₄ haloalkyl, C₂₋₄ alkenyl, C₂₋₄ alkynyl, C₃₋₅ cycloalkyl, and wherein said alkyl, alkenyl, alkynyl or cycloalkyl group is optionally substituted with 1 to 4 substituents independently selected from halo, C₁₋₄ alkyl, C₁₋₄ haloalkyl and —OR^{B2}.
3. R¹ is selected from: H, halo, —CN, C₁₋₄ alkyl, C₁₋₄ haloalkyl, C₂₋₄ alkenyl and C₂₋₄ alkynyl and wherein said alkyl, alkenyl or alkynyl group is optionally substituted with 1 or 2 substituents independently selected from halo and —OR^{B2}.
4. R¹ is selected from: H, —CN, C₁₋₄ alkyl, C₂₋₄ alkenyl, C₂₋₄ alkynyl, C₃₋₅ cycloalkyl, and wherein said alkyl, alkenyl, alkynyl or cycloalkyl group is optionally substituted with 1 to 4 substituents independently selected from C₁₋₄ alkyl and —OR^{B2}.
5. R¹ is selected from halo, —CN, C₁₋₄ alkyl, C₁₋₄ haloalkyl, C₂₋₄ alkenyl, C₂₋₄ alkynyl and C₃₋₆ cycloalkyl, and wherein said alkyl, alkenyl, alkynyl or cycloalkyl group in R¹ is optionally substituted with 1 or 2 substituents independently selected from halo, C₁₋₄ alkyl, C₁₋₄ haloalkyl and —OR^{B2}.
6. R¹ is selected from —CN, C₂₋₆alkenyl, C₂₋₆ alkynyl, C₃₋₆ cycloalkyl, C₃₋₆ cycloalkyl-C₁₋₆ alkyl and —C₁₋₆ alkyl-OR^{B2}, wherein said alkenyl and alkynyl group is substituted with 1 or 2 substituents selected from halo, C₁₋₄ haloalkyl and —OR^{B2}; and said cycloalkyl or cycloalkyl-alkyl group is optionally substituted with 1 to 4 substituents independently selected from halo, C₁₋₄ alkyl, C₁₋₄ haloalkyl and —OR^{B2}.
7. R¹ is selected from —CN, C₃₋₆ cycloalkyl, —C₁₋₄ alkyl-OR^{B2}, C₂₋₄ alkenyl, C₂₋₄ alkynyl, wherein said C₂₋₄ alkenyl or C₂₋₄ alkynyl is optionally substituted with a substituent selected from halo and —OR^{B2}.
8. R¹ is selected from H, —CN, C₁₋₃ alkyl, —C₁₋₃ alkyl-OR^{B2}, C₂₋₃ alkynyl, —C≡C—OCH₂—OR^{B2} and C₃₋₅ cycloalkyl.
9. R¹ is selected from H —CN and C₁₋₃ alkyl.
10. R¹ is selected from H, F, Cl, Br, —CN, methyl, ethyl, propyl, isopropyl, cyclopropyl, hydroxymethyl, 2-hydroxyethyl, 1-hydroxyethyl, methoxymethyl, 1-methoxyethyl, 2-methoxyethyl, ethynyl, propynyl and 3-hydroxypropynyl.
11. R¹ is selected from H, —CN, halo (e.g. F, C or Br, particularly Br) and methyl.
12. R¹ is selected from H, —CN and methyl.
13. R¹ is selected from —CN and C₁₋₃ alkyl (e.g. methyl).
14. R¹ is —CN.
15. R¹ is halo (e.g. F, C or Br).
16. R¹ is C₁₋₃ alkyl (e.g. methyl)
17. R¹ is H.
18. R² is independently at each occurrence selected from halo, C₁₋₄ alkyl and C₁₋₄ haloalkyl.
19. R² is selected from halo and C₁₋₃ alkyl.
20. R² is selected from F, C, Br and methyl.
21. R² is C₁₋₃ alkyl (e.g. methyl).
22. R² is selected from F, Cl and Br.
23. R² is selected from F and Cl.
24. m is 0.
25. m is 1.
26. R¹ is H, m is 1 and R² is as defined in any of (18) to (23).
27. R¹ is H and m is 0.
28. R³ is independently at each occurrence selected from: halo, —CN, C₁₋₄ alkyl, C₁₋₄ haloalkyl, —OC₁₋₄alkyl and —NR^{A3}R^{A3}.
29. R³ is independently at each occurrence selected from: halo, —CN and C₁₋₃alkyl.
30. R³ is independently at each occurrence selected from: F, Cl, Br, —CN, methyl, ethyl, isopropyl, propyl and methoxy.
31. R³ is independently at each occurrence selected from: F, Cl, Br, —CN and methyl.
32. R³ is F or Br.
33. n is 0, 1 or 2;
34. n is 1.
35. n is 0.
36. n is 1 and R³ is in the ortho or meta position relative to the group —C(R⁴)(NH₂)— in formula (I).
37. n is 1, R³ is in the ortho or meta position relative to the group —C(R⁴)(NH₂)— in formula (I) and R³ is as defined in any of (28) to (32) above.
38. R⁴, R⁵ and R⁶ are each independently selected from: H and C₁₋₃ alkyl, or R⁵ and R⁶ together with the carbon atom to which they are attached form cyclopropyl.
39. R⁴ is H or methyl.
40. R⁴ is H.
41. R⁵ and R⁶ are each independently selected from: H and C₁₋₃ alkyl.
42. R⁵ is H and R⁶ is selected from: H and C₁₋₃ alkyl.
43. R⁵ is H and R⁶ is C₁₋₃ alkyl (e.g. methyl).
44. R⁵ and R⁶ are H.
45. R⁴ and R⁵ are H and R⁶ is C₁₋₃ alkyl (e.g. methyl or ethyl).
46. R⁴, R⁵ and R⁶ are H.
47. One of R⁴, R⁵ and R⁶ is C₁₋₃ alkyl (e.g. methyl) and other two groups are H.
48. A is O.
49. A is NR⁹.
50. R⁹ is selected from: H, C₁₋₆ alkyl and C₃₋₆ cycloalkyl.
51. R⁹ is selected from: H, C₁ alkyl, cyclopropyl, cyclobutyl and cyclopentyl.
52. R⁹ is selected from: H and C₁₋₄ alkyl.
53. R⁹ is H.
54. R⁹ is C₁₋₄ alkyl.
55. R⁹ is methyl, ethyl, propyl or isopropyl.
56. X¹ is N.
57. X¹ is CR⁷.
58. R⁷ is selected from H, halo, —CN, C₁₋₄alkyl, C₁₋₄haloalkyl, C₃₋₅ cycloalkyl, —N(R^{A4})C(O)R^{B4} and —C(O)R^{B4}.
59. R⁷ is selected from: —CN, —N(R^{A4})C(O)R^{B4} and —C(O)R^{B4}.
60. R⁷ is selected from H, halo, —CN, C₁₋₄alkyl, C₁₋₄haloalkyl, C₃₋₅ cycloalkyl, and —C(O)R^{B4}.
61. R⁷ is selected from H, halo, —CN, C₁₋₄ alkyl and C₁₋₄ haloalkyl.
62. R⁷ is selected from halo, C₁₋₄ alkyl and C₁₋₄ haloalkyl.
63. R⁷ is selected from halo and C₁₋₄ alkyl.
64. R⁷ is selected from H, F, Cl, Br, —CN, methyl, ethyl and —CF₃.
65. R⁷ is selected from F and methyl.
66. R⁷ is not H.
67. R⁷ is H.
68. X¹ is CR⁷ and R⁷ is as defined in any of (58) to (67).
69. R³ is independently at each occurrence selected from: halo, —CN, C₁₋₆ alkyl, C₁₋₆ haloalkyl, C₂₋₆ alkenyl, C₂₋₆ alkynyl, —OR¹⁰, —NR¹⁰R¹¹, —S(O)ₓR¹⁰ (wherein x is 0, 1 or 2, preferably 1 or 2), —C(O)R¹⁰, —C(O)NR¹⁰R¹¹, —N(R¹¹)C(O)R¹⁰, —N(R¹¹)C(O)NR¹⁰R¹¹, —N(R¹¹)C(O)OR¹⁰, —N(R¹¹) SO₂R¹⁰, —SO₂NR¹⁰R¹¹, C₃₋₆ cycloalkyl, 4 to 6 membered heterocyclyl, phenyl and 5 or 6 membered heteroaryl containing at least one ring nitrogen atom;
   wherein said alkyl, alkenyl, alkynyl, cycloalkyl, or heterocyclyl group is optionally substituted with from 1 to 4 R¹² groups, and said phenyl or heteroaryl group is optionally substituted with from 1 to 4 R¹³ groups.
70. R³ is independently at each occurrence selected from: halo, —CN, C₁₋₆ alkyl, C₁₋₆ haloalkyl, —OR¹⁰, —NR¹⁰R¹¹, —S(O)ₓR¹⁰ (wherein x is 0, 1 or 2, preferably 1 or 2), —C(O)NR¹⁰R¹¹, —N(R¹¹)C(O)R¹⁰, —N(R¹¹)C(O)NR¹⁰R¹¹, —N(R¹¹)C(O)OR¹⁰, —N(R¹¹) SO₂R¹⁰, C₃₋₆ cycloalkyl, 4 to 6 membered heterocyclyl and 5 or 6 membered heteroaryl containing 1 or 2 ring nitrogen atoms;
   wherein said alkyl, cycloalkyl, or heterocyclyl group is optionally substituted with from 1 to 4 R¹² groups, and said heteroaryl group is optionally substituted with from 1 to 4 R¹³ groups.
71. R³ is independently at each occurrence selected from: halo, —CN, C₁₋₆ alkyl, C₁₋₆ haloalkyl, —OR¹⁰, —NR¹⁰R¹¹, —S(O)ₓR¹⁰ (wherein x is 0, 1 or 2, preferably 1 or 2), —C(O)NR¹⁰R¹¹, —N(R¹¹)C(O)R¹⁰, —N(R¹¹)C(O)NR¹⁰R¹¹, —N(R¹¹)C(O)OR¹⁰, —N(R¹¹) SO₂R¹⁰, C₃₋₅ cycloalkyl, 4 to 6 membered heterocyclyl containing 1 ring nitrogen atom and optionally 1 additional ring heteroatom selected from O, S and N, and heteroaryl, wherein said heteroaryl is selected from pyrrolyl, pyrazolyl, imidazolyl, oxazolyl, isoxazolyl, thiazolyl and isothiazolyl;
   wherein said alkyl, cycloalkyl, or heterocyclyl group is optionally substituted with from 1 to 4 R¹² groups, and said heteroaryl group is optionally substituted with from 1 to 4 R¹³ groups.
72. R³ is independently at each occurrence selected from: halo, —CN, C₁₋₆ alkyl, C₁₋₆ haloalkyl, —OR¹⁰, —NR¹⁰R¹¹, —S(O)ₓR¹⁰ (wherein x is 0, 1 or 2, preferably 1 or 2), —C(O)NR¹⁰R¹¹, —N(R¹¹)C(O)R¹⁰, —N(R¹¹)C(O)NR¹⁰R¹¹, —N(R¹¹)C(O)OR¹⁰, —N(R¹¹) SO₂R¹⁰, C₃₋₅ cycloalkyl, 4 to 6 membered heterocyclyl and heteroaryl, wherein said heteroaryl is selected from pyrrolyl, pyrazolyl, imidazolyl, oxazolyl and isoxazolyl, and wherein said 4 to 6 membered heterocyclyl is selected from, azetidinyl, pyrrolidinyl, piperidinyl, piperazinyl, morpholinyl and thiomorpholinyl;
   wherein said alkyl, cycloalkyl, or heterocyclyl group is optionally substituted with from 1 to 4 R¹² groups, and said heteroaryl group is optionally substituted with from 1 to 4 R¹³ groups.
73. R³ is independently at each occurrence selected from: halo, —CN, C₁₋₆ alkyl, C₁₋₆ haloalkyl, —OR¹⁰, —NR¹⁰R¹¹, —S(O)ₓR¹⁰ (wherein x is 0, 1 or 2, preferably 1 or 2), —C(O)NR¹⁰R¹¹, —N(R¹¹)C(O)R¹⁰, —N(R¹¹)C(O)NR¹⁰R¹¹, —N(R¹¹)C(O)OR¹⁰, —SO₂NR¹⁰R¹¹ and —N(R¹¹) SO₂R¹⁰;
   wherein said alkyl group is optionally substituted with from 1 or 2 substituents selected from halo, —CN, —OR^{B5}, —NR^{A5}R^{A5}, —S(O)₂R^{B5}, —C(O)R^{B5}, —NR^{A5}C(O)R^{B5}, —C(O)NR^{A5}R^{A5}, —NR^{A5}SO₂R^{B5} and —SO₂NR^{A5}R^{A5}
74. R³ is independently at each occurrence selected from, 4 to 6 membered heterocyclyl and heteroaryl, wherein said heteroaryl is selected from pyrrolyl, pyrazolyl, imidazolyl, oxazolyl and isoxazolyl; and wherein said heterocyclyl is selected from, azetidinyl, pyrrolidinyl, piperidinyl, piperazinyl, morpholinyl and thiomorpholinyl;
   wherein said 4 to 6 membered heterocyclyl group is optionally substituted with from 1 to 4 groups independently selected from halo, C₁₋₄ alkyl, —OH and ═O; and
   wherein said heteroaryl group is optionally substituted with from 1 to 4 groups independently selected from halo, —CN, C₁₋₄ alkyl, C₁₋₄ haloalkyl, —OR^{B6} and —NR^{A6}R^{A6}.
75. R³ is independently at each occurrence selected from: —CN, —OR¹⁰, —NR¹⁰R¹¹, —S(O)₂R¹⁰, —S(O)R¹⁰, —C(O)NR¹⁰R¹¹, —N(R¹¹)C(O)R¹⁰, —N(R¹¹)C(O)NR¹⁰R¹¹, —N(R¹¹)C(O)OR¹⁰, —SO₂NR¹⁰R¹¹, —N(R¹¹) SO₂R¹⁰, —C₁₋₄ alkyl-CN, —C₁₋₄ alkyl-OR^{B5}, —C₁₋₄ alkyl-NR^{A5}R^{A5}, —C₁₋₄ alkyl-S(O)₂R^{B5}, —C₁₋₄ alkyl-NR^{A5}C(O)R^{B5}, —C₁₋₄ alkyl-C(O)NR^{A5}R^{A5}, —C₁₋₄ alkyl-NR^{A5}SO₂R^{B5}, —C₁₋₄ alkyl-SO₂NR^{A5}R^{A5}.
76. R³ is independently at each occurrence selected from: —CN, —OH, —OC₁₋₄ alkyl, —OC₂₋₄ alkyl-OR^{B5}, —OC₂₋₄ alkyl-NR^{A5}R^{A5}, —NH₂, —N(R¹¹)C₁₋₄ alkyl, —N(R¹¹)C₂₋₄ alkyl-OR^{B5}, —N(R¹¹)C₂₋₄ alkyl-NR^{A5}R^{A5}, —S(O)₂C₁₋₄alkyl, —C(O)NH₂, —C(O)N(R¹¹)C₁₋₄ alkyl, —C(O)N(R¹)C₁₋₄ alkyl-C₃₋₆cycloalkyl, —C(O)N(R¹¹)C₂₋₄ alkyl-OR^{B5}, —C(O)N(R¹¹)C₂₋₄ alkyl-NR^{A5}R^{A5}, azetidin-1-yl-C(O)—, pyrrolidin-1-yl-C(O)—, piperidin-1-yl-C(O)—, piperarazin-1-yl-C(O)—, morpholin-1-yl-C(O)—, —N(R¹¹)C(O)—C₁₋₄alkyl, —N(R¹¹)C(O)NH₂, —N(R¹¹)C(O)NH(C₁₋₄alkyl), —N(R¹¹)C(O)N(C₁₋₄ alkyl)₂, —N(R¹¹)C(O)O(C₁₋₄alkyl), —SO₂NH₂, —SO₂N(R¹)C₁₋₄ alkyl, —N(R¹¹)SO₂(C₁₋₄ alkyl), —C₁₋₄ alkyl-CN, —C₁₋₄ alkyl-OR^{B5}, —C₁₋₄ alkyl-NR^{A5}R^{A5}, —C₁₋₄ alkyl-S(O)₂R^{B5}, —C₁₋₄alkyl-NR^{A5}C(O)R^{B5}, —C₁₋₄alkyl-C(O)NR^{A5}R^{A5}, —C₁₋₄alkyl-NR^{A5}SO₂R^{B5} and —C₁₋₄ alkyl-SO₂NR^{A5}R^{A5}.
77. R³ is independently at each occurrence selected from: —CN, —C₁₋₄ alkyl-OH, —C₁₋₄ alkyl-OMe, —C₁₋₄ alkyl-NH₂, —C₁₋₄ alkyl-NH(C₁₋₄alkyl), —C₁₋₄ alkyl-N(C₁₋₄alkyl)₂, —NH₂, —NH(C₁₋₄ alkyl), —N(C₁₋₄ alkyl)₂, —OH, —OC₁₋₄ alkyl, —OC₂₋₄ alkyl-OH, —OC₂₋₄ alkyl-OMe, —S(O)₂C₁₋₄ alkyl, —C(O)NH₂, —C(O)N(H)C₁₋₄alkyl and —C(O)N(C₁₋₄ alkyl)₂.
78. R³ is independently at each occurrence selected from: —C(O)NH₂, —C(O)N(R¹)C₁₋₄ alkyl, —C(O)N(R¹)C₁₋₄ alkyl-C₃₋₆cycloalkyl, —C(O)N(R¹¹)C₂₋₄ alkyl-OR^{B5}, —C(O)N(R¹¹)C₂₋₄ alkyl-NR^{A5}R^{A5}, azetidin-1-yl-C(O)—, pyrrolidin-1-yl-C(O)—, piperidin-1-yl-C(O)—, piperarazin-1-yl-C(O)— and morpholin-1-yl-C(O)—.
79. R³ is independently at each occurrence selected from: —C(O)NH₂, —C(O)N(H)C₁₋₃ alkyl and —C(O)N(C₁₋₃ alkyl)₂.
80. R³ is independently at each occurrence selected from: —S(O)₂R¹⁰, for example —S(O)₂C₁₋₄ alkyl or —S(O)₂C₃₋₆ cycloalkyl.
81. R³ is independently at each occurrence selected from —S(O)₂C₁₋₄ alkyl, preferably —S(O)₂Me.
82. R³ is independently at each occurrence selected from: —C₁₋₄alkyl-OR^{B5} and —O—C₁₋₄alkyl-OR^{B5}, for example —C₁₋₄alkyl-OH, —C₁₋₄alkyl-OMe, —OC₁₋₄alkyl-OH or —OC₁₋₄alkyl-OMe.
83. R³ is independently at each occurrence selected from: halo, —CN, C₁₋₄ alkyl, C₁₋₄ haloalkyl, —C₁₋₄alkyl-OR^{B5}, —C₁₄alkyl-C₃₋₆ cycloalkyl, —C₁₋₄alkyl-NR^{A5}C(O)R^{B5}, —C₁₄alkyl-C(O)NR^{A5}R^{A5}, —C₁₋₄ alkyl-NR^{A5}SO₂R^{B5}, —C₁ alkyl-SO₂NR^{A5}R^{A5}, —OH, —OC₁₋₄alkyl, —OC₂₋₄ alkyl-OR^{B5}, —OC₂₋₄alkyl-NR^{A5}R^{A5}, —NH₂, —N(R¹¹)C₁₋₄ alkyl, —N(R¹¹)C₂₋₄ alkyl-OR^{B5}, —N(R¹¹)C₂₋₄ alkyl-NR^{A5}R^{A5}, —S(O)₂C₁₋₄alkyl, —C(O)NH₂, —C(O)N(R¹)C₁₋₄ alkyl, —C(O)N(R¹)C₁₋₄ alkyl-C₃₋₆ cycloalkyl, —C(O)N(R¹¹)C₂₋₄ alkyl-OR^{B5}, —C(O)N(R¹¹)C₂₋₄ alkyl-NR^{A5}R^{A5}, azetidin-1-yl-C(O)—, pyrrolidin-1-yl-C(O)—, piperidin-1-yl-C(O)—, piperarazin-1-yl-C(O)—, morpholin-1-yl-C(O)—, —N(R¹¹)C(O)—C₁₋₄alkyl, —N(R¹¹)C(O)NH₂, —N(R¹¹)C(O)NH(C₁₋₄ alkyl), —N(R¹¹)C(O)N(C₁₋₄ alkyl)₂, —N(R¹¹)C(O)O(C₁₋₄alkyl), —N(R¹¹)SO₂(C₁₋₄ alkyl), C₃₋₆ cycloalkyl, 4 to 6 membered heterocyclyl and heteroaryl, wherein said heteroaryl is selected from pyrrolyl, pyrazolyl, imidazolyl, oxazolyl and isoxazolyl, and wherein said heterocyclyl is selected from, azetidinyl, pyrrolidinyl, piperidinyl, piperazinyl, morpholinyl and thiomorpholinyl;
   wherein said C₃₋₆ cycloalkyl, or 4 to 6 membered heterocyclyl group is optionally substituted with from 1 to 4 groups independently selected from halo, C₁₋₄ alkyl, —OH and ═O; and
   wherein said heteroaryl group is optionally substituted with from 1 to 4 groups independently selected from halo, —CN, C₁₋₄ alkyl, C₁₋₄ haloalkyl, —OR^{B6} and —NR^{A6}R^{A6}.
84. R³ is independently at each occurrence selected from: halo, —CN, C₁₋₄ alkyl, —C₁₋₄ alkyl-OH, —C₁₋₄ alkyl-OMe, —C₁₋₄ alkyl-C(O)NH₂, —C₁₋₄ alkyl-C(O)N(H)Me, —C₁₋₄ alkyl-C(O)N(Me)₂, —CF₃, methoxy, ethoxy, 2-hydroxyethoxy, 2-methoxyethoxy, —NH₂, —NH(C₁₋₄ alkyl), —N(C₁₋₄alkyl)₂, —NH((CH₂)₂—OH), —NH((CH₂)₃—OH), —NH((CH₂)₂-OMe), —NH((CH₂)₃-OMe), —SO₂C₁₋₄ alkyl, —C(O)NH₂, —C(O)N(H)C₁₋₄alkyl, —C(O)N(C₁₋₄ alkyl)₂, —C(O)NH((CH₂)₂—OH), —C(O)NH((CH₂)₃—OH), —C(O)NH((CH₂)₂-OMe), —C(O)NH((CH₂)₃—OMe), —C(O)NH(CH₂-cyclopropyl), azetidin-1-yl-C(O)—, pyrrolidin-1-yl-C(O)—, piperidin-1-yl-C(O)—, piperarazin-1-yl-C(O)—, morpholin-1-yl-C(O)—, —N(H)C(O)C₁₋₄alkyl, —NHC(O)NH₂, —NHC(O)NH(C₁₋₄alkyl), —NHC(O)N(C₁₋₄ alkyl)₂, —N(H)C(O)O(C₁₋₄alkyl) and —NHSO₂(C₁₋₄alkyl).
85. R³ is independently at each occurrence selected from: halo (e.g. F, Cl or Br), —CN, methyl, ethyl, methoxy, 2-hydroxyethyl, —CF₃, —NH₂, —N(H)Me, —N(Me)₂, —S(O)₂Me, —C(O)NH₂, —C(O)N(H)Me, —C(O)N(Me)₂, —NHC(O)OMe, —CH₂C(O)NH₂, —CH₂C(O)N(H)Me, —(CH₂)₂C(O)N(Me)₂, —(CH₂)₂C(O)NH₂, —(CH₂)₂C(O)N(H)Me, and —(CH₂)₂C(O)N(Me)₂.
86. R³ is independently at each occurrence selected from: halo (e.g. F or Br), —CN, methyl, ethyl, methoxy, —S(O)₂Me and —CF₃.
87. R³ is independently at each occurrence selected from: halo (e.g. F or Br), —CN, methyl, ethyl and —S(O)₂Me.
88. R³ is independently at each occurrence selected from: —CN, methyl, ethyl and —S(O)₂Me.
89. R³ is independently at each occurrence selected from: halo (e.g. F or Br), —CN, methyl, ethyl, 2-hydroxyethyl, methoxy, 2-hydroxyethoxy, —CF₃, —NH₂, —S(O)₂Me, —C(O)NH₂, —C(O)N(H)Me, —C(O)N(Me)₂, —(CH₂)₂C(O)NH₂, —(CH₂)₂C(O)N(H)Me, —(CH₂)₂C(O)N(Me)₂, —NHC(O)OMe, and pyrazolyl.
90. R⁸ is independently at each occurrence selected from: halo (e.g. F or Br), —CN, methyl, ethyl, methoxy, —CF₃, —NH₂ and —S(O)₂Me.
91. R⁸ is independently at each occurrence selected from: —CN, methyl, methoxy, —CF₃, —NH₂ and —S(O)₂Me.
92. R⁸ is independently at each occurrence selected from: halo (e.g. F or Br), —CN, methyl, ethyl, methoxy and —S(O)₂Me.
93. R⁸ is independently at each occurrence selected from: —CN, methyl, and —S(O)₂Me.
94. R⁸ is independently at each occurrence selected from: halo (e.g. F or Br), —CN, methyl, —S(O)₂Me and —C(O)NH₂.
95. R³ is independently at each occurrence selected from: —CN, —S(O)₂Me, 2-hydroxyethyl, 2-methoxyethoxy, —NH₂, —N(H)Me, —N(Me)₂, —C(O)NH₂, —C(O)N(H)Me, —C(O)N(Me)₂ and pyrazolyl.
96. R⁸ is —CN.
97. R⁸ is halo (e.g. F or Br).
98. p is 0.
99. p is 1.
100. p is 0 or 1.
101. p is 0, 1 or 2.
102. p is 0, 1 or 2 and R³ is independently at each occurrence as defined in any of (69) to (91).
103. p is 0 or 1 and R⁸ is as defined in any of (69) to (97).
104. p is 1 and R⁸ is as defined in any of (69) to (97).
105. p is 1 or 2 and R⁸ is independently at each occurrence as defined in any of (69) to (97).
106. p is 1 or 2, R⁸ is independently at each occurrence as defined in any of (69) to (97) and one R⁸ group is in the ortho position relative to the ring nitrogen in the group of the formula:

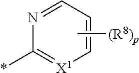

107. p is 1, R⁸ is as defined in any of (69) to (97) and the group of the formula:

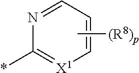

is

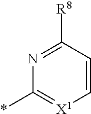

108. p is 1; R⁸ is independently at each occurrence as defined in any of (69) to (97); X¹ is CR⁷ and R⁷ is as defined in any of (58) to (67) (e.g. R⁷ is F or methyl); and the group of the formula:

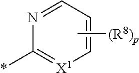

is

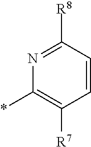

109. p is 1 or 2 (preferably 1) and R⁸ independently at each occurrence selected from: —CN, C₂₋₆ alkenyl, C₂₋₆ alkynyl, —C₁₋₆ alkylOC₁₋₄ alkyl, —C₁₋₆ alkyl-NR^{A5}R^{A5}, —C₁₋₆ alkyl-C(O)NR^{A5}R^{A5}, —C₁₋₆ alkyl-SO₂NR^{A5}R^{A5}, —OC₁₋₆ alkylOR^{B5}, —NR¹⁰¹R¹¹, —S(O)R¹⁰, —C(O)R¹⁰, —N(R¹¹)C(O)R¹⁰, —N(R¹¹)C(O)NR¹⁰R¹¹, —N(R¹¹)C(O)OR¹⁰, —N(R¹¹) SO₂R¹⁰, —C(O)NR¹⁰²R¹¹, 3 to 7 membered heterocyclyl; wherein said alkenyl, alkynyl, or heterocyclyl group is optionally substituted with from 1 to 4 R¹²
   R¹⁰¹ is selected from C₁₋₆ alkyl, C₁₋₆ haloalkyl and C₃₋₆ cycloalkyl; wherein said alkyl or cycloalkyl group is optionally substituted with from 1 to 4 R¹⁴ groups;
   R¹⁰² is selected from C₁₋₆ alkyl-R¹⁴¹, C₁₋₆ haloalkyl and C₃₋₆ cycloalkyl, wherein said cycloalkyl group is optionally substituted with from 1 to 4 R¹⁴ groups;
   R¹⁴¹ is selected from halo, —CN, C₃₋₆ cycloalkyl, —OR^{B5}, —NR^{A5}R^{A5} and —S(O)ₓR^{B5} (wherein x is 0, 1 or 2, preferably 1 or 2);
   or R¹⁰¹ and R¹¹ or R¹⁰² and R¹¹ together with the nitrogen to which they are attached form a 4 to 7 membered heterocyclyl, wherein said heterocyclyl is optionally substituted with 1 or 2 substituents selected from halo, ═O, C₁₋₄ alkyl, —OR^{B7} and C₁₋₄ haloalkyl.
110. p is 1 or 2 (preferably 1) and R⁸ independently at each occurrence selected from —CN, —C₁₋₆ alkyl-OC₁₋₄ alkyl, —C₁₋₆ alkyl-C(O)NR^{A5}R^{A5}, —OC₁₋₆ alkyl-OR^{B5}, —NR¹⁰¹R¹¹, —C(O)NR¹⁰²R¹¹, —N(R¹¹)C(O)OR¹⁰, —N(R¹) SO₂R¹⁰, —N(R¹¹)C(O)R¹⁰ and —N(R¹¹)C(O)NR¹⁰R¹¹;
   wherein R¹⁰¹ is selected from C₁₋₆ alkyl, C₁₋₆ haloalkyl, C₃₋₆ cycloalkyl and C₃₋₆ cycloalkyl-C₁₋₄ alkyl; wherein said alkyl or cycloalkyl group is optionally substituted with from 1 to 4 R¹⁴ groups selected from ═O, halo, C₁₋₄ alkyl, C₁₋₄ haloalkyl, —OR^{B5};
   R¹⁰² is selected from —C₂₋₄ alkyl-R¹⁴¹ and C₁₋₄ haloalkyl, wherein R¹⁴¹ is selected from —CN, —OR^{B5}, —NR^{A5}R^{A5} and —S(O), R^{B5} (wherein x is 0, 1 or 2, preferably 1 or 2); or R¹⁰¹ and R¹¹ or R¹⁰² and R¹¹ together with the nitrogen to which they are attached form a 4 to 7 membered heterocyclyl, wherein said heterocyclyl is optionally substituted with 1 or 2 substituents selected from halo, ═O, C₁₋₄ alkyl, C₁₋₄ haloalkyl, —OH and —OC₁₋₄ alkyl.
111. R¹⁰ is independently at each occurrence selected from: H and C₁₋₄ alkyl, wherein said alkyl is optionally substituted by halo, —CN, —OR^{B5} and —NR^{A5}R^{A5}.
   R¹¹ is independently at each occurrence selected from: H and C₁₋₄ alkyl;
   or R¹⁰ and R¹¹ together with the nitrogen to which they are attached form a 4 to 6 membered heterocyclyl selected from azetidinyl, pyrrolidinyl, piperidinyl, piperazinyl, morpholinyl and thiomorpholinyl, wherein said heterocyclyl is optionally substituted with 1 or 2 substituents selected from halo, ═O, C₁₋₄ alkyl, C₁₋₄ haloalkyl and —OR^{B7}.
112. At least one (e.g. one or two, preferably one) of R¹, R², R³, R⁷ and R⁸ is —CN with the provisos that (i) if R³ is —CN then n is not 0, and/or (ii) if R² is —CN, then m is not 0, and/or (iii) if R⁸ is —CN, then p is not 0.
113. R¹ and R³ are both —CN and n is 1.
114. R¹ is —CN, X¹ is CR⁷ and R⁷ is —CN.
115. R¹ is —CN, R⁸ is —CN and p is 1.
116. R¹ is H, p is 1 R⁸ is —CN.
117. R¹⁰ is independently at each occurrence selected from: H and C₁₋₄ alkyl, wherein said alkyl is optionally substituted by halo, —CN, —OR^{B5} and —NR^{A5}R^{A5}
118. R¹⁰ is independently at each occurrence selected from: C₁₋₄ alkyl.
119. R¹⁰ is independently at each occurrence selected from: H and C₁₋₄ alkyl, wherein said alkyl is optionally substituted by halo, —CN, —OR^{B5} and —NR^{A5}R^{A5}
120. R¹⁰ is independently at each occurrence selected from: H and C₁₋₄ alkyl, wherein said alkyl is optionally substituted by halo, —CN, —OR^{B5} and —NR^{A5}R^{A5}.
   R¹¹ is independently at each occurrence selected from: H and C₁₋₄ alkyl;
   or R¹⁰ and R¹¹ together with the nitrogen to which they are attached form a 4 to 6 membered heterocyclyl selected from azetidinyl, pyrrolidinyl, piperidinyl, piperazinyl, morpholinyl and thiomorpholinyl, wherein said heterocyclyl is optionally substituted with 1 or 2 substituents selected from halo, ═O, C₁₋₄ alkyl, C₁₋₄ haloalkyl and —OR^{B7}.
121. R¹⁰ and R¹¹ together with the nitrogen to which they are attached form a 4 to 6 membered heterocyclyl selected from azetidinyl, pyrrolidinyl, piperidinyl, piperazinyl, morpholinyl and thiomorpholinyl, wherein said heterocyclyl is optionally substituted with 1 or 2 substituents selected from halo, ═O, C₁₋₄ alkyl, C₁₋₄ haloalkyl and —OR^{B7}.
122. When R¹ is H, m is 0, n is 0 and X¹ is N or CH, then p is 1 or 2.
123. When p is 0 and X¹ is N or CH, then R¹ is not H or m is not 0.
124. When p is 0 and X¹ is N or CH, then n is 1 or 2.
125. When p is 0, m is 0 and X¹ is N or CH, then n is 1 or 2.
126. When R¹ is H, m is 0, n is 0 and X¹ is N or CH, then R⁵ is C₁₋₄alkyl.
127. When n is 0, A is O, X¹ is CR⁷ and R⁴, R⁵ and R⁶ are H; then p is 1 and R⁷ and R⁸ are not halo.
128. When n is 0, A is O, X¹ is CR⁷ and R⁴, R⁵ and R⁶ are H; then R¹ and R² are not halo (e.g. R¹ and R² are not Br).
129. R¹ and R² are not halo (e.g. R¹ and R² are not Br)
130. One or more hydrogen atoms in the compound is deuterium.
131. The group

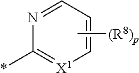

is selected from:

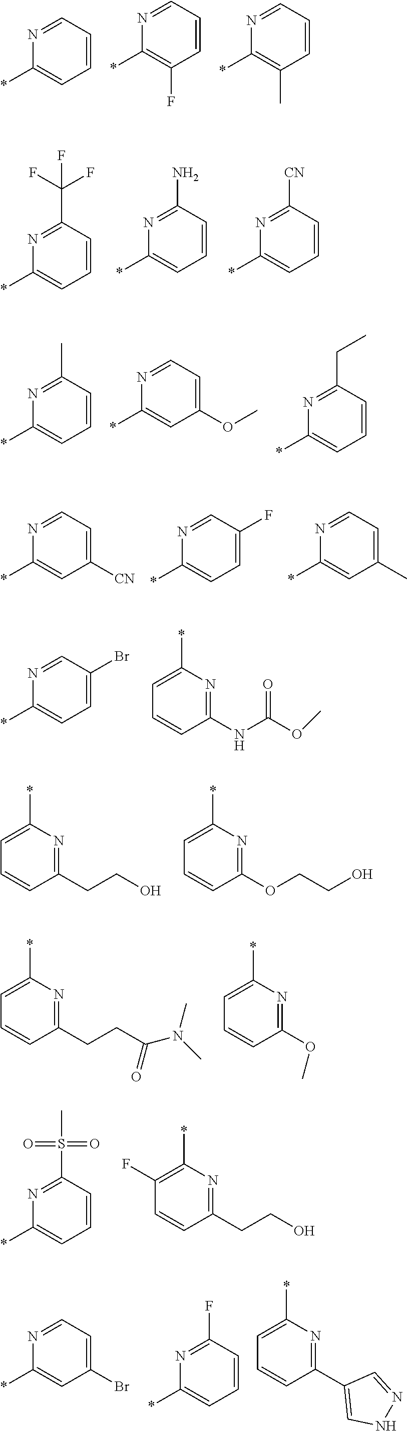

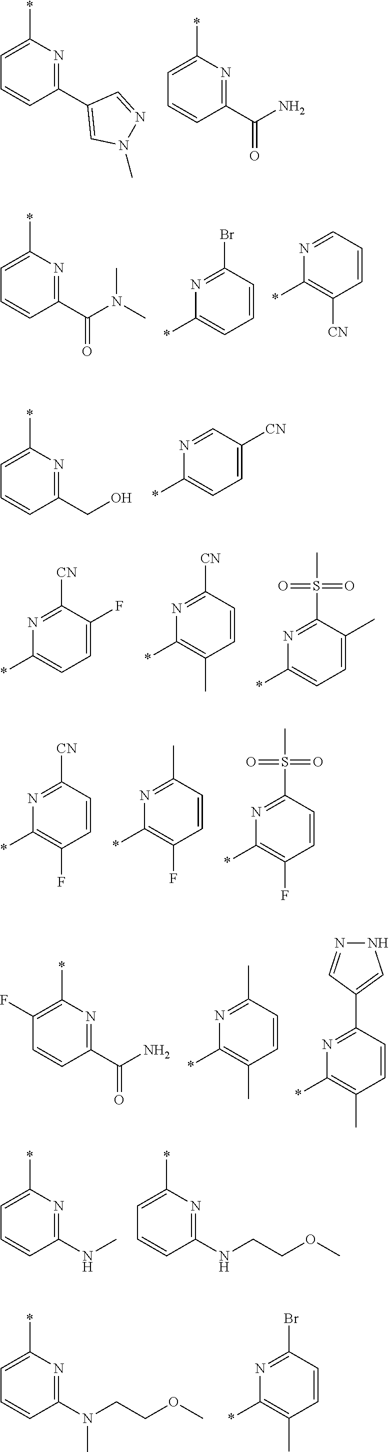

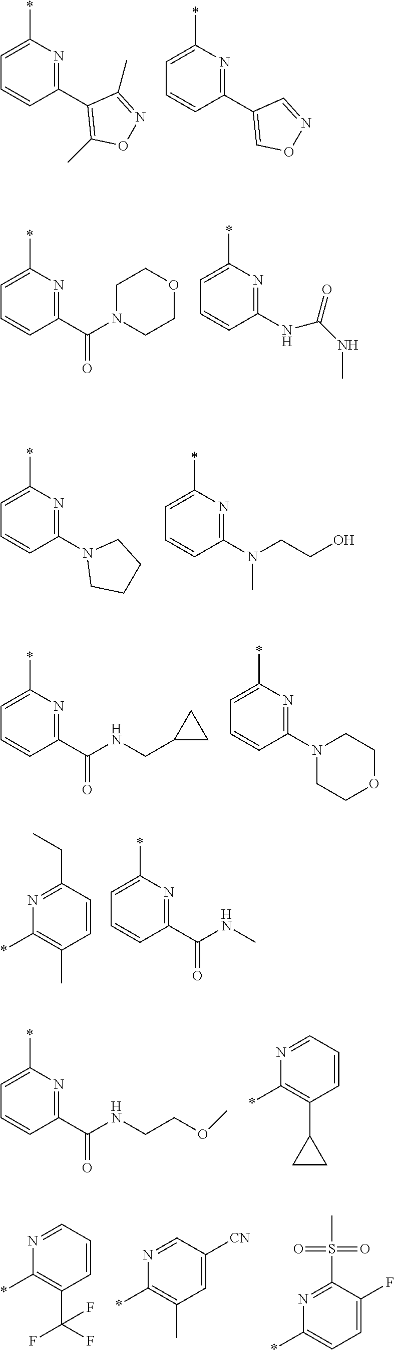

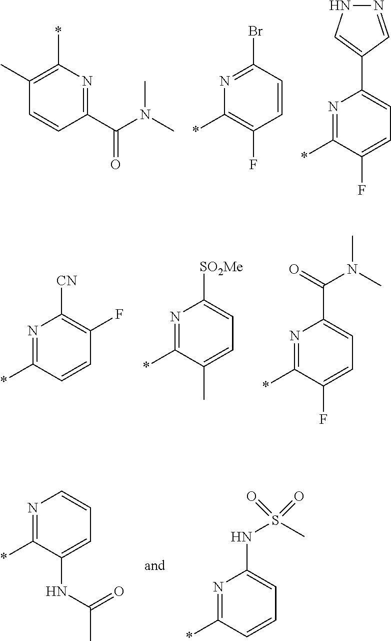

wherein * shows the point of attachment to the remainder of the molecule.
132. The group

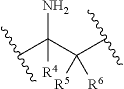

is of the formula

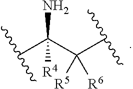

In some embodiments there is provided a compound of the formula (I), or a pharmaceutically acceptable salt thereof wherein:
R¹ is selected from —CN, C₂₋₆ alkenyl, C₂₋₆ alkynyl, C₃₋₆ cycloalkyl, C₃₋₆ cycloalkyl-C₁₋₆ alkyl and —C₁₋₆ alkyl-OR^{B2}, wherein said alkenyl or alkynyl group is substituted with 1 or 2 substituents selected from halo, C₁₋₄ haloalkyl and —OR^{B2}; and said cycloalkyl or cycloalkyl-alkyl group is optionally substituted with 1 to 4 substituents independently selected from halo, C₁₋₄ alkyl, C₁₋₄ haloalkyl and —OR^{B2};
m is 0 or 1 (preferably 0);
R^{B2} is independently at each occurrence selected from: H, C₁₋₄ alkyl and C₁₋₄ haloalkyl;
R² is selected from: halo, C₁₋₄ alkyl and C₁₋₄ haloalkyl;
A is O or NR⁹;
R⁹ is selected from H and C₁₋₄ alkyl;
n is 0, 1 or 2 (preferably 0 or 1) and R³ is independently at each occurrence selected from: halo, —CN and C₁₋₄ alkyl;
R⁴ is H, methyl or ethyl (preferably H or methyl, more preferably H);
R⁵ and R⁶ are each independently selected from: H and C₁₋₃ alkyl; X¹ is N or CR¹;
R⁷ is selected from: H, halo, —CN, C₁₋₄ alkyl and C₁₋₄ haloalkyl;
p is 0, 1 or 2; and
R⁸ has any of the values in any one of paragraphs (69) to (97) above.

In some embodiments there is provided a compound of the formula (I), or a pharmaceutically acceptable salt thereof wherein:
R¹ is selected from: H, halo, —CN, C₁₋₄ alkyl, C₁₋₄ haloalkyl, C₂₋₄ alkenyl, C₂₋₄ alkynyl, C₃₋₅ cycloalkyl, and wherein any alkyl, alkenyl, alkynyl or cycloalkyl group is optionally substituted with 1 to 4 substituents independently selected from halo, C₁₋₄ alkyl, C₁₋₄ haloalkyl and —OR^{B2};
m is 0 or 1 and R² is selected from: halo, C₁₋₄ alkyl and C₁₋₄ haloalkyl;
A is O or NR⁹;
R⁹ is selected from H and C₁₋₄ alkyl;
n is 0, 1 or 2 (preferably 0 or 1);
R³ is independently at each occurrence selected from: halo, —CN and C₁₋₄alkyl;
R⁴ is H, methyl or ethyl (preferably H or methyl, more preferably H);
R⁵ and R⁶ are each independently selected from: H and C₁₋₃ alkyl; X¹ is N or CR⁷;
R⁷ is selected from: H, halo, —CN, C₁₋₄ alkyl and C₁₋₄ haloalkyl;
p is 0, 1 or 2 (e.g. p is 1 or 2) and R⁸ has any of the values in any one of paragraphs (69) to (97) above;
with the proviso that at least one of R¹, R³, R⁷ and/or R⁸ is —CN.

In this embodiment it may be that R¹ is selected from H, halo, —CN and, C₁₋₃ alkyl.

In this embodiment it may be that R¹ is selected from H, —CN and, C₁₋₃alkyl.

In this embodiment it may be that R¹ is CN.

In this embodiment it may be that n is 1 and R³ is CN.

In this embodiment it may be that X¹ is N.

In this embodiment it may be that X¹ is CR⁷.

In this embodiment it may be that X¹ is CR⁷ and R⁷ is CN.

In this embodiment it may be that one R⁸ is —CN and p is 1 or 2.

In this embodiment it may be that p is 1 and R³ is —CN.

In this embodiment it may be that p is 2, one R⁸ is —CN and the other R⁸ is selected from any of the values in any one of paragraphs (69) to (97) above.

In this embodiment it may be that p is 2, one R⁸ is —CN and the other R⁸ is selected from halo, —CN, C₁₋₄ alkyl, C₁₋₄ haloalkyl, —C₁₋₄ alkyl-OH, —C₁₋₄ alkyl-OMe, —C₁₋₄ alkyl-C(O)NH₂, —C₁₋₄ alkyl-C(O)N(H)Me, —C₁₋₄ alkyl-C(O)N(Me)₂, —OC₁₋₄ alkyl, —OC₂₋₄ alkyl-OH, —OC₂₋₄alkyl-OMe, —NH₂, —NH(C₁₋₄alkyl), —N(C₁₋₄alkyl)₂, —SO₂C₁₋₄ alkyl, —C(O)NH₂, —C(O)N(H)C₁₋₄alkyl, —C(O)N(C₁₋₄alkyl)₂, azetidin-1-yl-C(O)—, pyrrolidin-1-yl-C(O)—, piperidin-1-yl-C(O)—, piperarazin-1-yl-C(O)—, morpholin-1-yl-C(O)—, —N(H)C(O)C₁₋₄alkyl, —N(H)C(O)O(C₁₋₄alkyl), —NHSO₂(C₁₋₄alkyl) and pyrazolyl.

In some embodiments there is provided a compound of the formula (I), or a pharmaceutically acceptable salt thereof wherein:
R¹ is selected from H, —CN, halo and C₁₋₃ alkyl;
m is 0;
A is O or NR⁹;
R⁹ is selected from H and C₁₋₄ alkyl (preferably R⁹ is C₁₋₃ alkyl, e.g. methyl, ethyl or isopropyl);
n is 0 or 1;
R³ is selected from: halo and C₁₋₃ alkyl;
R⁴ is H;
R⁵ and R⁶ are each independently selected from: H and C₁₋₃ alkyl (preferably R⁵ is H and R⁶ is H, methyl or ethyl);
X¹ is CR¹;
R⁷ is selected from: H, halo and C₁₋₄alkyl;
p is 0, 1 or 2;
R⁸ is independently at each occurrence selected from: halo, —CN, C₁₋₄ alkyl, C₁₋₄haloalkyl, —C₁₋₄ alkyl-OH, —C₁₋₄ alkyl-OMe, —C₁₋₄ alkyl-C(O)NH₂, —C₁₋₄ alkyl-C(O)N(H)Me, —C₁₋₄ alkyl-C(O)N(Me)₂, —OC₁₋₄ alkyl, —OC₂₋₄ alkyl-OH, —OC₂₋₄ alkyl-OMe, —NH₂, —NH(C₁₋₄ alkyl), —N(C₁₋₄ alkyl)₂, —SO₂C₁₋₄ alkyl and pyrazolyl.

In this embodiment it may be that R¹ is selected from selected from H, —CN and C₁₋₃ alkyl.

In this embodiment it may be that R¹ is H or C₁₋₃ alkyl (e.g. R¹ is H or methyl).

In this embodiment it may be that R¹ is —CN.

In this embodiment it may be that R¹ is halo (e.g. Br).

In this embodiment it may be that R¹ is H.

In this embodiment it may be that R⁸ is selected from halo, —CN, C₁₋₃alkyl, —CF₃, —C₁₋₃ alkyl-OH, —C₁₋₃ alkyl-OMe, —C₁₋₃ alkyl-C(O)NH₂, —C₁₋₃ alkyl-C(O)N(H)Me, —C₁₋₃ alkyl-C(O)N(Me)₂, —OC₁₋₃ alkyl, —OC₂₋₃ alkyl-OH, —OC₂₋₃ alkyl-OMe, —NH₂, —NH(C₁₋₃ alkyl), —N(C₁₋₃ alkyl)₂, —SO₂C₁₋₃ alkyl and pyrazolyl. For example, R⁸ may be selected from halo (e.g. F or Br), —CN, methyl, ethyl, 2-hydroxyethyl, methoxy, 2-hydroxyethoxy, —CF₃, —NH₂, —S(O)₂Me, —C(O)NH₂, —C(O)N(H)Me, —C(O)N(Me)₂, —(CH₂)₂C(O)NH₂, —(CH₂)₂C(O)N(H)Me, —(CH₂)₂C(O)N(Me)₂, —NHC(O)OMe, and pyrazol-4-yl.

In some embodiments there is provided a compound of the formula (I), or a pharmaceutically acceptable salt thereof wherein:
R¹ is selected from H, —CN and C₁₋₃alkyl;
m is 0;
A is O;
n is 0 or 1;
R³ is selected from: halo and C₁₋₃ alkyl;
R⁴ is H;
R⁵ and R⁶ are each independently selected from: H methyl and ethyl;
X¹ is CR¹;
R⁷ is selected from: H, F and C₁₋₃ alkyl;
p is 0, 1 or 2;
R⁸ is independently at each occurrence selected from: halo, —CN, C₁₋₄ alkyl, C₁₋₄ haloalkyl, —OC₁₋₃ alkyl, —NH₂, —NH(C₁₋₄ alkyl), —N(C₁₋₄ alkyl)₂ and —SO₂C₁₋₄ alkyl.

In this embodiment it may be that R⁷ is selected from H, F and methyl. For example, R⁷ is F or methyl.

In this embodiment it may be that R⁸ is selected from —CN, methyl, —CF₃, methoxy, —NH₂, and —SO₂Me.

In another embodiment there is provided a compound of the formula (I), or a pharmaceutically acceptable salt thereof wherein:
R¹ is selected from H, —CN, and C₁₋₃ alkyl;
A is O or NR⁹ (preferably A is O);
R⁹ is selected from H and C₁₋₄ alkyl (preferably R⁹ is C₁₋₃ alkyl);
n is 0 or 1;
R³ is selected from: halo, —CN, and C₁₋₃alkyl;
R⁴ is H;
R⁵ and R⁶ are each independently selected from: H and C₁₋₃ alkyl (preferably R⁵ is H and
R⁶ is H, methyl or ethyl); and
the group

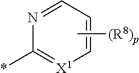

is selected from:

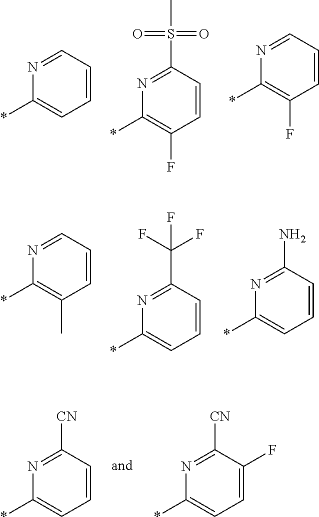

wherein * shows the point of attachment to the remainder of the molecule.

In this embodiment it may be that R¹ is selected from H, —CN and C₁₋₃alkyl.

In this embodiment it may be that R¹ is H or C₁₋₃ alkyl, for example R¹ is H or methyl.

In this embodiment it may be that R¹ is H.

In this embodiment it may be that R¹ is C₁₋₃alkyl, for example methyl.

In this embodiment it may be that R¹ is halo (e.g. F, Cl or Br).
In this embodiment it may be that the group

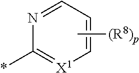

is selected from:

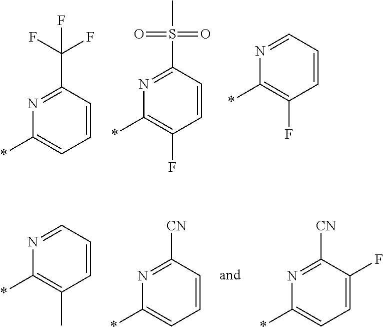

wherein * shows the point of attachment to the remainder of the molecule.

In another embodiment there is provided a compound of the formula (I), or a pharmaceutically acceptable salt thereof wherein:
R¹ is selected from H and halo (e.g. F, Cl or Br);
m is 0;
A is O or NR⁹;
R⁹ is selected from H and C₁₋₃ alkyl;
n is 0 or 1;
R³ is selected from: halo and C₁₋₃ alkyl;
R⁴ is H;
R⁵ and R⁶ are each independently selected from: H and C₁₋₃ alkyl (preferably R⁵ is H and R⁶ is H, methyl or ethyl);
X¹ is CR⁷;
R⁷ is H, halo or C₁₋₃ alkyl;
p is 0, 1 or 2;
R⁸ is independently at each occurrence selected from: halo, —CN, C₁₋₃ alkyl, —CF₃, —C₁₋₃ alkyl-OH, —OC₁₋₃ alkyl, —OC₂₋₃ alkyl-OH, and —SO₂C₁₋₃ alkyl.

In this embodiment it may be that R¹ is H.

In this embodiment it may be that R¹ is halo, for example F, Cl or Br.

In this embodiment it may be that:
R¹ is H;
A is O or NR⁹, wherein R⁹ is selected from methyl, ethyl and isopropyl;
R³ is methyl;
R⁴ and R⁵ are H;
R⁶ is H or methyl;
R⁷ is H, methyl or F;
m is 0;
n is 0 or 1;
p is 0, 1 or 2;
R⁸ is independently at each occurrence selected from: F, —CN, methyl, ethyl, —CF₃, methoxy, 2-hydroxyethyl and —SO₂Me. For example, R⁸ selected from —CN, methyl, ethyl and —S(O)₂Me.

In another embodiment there is provided a compound of the formula (I), or a pharmaceutically acceptable salt thereof wherein:
R¹ is selected from H, C₁₋₃ alkyl and CN;
m is 0;
A is O;
n is 0 or 1;
R³ is selected from: halo and C₁₋₃ alkyl;
R⁴ is H;
R⁵ and R⁶ are each independently selected from: H and C₁₋₃ alkyl (preferably R⁵ is H and
R⁶ is H, methyl or ethyl); and
the group

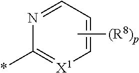

is selected from:

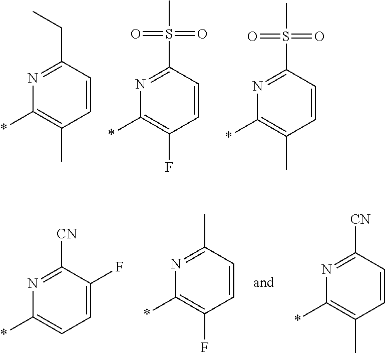

wherein * shows the point of attachment to the remainder of the molecule.

In another embodiment there is provided a compound of the formula (I), or a pharmaceutically acceptable salt thereof wherein:
R¹ is selected from H, C₁₋₃ alkyl and CN;
m is 0;
A is O;
n is 0 or 1;
R³ is selected from: halo and C₁₋₃ alkyl;
R⁴ is H;
R⁵ and R⁶ are each independently selected from: H and C₁₋₃ alkyl (preferably R⁵ is H and
R⁶ is H, methyl or ethyl); and
the group

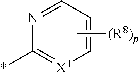

is selected from

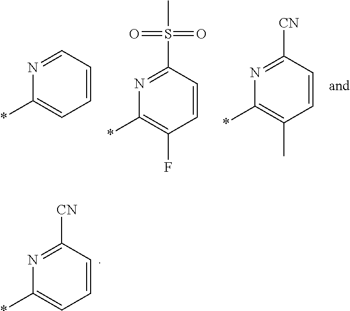

Thus it may be in this embodiment that the group

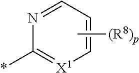

is selected from

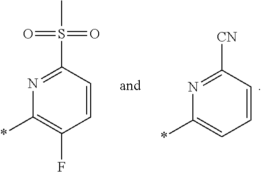 [SMILES: *c1nc(S(C)(=O)=O)ccc1F ; *c1cccc(C#N)n1]

In another embodiment there is provided a compound of the formula (I), or a pharmaceutically acceptable salt thereof wherein:
R¹ is selected from: H, halo, —CN, C₁₋₃ alkyl, C₂₋₃ alkenyl, C₂₋₃ alkynyl, C₃₋₅ cycloalkyl, and wherein any alkyl, alkenyl, alkynyl or cycloalkyl group is optionally substituted with 1 or 2 substituents independently selected from halo C₁₋₄ alkyl, —OH and —OC₁₋₂ alkyl;
m is 0;
A is O or NR⁹;
R⁹ is selected from H and C₁₋₄ alkyl;
n is 0, 1 or 2 (preferably 0 or 1);
R³ is independently at each occurrence selected from: halo, —CN and C₁₋₄alkyl;
R⁴ is H, methyl or ethyl (preferably H or methyl, more preferably H);
R⁵ and R⁶ are each independently selected from: H and C₁₋₃ alkyl;
X¹ is CR¹;
R⁷ is selected from: H, halo, —CN, C₁₋₄ alkyl and C₁₋₄ haloalkyl;
p is 1 or 2; and
R⁸ is selected from —C₁₋₄ alkyl-OR^{B5}, —OC₂₋₄ alkyl-OR^{B5}, —NH₂, —N(H)C₁₋₄ alkyl, —N(C₁₋₄ alkyl)₂, —S(O)₂C₁₋₄ alkyl, —C(O)NH₂, —C(O)N(H)C₁₋₄ alkyl, —C(O)N(C₁₋₄ alkyl)₂ and pyrazolyl.

In this embodiment it may be that R¹ is selected from: H, —CN and C₁₋₃alkyl.

In this embodiment it may be that R¹ is halo (e.g. F, Cl or Br).

In this embodiment it may be that R¹ —CN.

In this embodiment it may be that R¹ is H.

In this embodiment it may be that R⁷ is selected from: H, F and Me.

In this embodiment it may be that R⁷ is H.

In this embodiment it may be that the group:

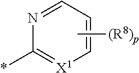

is

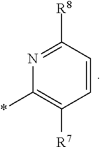

In this embodiment it may be that R⁸ is selected from: —(CH₂)₂OH, —O(CH₂)₂OH, —C(O)NH₂, —C(O)N(H)Me, —C(O)N(Me)₂, —NH₂, —N(H)Me, —N(Me)₂, —S(O)₂Me and pyrazolyl.

In this embodiment it may be that R⁸ is selected from: —(CH₂)₂OH, —O(CH₂)₂OH, —C(O)NH₂, —C(O)N(H)Me, —C(O)N(Me)₂, —NH₂, —N(H)Me, —N(Me)₂ and —S(O)₂Me.

### Compounds of the Formula (II)

In some embodiments the compound of formula (I) is a compound of the formula (II), or a pharmaceutically acceptable salt thereof as hereinbefore defined. In the compound of the formula (II) R¹, R², R³, R⁴, R⁵, R⁶, R⁸, A, X¹, m and n are as defined in relation to the compound of formula (I), or, unless stated otherwise, have any of the values defined herein including is one of more of (1) to (132) (in so far as those paragraphs are applicable to a compound of the formula (II)). The following embodiments are directed to compounds of the Formula (II).

In some embodiments in the compound of formula (II) one of R⁸¹ and R⁸² is H and the other is selected from halo, —CN, C₁₋₄ alkyl and C₁₋₄ haloalkyl, —OH and —OC₁₋₄ alkyl.

In some embodiments in the compound of formula (II) R⁸¹ and R⁸² are both H.

In some embodiments in the compound of formula (II) R⁸¹ and R⁸² are independently selected from: H, halo, —CN, CF₃, —OH and —OMe.

In some embodiments in the compound of formula (II) X¹ is CR⁷.

In some embodiments in the compound of formula (II) X¹ is CR⁷ and R⁷ is as defined in any one of (58) to (67).

In some embodiments in the compound of formula (II) X¹ is CR⁷ and R⁷ is selected from halo and C₁₋₃ alkyl (e.g. R⁷ is selected from F and Me).

In some embodiments in the compound of formula (II) R¹ and R² are not halo (e.g. they are not Br).

In some embodiments in the compound of formula (II) m is 0 and R¹ is selected from H, C₁₋₃ alkyl and —CN.

In some embodiments in the compound of formula (II) m is 0 and R¹ is H.

In some embodiments in the compound of formula (II) A is O.

In some embodiments in the compound of formula (II), including any one of the 10 embodiments, above it may be that R⁸ has any of the values defined in any one of (69) to (97).

In some embodiments in the compound of formula (II), including any one of the 11 embodiments above, R⁸ is selected from —CN, C₁₋₄ alkyl, C₁₋₄ haloalkyl, —C₁₋₄ alkyl-OH, —C₁₋₄ alkyl-OMe, —C₁₋₄ alkyl-C(O)NH₂, —C₁₋₄ alkyl-C(O)N(H)Me, —C₁₋₄ alkyl-C(O)N(Me)₂, —OC₁₋₄ alkyl, —OC₂₋₄alkyl-OH, —OC₂₋₄alkyl-OC₁₋₃ alkyl, —NH₂, —NH(C₁₋₄alkyl), —N(C₁₋₄ alkyl)₂, —C(O)NH₂, —C(O)N(H)C₁₋₃ alkyl, —C(O)N(C₁₋₃ alkyl)₂, —SO₂C₁₋₄ alkyl and pyrazolyl.

In some embodiments in the compound of formula (II), including any one of the 12 embodiments above, R⁸ is selected from —CN, —C₁₋₄ alkyl-OH, —C₁₋₄ alkyl-OMe, —OC₁₋₄ alkyl, —OC₂₋₄alkyl-OH, —OC₂₋₄alkyl-OMe, —NH₂, —NH(Me), —NH(Et), —N(Me)₂, —C(O)NH₂, —C(O)N(H)Me, —C(O)N(Me)₂, —SO₂Me and —SO₂Et.

### Compounds of the Formula (III)

In some embodiments the compound of formula (I) is a compound of the formula (III), or a pharmaceutically acceptable salt thereof as hereinbefore defined. In the compound of the formula (III) R¹, R², R³, R⁴, R⁵, R⁶, R⁷, R⁸, A, X¹, m, n and p are as defined in relation to the compound of formula (I), or, unless stated otherwise, have any of the values defined herein including is one of more of (1) to (132) (in so far as those paragraphs are applicable to a compound of the formula (III)). The following embodiments are directed to compounds of the formula (III).

In some embodiments in the compound of formula (III), R⁷ is selected from halo, —CN, C₁₋₄ alkyl and C₁₋₄ haloalkyl.

In some embodiments in the compound of formula (III), R⁷ is C₁₋₃ alkyl.

In some embodiments in the compound of formula (III), R⁷ is —CN.

In some embodiments in the compound of formula (III), R⁷ is selected from halo (e.g. F), methyl, ethyl and —CF₃.

In some embodiments in the compound of formula (III), R⁷ is selected from halo (e.g. F) and methyl.

In some embodiments in the compound of formula (III), R¹ and R² are not halo (e.g. they are not Br).

In some embodiments in the compound of formula (III), m is 0 and R¹ is selected from H, C₁₋₃ alkyl and —CN.

In some embodiments in the compound of formula (III), m is 0 and R¹ is selected H.

In some embodiments in the compound of formula (III), including any one of the 8 embodiments above, p is 1. In some embodiments in the compound of formula (III), including any one of the 8 embodiments above, p is 0.

### Compounds of the Formula (IV)

In some embodiments the compound of formula (I) is a compound of the formula (IV), or a pharmaceutically acceptable salt thereof as hereinbefore defined. In the compound of the formula (IV) R¹, R², R³, R⁴, R⁵, R⁶, R⁷, R⁸, A, X¹, m and n are as defined in relation to the compound of formula (I), or, unless stated otherwise, have any of the values defined herein including is one of more of (1) to (132) (in so far as those paragraphs are applicable to a compound of the formula (IV)). The following embodiments are directed to compounds of the formula (IV).

In some embodiments in the compound of formula (IV), R⁷ is selected from halo, —CN, C₁₋₄ alkyl and C₁₋₄ haloalkyl.

In some embodiments in the compound of formula (IV), R⁷ is selected from: halo (e.g. F), methyl, ethyl and —CF₃.

In some embodiments in the compound of formula (IV), R⁷ is C₁₋₃ alkyl.

In some embodiments in the compound of formula (IV), R⁷ is —CN.

In some embodiments in the compound of formula (IV), R⁷ is selected from: halo (e.g. F), methyl, ethyl and —CF₃; and
R⁸ is as defined in any one of (69) to (97).

In some embodiments in the compound of formula (IV), R⁷ is selected from: halo, —CN, C₁₋₄ alkyl and C₁₋₄ haloalkyl; and
R⁸ is selected from: halo, —CN, C₁₋₄ alkyl, C₁₋₄ haloalkyl, —C₁₋₄ alkyl-OH, —C₁₋₄ alkyl-OMe, —C₁₋₄ alkyl-C(O)NH₂, —CO₁₋₄ alkyl-C(O)N(H)—CO₁₋₄ alkyl, —CO₁₋₄ alkyl-C(O)N(C₁₋₄ alkyl)₂, —OC₁₋₄ alkyl, —OC₂₋₄ alkyl-OH, —OC₂₋₄ alkyl-O—C₁₋₄ alkyl, —NH₂, —NH(C₁₋₄ alkyl), —N(C₁₋₄ alkyl)₂, —C(O)NH₂, —C(O)N(H)—C₁₋₄ alkyl, —C(O)N(C₁₋₄ alkyl)₂, —SO₂C₁₋₄ alkyl and pyrazolyl.

In some embodiments in the compound of formula (IV), R⁷ is selected from halo (e.g. F), C₁₋₃alkyl and CF₃;
and R⁸ is selected from halo, —CN, C₁₋₃ alkyl, CF₃—C₁₋₃ alkyl-OH, —C₁₋₃ alkyl-OMe, —C₁₋₃ alkyl-C(O)NH₂, —C₁₋₃ alkyl-C(O)N(H)Me, —C₁₋₃ alkyl-C(O)N(Me)₂, —OC₁₋₃ alkyl, —OC₂₋₃ alkyl-OH, —OC₂₋₃ alkyl-OMe, —NH₂, —NH(C₁₋₄ alkyl), —N(C₁₋₄ alkyl)₂, —C(O)NH₂, —C(O)N(H)Me, —C(O)N(Me)₂, —SO₂C₁₋₃ alkyl and pyrazolyl.

In some embodiments in the compound of formula (IV), R⁷ is selected from F and methyl; and R⁸ is selected from —CN, —C₁₋₃ alkyl-OH, —C(O)NH₂, —C(O)N(H)Me, —C(O)N(Me)₂ and —SO₂Me.

In some embodiments in the compound of formula (IV), R⁷ and R⁸ are not halo.

In some embodiments in the compound of formula (IV), including any of the embodiments above, m is 0 and R¹ is selected from H, —CN and C₁₋₃alkyl.

In some embodiments in the compound of formula (IV), including any of the embodiments above, m is 0 and R¹ is H.

In some embodiments in the compound of formula (IV), including any of the embodiments above, A is O.

### Compounds of the Formula (V)

In some embodiments the compound of formula (I) is a compound of the formula (V), or a pharmaceutically acceptable salt thereof as hereinbefore defined. In the compound of the formula (V) R¹, R², R³, R⁴, R⁵, R⁶, R⁸, R¹⁰, A, X¹, m and n are as defined in relation to the compound of formula (I), or, unless stated otherwise, have any of the values defined herein including is one of more of (1) to (132) (in so far as those paragraphs are applicable to a compound of the formula (V)). The following embodiments are directed to compounds of the formula (V).

In some embodiments in the compound of formula (V), R¹⁰ is C₁₋₄ alkyl or C₃₋₆ cycloalkyl. For example, R¹⁰ is methyl, ethyl or cyclopropyl. It may be that R¹⁰ is C₁₋₄ alkyl. It may be that R¹⁰ is methyl. It may be that R¹⁰ is C₃₋₅ cycloalkyl. For example, R¹⁰ is cyclopropyl, cyclobutyl or cyclopentyl.

In some embodiments in the compound of formula (V), it may be that p1 is 0 and R¹⁰ is selected from C₁₋₄ alkyl, wherein said alkyl is optionally substituted by halo.

In some embodiments in the compound of formula (V), including any of the embodiments above, it may be that p1 is 0 and R¹⁰ is selected from methyl and ethyl.

In some embodiments in the compound of formula (V), p1 is 0 or 1. For example p1 is 0. For example, p is 1.

In some embodiments in the compound of formula (V), R⁸ is independently at each occurrence selected from halo, —CN, C₁₋₄ alkyl, C₁₋₄ haloalkyl, —OH and —OC₁₋₄ alkyl and p1 is 0 or 1.

In some embodiments in the compound of formula (V), p1 is 0 or 1 and R⁸ is selected from halo, —CN, C₁₋₃ alkyl, CF₃, —OH and —OC₁₋₃ alkyl.

In some embodiments in the compound of formula (V), including any of the embodiments above, it may be that X¹ is CR⁷. For example, it may be that X¹ is CR⁷ and R⁷ is selected from H, halo, —CN, C₁₋₄ alkyl and C₁₋₄ haloalkyl. For example, it may be that X¹ is CR⁷ and R⁷ is selected from F and Me. For example, it may be that X¹ is CR⁷ and R⁷ is H.

In some embodiments in the compound of formula (V), including any of the embodiments above, it may be that m is 0 and R¹ is selected from H, —CN and C₁₋₃ alkyl.

In some embodiments in the compound of formula (V), including any of the embodiments above, it may be that m is 0 and R¹ is H.

In some embodiments in the compound of formula (V), including any of the embodiments above, it may be that A is O.

In some embodiments in the compound of formula (V), including any of the embodiments above, it may be that m is 0 and R¹ is selected from H, —CN and C₁₋₃ alkyl. For example, m is 0 and R¹ is H.

In the compound of formula (V), including any of the embodiments above, it may be that X¹ is CR⁷ and R⁷ is as defined in any one of (58) to (67) above.

### Compounds of the Formula (VI)

In some embodiments the compound of formula (I) is a compound of the formula (VI), or a pharmaceutically acceptable salt thereof as hereinbefore defined. In the compound of the formula (VI) R¹, R², R³, R⁴, R⁵, R⁶, R⁸, R¹⁰, R¹¹, A, X¹, m and n are as defined in relation to the compound of formula (I), or, unless stated otherwise, have any of the values defined herein including is one of more of (1) to (132) (in so far as those paragraphs are applicable to a compound of the formula (VI)). The following embodiments are directed to compounds of the formula (VI).

In some embodiments in the compound of formula (VI), R¹⁰ and R¹¹ are independently H or C₁₋₄ alkyl;
or R¹⁰ and R¹¹ together with the nitrogen to which they are attached form a 4 to 6 membered heterocyclyl selected from azetidinyl, pyrrolidinyl, piperidinyl, piperazinyl, morpholinyl and thiomorpholinyl, wherein said heterocyclyl is optionally substituted with 1 or 2 substituents selected from halo, ═O, C₁₋₄ alkyl, C₁₋₄ haloalkyl and —OR^{B7}.

In some embodiments in the compound of formula (VI), R¹⁰ and R¹¹ are independently H or C₁₋₃ alkyl (e.g. H or methyl).

In some embodiments in the compound of formula (VI), p1 is 0 or 1. For example p1 is 0. For example, p1 is 1.

In some embodiments in the compound of formula (VI), R⁸ is independently at each occurrence selected from halo, —CN, C₁₋₄ alkyl, C₁₋₄ haloalkyl, —OH and —OC₁₋₄ alkyl.

In some embodiments in the compound of formula (VI), p1 is 0 or 1 and R⁸ is selected from halo, —CN, C₁₋₃ alkyl, CF₃, —OH and —OC₁₃ alkyl.

In the compound of formula (VI), including any of the embodiments above, it may be that X¹ is CR⁷. For example, it may be that X¹ is CR⁷ and R⁷ is selected from halo, —CN, C₁₋₄ alkyl and C₁₋₄ haloalkyl.

In the compound of formula (VI), including any of the embodiments above, it may be that m is 0 and R¹ is selected from H, —CN and C₁₋₃ alkyl. For example, m is 0, and R¹ is H.

In the compound of formula (VI), including any of the embodiments above, it may be that X¹ is CR⁷ and R⁷ is as defined in any one of (58) to (67) above.

In the compound of formula (VI), including any of the embodiments above, it may be that A is O.

### Compounds of the Formulae (VII) and (VIII)

In some embodiments the compound of formula (I) is a compound of the formula (VII) or (VIII), or a pharmaceutically acceptable salt thereof as hereinbefore defined. In the compound of the formulae (VII) and (VIII) R¹, R², R³, R⁴, R⁵, R⁶, R⁸, A, X¹, m, n and p are as defined in relation to the compound of formula (I), or, unless stated otherwise, have any of the values defined herein including is one of more of (1) to (132) (in so far as those paragraphs are applicable to a compound of the formulae (VII) and (VIII). The following embodiments are directed to compounds of the formulae (VII) and (VIII).

In some embodiments some embodiments in the compounds of formulae (VII) and (VIII), R³ is selected from halo, —CN, C₁₋₄alkyl and C₁₋₄haloalkyl.

In some embodiments in the compounds of formulae (VII) and (VIII), R³ is selected from halo and C₁₋₃ alkyl. For example, R³ is F, Cl, Br or methyl.

In some embodiments in the compounds of formulae (VII) and (VIII), including any of the embodiments above, it may be that m is 0 and R¹ is selected from H, —CN and —C₁₋₃ alkyl. For example, m is 0 and R¹ is H.

In some embodiments in the compounds of formulae (VII) and (VIII), including any of the embodiments above, it may be that A is O.

### Compounds of the Formula (IX)

In some embodiments the compound of formula (I) is a compound of the formula (IX), or a pharmaceutically acceptable salt thereof as hereinbefore defined. In the compound of the formula (IX) R², R³, R⁴, R⁵, R⁶, R⁸, A, X¹, n and p are as defined in relation to the compound of formula (I), or, unless stated otherwise, have any of the values defined herein including is one of more of (1) to (132) (in so far as those paragraphs are applicable to a compound of the formula (IX)). The following embodiments are directed to compounds of the formula (IX)

In some embodiments in the compound of formula (IX), R² is selected from halo, C₁₋₃ alkyl and C₁₋₃ haloalkyl.

In some embodiments in the compound of formula (IX), R² is halo. It may be that R² is F. It may be that R² is Br. It may be that R² is Cl. In some embodiments in the compound of formula (IX), R² is C₁₋₃ alkyl. It may be that R² is methyl. It may be that R² is C₁₋₃ haloalkyl. It may be that R² is CF₃.

In some embodiments in the compounds of formulae (I), (II), (Ill), (IV), (V), (VI), (VII), (VIII) or (IX), including any of the embodiments above, it may be that R⁴ and R⁵ are H and R⁶ is selected from: H and methyl.

In some embodiments in the compounds of formulae (I), (II), (Ill), (IV), (V), (VI), (VII), (VIII) or (IX), including any of the embodiments above, it may be that the group

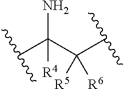

is of the formula

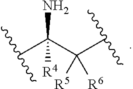

In some embodiments in the compounds of formulae (I), (II), (Ill), (IV), (V), (VI), (VII), (VIII) or (IX), including any of the embodiments above, it may be that the group

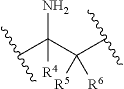

is of the formula

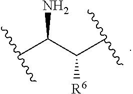

In some embodiments in the compounds of formulae (I), (II), (Ill), (IV), (V), (VI), (VII), (VIII) or (IX), including any of the embodiments above, it may be that the group

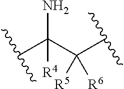

is of the formula

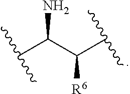

In some embodiments in the compounds of formulae (I), (II), (Ill), (IV), (V), (VI), (VII), (VIII) or (IX), including any of the embodiments above, it may be that the group

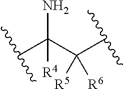

is:

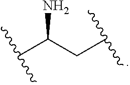 [SMILES: *C[C@@H](*)N]

In another embodiment there is provided a compound selected from any one of the Examples herein, or a pharmaceutically acceptable salt or prodrug thereof.

In another embodiment there is provided a compound selected from Table 1, or a pharmaceutically acceptable salt or prodrug thereof. In particular there is provided a compound selected from Table 1, or a pharmaceutically acceptable salt thereof:

TABLE 1

| Chemical name | Chemical structure |
|---|---|
| 1-[2-(Benzo[d]isoxazol-3-yl)phenyl]-2-(pyridin-2- yl)propan-1-amine (including diastereomers 1, 2 and 3) Exemplary salt: 1-[2-(Benzo[d]isoxazol-3-yl)phenyl]-2-(pyridin-2- yl)propan-1-amine hydrochloride | 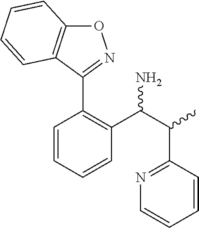 [SMILES: CC(c1ccccn1)C(N)c1ccccc1-c1noc2ccccc12] |
| 1-[2-(Benzo[d]isoxazol-3-yl)phenyl]-1-(pyridin-2- yl)propan-2-amine Exemplary salt: 1-[2-(Benzo[d]osoxazol-3-yl)phenyl]-1-(pyridin-2- yl)propan-2-amine hydrochloride | 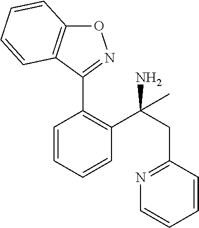 [SMILES: C[C@](N)(Cc1ccccn1)c1ccccc1-c1noc2ccccc12] |
| (S)-1-[2-(Benzo[d]osoxazol-3-yl)phenyl]-2-[3- fluoro-6-(methylsulfonyl)pyridine-2-yl]ethan-1- amine Exemplary salt: (S)-1-[2-(Benzo[d]osoxazol-3-yl)phenyl]-2-[3- fluoro-6-(methylsulfonyl)pyridine-2-yl]ethan-1- amine hydrochloride | 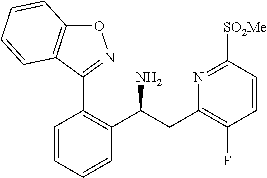 [SMILES: CS(=O)(=O)c1ccc(F)c(C[C@H](N)c2ccccc2-c2noc3ccccc23)n1] |
| (S)-1-[2-(Benzo[d]isoxazol-3-yl)phenyl]-2-(6- cyano-5-fluoropyridine-2-yl)ethan-1-amine Exemplary salt: (S)-1-[2-(Benzo[d]isoxazol-3-yl)phenyl]-2-(6- cyano-5-fluoropyridine-2-yl)ethan-1-amine hydrochloride | 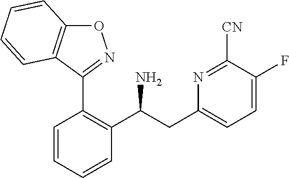 [SMILES: N#Cc1nc(C[C@H](N)c2ccccc2-c2noc3ccccc23)ccc1F] |
| (S)-1-[2-(Benzo[d]isoxazol-3-yl)phenyl]-2-(5- fluoro-6-methylsulfonylpyridine-2-yl)ethan-1- amine Exemplary salt: (S)-1-[2-(Benzo[d]isoxazol-3-yl)phenyl]-2-(5- fluoro-6-methylsulfonylpyridine-2-yl)ethan-1- amine hydrochloride | 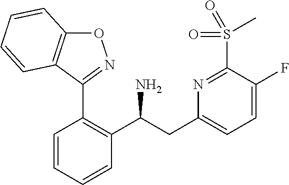 [SMILES: CS(=O)(=O)c1nc(C[C@H](N)c2ccccc2-c2noc3ccccc23)ccc1F] |
| (S)-6-{2-Amino-2-[2-(benzo[d]isoxazol-3- yl)phenyl]ethyl}-N,N,5-trimethylpyridine-2- carboxamide | 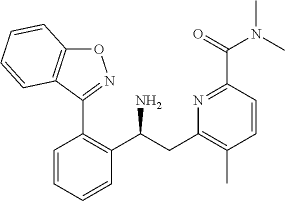 [SMILES: Cc1ccc(C(=O)N(C)C)nc1C[C@H](N)c1ccccc1-c1noc2ccccc12] |
| (S)-1-[2-(Benzo[d]isoxazol-3-yl)phenyl]-2-(6- ethyl-3-methylpyridine-2-yl)ethan-1-amine Exemplary salt: (S)-1-[2-(Benzo[d]isoxazol-3-yl)phenyl]-2-(6- ethyl-3-methylpyridine-2-yl)ethan-1-amine hydrochloride | 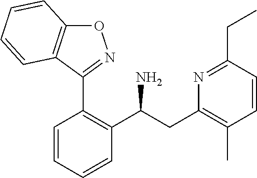 [SMILES: CCc1ccc(C)c(C[C@H](N)c2ccccc2-c2noc3ccccc23)n1] |
| (S)-2-(3-Fluoro-6-methylsulfonylpyridine-2-yl)-1- [2-(6-trifluoromethylbenzo[d]isoxazol-3- yl)phenyl]ethan-1-amine Exemplary salt: (S)-2-(3-Fluoro-6-methylsulfonylpyridine-2-yl)-1- [2-(6-trifluoromethylbenzo[d]isoxazol-3- yl)phenyl]ethan-1-amine hydrochloride | 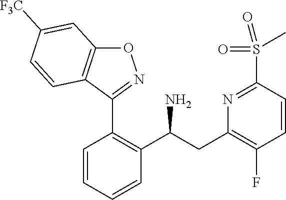 [SMILES: CS(=O)(=O)c1ccc(F)c(C[C@H](N)c2ccccc2-c2noc3cc(C(F)(F)F)ccc23)n1] |
| (S)-2-(3-Fluoro-6-methylsulfonylpyridine-2-yl)-1- [2-(6-methylbenzo[d]isoxazol-3-yl)phenyl]ethan- 1-amine Exemplary salt: (S)-2-(3-Fluoro-6-methylsulfonylpyridine-2-yl)-1- [2-(6-methylbenzo[d]isoxazol-3-yl)phenyl]ethan- 1-amine hydrochloride | 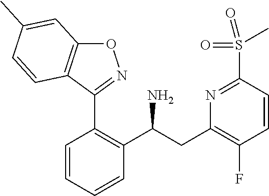 |
| (S)-2-(6-Cyano-3-fluoropyridine-2-yl)-1-[2-(6- methylbenzo[d]isoxazol-3-yl)phenyl]ethan-1- amine Exemplary salt: (S)-2-(6-Cyano-3-fluoropyridine-2-yl)-1-[2-(6- methylbenzo[d]isoxazol-3-yl)phenyl]ethan-1- amine hydrochloride | 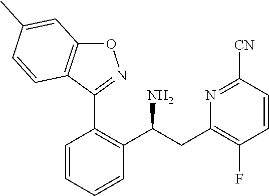 [SMILES: Cc1ccc2c(-c3ccccc3[C@@H](N)Cc3nc(C#N)ccc3F)noc2c1] |
| (S)-1-[2-(6-Chlorobenzo[d]isoxazol-3-yl)phenyl]- 2-(3-fluoro-6-methylsulfonylpyridine-2-yl)ethan- 1-amine Exemplary salt: (S)-1-[2-(6-Chlorobenzo[d]isoxazol-3-yl)phenyl]- 2-(3-fluoro-6-methylsulfonylpyridine-2-yl)ethan- 1-amine hydrochloride | 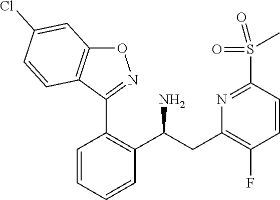 [SMILES: CS(=O)(=O)c1ccc(F)c(C[C@H](N)c2ccccc2-c2noc3cc(Cl)ccc23)n1] |
| (S)-1-[2-(6-Chlorobenzo[d]isoxazol-3-yl)phenyl]- 2-(6-cyano-3-fluoropyridine-2-yl)ethan-1-amine Exemplary salt: (S)-1-[2-(6-Chlorobenzo[d]isoxazol-3-yl)phenyl]- 2-(6-cyano-3-fluoropyridin-2-yl)ethan-1-amine hydrochloride | 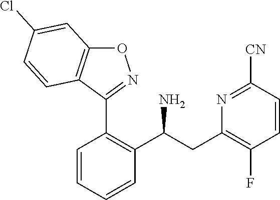 [SMILES: N#Cc1ccc(F)c(C[C@H](N)c2ccccc2-c2noc3cc(Cl)ccc23)n1] |
| (S)-2-(6-Bromo-3-fluoropyridine-2-yl)-1-[2-(6- fluorobenzo[d]isoxazol-3-yl)phenyl]-ethan-1- amine Exemplary salt: (S)-2-(6-Bromo-3-fluoropyridine-2-yl)-1-[2-(6- fluorobenzo[d]isoxazol-3-yl)phenyl]-ethan-1- amine hydrochloride | 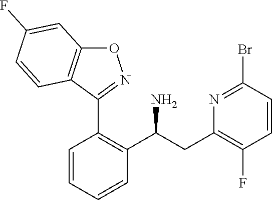 [SMILES: N[C@@H](Cc1nc(Br)ccc1F)c1ccccc1-c1noc2cc(F)ccc12] |
| (S)-2-(6-Bromo-3-fluoropyridine-2-yl)-1-[2-(6- bromobenzo[d]isoxazol-3-yl)phenyl]-ethan-1- amine | 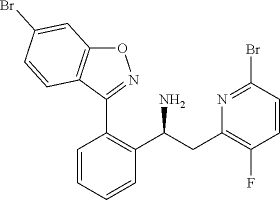 [SMILES: N[C@@H](Cc1nc(Br)ccc1F)c1ccccc1-c1noc2cc(Br)ccc12] |
| (S)-2-(6-Bromo-3-fluoropyridine-2-yl)-1-[2-(6- cyanobenzo[d]isoxazol-3-yl)phenyl]-ethan-1- amine Exemplary salt: (S)-2-(6-Bromo-3-fluoropyridine-2-yl)-1-[2-(6- cyanobenzo[d]isoxazol-3-yl)phenyl]-ethan-1- amine hydrochloride | 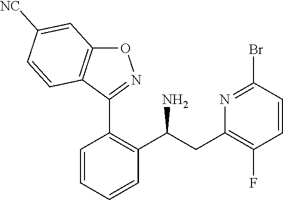 [SMILES: N#Cc1ccc2c(-c3ccccc3[C@@H](N)Cc3nc(Br)ccc3F)noc2c1] |
| (S)-6-{2-Amino-2-[2-(6-methylbenzo[d]isoxazol- 3-yl)phenyl]ethyl-2-cyano-5-methylpyridine Exemplary salt: (S)-6-{2-Amino-2-[2-(6-methylbenzo[d]isoxazol- 3-yl)phenyl]ethyl-2-cyano-5-methylpyridine hydrochloride | 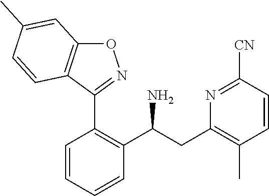 [SMILES: Cc1ccc2c(-c3ccccc3[C@@H](N)Cc3nc(C#N)ccc3C)noc2c1] |
| (S)-2-(3-Methyl-6-methylsulfonylpyridin-2-yl)-1- [2-(6-methylbenzo[d]isoxazol-3-yl)phenyl]ethan- 1-amine Exemplary salt: (S)-2-(3-Methyl-6-methylsulfonylpyridin-2-yl)-1- [2-(6-methylbenzo[d]isoxazol-3-yl)phenyl]ethan- 1-amine hydrochloride | 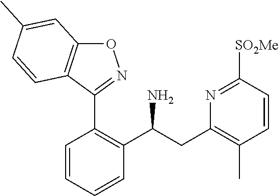 [SMILES: Cc1ccc2c(-c3ccccc3[C@@H](N)Cc3nc(S(C)(=O)=O)ccc3C)noc2c1] |
| (S)-2-(3-Fluoro-6-methylsulfonylpyridin-2-yl)-1- [2-(6-fluorobenzo[d]isoxazol-3-yl)phenyl]ethan- 1-amine Exemplary salt: (S)-2-(3-Fluoro-6-methylsulfonylpyridin-2-yl)-1- [2-(6-fluorobenzo[d]isoxazol-3-yl)phenyl]ethan- 1-amine hydrochloride | 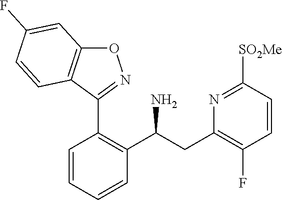 [SMILES: CS(=O)(=O)c1ccc(F)c(C[C@H](N)c2ccccc2-c2noc3cc(F)ccc23)n1] |
| (S)-1-[2-(6-Bromobenzo[d]isoxazol-3-yl)phenyl]- 2-(3-Fluoro-6-methylsulfonylpyridin-2-yl)ethan-1- amine Exemplary salt: (S)-1-[2-(6-Bromobenzo[d]isoxazol-3-yl)phenyl]- 2-(3-Fluoro-6-methylsulfonylpyridin-2-yl)ethan-1- amine hydrochloride | 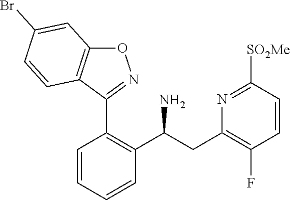 [SMILES: CS(=O)(=O)c1ccc(F)c(C[C@H](N)c2ccccc2-c2noc3cc(Br)ccc23)n1] |
| (S)-1-[2-(5-Fluorobenzo[d]isoxazol-3-yl)phenyl]- 2-(3-fluoro-6-methylsulfonylpyridin-2-yl)ethan-1- amine Exemplary salt: (S)-1-[2-(5-Fluorobenzo[d]isoxazol-3-yl)phenyl]- 2-(3-fluoro-6-methylsulfonylpyridin-2-yl)ethan-1- amine hydrochloride | 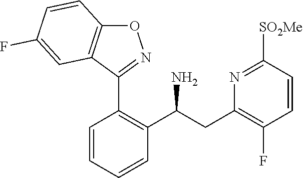 [SMILES: CS(=O)(=O)c1ccc(F)c(C[C@H](N)c2ccccc2-c2noc3ccc(F)cc23)n1] |
| (S)-1-[2-(6-Chlorobenzo[d]isoxazol-3-yl)phenyl]- 2-(3-methyl-6-methylsulfonylpyridin-2-yl)ethan- 1-amine Exemplary salt: (S)-1-[2-(6-Chlorobenzo[d]isoxazol-3-yl)phenyl]- 2-(3-methyl-6-methylsulfonylpyridin-2-yl)ethan- 1-amine hydrochloride | 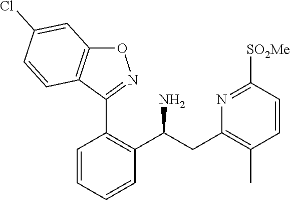 [SMILES: Cc1ccc(S(C)(=O)=O)nc1C[C@H](N)c1ccccc1-c1noc2cc(Cl)ccc12] |
| (S)-1-[2-(6-Fluorobenzo[d]isoxazol-3-yl)phenyl]- 2-(3-methyl-6-methylsulfonylpyridin-2-yl)ethan- 1-amine Exemplary salt: (S)-1-[2-(6-Fluorobenzo[d]isoxazol-3-yl)phenyl]- 2-(3-methyl-6-methylsulfonylpyridin-2-yl)ethan- 1-amine hydrochloride | 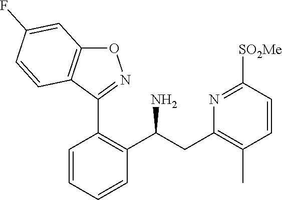 [SMILES: Cc1ccc(S(C)(=O)=O)nc1C[C@H](N)c1ccccc1-c1noc2cc(F)ccc12] |
| (S)-1-[2-(6-Chlorobenzo[d]isoxazol-3-yl)phenyl]- 2-(3-fluoro-6-isopropylsulfonylpyridin-2-yl)ethan- 1-amine Exemplary salt: (S)-1-[2-(6-Chlorobenzo[d]isoxazol-3-yl)phenyl]- 2-(3-fluoro-6-isopropylsulfonylpyridin-2-yl)ethan- 1-amine hydrochloride | 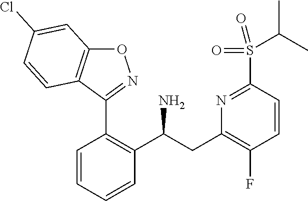 [SMILES: CC(C)S(=O)(=O)c1ccc(F)c(C[C@H](N)c2ccccc2-c2noc3cc(Cl)ccc23)n1] |
| (S)-1-[2-(7-Fluorobenzo[d]isoxazol-3-yl)phenyl]- 2-(3-fluoro-6-methylsulfonylpyridin-2-yl)ethan-1- amine Exemplary salt: (S)-1-[2-(7-Fluorobenzo[d]isoxazol-3-yl)phenyl]- 2-(3-fluoro-6-methylsulfonylpyridin-2-yl)ethan-1- amine hydrochloride | 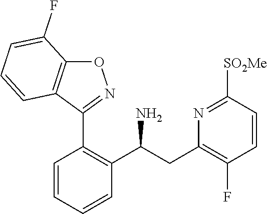 [SMILES: CS(=O)(=O)c1ccc(F)c(C[C@H](N)c2ccccc2-c2noc3c(F)cccc23)n1] |
| (S)-2-(2-Amino-2-[2-(6-bromobenzo[d]isoxazol- 3-yl)phenyl]ethyl}-3-fluoropyridine-6-carbonitrile | 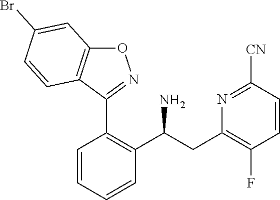 [SMILES: N#Cc1ccc(F)c(C[C@H](N)c2ccccc2-c2noc3cc(Br)ccc23)n1] |
| (S)-2-(2-Amino-2-[2-(6-bromobenzo[d]isoxazol- 3-yl)phenyl]ethyl}-3-methylpyridine-6-carbonitrile | 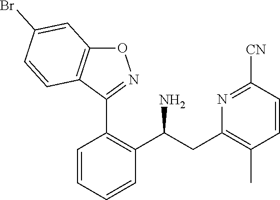 [SMILES: Cc1ccc(C#N)nc1C[C@H](N)c1ccccc1-c1noc2cc(Br)ccc12] |
| (S)-1-[2-(6-Cyanobenzo[d]isoxazol-3-yl)phenyl]- 2-(pyridine-2-yl)ethan-1-amine | 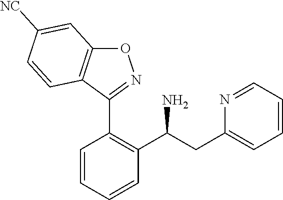 [SMILES: N#Cc1ccc2c(-c3ccccc3[C@@H](N)Cc3ccccn3)noc2c1] |
| (S)-1-[2-(6-Hyroxymethylbenzo[d]isoxazol-3- yl)phenyl]-2-(pyridine-2-yl)ethan-1-amine Exemplary salt: (S)-1-[2-(6-Hyroxymethylbenzo[d]isoxazol-3- yl)phenyl]-2-(pyridine-2-yl)ethan-1-amine hydrochloride | 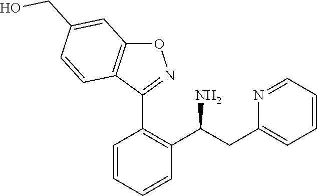 [SMILES: N[C@@H](Cc1ccccn1)c1ccccc1-c1noc2cc(CO)ccc12] |
| (S)-1-[2-(6-Ethynylbenzo[d]isoxazol-3-yl)phenyl]- 2-(pyridine-2-yl)ethan-1-amine Exemplary salt: (S)-1-[2-(6-Ethynylbenzo[d]isoxazol-3-yl)phenyl]- 2-(pyridine-2-yl)ethan-1-amine hydrochloride | 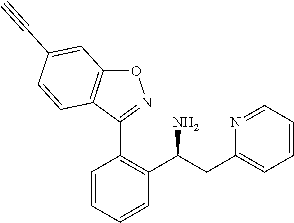 [SMILES: C#Cc1ccc2c(-c3ccccc3[C@@H](N)Cc3ccccn3)noc2c1] |
| (S)-1-[2-(6-[3-Hydroxyprop-1-yn-1- yl]benzo[d]isoxazol-3-yl)phenyl]-2-(pyridine-2- yl)ethan-1-amine Exemplary salt: (S)-1-[2-(6-[3-Hydroxyprop-1-yn-1- yl]benzo[d]isoxazol-3-yl)phenyl]-2-(pyridine-2- yl)ethan-1-amine hydrochloride | 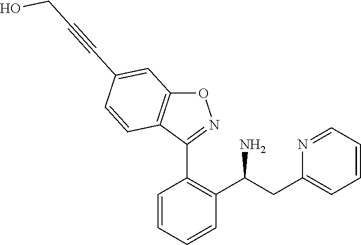 [SMILES: N[C@@H](Cc1ccccn1)c1ccccc1-c1noc2cc(C#CCO)ccc12] |
| (S)-1-[2-(6-Methylbenzo[d]isoxazol-3-yl)phenyl]- 2-(pyridine-2-yl)ethan-1-amine | 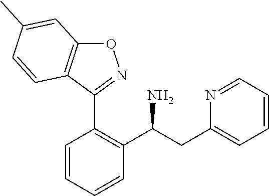 [SMILES: Cc1ccc2c(-c3ccccc3[C@@H](N)Cc3ccccn3)noc2c1] |
| (S)-1-[2-(6-Cyclopropylbenzo[d]isoxazol-3- yl)phenyl]-2-(pyridine-2-yl)ethan-1-amine | 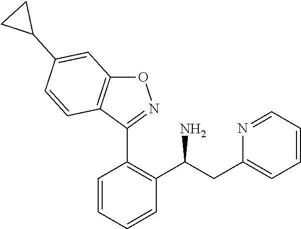 [SMILES: N[C@@H](Cc1ccccn1)c1ccccc1-c1noc2cc(C3CC3)ccc12] |
| (S)-1-[2-(6-[1-Hydroxyethyl]benzo[d]isoxazol-3- yl)phenyl]-2-(pyridine-2-yl)ethan-1-amine | 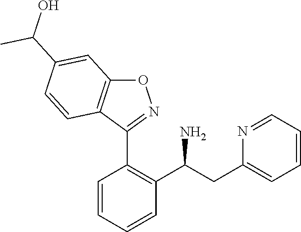 [SMILES: CC(O)c1ccc2c(-c3ccccc3[C@@H](N)Cc3ccccn3)noc2c1] |
| (S)-1-[2-(Benzo[d]isoxazol-3-yl)-5- methylphenyl]-2-(pyridine-2-yl)ethan-1-amine Exemplary salt: (S)-1-[2-(Benzo[d]isoxazol-3-yl)-5- methylphenyl]-2-(pyridine-2-yl)ethan-1-amine hydrochloride | 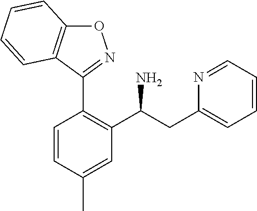 [SMILES: Cc1ccc(-c2noc3ccccc23)c([C@@H](N)Cc2ccccn2)c1] |
| (S)-1-[2-(Benzo[d]isoxazol-3-yl)-5-cyanophenyl]- 2-(pyridine-2-yl)ethan-1-amine Exemplary salt: (S)-1-[2-(Benzo[d]isoxazol-3-yl)-5-cyanophenyl]- 2-(pyridine-2-yl)ethan-1-amine hydrochloride | 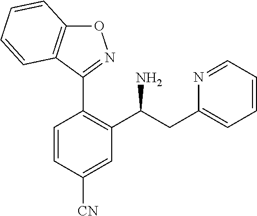 [SMILES: N#Cc1ccc(-c2noc3ccccc23)c([C@@H](N)Cc2ccccn2)c1] |
| (S)-1-[2-(Benzo[d]isoxazol-3-yl)-4- methylphenyl]-2-(pyridine-2-yl)ethan-1-amine Exemplary salt: (S)-1-[2-(Benzo[d]isoxazol-3-yl)-4- methylphenyl]-2-(pyridine-2-yl)ethan-1-amine hydrochloride | 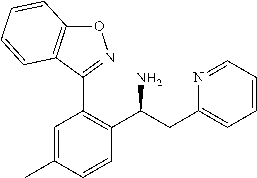 [SMILES: Cc1ccc([C@@H](N)Cc2ccccn2)c(-c2noc3ccccc23)c1] |
| (S)-1-[2-(6-Methoxymethylbenzo[d]isoxazol-3- yl)phenyl]-2-(pyridine-2-yl)ethan-1-amine | 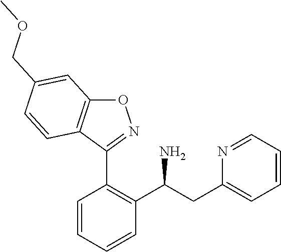 [SMILES: COCc1ccc2c(-c3ccccc3[C@@H](N)Cc3ccccn3)noc2c1] |
| (S)-1-[2-(6-[2-Hydroxyethyl]benzo[d]isoxazol-3- yl)phenyl]-2-(pyridine-2-yl)ethan-1-amine | 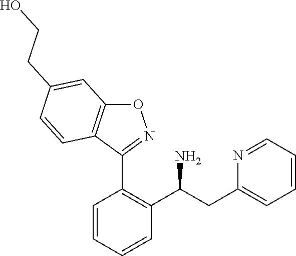 [SMILES: N[C@@H](Cc1ccccn1)c1ccccc1-c1noc2cc(CCO)ccc12] |
| 1-(6-{(S)-2-Amino-2-[2-(benzo[d]isoxazol-3- yl)phenyl]ethyl}pyridine-2-yl)ethan-1,2-diol Exemplary salt: 1-(6-{(S)-2-Amino-2-[2-(benzo[d]isoxazol-3- yl)phenyl]ethyl}pyridine-2-yl)ethan-1,2-diol hydrochloride | 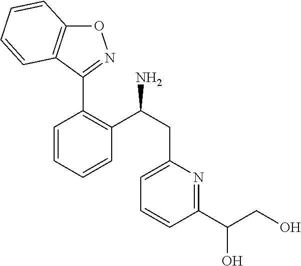 [SMILES: N[C@@H](Cc1cccc(C(O)CO)n1)c1ccccc1-c1noc2ccccc12] |
| (S)-1-[2-(Benzo[d]isoxazol-3-yl)phenyl]-2- (pyridine-2-yl)ethan-1-amine Exemplary salt: (S)-1-[2-(Benzo[d]isoxazol-3-yl)phenyl]-2- (pyridine-2-yl)ethan-1-amine hydrochloride | 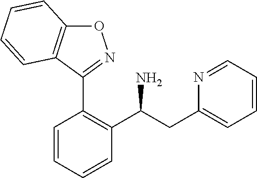 [SMILES: N[C@@H](Cc1ccccn1)c1ccccc1-c1noc2ccccc12] |
| (S)-1-[2-(4-Bromobenzo[d]isoxazol-3-yl)phenyl]- 2-(pyridine-2-yl)ethan-1-amine Exemplary salt: (S)-1-[2-(4-Bromobenzo[d]isoxazol-3-yl)phenyl]- 2-(pyridine-2-yl)ethan-1-amine hydrochloride | 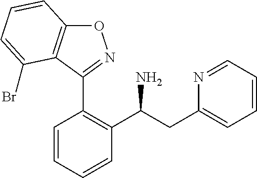 [SMILES: N[C@@H](Cc1ccccn1)c1ccccc1-c1noc2cccc(Br)c12] |
| (S)-1-[2-(Benzo[d]isoxazol-3-yl)phenyl]-2-(6- bromopyridine-2-yl)ethan-1-amine Exemplary salt: (S)-1-[2-(Benzo[d]isoxazol-3-yl)phenyl]-2-(6- bromopyridine-2-yl)ethan-1-amine hydrochloride | 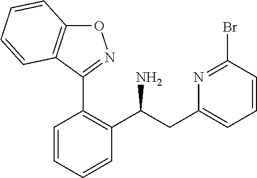 [SMILES: N[C@@H](Cc1cccc(Br)n1)c1ccccc1-c1noc2ccccc12] |
| (1S,2S)-1-[2-(Benzo[d]isoxazol-3-yl)phenyl]-2- (pyridin-2-yl)butan-1-amine and (1S,2R)-1-[2- (benzo[d]isoxazol-3-yl)phenyl]-2-(pyridin-2- yl)butan-1-amine Exemplary salt: (1S,2S)-1-[2-(Benzo[d]isoxazol-3-yl)phenyl]-2- (pyridin-2-yl)butan-1-amine hydrochloride and (1S,2R)-1-[2-(benzo[d]isoxazol-3-yl)phenyl]-2- (pyridin-2-yl)butan-1-amine hydrochloride | 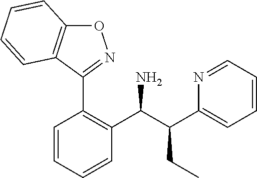 [SMILES: CC[C@H](c1ccccn1)[C@H](N)c1ccccc1-c1noc2ccccc12] |
| | 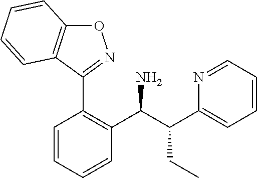 [SMILES: CC[C@@H](c1ccccn1)[C@H](N)c1ccccc1-c1noc2ccccc12] |
| (S)-1-[2-(Benzo[d]isoxazol-3-yl)phenyl]-2-(3- methylpyridine-2-yl)ethan-1-amine Exemplary salt: (S)-1-[2-(Benzo[d]isoxazol-3-yl)phenyl]-2-(3- methylpyridine-2-yl)ethan-1-amine hydrochloride | 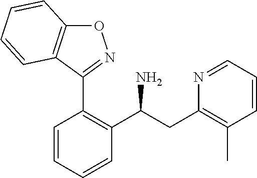 [SMILES: Cc1cccnc1C[C@H](N)c1ccccc1-c1noc2ccccc12] |
| (S)-1-[2-(Benzo[d]isoxazol-3-yl)phenyl]-3- methyl-2-(pyridine-2-yl)butan-1-amine Exemplary salt: (S)-1-[2-(Benzo[d]isoxazol-3-yl)phenyl]-3- methyl-2-(pyridine-2-yl)butan-1-amine hydrochloride | 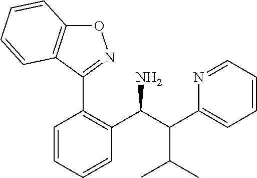 [SMILES: CC(C)C(c1ccccn1)[C@H](N)c1ccccc1-c1noc2ccccc12] |
| (R)-1-[2-(Benzo[d]isoxazol-3-yl)phenyl]-2- methyl-2-(pyridine-2-yl)propane-1-amine Exemplary salt: (R)-1-[2-(Benzo[d]isoxazol-3-yl)phenyl]-2- methyl-2-(pyridine-2-yl)propane-1-amine hydrochloride | 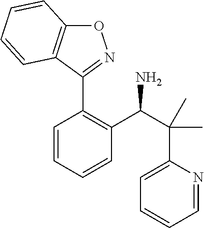 [SMILES: CC(C)(c1ccccn1)[C@H](N)c1ccccc1-c1noc2ccccc12] |
| (R)-1-(6-((S)-2-Amino-2-[2-(benzo[d]isoxazol-3- yl)phenyl]ethyl}pyridine-2-yl)pyrrolidin-3-ol Exemplary salt: (R)-1-(6-((S)-2-Amino-2-[2-(benzo[d]isoxazol-3- yl)phenyl]ethyl}pyridine-2-yl)pyrrolidin-3-ol hydrochloride | 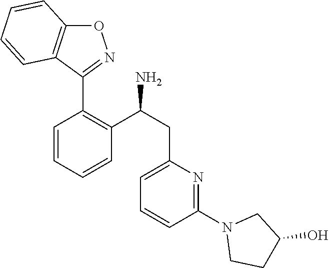 [SMILES: N[C@@H](Cc1cccc(N2CC[C@@H](O)C2)n1)c1ccccc1-c1noc2ccccc12] |
| (S)-6-(2-Amino-2-[2-(benzo[d]isoxazol-3- yl)phenyl]ethyl}-N-(2-methoxyethyl)-N- methylpyridin-2-amine Exemplary salt: (S)-6-{2-Amino-2-[2-(benzo[d]isoxazol-3- yl)phenyl]ethyl}-N-(2-methoxyethyl)-N- methylpyridin-2-amine hydrochloride | 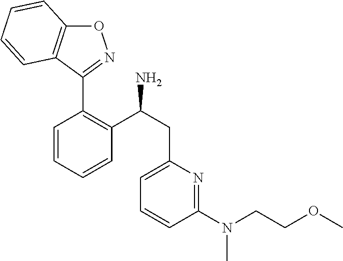 [SMILES: COCCN(C)c1cccc(C[C@H](N)c2ccccc2-c2noc3ccccc23)n1] |
| (S)-6-{2-Amino-2-[2-(benzo[d]isoxazol-3- yl)phenyl]ethyl}-N-(2-methoxyethyl)pyridin-2- amine Exemplary salt: (S)-6-{2-Amino-2-[2-(benzo[d]isoxazol-3- yl)phenyl]ethyl}-N-(2-methoxyethyl)pyridin-2- amine hydrochloride | 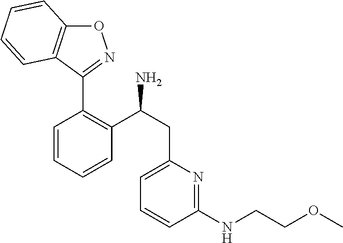 [SMILES: COCCNc1cccc(C[C@H](N)c2ccccc2-c2noc3ccccc23)n1] |
| (S)-1-(6-((S)-2-Amino-2-[2-(benzo[d]isoxazol-3- yl)phenyl]ethyl}pyridine-2-yl)pyrrolidin-3-ol Exemplary salt: (S)-1-(6-{(S)-2-Amino-2-[2-(benzo[d]isoxazol-3- yl)phenyl]ethyl}pyridine-2-yl)pyrrolidin-3-ol hydrochloride | 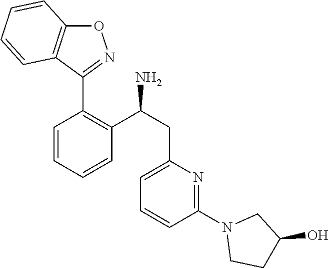 [SMILES: N[C@@H](Cc1cccc(N2CC[C@H](O)C2)n1)c1ccccc1-c1noc2ccccc12] |
| (S)-1-[2-(Benzo[d]isoxazol-3-yl)phenyl]-2-(6- morpholinopyridine-2-yl)ethane-1-amine Exemplary salt: (S)-1-[2-(Benzo[d]isoxazol-3-yl)phenyl]-2-(6- morpholinopyridine-2-yl)ethane-1-amine hydrochloride | 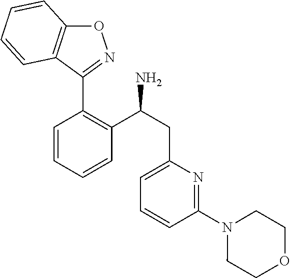 [SMILES: N[C@@H](Cc1cccc(N2CCOCC2)n1)c1ccccc1-c1noc2ccccc12] |
| (S)-2-[(6-{(S)-2-Amino-2-[2-(benzo[d]isoxazol-3- yl)phenyl]ethyl}pyridine-2- yl)(methyl)amino]ethan-1-ol Exemplary salt: (S)-2-[(6-{(S)-2-Amino-2-[2-(benzo[d]isoxazol-3- yl)phenyl]ethyl}pyridine-2- yl)(methyl)amino]ethan-1-ol hydrochloride | 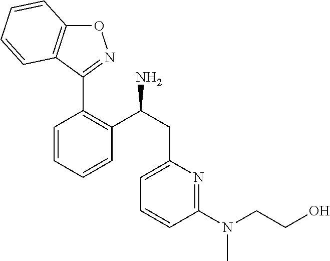 [SMILES: CN(CCO)c1cccc(C[C@H](N)c2ccccc2-c2noc3ccccc23)n1] |
| (S)-1-[2-(benzo[d]isoxazol-3-yl)phenyl]-2- (pyrimidine-2-yl)ethan-1-amine | 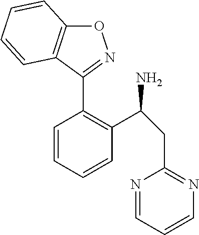 [SMILES: N[C@@H](Cc1ncccn1)c1ccccc1-c1noc2ccccc12] |
| (S)-1-[2-(Benzo[d]isoxazol-3-yl)phenyl]-2-(6- bromo-3-methylpyridin-2-yl)ethan-1-amine Exemplary salt: (S)-1-[2-(Benzo[d]isoxazol-3-yl)phenyl]-2-(6- bromo-3-methylpyridin-2-yl)ethan-1-amine hydrochloride | 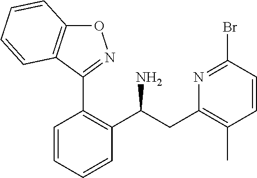 [SMILES: Cc1ccc(Br)nc1C[C@H](N)c1ccccc1-c1noc2ccccc12] |
| (S)-1-[2-(Benzo[d]isoxazol-3-yl)phenyl]-2-(6- cyano-3-methylpyridin-2-yl)ethan-1-amine | 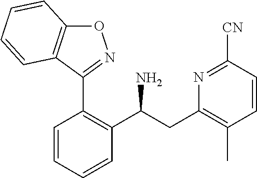 [SMILES: Cc1ccc(C#N)nc1C[C@H](N)c1ccccc1-c1noc2ccccc12] |
| (S)-1-[2-(Benzo[d]isoxazol-3-yl)phenyl]-2-(3,6- dimethylpyridin-2-yl)ethan-1-amine | 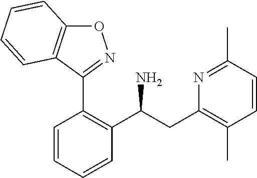 [SMILES: Cc1ccc(C)c(C[C@H](N)c2ccccc2-c2noc3ccccc23)n1] |
| (S)-1-[2-(1-Methyl-1H-indazole-3-yl)phenyl]-2- (pyridine-2-yl)ethan-1-amine Exemplary salt: (S)-1-[2-(1-Methyl-1H-indazole-3-yl)phenyl]-2- (pyridine-2-yl)ethan-1-amine hydrochloride | 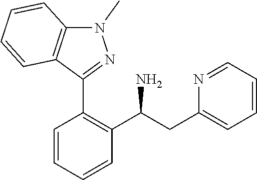 [SMILES: Cn1nc(-c2ccccc2[C@@H](N)Cc2ccccn2)c2ccccc21] |
| (S)-1-[2-(1-Methyl-1H-indazole-3-yl)phenyl]-2-(6- methylpyridine-2-yl)ethan-1-amine Exemplary salt: (S)-1-[2-(1-Methyl-1H-indazole-3-yl)phenyl]-2-(6- methylpyridine-2-yl)ethan-1-amine hydrochloride | 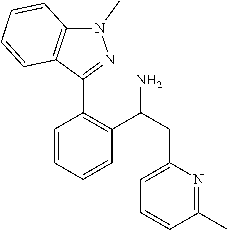 [SMILES: Cc1cccc(CC(N)c2ccccc2-c2nn(C)c3ccccc23)n1] |
| (S)-1-[2-(Benzo[d]isoxazol-3-yl)phenyl]-2-(6- trifluoromethylpyridin-2-yl)ethan-1-amine Exemplary salt: (S)-1-[2-(Benzo[d]isoxazol-3-yl)phenyl]-2-(6- trifluoromethylpyridin-2-yl)ethan-1-amine hydrochloride | 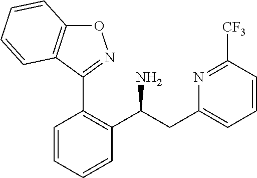 [SMILES: N[C@@H](Cc1cccc(C(F)(F)F)n1)c1ccccc1-c1noc2ccccc12] |
| (S)-1-[2-(Benzo[d]isoxazol-3-yl)phenyl]-2-(3- fluoropyridin-2-yl)ethan-1-amine Exemplary salt: (S)-1-[2-(Benzo[d]isoxazol-3-yl)phenyl]-2-(3- fluoropyridin-2-yl)ethan-1-amine hydrochloride | 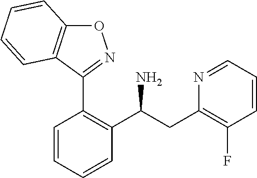 [SMILES: N[C@@H](Cc1ncccc1F)c1ccccc1-c1noc2ccccc12] |
| (S)-2-(6-Bromopyridine-2-yl)-1-[2-(1-isopropyl- 1H-indazole-3-yl)phenyl]ethan-1-amine Exemplary salt: (S)-2-(6-Bromopyridine-2-yl)-1-[2-(1-isopropyl- 1H-indazole-3-yl)phenyl]ethan-1-amine hydrochloride | 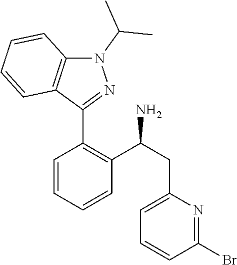 [SMILES: CC(C)n1nc(-c2ccccc2[C@@H](N)Cc2cccc(Br)n2)c2ccccc21] |
| (S)-1-[2-(1-isopropyl-1H-indazole-3-yl)phenyl]-2- (pyridine-2-yl)ethan-1-amine Exemplary salt: (S)-1-[2-(1-isopropyl-1H-indazole-3-yl)phenyl]-2- (pyridine-2-yl)ethan-1-amine hydrochloride | 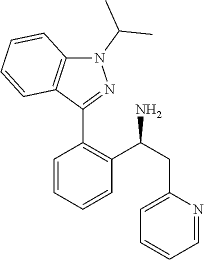 [SMILES: CC(C)n1nc(-c2ccccc2[C@@H](N)Cc2ccccn2)c2ccccc21] |
| (S)-1-[2-(1-isopropyl-1H-indazole-3-yl)phenyl]-2- (6-methylpyridine-2-yl)ethan-1-amine Exemplary salt: (S)-1-[2-(1-Isopropyl-1H-indazole-3-yl)phenyl]-2- (6-methylpyridine-2-yl)ethan-1-amine hydrochloride | 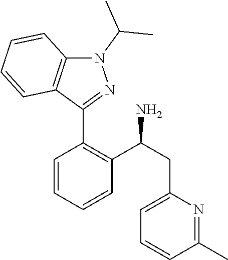 [SMILES: Cc1cccc(C[C@H](N)c2ccccc2-c2nn(C(C)C)c3ccccc23)n1] |
| (S)-2-(6-Cyanopyridine-2-yl)-1-[2-(1-isopropyl- 1H-indazol-3-yl)phenyl]ethan-1-amine | 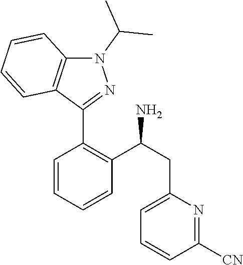 [SMILES: CC(C)n1nc(-c2ccccc2[C@@H](N)Cc2cccc(C#N)n2)c2ccccc21] |
| (S)-1-[2-(Benzo[d]isoxazol-3-yl)phenyl]-2-(6- fluoropyridin-2-yl)ethan-1-amine Exemplary salt: (S)-1-[2-(Benzo[d]isoxazol-3-yl)phenyl]-2-(6- fluoropyridin-2-yl)ethan-1-amine hydrochloride | 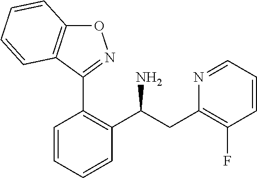 [SMILES: N[C@@H](Cc1ncccc1F)c1ccccc1-c1noc2ccccc12] |
| (S)-1-[2-(Benzo[d]isoxazol-3-yl)phenyl]-2-(5- fluoropyridin-2-yl)ethan-1-amine Exemplary salt: (S)-1-[2-(Benzo[d]isoxazol-3-yl)phenyl]-2-(5- fluoropyridin-2-yl)ethan-1-amine hydrochloride | 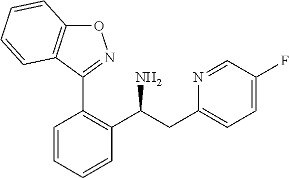 [SMILES: N[C@@H](Cc1ccc(F)cn1)c1ccccc1-c1noc2ccccc12] |
| (S)-1-[2-(Benzo[d]isoxazol-3-yl)phenyl]-2-(4- methoxypyridin-2-yl)ethan-1-amine | 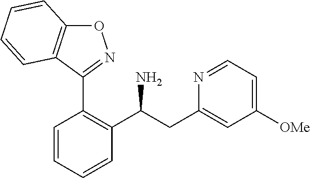 [SMILES: COc1ccnc(C[C@H](N)c2ccccc2-c2noc3ccccc23)c1] |
| (S)-1-[2-(Benzo[d]isoxazol-3-yl)phenyl]-2-(6- cyano-3-fluoropyridin-2-yl)ethan-1-amine Exemplary salt: (S)-1-[2-(Benzo[d]isoxazol-3-yl)phenyl]-2-(6- cyano-3-fluoropyridin-2-yl)ethan-1-amine formate | 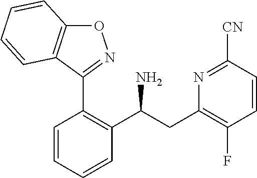 [SMILES: N#Cc1ccc(F)c(C[C@H](N)c2ccccc2-c2noc3ccccc23)n1] |
| (S)-6-{2-Amino-2-[2-(benzo[d]isoxazol-3- yl)phenyl]ethyl}-5-fluoropyridine-2-carboxamide Exemplary salt: (S)-6-{2-Amino-2-[2-(benzo[d]isoxazol-3- yl)phenyl]ethyl}-5-fluoropyridine-2-carboxamide formate | 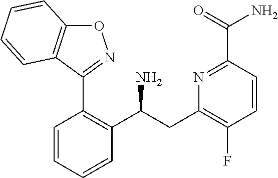 [SMILES: NC(=O)c1ccc(F)c(C[C@H](N)c2ccccc2-c2noc3ccccc23)n1] |
| (S)-1-{[2-(Benzo[d]isoxazol-3-yl)phenyl]-2-(3- fluoro-6-methylpyridin-2-yl)ethan-1-amine Exemplary salt: (S)-1-[2-(Benzo[d]isoxazol-3-yl)phenyl]-2-(3- fluoro-6-methylpyridin-2-yl)ethan-1-amine hydrochlroide | 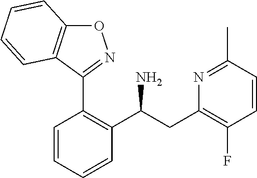 [SMILES: Cc1ccc(F)c(C[C@H](N)c2ccccc2-c2noc3ccccc23)n1] |
| (S)-6-{2-Amino-2-[2-(benzo[d]isoxazol-3- yl)phenyl]ethyl}-5-fluoro-N,N-dimethylpyridin-2- carboxamide Exemplary salt: (S)-6-{2-Amino-2-[2-(benzo[d]isoxazol-3- yl)phenyl]ethyl}-5-fluoro-N,N-dimethylpyridin-2- carboxamide hydrochloride | 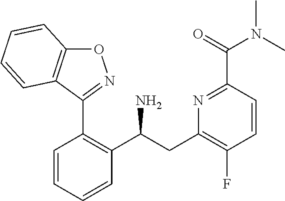 [SMILES: CN(C)C(=O)c1ccc(F)c(C[C@H](N)c2ccccc2-c2noc3ccccc23)n1] |
| (S)-1-[2-(Benzo[d]isoxazol-3-yl)phenyl]-2-(3- cyanopyridin-2-yl)ethan-1-amine | 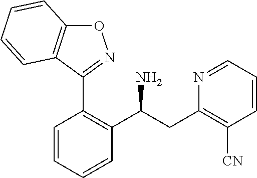 [SMILES: N#Cc1cccnc1C[C@H](N)c1ccccc1-c1noc2ccccc12] |
| (S)-N-(2-{2-Amino-2-[2-(benzo[d]isoxazol-3- yl)phenyl]ethyl}pyridin-3-yl)acetamide Exemplary salt: (S)-N-(2-{2-Amino-2-[2-(benzo[d]isoxazol-3- yl)phenyl]ethyl}pyridin-3-yl)acetamide hydrochloride | 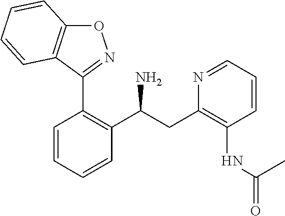 [SMILES: CC(=O)Nc1cccnc1C[C@H](N)c1ccccc1-c1noc2ccccc12] |
| (S)-2-(6-{2-Amino-2-(2-benzo[d]isoxazol-3- yl)phenyl]ethyl}-5-fluoropyridin-2-yl)ethan-1-ol Exemplary salt: (S)-2-(6-{2-Amino-2-(2-benzo[d]isoxazol-3- yl)phenyl]ethyl}-5-fluoropyridin-2-yl)ethan-1-ol hydrochloride | 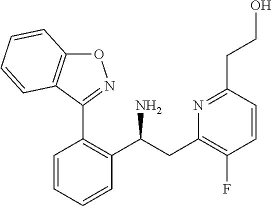 [SMILES: N[C@@H](Cc1nc(CCO)ccc1F)c1ccccc1-c1noc2ccccc12] |
| (S)-1-[2-(Benzo[d]isoxazol-3-yl)phenyl]-2-[3- fluoro-6-(1H-pyrazol-4-yl)pyridin-2-yl)ethan-1- amine Exemplary salt: (S)-1-[2-(Benzo[d]isoxazol-3-yl)phenyl]-2-[3- fluoro-6-(1H-pyrazol-4-yl)pyridin-2-yl)ethan-1- amine hydrochloride | 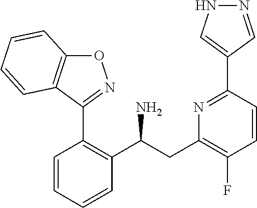 [SMILES: N[C@@H](Cc1nc(-c2cn[nH]c2)ccc1F)c1ccccc1-c1noc2ccccc12] |
| (S)-1-[2-(Benzo[d]isoxazol-3-yl)phenyl]-2-(3- cyclopropylpyridin-2-yl)ethan-1-amine Exemplary salt: (S)-1-[2-(Benzo[d]isoxazol-3-yl)phenyl]-2-(3- cyclopropylpyridin-2-yl)ethan-1-amine formate | 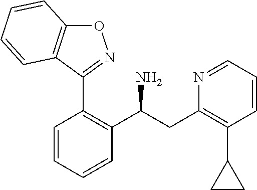 [SMILES: N[C@@H](Cc1ncccc1C1CC1)c1ccccc1-c1noc2ccccc12] |
| (S)-1-[2-(Benzo[d]isoxazol-3-yl)phenyl]-2-(3- methyl-5-cyanopyridin-2-yl)ethan-1-amine | 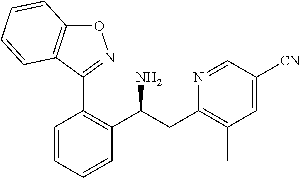 [SMILES: Cc1cc(C#N)cnc1C[C@H](N)c1ccccc1-c1noc2ccccc12] |
| (S)-1-[2-(6-Bromobenzo[d]isoxazol-3-yl)phenyl]- 2-(5-cyanopyridine-2-yl)ethan-1-amine | 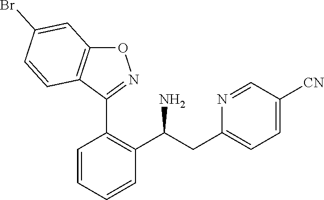 [SMILES: N#Cc1ccc(C[C@H](N)c2ccccc2-c2noc3cc(Br)ccc23)nc1] |
| (S)-1-[2-(Benzo[d]isoxazol-3-yl)-5- methoxyphenyl]-2-(pyridine-2-yl)ethan-1-amine Exemplary salt: (S)-1-[2-(Benzo[d]isoxazol-3-yl)-5- methoxyphenyl]-2-(pyridine-2-yl)ethan-1-amine hydrochloride | 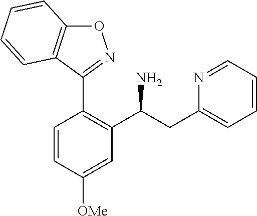 [SMILES: COc1ccc(-c2noc3ccccc23)c([C@@H](N)Cc2ccccn2)c1] |
| (S)-1-[2-(Benzo[d]isoxazol-3-yl)-5-bromophenyl]- 2-(pyridine-2-yl)ethan-1-amine Exemplary salt: (S)-1-[2-(Benzo[d]isoxazol-3-yl)-5-bromophenyl]- 2-(pyridine-2-yl)ethan-1-amine hydrochloride | 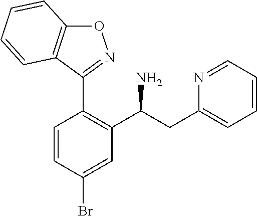 [SMILES: N[C@@H](Cc1ccccn1)c1cc(Br)ccc1-c1noc2ccccc12] |
| (S)-1-[2-(Benzo[d]isoxazol-3-yl)-6- methylphenyl]-2-(pyridine-2-yl)ethan-1-amine Exemplary salt: (S)-1-[2-(Benzo[d]isoxazol-3-yl)-6- methylphenyl]-2-(pyridine-2-yl)ethan-1-amine hydrochloride | 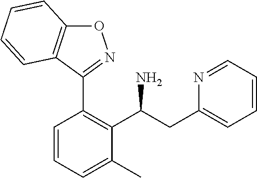 [SMILES: Cc1cccc(-c2noc3ccccc23)c1[C@@H](N)Cc1ccccn1] |
| (S)-1-[2-(Benzo[d]isoxazol-3-yl)-4-bromophenyl]- 2-(pyridine-2-yl)ethan-1-amine Exemplary salt: (S)-1-[2-(Benzo[d]isoxazol-3-yl)-4-bromophenyl]- 2-(pyridine-2-yl)ethan-1-amine hydrochlroide | 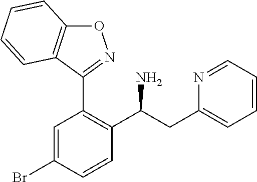 [SMILES: N[C@@H](Cc1ccccn1)c1ccc(Br)cc1-c1noc2ccccc12] |
| (S)-1-[2-(Benzo[d]isoxazol-3-yl)-3-bromophenyl]- 2-(pyridine-2-yl)ethan-1-amine Exemplary salt: (S)-1-[2-(Benzo[d]isoxazol-3-yl)-3-bromophenyl]- 2-(pyridine-2-yl)ethan-1-amine hydrochloride | 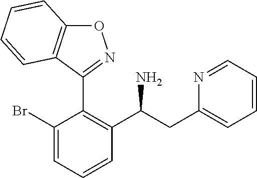 [SMILES: N[C@@H](Cc1ccccn1)c1cccc(Br)c1-c1noc2ccccc12] |
| (S)-1-[2-(Benzo[d]isoxazol-3-yl)-6-bromophenyl]- 2-(pyridine-2-yl)ethan-1-amine Exemplary salt: (S)-1-[2-(Benzo[d]isoxazol-3-yl)-6-bromophenyl]- 2-(pyridine-2-yl)ethan-1-amine hydrochloride | 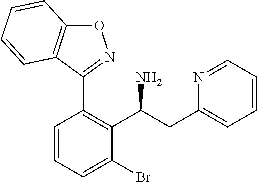 [SMILES: N[C@@H](Cc1ccccn1)c1c(Br)cccc1-c1noc2ccccc12] |
| (S)-1-[2-(Benzo[d]isoxazol-3-yl)phenyl]-2-(5- bromopyridine-2-yl)ethan-1-amine Exemplary salt: (S)-1-[2-(Benzo[d]isoxazol-3-yl)phenyl]-2-(5- bromopyridine-2-yl)ethan-1-amine hydrochloride | 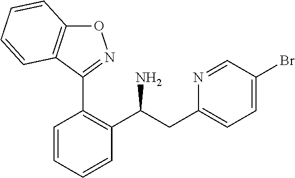 [SMILES: N[C@@H](Cc1ccc(Br)cn1)c1ccccc1-c1noc2ccccc12] |
| (S)-6-{2-Amino-2-[2-(Benzo[d]isoxazol-3- yl)phenyl]ethyl-N,N-dimethylpyridine-2- carboxamide Exemplary salt: (S)-6-{2-Amino-2-[2-(Benzo[d]isoxazol-3- yl)phenyl]ethyl-N,N-dimethylpyridine-2- carboxamide hydrochloride | 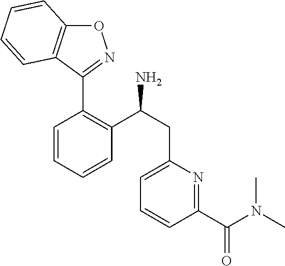 [SMILES: CN(C)C(=O)c1cccc(C[C@H](N)c2ccccc2-c2noc3ccccc23)n1] |
| (S)-1-[2-(Benzo[d]isoxazol-3-yl)phenyl]-2-[6-(1- methyl-1H-pyrazol-4-yl)pyridine-2-yl]ethan-1- amine Exemplary salt: (S)-1-[2-(Benzo[d]isoxazol-3-yl)phenyl]-2-[6-(1- methyl-1H-pyrazol-4-yl)pyridine-2-yl]ethan-1- amine hydrochloride | 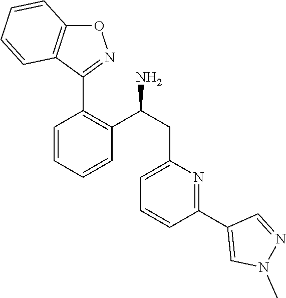 [SMILES: Cn1cc(-c2cccc(C[C@H](N)c3ccccc3-c3noc4ccccc34)n2)cn1] |
| (S)-2-[6-(1H-Pyrazol-4-yl)pyridine-2-yl])-1-[2- (benzo[d]isoxazol-3-yl)phenyl]ethan-1-amine Exemplary salt: (S)-2-[6-(1H-Pyrazol-4-yl)pyridine-2-yl])-1-[2- (benzo[d]isoxazol-3-yl)phenyl]ethan-1-amine hydrochloride | 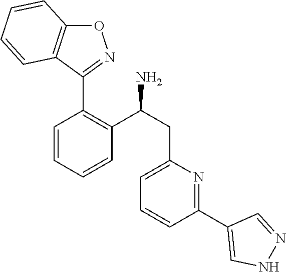 [SMILES: N[C@@H](Cc1cccc(-c2cn[nH]c2)n1)c1ccccc1-c1noc2ccccc12] |
| (S)-1-[2-(Benzo[d]isoxazol-3-yl)phenyl]-2-(5- methylpyridine-2-yl)ethan-1-amine Exemplary salt: (S)-1-[2-(Benzo[d]isoxazol-3-yl)phenyl]-2-(5- methylpyridine-2-yl)ethan-1-amine hydrochloride | 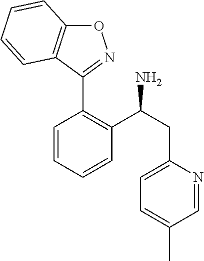 [SMILES: Cc1ccc(C[C@H](N)c2ccccc2-c2noc3ccccc23)nc1] |
| (S)-1-[2-(Benzo[d]isoxazol-3-yl)phenyl]-2-(5- cyanopyridine-2-yl)ethan-1-amine Exemplary salt: (S)-1-[2-(Benzo[d]isoxazol-3-yl)phenyl]-2-(5- cyanopyridine-2-yl)ethan-1-amine hydrochloride | 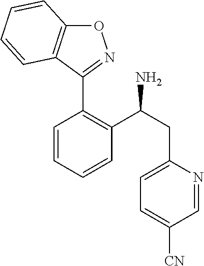 [SMILES: N#Cc1ccc(C[C@H](N)c2ccccc2-c2noc3ccccc23)nc1] |
| (S)-1-[2-(Benzo[d]isoxazol-3-yl)phenyl]-2-[6- (3,5-dimethylisoxazol-4-yl)pyridine-2-yl)-ethan-1- amine Exemplary salt: (S)-1-[2-(Benzo[d]isoxazol-3-yl)phenyl]-2-[6- (3,5-dimethylisoxazol-4-yl)pyridine-2-yl)-ethan-1- amine hydrochloride | 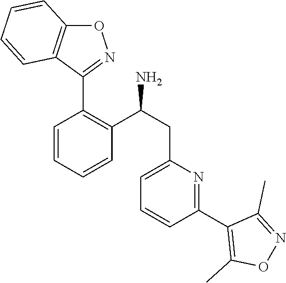 [SMILES: Cc1noc(C)c1-c1cccc(C[C@H](N)c2ccccc2-c2noc3ccccc23)n1] |
| (S)-1-[2-(6-Cyanobenzo[d]isoxazol-3-yl)phenyl]- 2-(6-cyanopyridine-2-yl)ethan-1-amine | 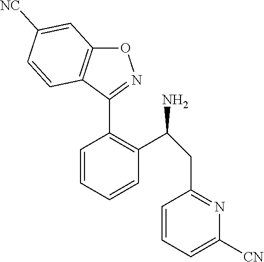 [SMILES: N#Cc1ccc2c(-c3ccccc3[C@@H](N)Cc3cccc(C#N)n3)noc2c1] |
| (S)-1-[2-(6-Cyanobenzo[d]isoxazol-3-yl)phenyl]- 2-(6-methylpyridine-2-yl)ethan-1-amine | 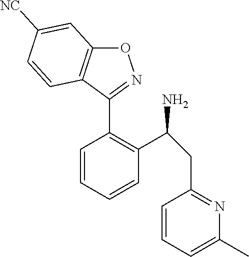 [SMILES: Cc1cccc(C[C@H](N)c2ccccc2-c2noc3cc(C#N)ccc23)n1] |
| (S)-1-[2-(6-Cyanobenzo[d]isoxazol-3-yl)phenyl]- 2-(6-methylaminopyridine-2-yl)ethan-1-amine Exemplary salt: (S)-1-[2-(Benzo[d]isoxazol-3-yl)phenyl]-2-(6- hydroxymethylpyridine-2-yl)ethan-1-amine hydrochloride | 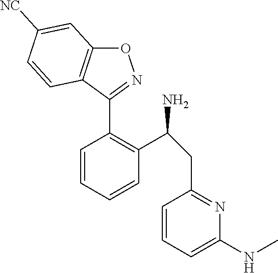 [SMILES: CNc1cccc(C[C@H](N)c2ccccc2-c2noc3cc(C#N)ccc23)n1] |
| (S)-1-[2-(Benzo[d]isoxazol-3-yl)phenyl]-2-(6- hydroxymethylpyridine-2-yl)ethan-1-amine Exemplary salt: (S)-1-[isoxazol-3-yl)phenyl]-2-(6- hydroxymethylpyridine-2-yl)ethan-1-amine hydrochloride | 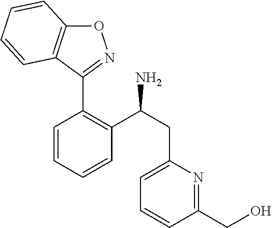 [SMILES: N[C@@H](Cc1cccc(CO)n1)c1ccccc1-c1noc2ccccc12] |
| (S)-1-[2-(6-Cyanobenzo[d]isoxazol-3-yl)phenyl]- 2-[6-(2-hydroxyethoxy)pyridine-2-yl]ethan-1- amine Exemplary salt: (S)-1-[2-(6-Cyanobenzo[d]isoxazol-3-yl)phenyl]- 2-[6-(2-hydroxyethoxy)pyridine-2-yl]ethan-1- amine hydrochloride | 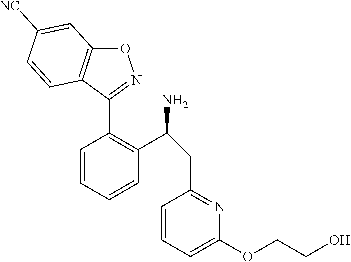 [SMILES: N#Cc1ccc2c(-c3ccccc3[C@@H](N)Cc3cccc(OCCO)n3)noc2c1] |
| (S)-1-[2-(Benzo[d]isoxazol-3-yl)phenyl]-2-[6-(2- hydroxyethyl)pyridine-2-yl]ethan-1-amine Exemplary salt: (S)-1-[2-(Benzo[d]isoxazol-3-yl)phenyl]-2-[6-(2- hydroxyethyl)pyridine-2-yl]ethan-1-amine hydrochloride | 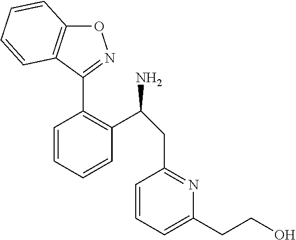 [SMILES: N[C@@H](Cc1cccc(CCO)n1)c1ccccc1-c1noc2ccccc12] |
| (S)-1-[2-(Benzo[d]isoxazol-3-yl)phenyl]-2-(6- ethylpyridine-2-yl)ethan-1-amine Exemplary salt: (S)-1-[2-(Benzo[d]isoxazol-3-yl)phenyl]-2-(6- ethylpyridine-2-yl)ethan-1-amine hydrochloride | 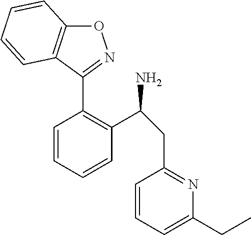 [SMILES: CCc1cccc(C[C@H](N)c2ccccc2-c2noc3ccccc23)n1] |
| (S)-1-[2-(Benzo[d]isoxazl-3-yl)phenyl]-2-(6- vinylpyridin-2-yl)ethan-1-amine | 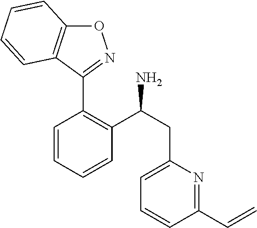 [SMILES: C=Cc1cccc(C[C@H](N)c2ccccc2-c2noc3ccccc23)n1] |
| (S)-1-[2-(Benzo[d]isoxazl-3-yl)phenyl]-2-[6- (methylsulfonyl)pyridin-2-yl]ethan-1-amine | 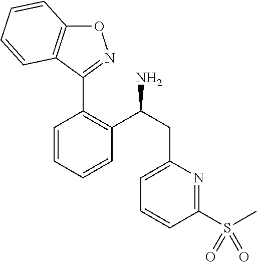 [SMILES: CS(=O)(=O)c1cccc(C[C@H](N)c2ccccc2-c2noc3ccccc23)n1] |
| (S)-6-{2-Amino-2-[2-(benzo[d]isoxazl-3- yl)phenyl]ethyl}pyridine-2-carboxamide Exemplary salt: (S)-6-{2-Amino-2-[2-(benzo[d]isoxazl-3- yl)phenyl]ethyl}pyridine-2-carboxamide hydrochloride | 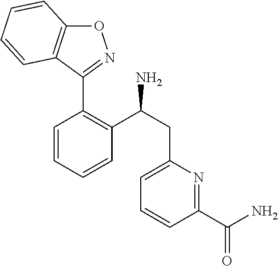 [SMILES: NC(=O)c1cccc(C[C@H](N)c2ccccc2-c2noc3ccccc23)n1] |
| (S)-6-{2-Amino-2-[2-(benzo[d]isoxazl-3- yl)phenyl]ethyl}-N-methylpyridine-2-carboxamide Exemplary salt: (S)-6-{2-Amino-2-[2-(benzo[d]isoxazl-3- yl)phenyl]ethyl}-N-methylpyridine-2-carboxamide hydrochloride | 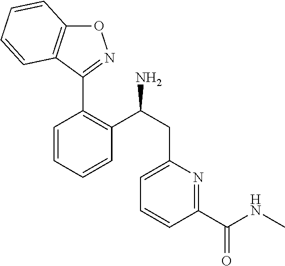 [SMILES: CNC(=O)c1cccc(C[C@H](N)c2ccccc2-c2noc3ccccc23)n1] |
| (S)-(6-{2-Amino-2-[2-(benzo[d]isoxazl-3- yl)phenyl]ethyl}pyridine-2- yl)(morpholino)methanone Exemplary salt: (S)-(6-{2-Amino-2-[2-(benzo[d]isoxazl-3- yl)phenyl]ethyl}pyridine-2- yl)(morpholino)methanone hydrochloride | 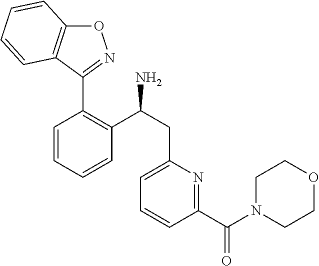 [SMILES: N[C@@H](Cc1cccc(C(=O)N2CCOCC2)n1)c1ccccc1-c1noc2ccccc12] |
| (S)-6-{2-Amino-2-[2-(benzo[d]isoxazl-3- yl)phenyl]ethyl}-N-(cyclopropylmethylpyridine-2- carboxamide Exemplary salt: (S)-6-{2-Amino-2-[2-(benzo[d]isoxazl-3- yl)phenyl]ethyl}-N-(cyclopropylmethyl)pyridine-2- carboxamide hydrochloride | 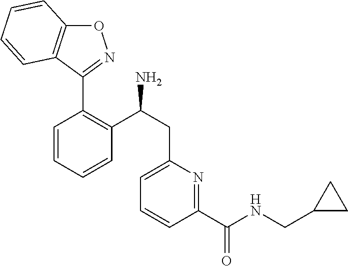 [SMILES: N[C@@H](Cc1cccc(C(=O)NCC2CC2)n1)c1ccccc1-c1noc2ccccc12] |
| (S)-6-{2-[Amino-2-(benzo[d]isoxazl-3- yl)phenyl]ethyl}-N-(2-methoxyethyl)pyridine-2- carboxamide Exemplary salt: (S)-6-{2-[Amino-2-(benzo[d]isoxazl-3- yl)phenyl]ethyl}-N-(2-methoxyethyl)pyridine-2- carboxamide hydrochloride | 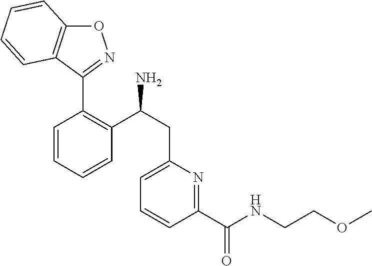 [SMILES: COCCNC(=O)c1cccc(C[C@H](N)c2ccccc2-c2noc3ccccc23)n1] |
| (S)-3-(6-{2-Amino-2-[2-(benzo[d]isoxazl-3- yl)phenyl]ethyl}pyridine-2-yl)-N,N- dimethylpropanamide Exempalry salt: (S)-3-(6-{2-Amino-2-[2-(benzo[d]isoxazl-3- yl)phenyl]ethyl}pyridine-2-yl)-N,N- dimethylpropanamide hydrochloride | 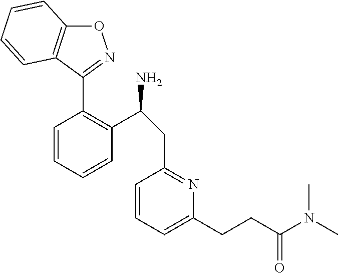 [SMILES: CN(C)C(=O)CCc1cccc(C[C@H](N)c2ccccc2-c2noc3ccccc23)n1] |
| (S)-6-{2-Amino-2-[2-(benzo[d]isoxazl-3-yl)-6- fluorophenyl]ethyl}-2-cyanopyridine Exemplary salt (S)-6-{2-Amino-2-[2-(benzo[d]isoxazl-3-yl)-6- fluorophenyl]ethyl}-2-cyanopyridine hydrohchloride | 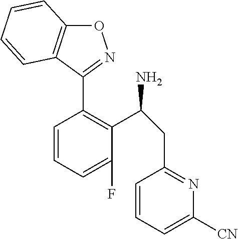 [SMILES: N#Cc1cccc(C[C@H](N)c2c(F)cccc2-c2noc3ccccc23)n1] |
| (S)-1-[2-(Benzo[d]isoxazl-3-yl)-6-fluorophenyl]-2- (pyridin-2-yl)ethan-1-amine Exemplary salt: (S)-1-[2-(Benzo[d]isoxazl-3-yl)-6-fluorophenyl]-2- (pyridin-2-yl)ethan-1-amine hydrochloride | 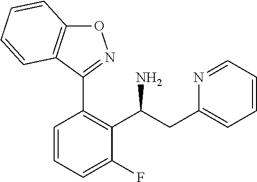 [SMILES: N[C@@H](Cc1ccccn1)c1c(F)cccc1-c1noc2ccccc12] |
| (S)-1-[2-(Benzo[d]osoxazl-3-yl)-6-fluorophenyl]-2- (6-methylpyridin-2-yl)ethan-1-amine Exemplary salt: (S)-1-[2-(Benzo[d]isoxazl-3-yl)-6-fluorophenyl]-2- (6-methylpyridin-2-yl)ethan-1-amine hydrochloride | 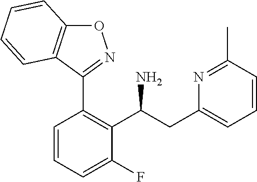 [SMILES: Cc1cccc(C[C@H](N)c2c(F)cccc2-c2noc3ccccc23)n1] |
| (S)-1-[2-(5-Bromobenzo[d]isoxazol-3-yl)phenyl]- 2-(pyridine-2-yl)ethan-1-amine Exemplary salt: (S)-1-[2-(5-Bromobenzo[d]isoxazol-3-yl)phenyl]- 2-(pyridine-2-yl)ethan-1-amine hydrochloride | 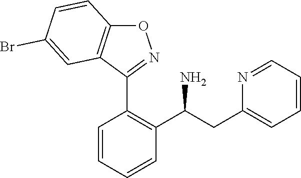 [SMILES: N[C@@H](Cc1ccccn1)c1ccccc1-c1noc2ccc(Br)cc12] |
| (S)-1-[2-(Benzo[d]isoxazol-3-yl)phenyl]-2-(6- methylpyridine-2-yl)ethan-1-amine Exemplary salt: (S)-1-[2-(Benzo[d]isoxazol-3-yl)phenyl]-2-(6- methylpyridine-2-yl)ethan-1-amine hydrochloride | 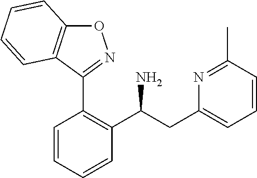 [SMILES: Cc1cccc(C[C@H](N)c2ccccc2-c2noc3ccccc23)n1] |
| (S)-1-[2-(Benzo[d]isoxazol-3-yl)phenyl]-2-(6- methoxypyridine-2-yl)ethan-1-amine Exemplary salt: (S)-1-[2-(Benzo[d]isoxazol-3-yl)phenyl]-2-(6- methoxypyridine-2-yl)ethan-1-amine hydrochloride | 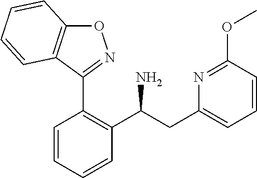 [SMILES: COc1cccc(C[C@H](N)c2ccccc2-c2noc3ccccc23)n1] |
| (S)-1-[2-(Benzo[d]isoxazol-3-yl)phenyl]-2-(6- cyanopyridine-2-yl)ethan-1-amine Exemplary salt: (S)-1-[2-(Benzo[d]isoxazol-3-yl)phenyl]-2-(6- cyanopyridine-2-yl)ethan-1-amine hydrochloride | 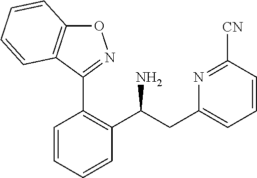 [SMILES: N#Cc1cccc(C[C@H](N)c2ccccc2-c2noc3ccccc23)n1] |
| (S)-1-[2-(Benzo[d]isoxazol-3-yl)phenyl]-2-(4- bromopyridine-2-yl)ethan-1-amine Exemplary salt: (S)-1-[2-(Benzo[d]isoxazol-3-yl)phenyl]-2-(4- bromopyridine-2-yl)ethan-1-amine hydrochloride) | 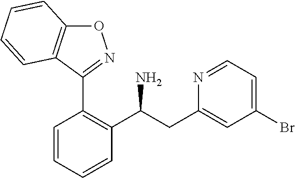 [SMILES: N[C@@H](Cc1cc(Br)ccn1)c1ccccc1-c1noc2ccccc12] |
| (S)-1-[2-(Benzo[d]isoxazol-3-yl)phenyl]-2-[6- (oxazol-2-yl)pyridine-2-yl]ethan-1-amine Exemplary salt: (S)-1-[2-(Benzo[d]isoxazol-3-yl)phenyl]-2-[6- (oxazol-2-yl)pyridine-2-yl]ethan-1-amine hydrochloride | 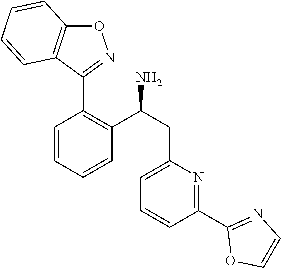 [SMILES: N[C@@H](Cc1cccc(-c2ncco2)n1)c1ccccc1-c1noc2ccccc12] |
| (S)-1-[2-(Benzo[d]isoxazol-3-yl)phenyl]-2-[6- (pyrrolidin-1-yl)pyridine-2-yl]ethan-1-amine Exemplary salt: (S)-1-[2-(Benzo[d]isoxazol-3-yl)phenyl]-2-[6- (pyrrolidin-1-yl)pyridine-2-yl]ethan-1-amine hydrochloride | 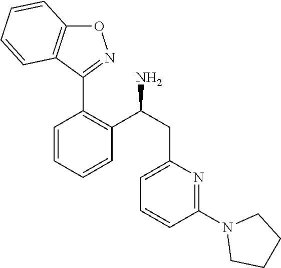 [SMILES: N[C@@H](Cc1cccc(N2CCCC2)n1)c1ccccc1-c1noc2ccccc12] |
| (S)-{2-Amino-[2-(6-bromobenzo[d]isoxazol-3- yl)phenyl]ethyl}-6-cyanopyridine | 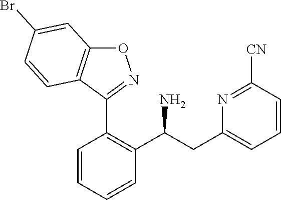 [SMILES: N#Cc1cccc(C[C@H](N)c2ccccc2-c2noc3cc(Br)ccc23)n1] |
| (S)-6-{2-Amino-2-[2-(benzo[d]isoxazol-3- yl)phenyl]ethyl}pyridine-2-amine Exemplary salt: (S)-6-{2-Amino-2-[2-(benzo[d]isoxazol-3- yl)phenyl]ethyl}pyridine-2-amine hydrochloride | 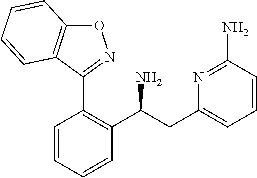 [SMILES: Nc1cccc(C[C@H](N)c2ccccc2-c2noc3ccccc23)n1] |
| (S)-1-(6-{2-Amino-2-[2-(benzo[d]isoxazol-3- yl)phenyl]ethyl}pyridine-2-yl)-3-methylurea Exemplary salt: (S)-1-(6-{2-Amino-2-[2-(benzo[d]isoxazol-3- yl)phenyl]ethyl}pyridine-2-yl)-3-methylurea hydrochloride | 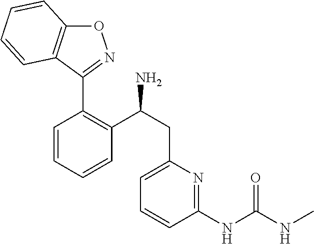 [SMILES: CNC(=O)Nc1cccc(C[C@H](N)c2ccccc2-c2noc3ccccc23)n1] |
| (S)-N-(6-{2-Amino-2-[2-(benzo[d]isoxazol-3- yl)phenyl]ethyl}pyridine-2- yl)methanesulfonamide Exemplary salt: (S)-N-(6-{2-Amino-2-[2-(benzo[d]isoxazol-3- yl)phenyl]ethyl}pyridine-2- yl)methanesulfonamide hydrochloride | 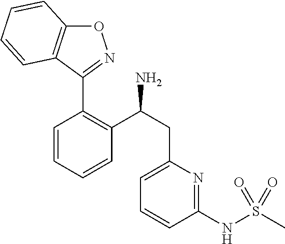 [SMILES: CS(=O)(=O)Nc1cccc(C[C@H](N)c2ccccc2-c2noc3ccccc23)n1] |
| Methyl (S)-(6-{2-Amino-2-[2-(benzo[d]isoxazol- 3-yl)phenyl]ethyl}pyridine-2-yl)carbamate Exemplary salt: Methyl (S)-(6-{2-Amino-2-[2-(benzo[d]isoxazol- 3-yl)phenyl]ethyl}pyridine-2-yl)carbamate hydrochloride | 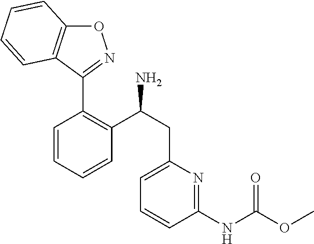 [SMILES: COC(=O)Nc1cccc(C[C@H](N)c2ccccc2-c2noc3ccccc23)n1] |
| (S)-1-[2-(Benzo[d]isoxazol-3-yl)phenyl]-2-[3- methyl-6-(methylsulfonyl)pyridine-2-yl]ethan-1- amine Exemplary salt: (S)-1-[2-(Benzo[d]isoxazol-3-yl)phenyl]-2-[3- methyl-6-(methylsulfonyl)pyridine-2-yl]ethan-1- amine hydrochloride | 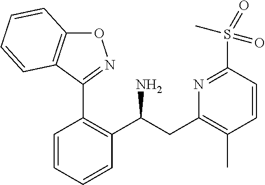 [SMILES: Cc1ccc(S(C)(=O)=O)nc1C[C@H](N)c1ccccc1-c1noc2ccccc12] |

The “Exemplary salts” in Table 1 represent one example of a pharmaceutically acceptable salt of the compound shown in the table. Other pharmaceutically acceptable salts of the compounds are also included, for example any of the pharmaceutically acceptable salts disclosed herein.

Also provided is any one of the Examples disclosed herein.

### Pharmaceutical Compositions

In accordance with another aspect, the present invention provides a pharmaceutical composition comprising a compound of the invention, except the compounds of the formulae (A) and (B) are not excluded, or a pharmaceutically acceptable salt thereof, and a pharmaceutically acceptable excipient.

Conventional procedures for the selection and preparation of suitable pharmaceutical compositions are described in, for example, “Pharmaceuticals—The Science of Dosage Form Designs”, M. E. Aulton, Churchill Livingstone, 1988.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intraperitoneal dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of a condition is an amount sufficient to symptomatically relieve in a warm-blooded animal, particularly a human the symptoms of the condition or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.1 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, a daily dose selected from 0.05 mg/kg to 100 mg/kg, 0.1 mg/kg to 100 mg/kg, 1 mg/kg to 75 mg/kg, 1 mg/kg to 50 mg/kg, 1 mg/kg to 20 mg/kg, 5 mg/kg to 10 mg/kg, 0.1 mg/kg to 5 mg/kg, 0.1 mg/kg to 2 mg/kg or 0.1 mg/kg to 1 mg/kg body weight is received, given if required in divided doses. In general lower doses will be administered when a parenteral route is employed. Thus, for example, for intravenous, subcutaneous, intramuscular or intraperitoneal administration, a dose in the range, for example, 0.05 mg/kg to 30 mg/kg, 0.1 mg/kg to 30 mg/kg, 0.1 mg/kg to 5 mg/kg, 0.1 mg/kg to 2 mg/kg or 0.1 mg/kg to 1 mg/kg body weight will generally be used. Similarly, for administration by inhalation, a dose in the range, for example, 0.05 mg/kg to 25 mg/kg body weight will be used. Suitably the compound of the invention is administered orally, for example in the form of a tablet, or capsule dosage form. The daily dose administered orally may be, for example a total daily dose selected from 1 mg to 1000 mg, 5 mg to 1000 mg, 10 mg to 750 mg, 25 mg to 500 mg, 1 mg to 100 mg, 5 mg to 75 mg, or 10 mg to 50 mg. Typically, unit dosage forms will contain about 0.5 mg to 0.5 g of a compound of this invention. In a particular embodiment the compound of the invention is administered parenterally, for example by intravenous administration. In another particular embodiment the compound of the invention is administered orally.

The compounds of the invention may be administered at a dosage interval of, for example, once every hour, once every 2 hours, once every 4 hours, once every 6 hours, once every 8 hours, or once every 12 hours. In some embodiments the compound is administered once per day, twice per day, three times per day, four times per day, once every 2 days, or once per week. Suitably the compound of the invention is administered once or twice per day.

Regular dosing of the compound of the invention may provide a cumulative, and sustained analgesic effect. The Examples herein show that a single injection of a compound of the invention results in analgesia, but the analgesic effect reduces towards the baseline level within a few hours of administration. Regular repeated dosing of a compound of the invention may provide a cumulative and sustained analgesic effect as illustrated in Examples 137 and 138 herein. The cumulative effect on analgesia provided by the compounds of the invention may enable the compound to be administered at a dose which is lower than the dose required to give a full analgesic effect administered as a single bolus dose. Accordingly, regular administration of a low dose of a compound of the invention may provide a greater therapeutic window between analgesia and undesirable side-effects which might be associated with higher doses, for example bradycardia or tremors.

In certain embodiments a compound of the invention is administered regularly so as to provide a plasma concentration of 10% to 120% of the analgesic ED₅₀ for the compound. For example the compound may be administered at a dose which provides from 10% to 100%, from 10% to 80%, from 10% to 60%, from 15% to 50%, from 20% to 50%, from 25% to 50% or from 25% to 45% of the analgesic ED₅₀ of the compound. The regular dosage interval may be, for example, any of the dosage intervals set out above.

### Therapeutic Uses and Applications

In accordance with another aspect, the present invention provides a compound of the invention, except the compounds of the formulae (A) and (B) are not excluded, or a pharmaceutically acceptable salt thereof, or a pharmaceutical composition of the invention, for use as a medicament.

A further aspect of the invention provides a compound of the invention, except the compounds of the formulae (A) and (B) are not excluded, or a pharmaceutically acceptable salt thereof, or a pharmaceutical composition of the invention, for use in the treatment of a disease or medical condition mediated by hyperpolarisation activated cyclic-nucleotide modulated ion channel 2 (HCN2).

Also provided is the use of a compound of the invention, except the compounds of the formulae (A) and (B) are not excluded, or a pharmaceutically acceptable salt thereof, or a pharmaceutical composition of the invention, in the manufacture of a medicament for the treatment of a disease or medical condition mediated by HCN2.

Also provided is a method of treating a disease or medical condition mediated by HCN2 in a subject in need thereof, the method comprising administering to the subject an effective amount of: (i) a compound of the invention, except the compounds of the formulae (A) and (B) are not excluded, or a pharmaceutically acceptable salt thereof; or (ii) a pharmaceutical composition of the invention.

The conditions mediated by HCN2 may be, for example, any of the conditions disclosed herein.

In some embodiments in the therapeutic uses and applications described herein the compound of the invention is not a compound of the formula (A) or formula (B).

The compounds of the invention are HCN2 inhibitors, useful in the treatment of a conditions in which inhibition of HCN2 ion channels is beneficial. As discussed in the Background to the Invention, the disclosure of which is incorporated into the main description, HCN4 is highly expressed in cardiac tissue and is the major regulator of cardiac pacemaking. Inhibition of HCN4 induces bradycardia and deletion of HCN4 in mice, either globally, or locally in the heart, is lethal. Accordingly, compounds which significantly inhibit HCN4 in addition to HCN2 would not be suitable as a chronic treatment, for example as an analgesic used for the chronic treatment of pain. Preferred compounds of the invention selectively inhibit HCN2 over HCN4. HCN2 selective compounds are expected to reduce or eliminate the risks of undesirable cardiac side-effects associated with the use of a compound of the invention as a medicament for the treatment of conditions mediated by HCN2. In preferred embodiments a compound of the invention exhibits an IC₅₀ in the HCN2 assay described herein (see Example 126) which is at least 2 times, for example at least 5 times, at least 10 times, or at least 20 times lower than the IC₅₀ of the same compound measured in the HCN4 assay described herein (see Example 126).

HCN1 channels are also expressed in cardiac tissue and are associated with cardiac function. Accordingly, preferred compounds of the invention selectively inhibit HCN2 over HCN1. In some embodiments a compound of the invention exhibits an IC₅₀ in the HCN2 assay described herein (see Example 126) which is at least 2 times, for example at least 5 times, at least 10 times or at least 20 times lower than the IC₅₀ of the same compound measured in the HCN1 assay described herein (see Example 126).

The voltage-gated Na⁺ channel Naᵥ1.5 is found predominantly in cardiac muscle. It initiates the cardiac action potential in the heart and is essential for conduction of the electrical impulse, as well as the action potential duration. In preferred embodiments a compound of the invention selectively inhibits HCN2 over Naᵥ1.5. In some embodiments a compound of the invention exhibits an IC₅₀ in the HCN2 assay described herein (see Example 126) which is at least 2 times, for example at least 5 times, at least 10 times, at least 20 times or at least 50 times lower than the IC₅₀ of the same compound measured in the Naᵥ1.5 assay described herein (see Example 128).

It is well known that drugs which inhibit the hERG potassium channel in the hearts can result in delayed ventricular repolarization (QT interval prolongation). Preferred compounds of the invention are those with a low hERG liability. In some embodiments a compound of the invention exhibits an IC₅₀ in the HCN2 assay described herein which is at least 2 times, for example at least 5 times, at least 10 times or at least 20 times lower than the IC₅₀ of the same compound measured in the hERG assay described herein (see Example 127).

Accordingly, in preferred embodiments a compound of the invention has a high therapeutic window between the concentration required for inhibition of HCN2 and ion channels associated with cardiac function. In some embodiments compounds of the invention are selective for HCN2 over one or more of HCN4, HCN1, Naᵥ1.5 or hERG. In particular embodiments preferred compounds of the invention selectively inhibit HCN2 over HCN4 and/or HNC1.

HCN2 channels are widely expressed in the brain and significant inhibition of HCN2 in the brain could induce undesirable CNS side-effects such as tremors or ataxia. In preferred embodiments, compounds of the invention are peripherally restricted HCN2 inhibitors such that when present at therapeutically effective concentrations in peripheral tissues, only low levels of the compound are present in the brain at a concentration below that necessary to induce undesirable CNS associated side effects. In some embodiments the compound of the invention is a substrate for the transporter P-glycoprotein (P-gp). P-gp substrates are generally effluxed at the brain endothelium. Accordingly, compounds which are P-gp substrates are expected to exhibit low concentrations in brain tissue. In some embodiments a compound of the invention has a high efflux ratio when measured in the MDCK-MDR1 permeability assay described herein (see Example 129). The MDCK-MDR1 assay described in Example 129 run in the absence and presence of a P-gp inhibitor can be used to identify compounds having the potential to be peripherally restricted. A net flux value>5 (i.e. efflux ratio without inhibitor divided by efflux ratio plus inhibitor) is indicative of compounds being substrates for the transporter P-gp and would therefore have a greater likelihood of being restricted from the CNS (i.e. compounds with low CNS penetration). In some embodiments a compound of the invention with low CNS penetration has a net flux of 5 or more, for example 10 or more, 15 or more, or 20 or more when measured in the MDCK-MDR1 permeability assay described herein. Compounds of the invention which exhibit low CNS penetration following administration, are referred to herein as “peripherally restricted compounds” or “peripherally restricted HCN2 inhibitors”.

In the following sections of the application reference is made to a compound of the invention, or a pharmaceutically acceptable salt thereof for use in the treatment of certain diseases or conditions. It is to be understood that any reference herein to a compound for a particular use is also intended to be a reference to (i) the use of the compound of the invention, or pharmaceutically acceptable salt thereof, in the manufacture of a medicament for the treatment of that disease or condition; and (ii) a method of treating the disease or condition in a subject, the method comprising administering to the subject a therapeutically effective amount of the compound of the invention, or pharmaceutically acceptable salt thereof.

The disease or medical condition mediated by HCN2 may be any of the diseases or medical conditions listed in this application.

### Pain

In some embodiments a compound of the invention is for use in the treatment or prevention of pain generally, including, but not limited to NP and IP.

### Neuropathic Pain

In some embodiments a compound of the invention is for use in the treatment or prevention of neuropathic pain. In some embodiments a compound of the invention is for use in the treatment or prevention of peripheral neuropathic pain. Examples of NP include, but are not limited to neuropathic pain selected from painful diabetic neuropathy (PDN), postherpetic neuralgia (PHN), pain associated with cancer, chemotherapy induced pain including, chemotherapy-induced peripheral neuropathy, post-operative pain (e.g. postmastectomy syndrome, post-thoracotomy syndrome or phantom pain), trigeminal neuralgia, complex regional pain syndrome (CRPS), opioid resistant pain, pudendal neuralgia and neuropathic pain associated with lower back pain, nerve damage following traumatic injury (e.g. whiplash injury in car crash) and carpal tunnel syndrome.

In some embodiments a compound of the invention is for use in the treatment or prevention of neuropathic pain associated with or resulting from: neurological disorders, spine and peripheral nerve surgery, spinal cord trauma, chronic pain syndrome, fibromyalgia, chronic fatigue syndrome, neuralgias (e.g. trigeminal neuralgia, glossopharyngeal neuralgia, postherpetic neuralgia and causalgia), lupus, HIV infection, sarcoidosis, peripheral neuropathy, bilateral peripheral neuropathy, diabetic neuropathy, sciatic neuritis, mandibular joint neuralgia, peripheral neuritis, polyneuritis, stump pain, phantom limb pain, bony fractures, oral neuropathic pain, Charcot's pain, complex regional pain syndrome I and II (CRPS VIT), radiculopathy, Guillain-Barre syndrome, meralgia paresthetica, burning-mouth syndrome, optic neuritis, postfebrile neuritis, migrating neuritis, segmental neuritis, Gombault's neuritis, neuronitis, cervicobrachial neuralgia, cranial neuralgia, geniculate neuralgia, glossopharyngial neuralgia, idiopathic neuralgia, intercostals neuralgia, mammary neuralgia, Morton's neuralgia, nasociliary neuralgia, occipital neuralgia, red neuralgia, Sluder's neuralgia, splenopalatine neuralgia, supraorbital neuralgia, vulvodynia, or vidian neuralgia. In one embodiment the compound of the invention is for use in the treatment of postherpetic neuralgia.

In some embodiments a compound of the invention is for use in the prevention or relief of one or more of the symptoms of NP, for example dysesthesia (spontaneous or evoked burning pain, often with a superimposed lancinating component), deep pain, aching pain, hyperesthesia, hyperalgesia, allodynia and hyperpathia.

### Inflammatory Pain

In some embodiments a compound of the invention is for use in the treatment or prevention of inflammatory pain. In some embodiments the pain is chronic inflammatory pain. In some embodiments the pain is acute inflammatory pain. In some embodiments a compound of the invention is for use in the treatment or prevention of inflammatory pain, especially chronic inflammatory pain, resulting from or associated with one or more of: inflammatory bowel disease, visceral pain, post-operative pain, osteoarthritis, rheumatoid arthritis, back pain, lower back pain, joint pain, abdominal pain, chest pain, labour, musculoskeletal diseases, skin diseases, toothache, pyresis, burn, sunburn, animal or insect bite or sting, neurogenic bladder, interstitial cystitis, urinary tract infection, rhinitis, dermatitis including contact dermatitis and atopic dermatitis, pharyngitis, mucositis, enteritis, irritable bowel syndrome, cholecystitis, pancreatitis, postmastectomy pain syndrome, menstrual pain, endometriosis, sinus headache, tension headache, or arachnoiditis.

In some embodiments a compound of the invention is for use in the treatment of inflammatory hyperalgesia, including inflammatory somatic hyperalgesia or inflammatory visceral hyperalgesia. Inflammatory somatic hyperalgesia can be characterized by the presence of an inflammatory hyperalgesic state in which a hypersensitivity to thermal, mechanical and/or chemical stimuli exists. Inflammatory visceral hyperalgesia can also be characterized by the presence of an inflammatory hyperalgesic state, in which an enhanced visceral irritability exists.

### Tinnitus

As set out in the Background to the Invention, and illustrated in the Examples, the inventors have for the first time shown that tinnitus can be treated using an HCN2 inhibitor in animal models. The Examples suggest that the effects observed are applicable to any HCN2 inhibitor and are not limited to a compound of the invention.

In one embodiment of the invention there is provided an HCN2 inhibitor for use in the treatment of tinnitus or a related condition. In a preferred embodiment the HCN2 inhibitor is a compound of the invention. Accordingly there is provided a compound of the invention, except the compounds of Formulae (A) and (B) are not excluded, for use in the prevention or treatment of tinnitus or a related condition.

Ivabradine is a peripherally restricted compound, with pan-HCN inhibitory action. The Examples herein show that despite being peripherally restricted the compound successfully treated tinnitus. Similar results were obtained using a peripherally restrictive compound of the invention. The experiments therefore suggest that tinnitus may be treated without the need for CNS penetration, thereby avoiding undesirable side effects that might be associated with HCN2 inhibition in the CNS such as tremors or ataxia.

Accordingly, also provided is a peripherally restricted HCN2 inhibitor for use in the treatment of tinnitus or a related condition. In some embodiments the peripherally restricted HCN2 inhibitor is a peripherally restricted HCN2 inhibitor, for example ivabradine. In preferred embodiments the peripherally restricted HCN2 inhibitor is peripherally restricted compound of the invention.

Tinnitus may occur as objective tinnitus, or subjective tinnitus. Subjective tinnitus is the most common type of tinnitus. Subjective tinnitus, also known as sensorineural tinnitus can only be heard by the affected person. Objective tinnitus, on the other hand, can be detected by other people and is usually caused by myoclonus or a vascular condition, although in some cases, tinnitus is generated by a self-sustained oscillation within the ear. In preferred embodiments the HCN2 inhibitor (preferably a compound of the invention) is for use in the treatment of subjective tinnitus. The tinnitus may be acute tinnitus, however, in preferred embodiments the tinnitus is chronic tinnitus, for example tinnitus that persists for more than 2 weeks, more than 1 month or more than 6 months.

In some embodiments the HCN2 inhibitor (preferably a compound of the invention) is for use in the treatment or prevention of tinnitus caused by or associated with one of more of: exposure to loud noise; presbyacusis (hearing loss); ear or head injuries, ear infections; tumours which impact on auditory nerves; Meniere's disease; cardiovascular disease, cerebrovascular disease; hyperthyroidism; hypothyroidism; side-effects of a drug therapy (for example salicylates (including mesalamine or aspirin), particularly when taken in high doses), quinine anti-malarial agents, aminoglycoside antibiotics, chemotherapy (including, but not limited to platinum cytotoxic agents (e.g. cisplatin, carboplatin and oxaliplatin)) or loop diuretics (e.g. furosemide, ethacrynic acid and torsemide); or an auditory dysfunction (e.g. hyperacusis, distortion of sounds, misophonia, phonophobia and central auditory processing disorders).

In some embodiments the HCN2 inhibitor (preferably a compound of the invention) is for use in the treatment or prevention of tinnitus, Meniere's disease or hyperacusis. In some embodiments the HCN2 inhibitor is for use in the treatment or prevention of tinnitus or Meniere's disease. In a particular embodiment there is provided a compound of the invention, except the compounds of the formulae A and B are not excluded for use in the treatment or prevention of tinnitus.

### Migraine

The debilitating pain of migraine imposes a significant personal and economic burden. Actual or potential promise as therapeutics in migraine is shown by the triptan family, by the “gepant” family of antagonists to the CGRP receptor and by monoclonal antibodies against CGRP, amongst others. All have significant disadvantages, including the promotion of medication overuse headaches by triptans, liver toxicity in gepants and the need for regular injection of monoclonals. However, a significant fraction of migraine patients do not achieve relief with these treatments. There remains a need for new treatments for migraine.

Triptans are agonists at 5HT1B/D receptors, which couple to Gi/o and therefore inhibit production of cAMP5 (Alexander et al., Br. J. Pharmacol. 174 Suppl. 1, S17-S129, (2017)). The receptor for CGRP, which is emerging as a critical mediator of migraine, couples to Gs and therefore increases cAMP (Alexander et al. supra). These considerations suggest that cAMP in trigeminal nociceptive afferents innervating the meninges and dura may be a critical downstream mediator of migraine (Schytz et al., Curr. Opin. Neurol. 23, 259-265, (2010)).

As discussed herein, it has been shown that the HCN2 ion channel isoform, whose activation is potentiated by cAMP, promotes firing in nociceptive afferent neurons and, as a result, is a critical final effector of pain in animal models of nerve injury pain, of chemotherapy-induced pain and of painful diabetic neuropathy ((Tsantoulas, et al., Sci Transl Med 9, eaam6072, (2017); Tsantoulas et al., Biochem J 473, 2717-2736, 2016); Young et al., Pain 155, 1708-1719, (2014); and Emery et al., Science 333, 1462-1466, (2011)). Accordingly, HCN2 ion channels may be a critical downstream mediator of migraine pain. A HCN2 inhibitor may be useful in the treatment or prevention of migraine, particularly in the treatment or prevention of migraine pain.

In certain embodiments there is provided an HCN2 inhibitor for use in the prevention or treatment of migraine. In certain embodiments there is provided an HCN2 inhibitor for use in the treatment or prevention of migraine pain. In a preferred embodiment the HCN2 inhibitor is a compound of the invention. Accordingly there is provided a compound of the invention, except the compounds of Formulae (A) and (B) are not excluded, for use in the prevention or treatment of migraine. Also provided is a compound of the invention, except the compounds of Formulae (A) and (B) are not excluded, for use in the prevention or treatment of migraine pain.

### Treatment of Specific Pain Syndromes and Conditions

In certain embodiments a compound of the invention is for use in the treatment of a condition selected from: painful diabetic neuropathy; migraine rheumatoid arthritis (RA), osteoarthritis (OA), pain associated with long-term use of opioids (Opioid-induced hyperalgesia, OIH), cancer-associated bone pain and fibromyalgia (FMS, fibromyalgia syndrome).

### Subjects

A compound of the invention may be for use in the treatment of a human or animal subject affected by any of the medical conditions disclosed herein. The subject may be a warm-blooded mammal such as a farm animal (e.g. cow, sheep or pig) or a companion animal or pet (e.g. a dog, cat or horse). Preferably, the subject is a human.

### Combination Therapies

The methods of treatment according to the invention or the compound of the invention for use in the treatment of conditions mediated by HCN2 as defined herein may be applied as a sole therapy or be a combination therapy with an additional active agent.

For example, where the condition is pain (e.g. NP or IP) a compound of the invention may be used in combination with another analgesic agent. Examples of analgesic agents include, but are not limited to an opioid (e.g. morphine and other opiate receptor agonists; nalbuphine or other mixed opioid agonist/antagonists; or tramadol); a non-steroidal anti-inflammatory agent (NSAIDs) (e.g. aspirin, ibuprofen, naproxen, or a selective COX2 inhibitor such as celecoxib); paracetamol; baclofen, pregabalin, gabapentin, a tricyclic antidepressant (e.g. clomipramine or amitriptyline), or a local anaesthetic (e.g. lidocaine), or a combination of two or more thereof.

The combination therapies defined herein may be achieved by way of the simultaneous, sequential or separate dosing of the individual components of the treatment. Such combination products employ the compounds of this invention within a therapeutically effective dosage range described herein and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of the invention and an approved or otherwise published dosage range(s) of the other pharmaceutically active compound(s).

According to a further aspect of the invention there is provided a pharmaceutical product comprising a compound of the invention, or a pharmaceutically acceptable salt thereof as defined herein and an additional active agent for the treatment of pain (e.g. NP or IP). The additional active agent may be an analgesic agent as defined herein.

In an embodiment there is provided a pharmaceutical product comprising a compound of the invention, or a pharmaceutically acceptable salt thereof as defined herein and an additional active agent for the treatment of a condition which is modulated by HCN2. The additional active agent may be an analgesic agent as defined herein.

According to a further aspect of the invention there is provided a compound of the invention, or a pharmaceutically acceptable salt thereof for use simultaneously, sequentially or separately with an analgesic agent as defined herein, in the treatment of pain (e.g. NP or IP).

### Synthesis

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl or trifluoroacetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃·OEt₂. A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

### General Synthetic Routes

Compounds of Formula (I) in which A is oxygen may be prepared according to Scheme 1.

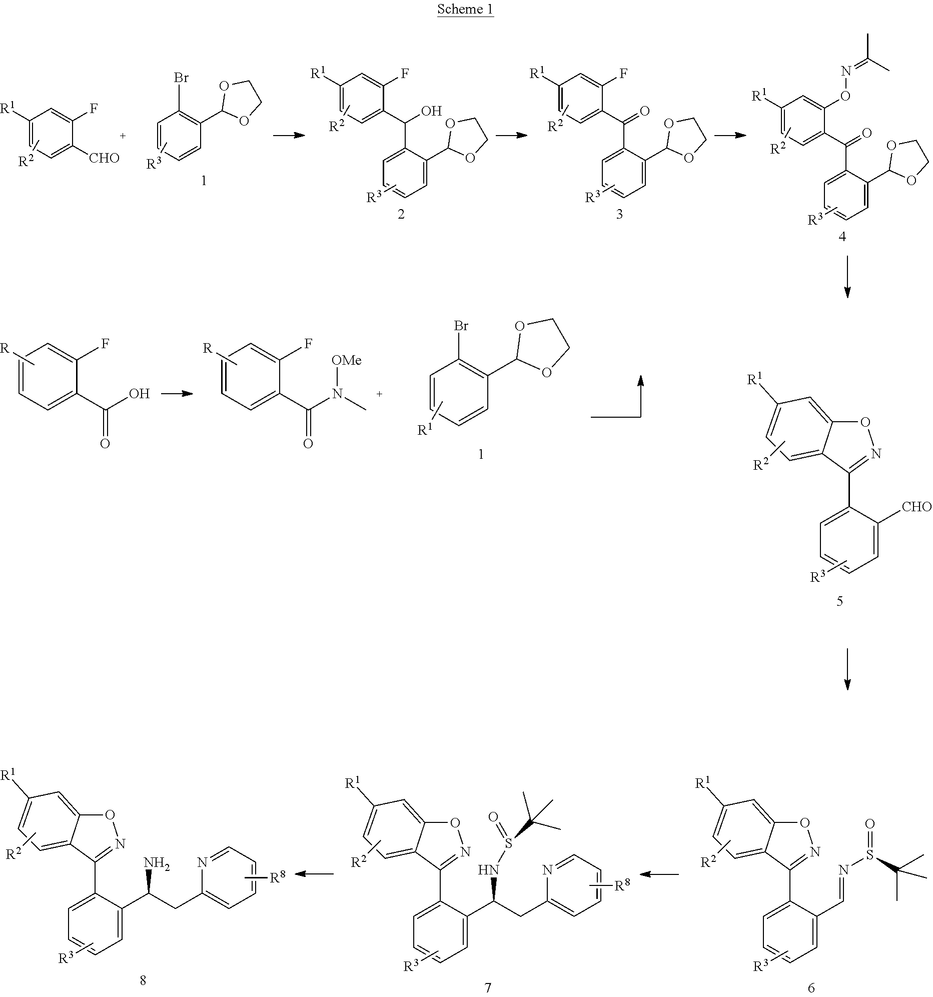

Intermediate alcohols 2 may be prepared by the reaction of an anion generated from an appropriately substituted protected benzaldehyde (1) using an alkyllithium (such as n-butyllithium) with an appropriately substituted benzaldehyde in a solvent such as diethyl ether or THF at a temperature between −78° C. and 0° C. The resultant alcohol 2 may be oxidised to give the ketone 3 under standard conditions known to one skilled in the art. For example the oxidation may be carried out using TEMPO and 1,3-dibromo-4,4-dimethylhydantoin in a mixture of tert-butanol and water, in the presence of a base such as sodium bicarbonate at a temperature between room temperature and the 80° C. Alternatively, ketones 3 may be prepared by the reaction of the same anion generated from 1 and an appropriately substituted Weinreb's amide (prepared according to standard conditions known to those skilled in the art from the corresponding benzoic acid) using similar conditions to those employed in the reaction with the aldehyde. The oximes 4 may be generated from the fluoro ketones 3 by reaction with acetone oxime in the presence of a strong base (such as sodium hydride or potassium t-butoxide) in an ether solvent (such as dry diethyl ether or THF) at a temperature between 0° C. and the reflux temperature of the solvent. Treatment of 4 with mineral acid such as hydrochloric acid in a solvent such as ethanol or isopropanol at the reflux temperature of the solvent provides the cyclised benzisoxazole 5. Conversion of the aldehyde 5 to the imine 6 was achieved by treatment with the appropriate chiral single enantiomer of (S)-2-methylpropane-2-sulfinamide in the presence of a base such as cesium carbonate in a chlorinated solvent such as dichloromethane, at the reflux temperature of the solvent. Alternatively, reaction with (S)-2-methylpropane-2-sulfinamide may be carried out in the presence of for example titanium ethoxide in an appropriate solvent such as ethanol or THF at a temperature between room temperature and the reflux temperature of the solvent. Reaction of the generated single enantiomer of the sulfinamide 6 with the anion generated from the appropriately substituted 2-alkyl pyridine and an organolithium reagent such as n-butyl lithium, lithium di-isopropylamide or lithium hexamethyl disilazide in a solvent such as THF at a temperature between −78° C. and 0° C. gives preferentially the desired diastereomeric isomer of the intermediate 7 which can be purified by chromatography to remove the undesired minor diastereomer. Deprotection under acidic conditions using for example HCl or trifluoroacetic acid in a solvent such as dichloromethane, ethyl acetate methanol or dioxane then provides the target compounds as single enantiomers.

One skilled in the art will recognise that interconversion of various groups R¹, R², R³ or R⁸ may be carried out at different stages of the synthesis and that protection of various functionalities may be required in order to complete the required syntheses.

An alternative synthetic approach to prepare compounds of Formula (I) in which A is oxygen is shown in Scheme 2.

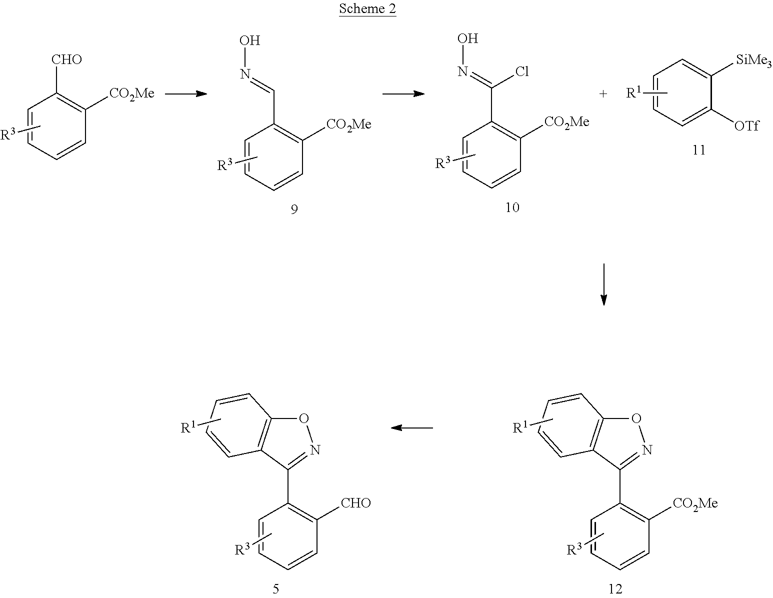

An appropriately substituted 2-formylbenzoate ester may be converted to the corresponding oxime 9 by treatment with hydroxylamine hydrochloride in the presence of sodium acetate in a solvent such as aqueous alcohol at a temperature between room temperature and the reflux temperature of the solvent. Treatment of oxime 9 with a chlorinating reagent such as chlorine gas or N-chlorosuccinimide in a solvent such as dichloromethane provides the chloro-oxime 10. 3+2 cycloaddition of the nitrile oxide generated in situ from 10 with benzyne generated in situ from 11 using cesium fluoride gives the benzisoxazole ester 12. Conversion of the ester 12 to the aldehyde 5 may be achieved in a number of different ways by methods known to one skilled in the art. Examples of such methods include the direct reduction using a reducing agent such as DIBAL in a variety of solvents such as dichloromethane, toluene or THF at temperatures between 0° C. and the reflux temperature of the solvent. Alternatively, the generation of the aldehyde may be achieved in a two-step process by reducing the ester 12 to the alcohol using for example lithium aluminium hydride in an ether solvent such as diethyl ether of THF at a temperature between 0° C. and room temperature followed by oxidation of the alcohol to the aldehyde using for example TEMPO (as described in Scheme 1) or using activated manganese oxide in a chlorinated solvent such as dichloromethane. With the aldehyde in hand, conversion to the compounds of Formula (I) may be achieved using the same methods as described in Scheme 1.

Compounds of Formula (I) in which A is NR⁹ may be prepared according to Scheme 3.

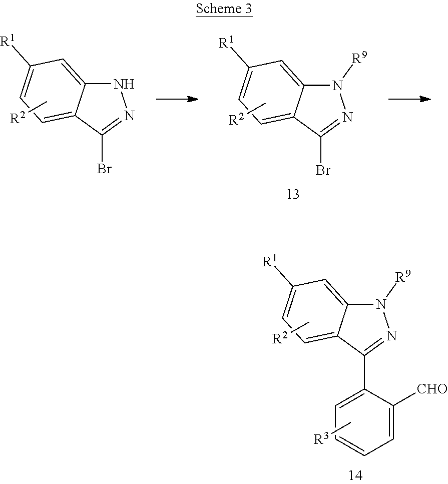

The appropriately substituted N-alkylated indazoles may be prepared by alkylation of the corresponding 3-bromoindazole using the appropriate alkyl halide (alkyl bromide or iodide) and a strong base such as sodium hydride in a solvent such as THF at a temperature between 0° C. and the reflux temperature of the solvent. The desired isomer of the indazole can be isolated from the mixture by chromatographic separation from the undesired regio-isomer. The desired aldehyde 14 may then be achieved by Suzuki coupling of the bromoindazole 13 with the appropriate 2-formylphenylboronic acid or boronate using a palladium catalyst such as tetrakis-triphenylphosphine palladium, bis-triphenylphosphine palladium chloride or palladium chloride dppf in the presence of a base such as sodium carbonate or cesium carbonate in a mixture or water and an appropriate solvent such as dioxane, THF or DME at a temperature between room temperature and the reflux temperature of the solvent. Conversion of the aldehyde 14 to the compounds of Formula (I) may be achieved in a manner similar to that shown in Scheme 1.

### EXAMPLES

### Abbreviations:

The following abbreviations are used:
DCM: Dichloromethane
DMF: N,N-Dimethyl formamide
DMP: Dess Martin periodinane
DMSO: Dimethyl sulfoxide
EGTA: Ethylene glycol-bis(2-aminoethylether)-N,N,N′,N′-tetraacetic acid
FCC: Flash column chromatography
HATU: O-(7-Azabenzotriazol-1-yl)-N,N,N,N-tetramethyl uronium
hexafluorophosphate
HEPES: 4-(2-hydroxyethyl)-1-piperazineethanesulfonic acid
HPLC: High performance liquid chromatography
IPA: 2-Propanol
LDA: Lithium di-isopropylamide
MDAP: Mass-directed autopurification
Min: Minutes
RT: Retention time
TFA: Trifluoroacetic acid
THF: Tetrahydrofuran

In the procedures that follow, after each starting material, reference to an Intermediate/Example number is usually provided. This is provided merely for assistance to the skilled chemist. When reference is made to the use of a “similar” or “analogous” procedure, as will be appreciated by those skilled in the art, such a procedure may involve minor variations, for example reaction temperature, reagent/solvent amount, reaction time, work-up conditions or chromatographic purification conditions.

NMR spectra were obtained on a Varian Unity Inova 400 spectrometer with a 5 mm inverse detection triple resonance probe operating at 400 MHz or on a Bruker Avance DRX 400 spectrometer with a 5 mm inverse detection triple resonance TXI probe operating at 400 MHz or on a Bruker Avance DPX 300 spectrometer with a standard 5 mm dual frequency probe operating at 300 MHz or on a Bruker Fourier 300 spectrometer with a 5 mm dual frequency probe operating at 300 MHz. Shifts are given in ppm relative to tetramethylsilane (δ=0 ppm). J values are given in Hz through-out. NMR spectra were assigned using CMC-Assist Version 2.3 or SpinWorks version 3.

Liquid chromatography mass spectroscopy (LCMS) methods used are as follows.

Method 1:

| Instrumentation | HQC—Acquity UPLC (binary pump/PDA | | |
|---|---|---|---|
| | detector) + ZQ Mass Spectrometer | | |
| Column | ACQUITY UPLC BEH C₁₈ 1.7 μm, | | |
| | 100 × 2.1 mm, maintained at 40° C. | | |
| Mobile Phase A | 0.1% Aqueous formic acid (v/v) | | |
| Mobile Phase B | 0.1% Formic acid in acetonitrile (v/v) | | |
| Flow | 0.4 ml/min | | |
| Gradient Program | Time (mins) | % A | % B |
| | 0.0 | 95 | 05 |
| | 0.4 | 95 | 05 |
| | 6.0 | 05 | 95 |
| | 6.8 | 05 | 95 |
| | 7.0 | 95 | 05 |
| | 8.0 | 95 | 05 |

| | |
|---|---|
| Sample | 1 μl injection of a 0.2-0.5 mg/ml solution |
| | in an appropriate solvent at 20° C. |
| Detectors | UV, diode array 200-500 nm |
| | MS, mass 100-800 (or −1500 for HM method) |
| | in ES+ & ES− (no split to MS) |
| Data Analysis | Peak area percentage (APCT) with an |
| | integration threshold of 0.2% (relative) |

Method 2:

| Instrumentation | HPS—Acquity i-Class (quarternary pump/PDA | | |
|---|---|---|---|
| | detector) + Quattro Micro Mass Spectrometer | | |
| Column | ACQUITY UPLC BEH C₁₈ 1.7 μm, | | |
| | 100 × 2.1 mm, maintained at 40° C. | | |
| Mobile Phase A | 0.1% Aqueous formic acid (v/v) | | |
| Mobile Phase B | 0.1% Formic acid in acetonitrile (v/v) | | |
| Flow | 0.4 ml/min | | |
| Gradient Program | Time (mins) | % A | % B |
| | 0.0 | 95 | 05 |
| | 0.4 | 95 | 05 |
| | 6.0 | 05 | 95 |
| | 6.8 | 05 | 95 |
| | 7.0 | 95 | 05 |
| | 8.0 | 95 | 05 |

| | |
|---|---|
| Sample | 1 μl injection of a 0.5 mg/ml solution |
| | in an appropriate solvent at 20° C. |
| Detectors | UV, diode array 200-500 nm |
| | MS, mass 100-800 (or −1500 for HM method) |
| | in ES+ & ES− (no split to MS) |

Method 3:

| Instrumentation | Acquity H-Class (quaternary pump/PDA | | |
|---|---|---|---|
| | detector) + QDa Mass Spectrometer | | |
| Column | Acquity UPLC CSH C18 1.7 μm, | | |
| | 50 × 2.1 mm at 40° C. | | |
| Mobile Phase A | 0.1% Aqueous formic acid (v/v) | | |
| Mobile Phase B | 0.1% Formic acid in acetonitrile (v/v) | | |
| Flow | 1.0 ml/min | | |
| Gradient Program | Time (mins) | % A | % B |
| | 0.0 | 97 | 03 |
| | 4 | 01 | 99 |
| | 4.4 | 01 | 99 |
| | 4.5 | 97 | 03 |
| | 5 | 97 | 03 |

| | |
|---|---|
| Sample | 1 μl injection (Open Access) |
| Detectors | UV, diode array 190-450 nm |
| | MS, mass 160-1000 (or 100-800 for LM or |
| | 160-1250 for HM method) in ES+ & ES− |

Method 4:

| Instrumentation | Waters Acquity Classic + 996 PDA detector + | | |
|---|---|---|---|
| | Waters ZMD Mass Spectrometer | | |
| Column | Acquity UPLC CSH C18 1.7 μm, | | |
| | 50 × 2.1 mm at 40° C. | | |
| Mobile Phase A | 0.1% Aqueous formic acid (v/v) | | |
| Mobile Phase B | 0.1% Formic acid in acetonitrile (v/v) | | |
| Flow | 1.0 ml/min | | |
| Gradient Program | Time (mins) | % A | % B |
| | 0.0 | 97 | 3 |
| | 0.1 | 97 | 3 |
| | 0.15 | 97 | 3 |
| | 2.3 | 1 | 99 |
| | 2.4 | 1 | 99 |
| | 2.5 | 97 | 3 |

| | |
|---|---|
| Sample | 1 μl injection (Open Access) |
| Detectors | UV, diode array 190-450 nm |
| | MS, mass 160-1000 (or 160-1500 for HM method) |
| | in ES+ & ES− (200 μl/min split to MS) |

Method 5:

| Instrumentation | Hewlett Packard HP1100 LC system + Waters | | |
|---|---|---|---|
| | ZMD quadrupole mass spectrometer | | |
| Column | Phenomenex Luna 3micron C18(2) | | |
| | 30 × 4.6 mm or equivalent | | |
| Mobile Phase A | 0.1% Aqueous formic acid (v/v) | | |
| Mobile Phase B | 0.1% Formic acid in acetonitrile (v/v) | | |
| Flow | 2.0 ml/min | | |
| Gradient Program | Time (mins) | % A | % B |
| | 0.0 | 95 | 5 |
| | 0.5 | 95 | 5 |
| | 4.5 | 5 | 95 |
| | 5.5 | 5 | 95 |
| | 6.0 | 95 | 5 |

| | |
|---|---|
| Detectors | UV, diode array + Sedex 85 evaporative |
| | light scattering detector |

Method 6:

| Instrumentation | Waters 1525 LC system + Waters ZMD | | |
|---|---|---|---|
| | quadrupole mass spectrometer | | |
| Column | Phenomenex Luna 3micron C18(2) | | |
| | 30 × 4.6 mm or equivalent | | |
| Mobile Phase A | 0.1% Aqueous formic acid (v/v) | | |
| Mobile Phase B | 0.1% Formic acid in acetonitrile (v/v) | | |
| Flow | 2.0 ml/min | | |
| Gradient Program | Time (mins) | % A | % B |
| | 0.0 | 95 | 5 |
| | 0.5 | 95 | 5 |
| | 4.5 | 5 | 95 |
| | 5.5 | 5 | 95 |
| | 6.0 | 95 | 5 |

| | |
|---|---|
| Detectors | Waters 996 diode array + Sedex 85 |
| | evaporative light scattering detector |

Method 7:

| Instrumentation | Hewlett Packard HP11050 LC system + | | |
|---|---|---|---|
| | VG Platform II quadrupole mass spectrometer | | |
| Column | Phenomenex Luna 3micron C18(2) | | |
| | 30 × 4.6 mm or equivalent | | |
| Mobile Phase A | 0.1% Aqueous formic acid (v/v) | | |
| Mobile Phase B | 0.1% Formic acid in acetonitrile (v/v) | | |
| Flow | 2.0 ml/min | | |
| Gradient Program | Time (mins) | % A | % B |
| | 0.0 | 95 | 5 |
| | 0.3 | 95 | 5 |
| | 4.3 | 5 | 95 |
| | 5.3 | 5 | 95 |
| | 5.8 | 95 | 5 |
| | 6.0 | 95 | 5 |

| | |
|---|---|
| Detectors | UV, diode array + Sedex 85 |
| | evaporative light scattering detector |

Method 8:

| Instrumentation | HP1100 (quaternary pump/PDA detector) + | | |
|---|---|---|---|
| | ZQ Mass Spectrometer | | |
| Column | Phenomenex Luna C18(2) 3μ, 30 × 4.6 mm | | |
| Mobile Phase A | 0.1% Aqueous formic acid | | |
| Mobile Phase B | 0.1% Formic acid in acetonitrile | | |
| Flow | 2.0 ml/min | | |
| Gradient Program | Time (mins) | % A | % B |
| | 0.0 | 95 | 05 |
| | 0.3 | 95 | 05 |
| | 4.3 | 05 | 95 |
| | 5.3 | 05 | 95 |
| | 5.8 | 95 | 05 |
| | 6.0 | 95 | 05 |

| | |
|---|---|
| Detectors | UV, diode array 190-450 nm |
| | MS, mass 160-1000 (or 100-800, or 160-1300) |
| | in ES+ & ES− (200 μl/min split to MS) |

Method 9:

| Instrumentation | UPLC + Waters DAD + Waters SQD2, single quadrapole UPLC-MS | | |
|---|---|---|---|
| Column | Acquity UPLC HSS C18 1.8 um 100 × 2.1 mm. | | |
| | (Plus guard cartridge), maintained at temp | | |
| Mobile Phase A | Water (High purity via PureLab Option unit) with 0.1% formic acid | | |
| Mobile Phase B | Acetonitrile (Far UV grade) with 0.1% (V/V) formic acid | | |
| Flow | 0.5 ml/min | | |
| Gradient Program | Time (mins) | % A | % B |
| | 0.0 | 95 | 05 |
| | 1.2 | 95 | 05 |
| | 3.5 | 0 | 100 |
| | 4.9 | 0 | 100 |
| | 5 | 95 | 05 |
| | 6 | 95 | 05 |

| | |
|---|---|
| Sample | 0.5-2 ul (concentration ~0.2-1 mg/ml). |
| Detectors | UV, diode array 210 nm-400 nm Resolution 1.2 nm |
| | Other wavelength traces are extracted from the DAD data |
| | MS, mass 100-700 (or −1500 for HM method) in ES+ & ES− |
| | (300 μl/min split to MS) |

Method 10:

| Instrumentation | UPLC + Waters DAD + Waters SQD2, single quadrapole UPLC-MS | | |
|---|---|---|---|
| Column | Acquity UPLC BEH Shield RP18 1.7 um 100 × 2.1 mm. | | |
| | (Plus guard cartridge), maintained at temp | | |
| Mobile Phase A | Water (High purity via PureLab Option unit) with 10 mM ammonium | | |
| | bicarbonate (ammonium hydrogen carbonate) | | |
| Mobile Phase B | Acetonitrile (Far UV grade) | | |
| Flow | 0.5 ml/min | | |
| Gradient Program | Time (mins) | % A | % B |
| | 0.0 | 95 | 05 |
| | 1.2 | 95 | 05 |
| | 3.5 | 0 | 100 |
| | 4.9 | 0 | 100 |
| | 5 | 95 | 05 |
| | 6 | 95 | 05 |

| | |
|---|---|
| Sample | 0.5-2 ul (concentration ~0.2-1 mg/ml). |
| Detectors | UV, diode array 210 nm-400 nm Resolution 1.2 nm |
| | Other wavelength traces are extracted from the DAD data |
| | MS, mass 100-700 (or −1500 for HM method) in ES+ & ES− |
| | (300 μl/min split to MS) |

MDAP methods used were as follows.

MDAP Method (Standard—Acidic):
Agilent Technologies 1260 Infinity purification system with an XSELECT CSH Prep
C18 column (19×250 mm, 5 μm OBD) maintained at RT
Mobile Phase A: 0.1% aqueous formic acid
Mobile Phase B: 0.1% formic acid in acetonitrile
Flow Rate: 20 ml/min
Gradient Program: 10%-95%, 22 min, centered around a specific focused gradient
   Sample: Injection of a 20-60 mg/mL solution in DMSO (+optional formic acid and water)

MDAP Method (Basic):
Agilent Technologies 1260 Infinity purification system with an XBridge Prep C18
OBD column (19×250 mm, 5 μm OBD) maintained at RT
Mobile Phase A: 0.1% aqueous ammonia
Mobile Phase B: 0.1% ammonia in acetonitrile
Flow Rate: 20 ml/min
Gradient Program: 10%-95%, 22 min, centered around a specific focused gradient
Sample: Injection of a 20-60 mg/ml solution in DMSO+optional formic acid and water)

### Intermediate 1A: (4-Bromo-2-fluorophenyl)[2-(diethoxymethyl)phenyl]methanone

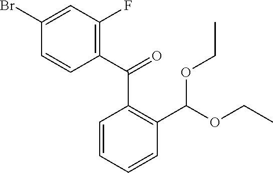 [SMILES: CCOC(OCC)c1ccccc1C(=O)c1ccc(Br)cc1F]

n-Butyllithium (1.6M in hexanes, 53.2 mL) was added dropwise over 10 minutes to a stirred, cooled solution of 1-bromo-2-diethoxymethylbenzene (22.0 g) in anhydrous diethyl ether (120 mL) while maintaining the temperature below −60° C. On completion of the addition, the mixture was stirred at below −70° C. for 1 hour. A solution of 4-bromo-2-fluoro-N-methoxy-N-methylbenzamide (22.3 g) in anhydrous diethyl ether (120 mL) was added dropwise while maintaining the temperature below −70° C. After stirring at below −70° C. for a further 1 hour, the mixture was allowed to come to room temperature overnight. A saturated aqueous solution of ammonium chloride was added and the two phases were separated. The aqueous phase was extracted with diethyl ether and the combined organic phases were washed with water, dried (MgSO₄) and filtered. The filtrate was concentrated in vacuo to give a brown oil which was purified by FCC eluted with 0-50% ethyl acetate in isohexane to give the title compound as a pale yellow oil (19.8 g). ¹H NMR (400 MHz, CDCl₃) 7.72-7.70 (1H, m), 7.59-7.47 (2H, m), 7.40-7.26 (4H, m), 5.74 (1H, s), 3.62-3.55 (2H, m), 3.50-3.42 (2H, m), 1.13-1.08 (6H, m).

By proceeding in a similar manner to Intermediate 1A, the following compounds were prepared:

### Intermediate 1B: (2-Fluorophenyl) 2-(diethoxymethylphenyl)methanone

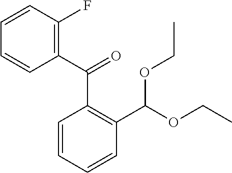 [SMILES: CCOC(OCC)c1ccccc1C(=O)c1ccccc1F]

Starting from 1-bromo-2-diethoxymethylbenzene and 2-fluoro-N-methoxy-N-methylbenzamide. ¹H NMR (400 MHz, CDCl₃) 7.74-7.66 (2H, m), 7.56-7.47 (2H, m), 7.39-7.30 (2H, m), 7.24-7.19 (1H, m), 7.14-7.08 (1H, m), 5.78 (1H, s), 3.65-3.56 (2H, m), 3.50-3.41 (2H, m), 1.11 (6H, t, J=7.4 Hz).

### Intermediate 1C: [2-(1,3-Dioxolan-2-yl)-3-methylphenyl](2-fluorophenyl)methanone

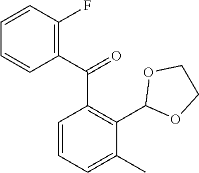 [SMILES: Cc1cccc(C(=O)c2ccccc2F)c1C1OCCO1]

Starting from 2-(2-bromo-6-methylphenyl)-1,3-dioxolane (Intermediate 28A) and 2-fluoro-N-methoxy-N-methylbenzamide. LCMS (Method 5) RT 3.88 min m/z 287 [MH⁺]

### Intermediate 1D: [2-(1,3-Dioxolan-2-yl)-3-fluorophenyl](2-fluorophenyl)methanone

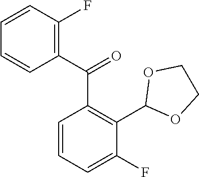 [SMILES: O=C(c1ccccc1F)c1cccc(F)c1C1OCCO1]

Starting from 2-(2-bromo-6-fluorophenyl)-1,3-dioxolane (Intermediate 28C) and 2-fluoro-N-methoxy-N-methylbenzamide. LCMS (Method 4) RT 1.38 min m/z 313 [M+Na⁺]

### Intermediate 1E: (4-Chloro-2-fluorophenyl)[2-(diethoxymethyl)phenyl]methanone

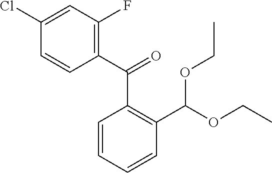 [SMILES: CCOC(OCC)c1ccccc1C(=O)c1ccc(Cl)cc1F]

Starting from 1-bromo-2-diethoxymethylbenzene and 4-chloro-2-fluoro-N-methoxy-N-methylbenzamide. ¹H NMR (400 MHz, CDCl₃) 7.74-7.68 (1H, m), 7.67-7.62 (1H, m), 7.51-7.46 (1H, m), 7.40-7.34 (1H, m), 7.31-7.26 (1H, m), 7.24-7.19 (1H, m), 7.17-7.12 (1H, m), 5.74 (1H, s), 3.62-3.56 (2H, m), 3.50-3.43 (2H, m), 1.10 (6H, t, J=7.1 Hz).

### Intermediate 2A: {4-Bromo-2-[(propan-2-ylideneamino)oxy]phenyl}[2-(diethoxymethyl)phenyl]methanone

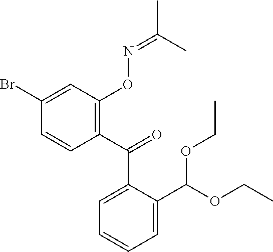 [SMILES: CCOC(OCC)c1ccccc1C(=O)c1ccc(Br)cc1ON=C(C)C]

A solution of potassium tert-butoxide in THF (1M, 56 mL) was added dropwise over 15 minutes to a stirred and cooled solution of acetone oxime (3.99 g) in dry THF (165 mL) at 0° C. under an atmosphere of nitrogen. The resultant white suspension was stirred at 0° C. for minutes then a solution of (4-bromo-2-fluorophenyl)[2-(diethoxymethyl)phenyl]methanone (Intermediate 1A, 19.8 g) in dry THF (100 mL) was slowly added over 15 minutes at 0° C. The resultant mixture was allowed to come to room temperature and stirred for 2 hours. The dark solution was partitioned between ethyl acetate and brine and the organic phase was dried (Na₂SO₄) and filtered. The filtrate was concentrated in vacuo and the residue was purified by FCC eluted with 0-45% ethyl acetate in isohexane to give the title compound as a cream solid (17.9 g). ¹H NMR (400 MHz, CDCl₃) 7.75 (1H, d, J=1.9 Hz), 7.73-7.70 (1H, m), 7.52 (1H, d, J=8.3 Hz), 7.47-7.41 (1H, m), 7.34-7.30 (2H, m), 7.19 (1H, dd, J=1.9, 8.3 Hz), 5.81 (1H, s), 3.70-3.62 (2H, m), 3.51-3.42 (2H, m), 1.89 (3H, s), 1.40 (3H, s) 1.13 (6H, t, J=7.1 Hz).

By proceeding in a similar manner to Intermediate 2A, the following compounds were prepared:

### Intermediate 2B: [2-(Diethoxymethyl)phenyl]{2-[(propan-2-ylideneamino)oxy]-phenyl}methanone

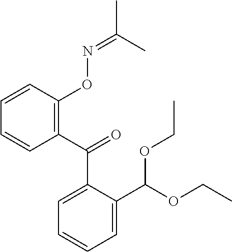 [SMILES: CCOC(OCC)c1ccccc1C(=O)c1ccccc1ON=C(C)C]

Starting from (2-fluorophenyl)[2-(diethoxymethyl)phenyl]methanone (Intermediate 1B). ¹H NMR (400 MHz, CDCl₃) 7.73 (1H, d, J=7.7 Hz), 7.65 (1H, dd, J=1.6, 7.7 Hz), 7.54-7.41 (3H, m), 7.38-7.28 (2H, m), 7.08-7.04 (1H, m), 5.85 (1H, s), 3.71-3.63 (2H, m), 3.51-3.42 (2H, m), 1.88 (3H, s), 1.42 (3H, s), 1.14 (6H, t, J=7.1 Hz).

### Intermediate 2C: [2-(1,3-Dioxolan-2-yl)-3-methylphenyl]{2-[(propan2-ylideneamino)-oxy]phenyl}methanone

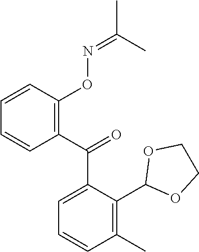 [SMILES: CC(C)=NOc1ccccc1C(=O)c1cccc(C)c1C1OCCO1]

Starting from [2-(1,3-dioxolan-2-yl)-3-methylphenyl](2-fluorophenylmethanone (Intermediate 1C). LCMS (Method 5) RT 4.20 min m/z 340 [MH⁺]

### Intermediate 2D: {3-Bromo-2-[(propan-2-ylideneamino)oxy]phenyl}[2-(diethoxy-methyl)phenyl]methanone

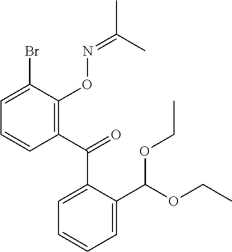 [SMILES: CCOC(OCC)c1ccccc1C(=O)c1cccc(Br)c1ON=C(C)C]

Starting from (3-bromo-2-fluorophenyl)[2-(diethoxymethyl)phenyl]methanone (Intermediate 39A). LCMS (Method 5) RT 4.77 min m/z 456/458 [M+Na⁺].

### Intermediate 2E: [3-Bromo-2-(1,3-dioxolan-2-yl)phenyl]{2-[(propan-2-ylidieneamino)-oxy]phenyl}methanone

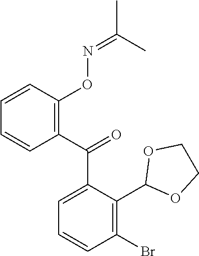 [SMILES: CC(C)=NOc1ccccc1C(=O)c1cccc(Br)c1C1OCCO1]

Starting from [3-bromo-2-(1,3-dioxolan-2-yl)phenyl](2-fluorophenyl)methanone (Intermediate 39B). LCMS (Method 5) RT 4.23 min m/z 404/406 [MH⁺].

### Intermediate 2F: [2-(1,3-Dioxolan-2-yl)-3-fluorophenyl]{2-[(propan-2-ylidieneamino)-oxy]phenyl}methanone

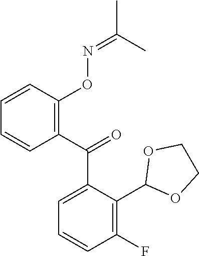 [SMILES: CC(C)=NOc1ccccc1C(=O)c1cccc(F)c1C1OCCO1]

Starting from [3-fluoro-2-(1,3-dioxolan-2-yl)phenyl](2-fluorophenyl)methanone (Intermediate 1D). LCMS (Method 6) RT 3.70 min m/z 344 [MH⁺].

### Intermediate 2G: (2-Diethoxymethylphenyl){2-[(propan-2-ylideneamino)oxy]-4-(trifluoromethyl)phenyl}methanone

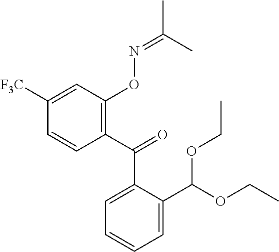 [SMILES: CCOC(OCC)c1ccccc1C(=O)c1ccc(C(F)(F)F)cc1ON=C(C)C]

Starting from (2-fluoro-4-trifluoromethylphenyl)[2-(diethoxymethyl)phenyl]-methanone (Intermediate 39C). ¹H NMR (400 MHz, CDCl₃) 7.84-7.82 (1H, m), 7.77-7.70 (2H, m), 7.50-7.44 (1H, m), 7.37-7.28 (3H, m), 5.87 (1H, s), 3.73-7.63 (2H, m), 3.52-3.43 (2H, m), 1.90 (3H, s), 1.43 (3H, s), 1.15 (6H, t, J=7.1 Hz).

### Intermediate 2H: {4-chloro-2-[(propan-2-ylideneamino)oxy]phenyl}[2-(diethoxymethyl)phenyl]methanone

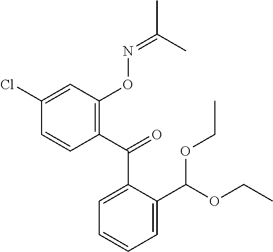 [SMILES: CCOC(OCC)c1ccccc1C(=O)c1ccc(Cl)cc1ON=C(C)C]

Starting from (4-chloro-2-fluorophenyl)[2-(diethoxymethyl)phenyl]methanone (Intermediate 1E). LCMS (Method 8) RT 4.28 min m/z 412 [M+Na⁺].

### Intermediate 21: [2-(Diethoxymethyl)phenyl]{4-fluoro-2-[(propan-2-ylideneamino)-oxy]phenyl}methanone

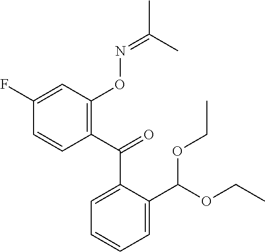 [SMILES: CCOC(OCC)c1ccccc1C(=O)c1ccc(F)cc1ON=C(C)C]

Starting from [2-(diethoxymethylphenyl)(2,4-difluorophenyl)methanone (Intermediate 39D). ¹H NMR (400 MHz, CDCl₃) 7.73-7.65 (2H, m), 7.46-7.40 (1H, m), 7.35-7.27 (3H, m), 6.77-6.70 (1H, m), 5.80 (1H, s), 3.70-3.61 (2H, m), 3.50-3.41 (2H, m), 1.89 (3H, s), 1.42 (3H, s), 1.13 (6H, t, J=7.0 Hz).

### Intermediate 2J: [2-(Diethoxymethyl)phenyl]{5-fluoro-2-[(propan-2-ylideneamino)-oxy]phenyl}methanone

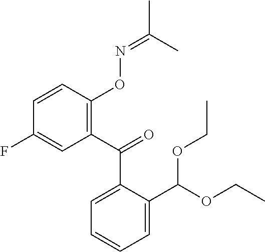 [SMILES: CCOC(OCC)c1ccccc1C(=O)c1cc(F)ccc1ON=C(C)C]

Starting from [2-(diethoxymethylphenyl)(2,5-difluorophenyl)methanone (Intermediate 39E). ¹H NMR (400 MHz, CDCl₃) 7.75-7.70 (1H, m), 7.49-7.42 (2H, m), 7.39-7.28 (3H, m), 7.22-7.15 (1H, m), 5.84 (1H, s), 3.72-3.61 (2H, m), 3.53-3.41 (2H, m), 1.85 (3H, s), 1.36 (3H, s), 1.14 (6H, t, J=7.0 Hz).

### Intermediate 2K: [2-(diethoxymethyl)phenyl]{3-fluoro-2-[(propan-2-ylideneamino)-oxy]phenyl}methanone

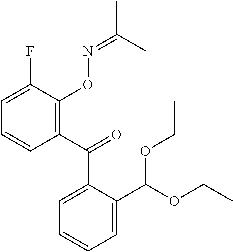 [SMILES: CCOC(OCC)c1ccccc1C(=O)c1cccc(F)c1ON=C(C)C]

Starting from [2-(diethoxymethylphenyl)(2,3-difluorophenyl)methanone (Intermediate 39F). ¹H NMR (400 MHz, CDCl₃) 7.76-7.72 (1H, m), 7.49-7.44 (1H, m), 7.42-7.36 (2H, m), 7.33-7.25 (2H, m), 7.18-7.12 (1H, m), 5.89 (1H, s), 3.73-3.64 (2H, m), 3.54-3.46 (2H, m), 1.74 (3H, s), 1.56 (3H, s), 1.17 (6H, t, J=7.0 Hz).

### Intermediate 3A: 2-(6-Bromobenzo[d]isoxazol-3-yl)benzaldehyde

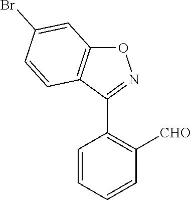 [SMILES: O=Cc1ccccc1-c1noc2cc(Br)ccc12]

Aqueous hydrochloric acid (2M, 24.8 mL) was added to a stirred suspension of {4-bromo-2-[(propan-2-ylideneamino)oxy]phenyl}[2-(diethoxymethyl)phenyl]-methanone (Intermediate 2A, 17.98 g) in IPA (50 mL) and the resultant mixture was stirred and heated at 80° C. for 3 hours. The mixture was cooled to room temperature and then in an ice bath and the resultant precipitate was collected by filtration, washed with a little IPA and dried in vacuo to give the title compound as a cream solid (12.04 g). ¹H NMR (400 MHz, CDCl₃) 10.19 (1H, s), 8.19-8.16 (1H, m), 7.91 (1H, d, J=0.7 Hz), 7.84-7.71 (3H, m), 7.54-7.48 (2H, m).

By proceeding in a similar manner to Intermediate 3A, the following compounds were prepared:

### Intermediate 3B: 2-(Benzo[d]isoxazol-3-yl)benzaldehyde

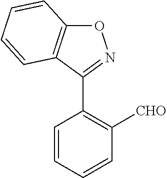 [SMILES: O=Cc1ccccc1-c1noc2ccccc12]

Starting from [2-(diethoxymethyl)phenyl]{2-[(propan-2-ylideneamino)oxy]-phenyl}methanone (Intermediate 2B). ¹H NMR (400 MHz, CDCl₃) 10.22 (1H, s), 8.21-8.18 (1H, m), 7.81-7.79 (2H, m), 7.73-7.63 (4H, m), 7.43-7.38 (1H, m).

### Intermediate 3C: 2-(Benzo[d]isoxazol-3-yl)-6-methylbenzaldehyde

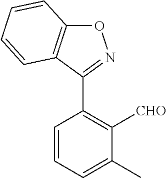 [SMILES: Cc1cccc(-c2noc3ccccc23)c1C=O]

Starting from [2-(diethoxymethyl)phenyl]{2-[(propan-2-ylideneamino)oxy]-3-methylphenyl}methanone (Intermediate 2C). LCMS (Method 5) RT 4.04 m/z 238 [MH⁺].

### Intermediate 3D: 2-(7-Bromobenzo[d]isoxazol-3-yl) benzaldehyde

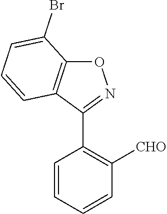 [SMILES: O=Cc1ccccc1-c1noc2c(Br)cccc12]

Starting from {3-bromo-2-[(propan-2-ylideneamino)oxy]phenyl}[2-(diethoxymethyl)phenyl]methanone (Intermediate 2D). LCMS (Method 5) RT 4.15 m/z 302/304 [MH⁺].

### Intermediate 3E: 2-(Benzo[d]isoxazol-3-yl)-6-bromobenzaldehyde

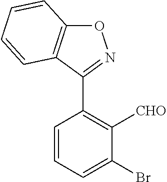 [SMILES: O=Cc1c(Br)cccc1-c1noc2ccccc12]

Starting from [3-bromo-2-(1,3-dioxolan-2-yl)phenyl]{2-[(propan-2-ylidieneamino)oxy]phenyl}methanone (Intermediate 2E). LCMS (Method 5) RT 4.03 m/z 302/304 [MH⁺].

### Intermediate 3F: 2-(Benzo[d]isoxazol-3-yl)-6-fluorobenzaldehyde

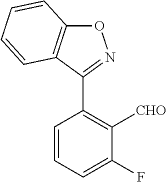 [SMILES: O=Cc1c(F)cccc1-c1noc2ccccc12]

Starting from [2-(1,3-dioxolan-2-yl)-3-fluorophenyl]{2-[(propan-2-ylidieneamino)oxy]phenyl}methanone (Intermediate 2F). LCMS (Method 6) RT 3.34 m/z 242 [MH⁺].

### Intermediate 3G: 2-(6-Trifluoromethylbenzo[d]isoxazol-3-yl)benzaldehyde

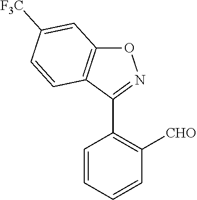 [SMILES: O=Cc1ccccc1-c1noc2cc(C(F)(F)F)ccc12]

Starting from [2-(diethoxymethyl)phenyl]{2-[(propan-2-ylideneamino)oxy]-4-(trifluoromethyl)phenyl}methanone (Intermediate 2G). LCMS (Method 3) RT 1.44 m/z 292 [MH⁺].

### Intermediate 3H: 2-(6-Chlorobenzo[d]isoxazol-3-yl)benzaldehyde

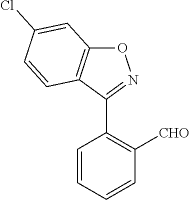 [SMILES: O=Cc1ccccc1-c1noc2cc(Cl)ccc12]

Starting from {4-chloro-2-[(propan-2-ylideneamino)oxy]phenyl}[2-(diethoxymethyl)phenyl]methanone (Intermediate 2H). LCMS (Method 3) RT 1.40 m/z 258/260 [MH⁺].

### Intermediate 31: 2-(6-Fluorobenzo[d]isoxazol-3-yl)benzaldehyde

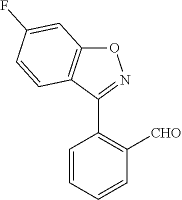 [SMILES: O=Cc1ccccc1-c1noc2cc(F)ccc12]

Starting from [2-(diethoxymethyl)phenyl]{4-fluoro-2-[(propan-2-ylideneamino)oxy]phenyl}methanone (Intermediate 21). ¹H NMR (400 Mz, CDCl₃) 10.20 (1H, s), 8.20-8.16 (1H, m), 7.83-7.70 (3H, m), 7.62-7.56 (1H, m), 7.39 (1H, dd, J=2.1 Hz, 8.3 Hz), 7.16 (1H, dt, J=2.1, 8.8 Hz).

### Intermediate 3J: 2-(5-Fluorobenzo[d]isoxazol-3-yl)benzaldehyde

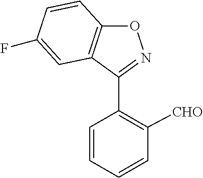 [SMILES: O=Cc1ccccc1-c1noc2ccc(F)cc12]

Starting from [2-(diethoxymethyl)phenyl]{5-fluoro-2-[(propan-2-ylideneamino)oxy]phenyl}methanone (Intermediate 2J). LCMS (Method 3) RT 1.39 m/z 242 [MH⁺].

### Intermediate 3K: 2-(7-Fluorobenzo[d]isoxazol-3-yl)benzaldehyde

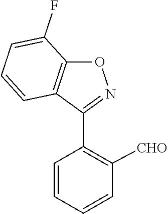 [SMILES: O=Cc1ccccc1-c1noc2c(F)cccc12]

Starting from [2-(diethoxymethyl)phenyl]{3-fluoro-2-[(propan-2-ylideneamino)oxy]phenyl}methanone (Intermediate 2K). ¹H NMR (400 Mz, CDCl₃) 10.20 (1H, s), 8.22-8.16 (1H, m), 7.85-7.70 (3H, m), 7.43-7.30 (3H, m).

### Intermediate 4A: (S,E)-N-[2-(6-Bromobenzo[d]isoxazol-3-yl)benzylidene]-2-methylpropane-2-sulfinamide

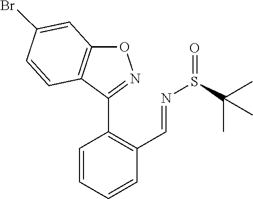 [SMILES: CC(C)(C)[S@](=O)/N=C/c1ccccc1-c1noc2cc(Br)ccc12]

A mixture of 2-(6-bromobenzo[d]isoxazol-3-yl)benzaldehyde (Intermediate 3A, 4.0 g), (S)-2-methylpropane-2-sulfinamide (1.93 g) and cesium carbonate (5.18 g) in DCM (80 mL) was stirred and heated at reflux for 2 hours under an atmosphere of nitrogen. The mixture was stirred at room temperature overnight then again heated at reflux for a further 4 hours. After cooling, the mixture was diluted with DCM, washed with water, dried (Na₂SO₄) and filtered. The filtrate was concentrated in vacuo and the residue was purified by FCC, eluting with 0-50% ethyl acetate in isohexane to give the title compound as a cream solid (4.86 g). LCMS (Method 3): RT 1.62 min m/z 405, 407.

By proceeding in a similar manner to Intermediate 4A, the following compounds were prepared:

### Intermediate 4B: (S,E)-N-[2-(Benzo[d]isoxazol-3-yl)benzylidene]-2-methylpropane-2-sulfinamide (CWE4150-038)

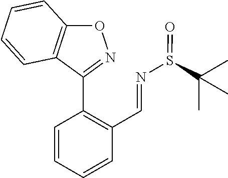 [SMILES: CC(C)(C)[S@](=O)/N=C/c1ccccc1-c1noc2ccccc12]

Starting from 2-(benzo[d]isoxazol-3-yl)benzaldehyde (Intermediate 3B) and (S)-2-methylpropane-2-sulfinamide. LCMS (Method 4): RT 1.58 m/z 327.

### Intermediate 4C: (S,E)-N-[2-(4-Bromobenzo[d]isoxazol-3-yl)benzylidene]-2-methylpropane-2-sulfinamide

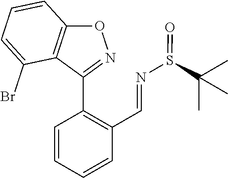 [SMILES: CC(C)(C)[S@](=O)/N=C/c1ccccc1-c1noc2cccc(Br)c12]

Starting from 2-(4-bromobenzo[d]isoxazol-3-yl)benzaldehyde (Intermediate 25A) and (S)-2-methylpropane-2-sulfinamide. LCMS (Method 6) RT 3.92 m/z 405/407.

### Intermediate 4D: (S,E)-N-[2-(Benzo[d]isoxazol-3-yl)-5-methoxybenzylidene]-2-methylpropane-2-sulfinamide

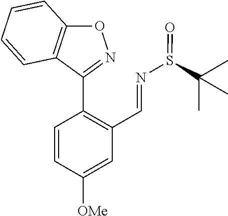 [SMILES: COc1ccc(-c2noc3ccccc23)c(/C=N/[S@@](=O)C(C)(C)C)c1]

Starting from 2-(benzo[d]isoxazol-3-yl)-5-methoxybenzaldehyde (Intermediate 26A) and (S)-2-methylpropane-2-sulfinamide. LCMS (Method 5) RT 4.35 m/z 357 [MH⁺].

### Intermediate 4E: (S,E)-N-[2-(Benzo[d]isoxazol-3-yl)-5-bromobenzylidene]-2-methylpropane-2-sulfinamide

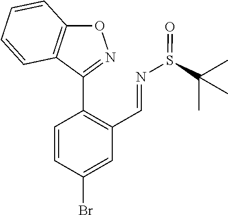 [SMILES: CC(C)(C)[S@](=O)/N=C/c1cc(Br)ccc1-c1noc2ccccc12]

Starting from 2-(benzo[d]isoxazol-3-yl)-5-bromobenzaldehyde (Intermediate 26B) and (S)-2-methylpropane-2-sulfinamide. ¹H NMR (300 MHz, CDCl₃) 8.71 (1H, s), 8.38 (1H, d, J=2.0 Hz), 7.81 (1H, dd, J=2.1, 8.2 Hz), 7.70-7.53 (4H, m), 7.40-7.33 (1H, m), 1.23-1.22 (9H, m).

### Intermediate 4F: (S,E)-N-[2-(Benzo[d]isoxazol-3-yl)-6-methylbenzylidene]-2-methylpropane-2-sulfinamide

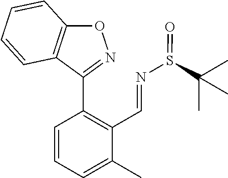 [SMILES: Cc1cccc(-c2noc3ccccc23)c1/C=N/[S@@](=O)C(C)(C)C]

Starting from 2-(benzo[d]isoxazol-3-yl)-6-methylbenzaldehyde (Intermediate 3C) and (S)-2-methylpropane-2-sulfinamide. LCMS (Method 5) RT 4.33 m/z 341 [MH⁺].

### Intermediate 4G: (S,E)-N-[2-(Benzo[d]isoxazol-3-yl)-4-bromobenzylidene]-2-methylpropane-2-sulfinamide

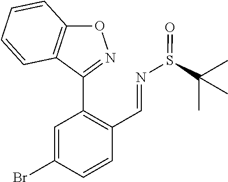 [SMILES: CC(C)(C)[S@](=O)/N=C/c1ccc(Br)cc1-c1noc2ccccc12]

Starting from 2-(benzo[d]isoxazol-3-yl)-4-bromobenzaldehyde (Intermediate 26C) and (S)-2-methylpropane-2-sulfinamide. LCMS (Method 5) RT 4.66 m/z 405/407 [MH⁺].

### Intermediate 4H: (S,E)-N-[2-(Benzo[d]isoxazol-3-yl)-3-bromobenzylidene]-2-methylpropane-2-sulfinamide

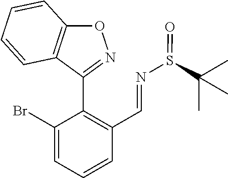 [SMILES: CC(C)(C)[S@](=O)/N=C/c1cccc(Br)c1-c1noc2ccccc12]

Starting from 2-(benzo[d]isoxazol-3-yl)-3-bromobenzaldehyde (Intermediate 26D) and (S)-2-methylpropane-2-sulfinamide. LCMS (Method 5) RT 4.34 m/z 405/407 [MH⁺].

### Intermediate 41: (S,E)-N-[2-(7-Bromobenzo[d]isoxazol-3-yl)benzylidene]-2-methylpropane-2-sulfinamide

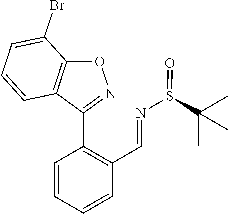 [SMILES: CC(C)(C)[S@](=O)/N=C/c1ccccc1-c1noc2c(Br)cccc12]

Starting from 2-(7-bromobenzo[d]isoxazol-3-yl)benzaldehyde (Intermediate 3D) and (S)-2-methylpropane-2-sulfinamide. LCMS (Method 5) RT 4.50 m/z 405/407 [MH⁺].

### Intermediate 4J: (S,E)-N-[2-(Benzo[d]isoxazol-3-yl)-6-bromobenzylidene]-2-methylpropane-2-sulfinamide

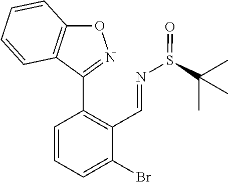 [SMILES: CC(C)(C)[S@](=O)/N=C/c1c(Br)cccc1-c1noc2ccccc12]

Starting from 2-(benzo[d]isoxazol-3-yl)-6-bromobenzaldehyde (Intermediate 3E) and (S)-2-methylpropane-2-sulfinamide. LCMS (Method 5) RT 4.30 m/z 405/407 [MH⁺].

### Intermediate 4K: (S,E)-N-[2-(Benzo[d]isoxazol-3-yl)-6-fluorobenzylidene]-2-methylpropane-2-sulfinamide

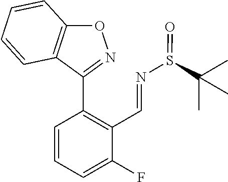 [SMILES: CC(C)(C)[S@](=O)/N=C/c1c(F)cccc1-c1noc2ccccc12]

Starting from 2-(benzo[d]isoxazol-3-yl)-6-fluorobenzaldehyde (Intermediate 3F) and (S)-2-methylpropane-2-sulfinamide. LCMS (Method 4) RT 1.57 m/z 367 [M+Na⁺].

### Intermediate 4L: (S,E)-2-Methyl-N-[2-(1-methyl-1H-indazol-3-yl)benzylidene]propane-2-sulfinamide

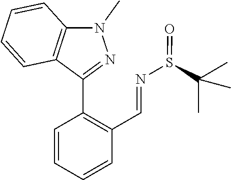 [SMILES: Cn1nc(-c2ccccc2/C=N/[S@@](=O)C(C)(C)C)c2ccccc21]

Starting from 2-(1-methyl-1H-indazole-3-yl)benzaldehyde (Intermediate 68A) and (S)-2-methylpropane-2-sulfinamide. ¹H NMR (400 MHz, CDCl₃) 8.90 (1H, s), 8.23 (1H, dd, J=1.3, 7.9 Hz), 7.79-7.60 (3H, m), 7.53-7.49 (1H, m), 7.46-7.43 (2H, m), 7.23-7.17 (1H, m), 4.15 (3H, s), 1.26 (9H, s).

### Intermediate 4M: (S,E)-N-[2-(1-isopropyl-1H-indazol-3-yl)benzylidene]-2-methylpropane-2-sulfinamide

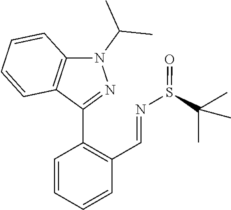 [SMILES: CC(C)n1nc(-c2ccccc2/C=N/[S@@](=O)C(C)(C)C)c2ccccc21]

Starting from 2-(1-Iso-propyl-1H-indazole-3-yl)benzaldehyde (Intermediate 68B) and (S)-2-methylpropane-2-sulfinamide. ¹H NMR (400 MHz, CDCl₃) 8.97 (1H, s), 8.26-8.24 (1H, m), 7.84-7.77 (2H, m), 7.65-7.60 (1H, m), 7.53-7.47 (2H, m), 7.42 (1H, t, J=7.6 Hz), 7.20 (1H, t, J=7.5 Hz), 4.95-4.85 (1H, m), 1.67 (6H, d, J=6.9 Hz), 1.28 (9H, s).

### Intermediate 4N: (S,E)-2-Methyl-N-[2-(6-trifluoromethylbenzo[d]isoxazol-3-yl)benzylidene]propane-2-sulfinamide

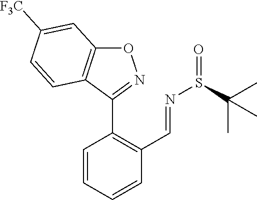 [SMILES: CC(C)(C)[S@](=O)/N=C/c1ccccc1-c1noc2cc(C(F)(F)F)ccc12]

Starting from 2-(6-trifluoromethylbenzo[d]isoxazol-3-yl)benzaldehyde (Intermediate 3G) and (S)-2-methylpropane-2-sulfinamide. ¹H NMR (400 MHz, CDCl₃) 8.73 (1H, s), 8.25-8.21 (1H, m), 7.98-7.95 (1H, m), 7.73-7.66 (4H, m), 7.63-7.59 (1H, m), 1.15 (9H, s).

### Intermediate 40: (S,E)-2-Methyl-N-[2-(6-methylbenzo[d]isoxazol-3-yl)benzylidene]propane-2-sulfinamide

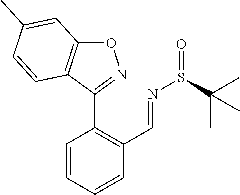 [SMILES: Cc1ccc2c(-c3ccccc3/C=N/[S@@](=O)C(C)(C)C)noc2c1]

Starting from 2-(6-methylbenzo[d]isoxazol-3-yl)benzaldehyde (Intermediate 11I) and (S)-2-methylpropane-2-sulfinamide. LCMS (Method 3) RT 1.57 m/z 341 [MH⁺].

### Intermediate 4P: (S,E)-N-[2-(6-Chlorobenzo[d]isoxazol-3-yl)benzylidene]-2-methylpropane-2-sulfinamide

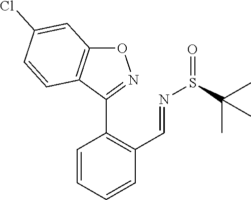 [SMILES: CC(C)(C)[S@](=O)/N=C/c1ccccc1-c1noc2cc(Cl)ccc12]

Starting from 2-(6-chlorobenzo[d]isoxazol-3-yl)benzaldehyde (Intermediate 3H) and (S)-2-methylpropane-2-sulfinamide. LCMS (Method 3) RT 1.55 m/z 361 [MH⁺].

### Intermediate 4Q: (S,E)-N-[2-(6-Fluorobenzo[d]isoxazol-3-yl)benzylidene]-2-methylpropane-2-sulfinamide

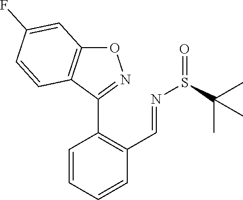 [SMILES: CC(C)(C)[S@](=O)/N=C/c1ccccc1-c1noc2cc(F)ccc12]

Starting from 2-(6-fluorobenzo[d]isoxazol-3-yl)benzaldehyde (Intermediate 31) and (S)-2-methylpropane-2-sulfinamide. LCMS (Method 3) RT 1.49 m/z 345 [MH⁺].

### Intermediate 4R: (S,E)-N-[2-(5-fluorobenzo[d]isoxazol-3-yl)benzylidene]-2-methylpropane-2-sulfinamide

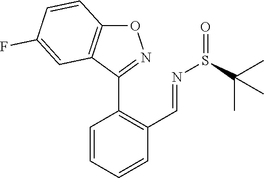 [SMILES: CC(C)(C)[S@](=O)/N=C/c1ccccc1-c1noc2ccc(F)cc12]

Starting from 2-(5-fluorobenzo[d]isoxazol-3-yl)benzaldehyde (Intermediate 3J) and (S)-2-methylpropane-2-sulfinamide. LCMS (Method 3) RT 1.62 m/z 345 [MH⁺]

### Intermediate 4S: (S,E)-N-[2-(7-Fluorobenzo[d]isoxazol-3-yl)benzylidene]-2-methylpropane-2-sulfinamide

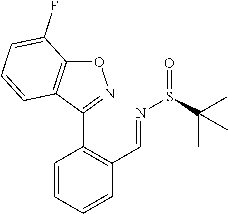 [SMILES: CC(C)(C)[S@](=O)/N=C/c1ccccc1-c1noc2c(F)cccc12]

Starting from 2-(7-fluorobenzo[d]isoxazol-3-yl)benzaldehyde (Intermediate 3K) and (S)-2-methylpropane-2-sulfinamide. ¹H NMR (400 MHz, CDCl₃) 8.72 (1H, s), 8.25-8.20 (1H, m), 7.71-7.67 (3H, m), 7.36-7.26 (3H, m), 1.17 (9H, s).

### Intermediate 5A: (S)—N—{(S)-1-[2-(6-Bromobenzo[d]isoxazol-3-yl)phenyl]-2-(pyridine-2-yl)ethyl}-2-methylpropane-2-sulfinamide

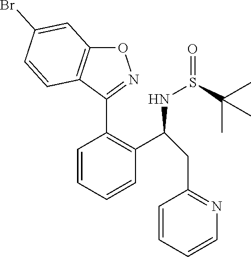 [SMILES: CC(C)(C)[S@](=O)N[C@@H](Cc1ccccn1)c1ccccc1-c1noc2cc(Br)ccc12]

n-Butyllithium (2.5M in hexanes, 4.8 mL) was added dropwise over 10 minutes to a stirred, cooled solution of 2-methylpyridine (1.06 g) in anhydrous THF (13 mL), while maintaining the temperature below −60° C. The resultant mixture was stirred at below −70° C. for 30 minutes then a solution of (S,E)-N-[2-(6-bromobenzo[d]isoxazol-3-yl)benzylidene]-2-methylpropane-2-sulfinamide (Intermediate 4A, 2.31 g) in anhydrous THF (10 mL) was added dropwise over 5 minutes while maintaining the temperature below −60° C. After stirring at −78° C. for a further 30 minutes, the mixture was allowed to warm to −30° C. and a saturated solution of ammonium chloride was added and the temperature was then allowed to warm to room temperature. The mixture was extracted with ethyl acetate and the organic phase was washed with brine, dried (MgSO₄) and filtered. The filtrate was concentrated in vacuo and the residue was purified by FCC eluting with 75-100% ethyl acetate in pentane then 0-5% methanol in ethyl acetate to give the faster running component as the title compound as a pale yellow gum (0.556 g). ¹H NMR (400 MHz, CDCl₃) 8.46-8.45 (1H, m), 7.87 (1H, d, J=0.8 Hz), 7.70-7.66 (1H, m), 7.55-7.42 (6H, m), 7.11-7.08 (1H, m), 7.03-6.99 (1H, m), 5.42 (1H, d, J=6.6 Hz), 5.07-5.00 (1H, m), 3.34 (1H, dd, J=4.5, 13.9 Hz), 3.24-3.14 (1H, m), 1.01 (9H, s).

By proceeding in a similar manner to Intermediate 5A the following compounds were prepared:

### Intermediate 5B: (S)—N—{(S)-1-[2-(4-Bromobenzo[d]isoxazol-3-yl)phenyl]-2-(pyridine-2-yl)ethyl}-2-methylpropane-2-sulfinamide

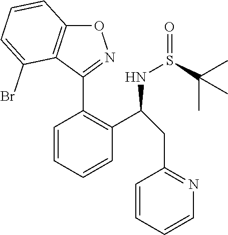 [SMILES: CC(C)(C)[S@](=O)N[C@@H](Cc1ccccn1)c1ccccc1-c1noc2cccc(Br)c12]

Starting from (S,E)-N-[2-(4-bromobenzo[d]isoxazol-3-yl)benzylidene]-2-methylpropane-2-sulfinamide (Intermediate 4C) and 2-methylpyridine. LCMS (Method 6) RT 2.75 m/z 498/500 [MH⁺].

### Intermediate 5C: (S)—N—{(S)-1-[2-(Benzo[d]isoxazol-3-yl)-5-methoxyphenyl]-2-(pyridine-2-yl)ethyl}-2-methylpropane-2-sulfinamide

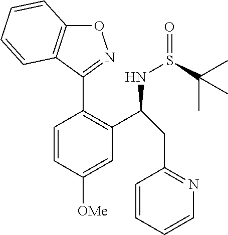 [SMILES: COc1ccc(-c2noc3ccccc23)c([C@H](Cc2ccccn2)N[S@@](=O)C(C)(C)C)c1]

Starting from (S,E)-N-[2-(benzo[d]isoxazol-3-yl)-5-methoxybenzylidene]-2-methylpropane-2-sulfinamide (Intermediate 4D) and 2-methylpyridine. LCMS (Method 5) RT 3.11 m/z 450 [MH⁺].

### Intermediate 5D: (S)—N—{(S)-1-[2-(Benzo[d]isoxazol-3-yl)-5-bromophenyl]-2-(pyridine-2-yl)ethyl}-2-methylpropane-2-sulfinamide

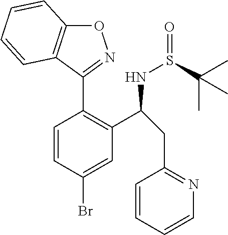 [SMILES: CC(C)(C)[S@](=O)N[C@@H](Cc1ccccn1)c1cc(Br)ccc1-c1noc2ccccc12]

Starting from (S,E)-N-[2-(benzo[d]isoxazol-3-yl)-5-bromobenzylidene]-2-methylpropane-2-sulfinamide (Intermediate 4E) and 2-methylpyridine. LCMS (Method 5) RT 3.43 m/z 498/500 [MH⁺].

### Intermediate 5E: (S)—N—{(S)-1-[2-(Benzo[d]isoxazol-3-yl)-6-methylphenyl]-2-(pyridine-2-yl)ethyl}-2-methylpropane-2-sulfinamide

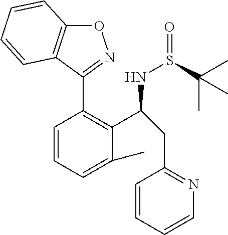 [SMILES: Cc1cccc(-c2noc3ccccc23)c1[C@H](Cc1ccccn1)N[S@@](=O)C(C)(C)C]

Starting from (S,E)-N-[2-(benzo[d]isoxazol-3-yl)-6-methylbenzylidene]-2-methylpropane-2-sulfinamide (Intermediate 4F) and 2-methylpyridine. LCMS (Method 5) RT 3.32 m/z 434 [MH⁺].

### Intermediate 5F: (S)—N—{(S)-1-[2-(Benzo[d]isoxazol-3-yl)-4-bromophenyl]-2-(pyridine-2-yl)ethyl}-2-methylpropane-2-sulfinamide

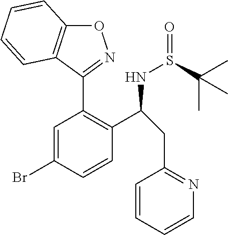 [SMILES: CC(C)(C)[S@](=O)N[C@@H](Cc1ccccn1)c1ccc(Br)cc1-c1noc2ccccc12]

Starting from (S,E)-N-[2-(benzo[d]isoxazol-3-yl)-4-bromobenzylidene]-2-methylpropane-2-sulfinamide (Intermediate 4G) and 2-methylpyridine. LCMS (Method 5) RT 3.40 m/z 498/500 [MH⁺].

### Intermediate 5G: (S)—N-[2-(Benzo[d]isoxazol-3-yl)phenyl]-1-(pyridin-2-yl)propan-2-yl]-2-methylpropan-2-sulfinamide

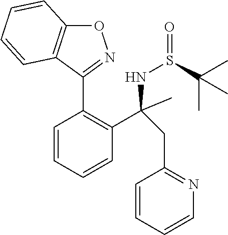 [SMILES: CC(C)(C)[S@](=O)N[C@@](C)(Cc1ccccn1)c1ccccc1-c1noc2ccccc12]

Starting from (S,E)-N-{1-[2-(benzo[d]isoxazol-3-yl)phenyl]ethylidene}-2-methylpropane-2-sulfinamide (Intermediate 32A) and 2-methylpyridine. LCMS (Method 6) RT 3.05 m/z 434 [MH⁺].

### Intermediate 5H: (S)—N—{(S)-1-[2-(Benzo[d]isoxazol-3-yl)-3-bromophenyl]-2-(pyridine-2-yl)ethyl}-2-methylpropane-2-sulfinamide

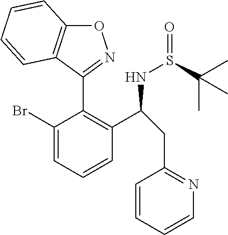 [SMILES: CC(C)(C)[S@](=O)N[C@@H](Cc1ccccn1)c1cccc(Br)c1-c1noc2ccccc12]

Starting from (S,E)-N-[2-(benzo[d]isoxazol-3-yl)-3-bromobenzylidene]-2-methylpropane-2-sulfinamide (Intermediate 4H) and 2-methylpyridine. LCMS (Method 5) RT 3.19 m/z 498/500 [MH⁺].

### Intermediate 51: (S)—N—{(S)-1-[2-(7-Bromobenzo[d]isoxazol-3-yl)phenyl]-2-(pyridine-2-yl)ethyl}-2-methylpropane-2-sulfinamide

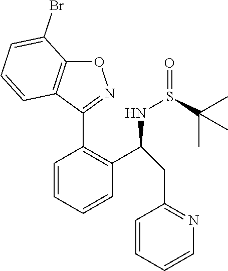 [SMILES: CC(C)(C)[S@](=O)N[C@@H](Cc1ccccn1)c1ccccc1-c1noc2c(Br)cccc12]

Starting from (S,E)-N-[2-(7-bromobenzo[d]isoxazol-3-yl)benzylidene]-2-methylpropane-2-sulfinamide (Intermediate 41) and 2-methylpyridine. LCMS (Method 5) RT 3.38 m/z 498/500 [MH⁺].

### Intermediate 5J: (S)—N—{(S)-1-[2-(Benzo[d]isoxazol-3-yl)-6-bromophenyl]-2-(pyridine-2-yl)ethyl}-2-methylpropane-2-sulfinamide

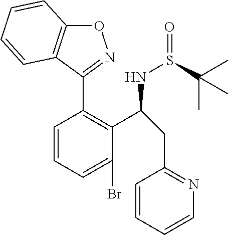 [SMILES: CC(C)(C)[S@](=O)N[C@@H](Cc1ccccn1)c1c(Br)cccc1-c1noc2ccccc12]

Starting from (S,E)-N-[2-(benzo[d]isoxazol-yl)-6-bromobenzylidene]-2-methylpropane-2-sulfinamide (Intermediate 4J). LCMS (Method 5) RT 3.22 m/z 498/500 [MH⁺].

### Intermediate 5K: (S)—N—{(S)-1-[2-(Benzo[d]isoxazol-3-yl)phenyl]-2-(pyridine-2-yl)butyl}-2-methylpropane-2-sulfinamide (TOJ 3830-012)

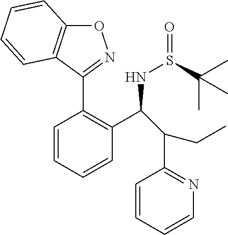 [SMILES: CCC(c1ccccn1)[C@H](N[S@@](=O)C(C)(C)C)c1ccccc1-c1noc2ccccc12]

Starting from (S,E)-N-[2-(benzo[d]isoxazol-3-yl)benzylidene]-2-methylpropane-2-sulfinamide (Intermediate 4B) and 2-propylpyridine and obtained as a diastereomeric mixture. LCMS (Method 6) RT 2.91 m/z 448 [MH⁺] and RT 3.12 m/z 448 [MH⁺].

### Intermediate 5L: (S)—N—{(S)-1-[2-(6-Bromobenzo[d]isoxazol-3-yl)phenyl]-2-(6-methylpyridine-2-yl)ethyl}-2-methylpropane-2-sulfinamide

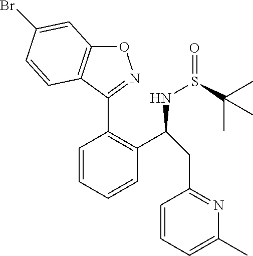 [SMILES: Cc1cccc(C[C@H](N[S@@](=O)C(C)(C)C)c2ccccc2-c2noc3cc(Br)ccc23)n1]

Starting from (S,E)-N-[2-(6-bromobenzo[d]isoxazol-3-yl)benzylidene]-2-methylpropane-2-sulfinamide (Intermediate 4A) and 2,6-dimethylpyridine. LCMS (Method 6) RT2.79 m/z 512/514 [MH⁺].

### Intermediate 5M: (S)-2-Methyl-N-{1-[2-(1-methyl-1H-indazol-3-yl)phenyl]-2-(pyridine-2-yl)ethyl}propane-2-sulfinamide

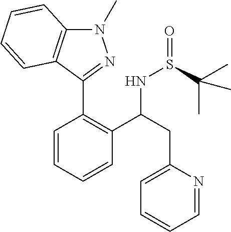 [SMILES: Cn1nc(-c2ccccc2C(Cc2ccccn2)N[S@@](=O)C(C)(C)C)c2ccccc21]

Starting from (S,E)-2-Methyl-N-[2-(1-methyl-1H-indazol-3-yl)benzylidene]propane-2-sulfinamide (Intermediate 4L) and 2-methylpyridine. LCMS (Method 4) RT 1.36 m/z 433 [MH⁺].

### Intermediate 5N: (S)-2-Methyl-N-{1-[2-(1-methyl-1H-indazol-3-yl)phenyl]-2-(6-methylpyridine-2-yl)ethyl}propane-2-sulfinamide

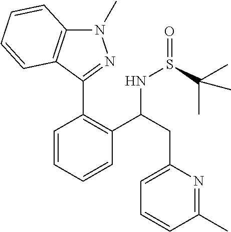 [SMILES: Cc1cccc(CC(N[S@@](=O)C(C)(C)C)c2ccccc2-c2nn(C)c3ccccc23)n1]

Starting from (S,E)-2-methyl-N-[2-(1-methyl-1H-indazol-3-yl)benzylidene]propane-2-sulfinamide (Intermediate 4L) and 2,6-dimethylpyridine. ¹H NMR (400 MHz, CDCl₃) 7.71-7.68 (1H, m), 7.60-7.59 (1H, m), 7.54-7.50 (1H, m), 7.46-7.44 (21H, m), 7.39-7.29 (3H, m), 7.21-7.16 (1H, m), 6.89 (1H, d, J=7.8 Hz), 6.64-6.60 (1H, m), 5.80-5.79 (1H, m), 5.14-5.07 (1H, m), 4.17 (3H, s), 3.22-3.17 (1H, m), 3.07 (1H, dd, J=8.3, 13.7 Hz), 2.44 (3H, s), 1.05 (9H, s).

### Intermediate 6A: (S)—N—{(S)-1-[2-(Benzo[d]isoxazol-3-yl)phenyl]-2-(pyridine-2-yl)ethyl}-2-methylpropane-2-sulfinamide

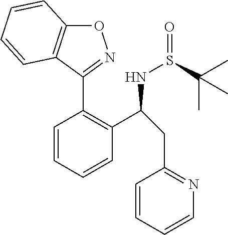 [SMILES: CC(C)(C)[S@](=O)N[C@@H](Cc1ccccn1)c1ccccc1-c1noc2ccccc12]

LDA (2M solution in THF, heptane and ethylbenzene, 0.65 mL) was added dropwise to a stirred, cooled solution of 2-methylpyridine (0.13 g) in THF (6 mL) while maintaining the temperature below −60° C. The resultant red mixture was stirred at below −70° C. for 2 hours then transferred by cannula to a cooled solution of (S,E)-N-[2-(benzo[d]isoxazol-3-yl)benzylidene]-2-methylpropane-2-sulfinamide (Intermediate 4B, 0.158 g) in THF (2 mL). The mixture was stirred at below −70° C. for 1 hour then allowed to warm to room temperature, then diluted with water and extracted with ethyl acetate. The organic phase was dried (MgSO₄) and filtered. The filtrate was concentrated in vacuo and the residue was purified by FCC eluted with 20-100% ethyl acetate in cyclohexane to give the slower running component as a clear gum (0.11 g). ¹H NMR (400 MHz, CDCl₃) 8.47-8.45 (1H, m), 7.72-7.60 (4H, m), 7.57-7.48 (3H, m), 7.45-7.35 (2H, m), 7.13-7.09 (1H, m), 7.01 (1H, d, J=8.1 Hz), 5.51 (1H, d, J=6.7 Hz), 5.10-5.03 (1H, m), 3.38-3.33 (1H, m), 3.20-3.14 (1H, m), 0.98-0.97 (9H, m).

By proceeding in a similar manner to Intermediate 6A, the following compounds were prepared:

### Intermediate 6B: (S)—N—{(S)-1-[2-(Benzo[d]isoxazol-3-yl)phenyl]-2-(6-bromopyridine-2-yl)ethyl}-2-methylpropane-2-sulfinamide

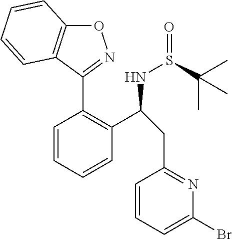 [SMILES: CC(C)(C)[S@](=O)N[C@@H](Cc1cccc(Br)n1)c1ccccc1-c1noc2ccccc12]

Starting from (S,E)-N-[2-(benzo[d]isoxazol-3-yl)benzylidene]-2-methylpropane-2-sulfinamide (Intermediate 4B) and 2-bromo-6-methylpyridine. LCMS (Method 5) RT 4.16 m/z 498/500 [MH⁺].

### Intermediate 6C: (S)—N—{(S)-1-[2-(Benzo[d]isoxazol-3-yl)phenyl]-2-(4-bromopyridine-2-yl)ethyl}-2-methylpropane-2-sulfinamide

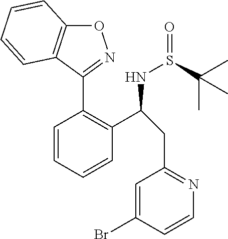 [SMILES: CC(C)(C)[S@](=O)N[C@@H](Cc1cc(Br)ccn1)c1ccccc1-c1noc2ccccc12]

Starting from (S,E)-N-[2-(benzo[d]isoxazol-3-yl)benzylidene]-2-methylpropane-2-sulfinamide (Intermediate 4B) and 4-bromo-2-methylpyridine. LCMS (Method 5) RT 4.02 m/z 498/500 [MH⁺].

### Intermediate 6D: (S)—N—{(S)-1-[2-(Benzo[d]isoxazol-3-yl)phenyl]-2-(5-bromopyridine-2-yl)ethyl}-2-methylpropane-2-sulfinamide

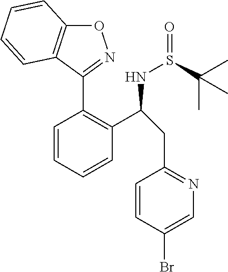 [SMILES: CC(C)(C)[S@](=O)N[C@@H](Cc1ccc(Br)cn1)c1ccccc1-c1noc2ccccc12]

Starting from (S,E)-N-[2-(benzo[d]isoxazol-3-yl)benzylidene]-2-methylpropane-2-sulfinamide (Intermediate 4B) and 5-bromo-2-methylpyridine. LCMS (Method 5) RT 4.13 m/z 498/500 [MH⁺].

### Intermediate 6E: (S)—N—{(S)-1-[2-(6-Bromobenzo[d]isoxazol-3-yl)phenyl]-2-(6-bromopyridine-2-yl)ethyl}-2-methylpropane-2-sulfinamide

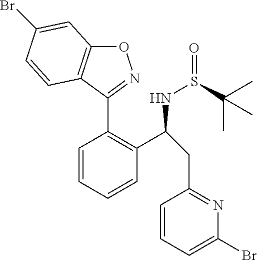 [SMILES: CC(C)(C)[S@](=O)N[C@@H](Cc1cccc(Br)n1)c1ccccc1-c1noc2cc(Br)ccc12]

Starting from (S,E)-N-[2-(6-bromobenzo[d]isoxazol-3-yl)benzylidene]-2-methylpropane-2-sulfinamide (Intermediate 4A) and 2-bromo-6-methylpyridine. LCMS (Method 7) RT 3.9 m/z 576/579/580 [MH⁺].

### Intermediate 6F: (S)—N—{(S)-1-[2-(6-Bromobenzo[d]isoxazol-3-yl)phenyl]-2-(6-fluoropyridine-2-yl)ethyl}-2-methylpropane-2-sulfinamide

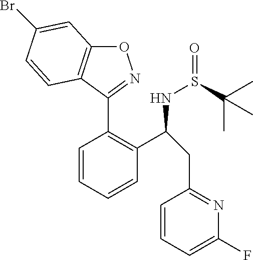 [SMILES: CC(C)(C)[S@](=O)N[C@@H](Cc1cccc(F)n1)c1ccccc1-c1noc2cc(Br)ccc12]

Starting from (S,E)-N-[2-(6-bromobenzo[d]isoxazol-3-yl)benzylidene]-2-methylpropane-2-sulfinamide (Intermediate 4A) and 2-fluoro-6-methylpyridine. LCMS (Method 6) RT 3.84 m/z 516/518 [MH⁺]

### Intermediate 6G: (S)—N-{(1S)-1-[2-(6-Bromobenzo[d]isoxazol-3-yl)phenyl]-2-[6-{2-[(tetrahydro-2H-pyran-2-yl)oxy]ethoxy}pyridine-2-yl]ethyl}-2-methylpropane-2-sulfinamide

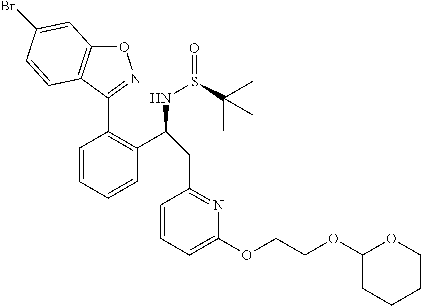 [SMILES: CC(C)(C)[S@](=O)N[C@@H](Cc1cccc(OCCOC2CCCCO2)n1)c1ccccc1-c1noc2cc(Br)ccc12]

Starting from (S,E)-N-[2-(6-bromobenzo[d]isoxazol-3-yl)benzylidene]-2-methylpropane-2-sulfinamide (Intermediate 4A) and 2-methyl-6-{2-[(tetrahydro-2H-pyran-2-yl)oxy]ethoxy}pyridine (Intermediate 52A). LCMS (Method 6) RT 4.42 m/z 558/560 [MH⁺-84] (loss of THP).

### Intermediate 6H: (S)—N—{(S)-1-[2-(Benzo[d]isoxazol-3-yl)phenyl]-2-(pyrimidin-2-yl)ethyl}-2-methylpropane-2-sulfinamide

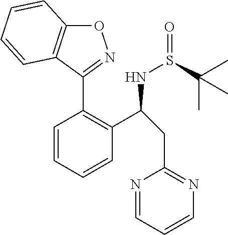 [SMILES: CC(C)(C)[S@](=O)N[C@@H](Cc1ncccn1)c1ccccc1-c1noc2ccccc12]

Starting from (S,E)-N-[2-(benzo[d]isoxazol-3-yl)benzylidene]-2-methylpropane-2-sulfinamide (Intermediate 4B) and 2-methylpyrimidine. LCMS (Method 8) RT 2.95 m/z 421 [MH⁺].

### Intermediate 61: (S)—N—{(S)-1-[2-(Benzo[d]isoxazol-3-yl)phenyl]-2-(6-bromo-3-methylpyridin-2-yl)ethyl}-2-methylpropane-2-sulfinamide

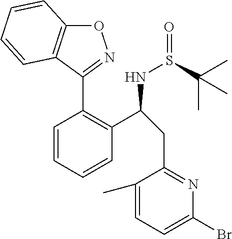 [SMILES: Cc1ccc(Br)nc1C[C@H](N[S@@](=O)C(C)(C)C)c1ccccc1-c1noc2ccccc12]

Starting from (S,E)-N-[2-(benzo[d]isoxazol-3-yl)benzylidene]-2-methylpropane-2-sulfinamide (Intermediate 4B) and 6-bromo-2,3-dimethylpyridine. LCMS (Method 8) RT 3.80 m/z 512/514 [MH⁺].

### Intermediate 6J: (S)—N—{(S)-1-[2-(Benzo[d]isoxazol-3-yl)-6-fluorophenyl]-2-(6-bromopyridin-2-yl)ethyl}-2-methylpropane-2-sulfinamide

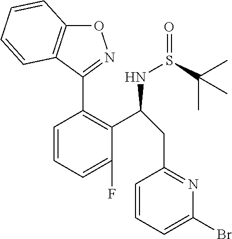 [SMILES: CC(C)(C)[S@](=O)N[C@@H](Cc1cccc(Br)n1)c1c(F)cccc1-c1noc2ccccc12]

Starting from (S,E)-N-[2-(benzo[d]isoxazol-3-yl)-6-fluorobenzylidene]-2-methylpropane-2-sulfinamide (Intermediate 4K) and 2-bromo-6-methylpyridine. LCMS (Method 4) RT 1.66 m/z 516/518 [MH⁺].

### Intermediate 6K: (S)—N—{(S)-1-[2-(Benzo[d]isoxazol-3-yl)-6-fluorophenyl]-2-(pyridin-2-yl)ethyl}-2-methylpropane-2-sulfinamide

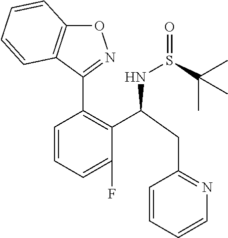 [SMILES: CC(C)(C)[S@](=O)N[C@@H](Cc1ccccn1)c1c(F)cccc1-c1noc2ccccc12]

Starting from (S,E)-N-[2-(benzo[d]isoxazol-3-yl)-6-fluorobenzylidene]-2-methylpropane-2-sulfinamide (Intermediate 4K) and 2-methylpyridine. LCMS (Method 4) RT 1.22 m/z 438 [MH⁺].

### Intermediate 6L: (S)—N—{(S)-1-[2-(Benzo[d]isoxazol-3-yl)-6-fluorophenyl]-2-(6-methylpyridin-2-yl)ethyl}-2-methylpropane-2-sulfinamide

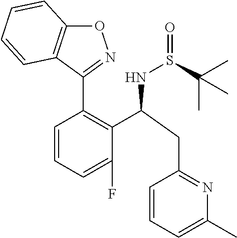 [SMILES: Cc1cccc(C[C@H](N[S@@](=O)C(C)(C)C)c2c(F)cccc2-c2noc3ccccc23)n1]

Starting from (S,E)-N-[2-(benzo[d]isoxazol-3-yl)-6-fluorobenzylidene]-2-methylpropane-2-sulfinamide (Intermediate 4K) and 2,6-dimethylpyridine. LCMS (Method 4) RT 1.11 m/z 452 [MH⁺]

### Intermediate 6M: (S)—N—{(S)-1-[2-(Benzo[d]isoxazol-3-yl)phenyl]-2-(6-trifluoromethylpyridin-2-yl)ethyl}-2-methylpropane-2-sulfinamide

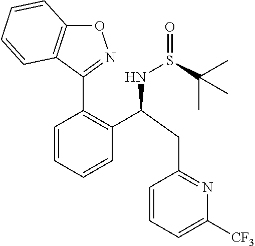 [SMILES: CC(C)(C)[S@](=O)N[C@@H](Cc1cccc(C(F)(F)F)n1)c1ccccc1-c1noc2ccccc12]

Starting from (S,E)-N-[2-(benzo[d]isoxazol-3-yl)benzylidene]-2-methylpropane-2-sulfinamide (Intermediate 4B) and 2-methyl-6-trifluoromethylpyridine. ¹H NMR (300 MHz, CDCl₃) 7.74-7.60 (5H, m), 7.59-7.54 (1H, m), 7.54-7.37 (4H, m), 7.29 (1H, d, J=6.9 Hz), 5.15-5.09 (2H, m), 3.53-3.45 (1H, m), 3.29 (1H, dd, J=8.1, 13.8 Hz), 0.98 (9H, s).

### Intermediate 6N: (S)—N—{(S)-1-[2-(Benzo[d]isoxazol-3-yl)phenyl]-2-(3-fluoropyridin-2-yl)ethyl}-2-methylpropane-2-sulfinamide

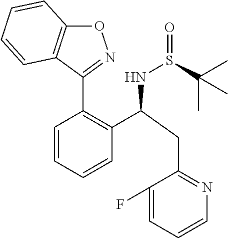 [SMILES: CC(C)(C)[S@](=O)N[C@@H](Cc1ncccc1F)c1ccccc1-c1noc2ccccc12]

Starting from (S,E)-N-[2-(benzo[d]isoxazol-3-yl)benzylidene]-2-methylpropane-2-sulfinamide (Intermediate 4B) and 3-fluoro-2-methylpyridine. ¹H NMR (300 MHz, CDCl₃) 8.27-8.23 (1H, m), 7.72-7.58 (4H, m), 7.56-7.40 (3H, m), 7.40-7.33 (1H, m), 7.24-7.16 (1H, m), 7.13-7.06 (1H, m), 5.33-5.28 (1H, m), 5.23-5.14 (1H, m), 3.32-3.26 (2H, m), 1.01-1.00 (9H, m).

### Intermediate 60: (S)—N-{2-[6-Bromopyridine-2-yl]-1-[2-(1-isopropyl-1H-indazol-3-yl)phenyl]ethyl}-2-methylpropane-2-sulfinamide

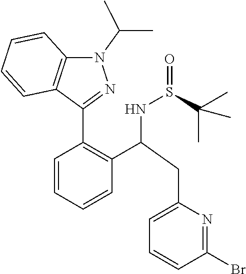 [SMILES: CC(C)n1nc(-c2ccccc2C(Cc2cccc(Br)n2)N[S@@](=O)C(C)(C)C)c2ccccc21]

Starting form (S,E)-2-methyl-N-[2-(1-methyl-1H-indazol-3-yl)benzylidene]propane-2-sulfinamide (Intermediate 4M) and 2-bromo-6-methylpyridine. LCMS (Method 4) RT 1.69 m/z 539/541 [MH⁺].

### Intermediate 6P: (S)—N-{1-[2-(1-Isopropyl-1H-indazol-3-yl)phenyl]-2-(pyridine-2-yl)ethyl}-2-methylpropane-2-sulfinamide

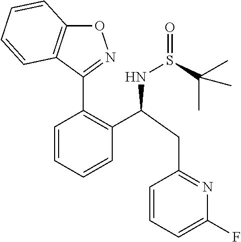 [SMILES: CC(C)(C)[S@](=O)N[C@@H](Cc1cccc(F)n1)c1ccccc1-c1noc2ccccc12]

Starting from (S,E)-2-methyl-N-[2-(1-isopropyl-1H-indazol-3-yl)benzylidene]propane-2-sulfinamide (Intermediate 4M) and 2-methylpyridine. LCMS (Method 4) RT 1.31 m/z 461 [MH⁺].

### Intermediate 6Q: (S)—N—{(S)-1-[2-(Benzo[d]isoxazol-3-yl)phenyl]-2-(6-fluoropyridine-2-yl)ethyl}-2-methylpropane-2-sulfinamide

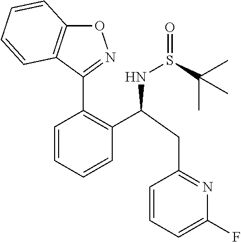 [SMILES: CC(C)(C)[S@](=O)N[C@@H](Cc1cccc(F)n1)c1ccccc1-c1noc2ccccc12]

Starting from (S,E)-N-[2-(benzo[d]isoxazol-3-yl)benzylidene]-2-methylpropane-2-sulfinamide (Intermediate 4B) and 6-fluoro-2-methylpyridine. LCMS (Method 4) RT 1.93 m/z 438 [MH⁺].

### Intermediate 6R: (S)—N—{(S)-1-[2-(Benzo[d]isoxazol-3-yl)phenyl]-2-(4-fluoropyridine-2-yl)ethyl}-2-methylpropane-2-sulfinamide

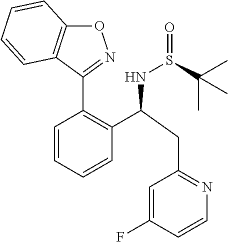 [SMILES: CC(C)(C)[S@](=O)N[C@@H](Cc1cc(F)ccn1)c1ccccc1-c1noc2ccccc12]

Starting from (S,E)-N-[2-(benzo[d]isoxazol-3-yl)benzylidene]-2-methylpropane-2-sulfinamide (Intermediate 4B) and 4-fluoro-2-methylpyridine. LCMS (Method 4) RT 1.44 m/z 438 [MH⁺].

### Intermediate 6S: (S)—N—{(S)-1-[2-(Benzo[d]isoxazol-3-yl)phenyl]-2-[6-bromo-3-fluoro-4-(trimethylsilyl)pyridine-2-yl]ethyl}-2-methylpropane-2-sulfinamide

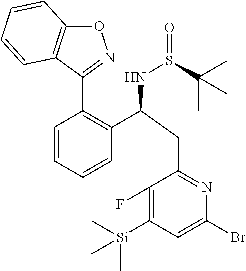 [SMILES: CC(C)(C)[S@](=O)N[C@@H](Cc1nc(Br)cc([Si](C)(C)C)c1F)c1ccccc1-c1noc2ccccc12]

Starting from (S,E)-N-[2-(benzo[d]isoxazol-3-yl)benzylidene]-2-methylpropane-2-sulfinamide (Intermediate 4B) and 6-bromo-3-fluoro-2-methyl-4-(trimethylsilyl)-pyridine (Intermediate 72A). LCMS (Method 4) RT 1.82 m/z 588/590 [MH⁺].

### Intermediate 6T: (S)—N—{(S)-2-(3-Bromopyridine-2-yl)-1-[2-(benzo[d]isoxazol-3-yl)phenyl]ethyl}-2-methylpropane-2-sulfinamide

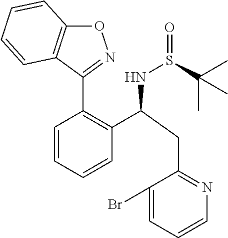 [SMILES: CC(C)(C)[S@](=O)N[C@@H](Cc1ncccc1Br)c1ccccc1-c1noc2ccccc12]

Starting from (S,E)-N-[2-(benzo[d]isoxazol-3-yl)benzylidene]-2-methylpropane-2-sulfinamide (Intermediate 4B) and 3-bromo-2-methylpyridine. ¹H NMR (400 MHz, CDCl₃) 8.38-8.35 (1H, m), 7.77-7.72 (1H, m), 7.71-7.64 (3H, m), 7.64-7.59 (1H, m), 7.58-7.49 (2H, m), 7.48-7.42 (1H, m), 7.39-7.33 (1H, m), 6.97-6.93 (1H, m), 5.25-5.19 (2H, m), 3.49-3.42 (1H, m), 3.33-3.26 (1H, m), 0.99 (9H, s).

### Intermediate 6U: (S)—N—{(S)-1-[2-(Benzo[d]isoxazol-3-yl)phenyl]-2-(3-cyclopropylpyridine-2-yl)ethyl}-2-methylpropane-2-sulfinamide

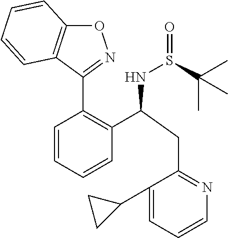 [SMILES: CC(C)(C)[S@](=O)N[C@@H](Cc1ncccc1C1CC1)c1ccccc1-c1noc2ccccc12]

Starting from (S)—N-[2-(benzo[d]isoxazol-yl)benzylidene]-2-methylpropane-2-sulfinamide (Intermediate 4B) and 3-cyclopropyl-2-methylpyridine and used directly in the next step.

### Intermediate 6V: (S)—N—{(S)-1-[2-(Benzo[d]isoxazol-3-yl)phenyl]-2-(5-bromo-3-methylpyridine-2-yl)ethyl}-2-methylpropane-2-sulfinamide

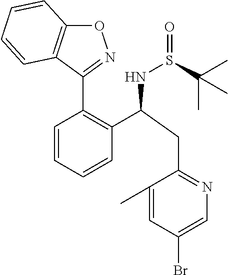 [SMILES: Cc1cc(Br)cnc1C[C@H](N[S@@](=O)C(C)(C)C)c1ccccc1-c1noc2ccccc12]

Starting from (S,E)-N-[2-(benzo[d]isoxazol-3-yl)benzylidene]-2-methylpropane-2-sulfinamide (Intermediate 4B) and 5-bromo-2,3-dimethylpyridine. ¹H NMR (400 MHz, CDCl₃) 8.34-8.31 (1H, m), 7.69-7.60 (4H, m), 7.54-7.35 (5H, m), 5.54-5.51 (1H, m), 5.14-5.08 (1H, m), 3.31-3.24 (1H, m), 3.22-3.15 (1H, m), 1.96 (3H, s), 1.01 (9H, s).

### Intermediate 6W: (S)—N—{(S)-1-[2-(Benzo[d]isoxazol-3-yl)phenyl]-2-[6-bromo-5-fluoro-4-(trimethylsilyl)pyridine-2-yl]ethyl}-2-methylpropane-2-sulfinamide

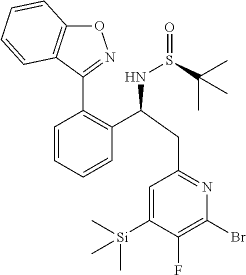 [SMILES: CC(C)(C)[S@](=O)N[C@@H](Cc1cc([Si](C)(C)C)c(F)c(Br)n1)c1ccccc1-c1noc2ccccc12]

Starting from (S,E)-N-[2-(benzo[d]isoxazol-3-yl)benzylidene]-2-methylpropane-2-sulfinamide (Intermediate 4B) and 2-bromo-3-fluoro-6-methyl-4-(trimethylsilyl)pyridine (Intermediate 72B). LCMS (Method 6) RT 1.97 m/z 588/590 [MH⁺].

### Intermediate 6X: (S)—N—{(S)-2-[6-Bromo-3-fluoro-4-(trimethylsilyl)pyridine-2-yl]-1-[2-(6-trifluoromethylbenzo[d]isoxazol-3-yl)phenyl]ethyl}-]-2-methylpropane-2-sulfinamide

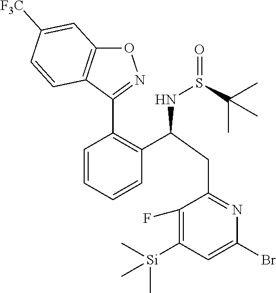 [SMILES: CC(C)(C)[S@](=O)N[C@@H](Cc1nc(Br)cc([Si](C)(C)C)c1F)c1ccccc1-c1noc2cc(C(F)(F)F)ccc12]

Starting from (S,E)-2-methyl-N-[2-(6-trifluoromethylbenzo[d]isoxazol-3-yl)benzylidene]propane-2-sulfinamide (Intermediate 4N) and 6-Bromo-3-fluoro-2-methyl-4-(trimethylsilyl)pyridine (Intermediate 72A). LCMS (Method 3) RT 1.81 m/z 656/658 [MH⁺].

### Intermediate 6Y: (S)—N—{(S)-2-[6-Bromo-3-fluoro-4-(trimethylsilyl)pyridine-2-yl]-1-[2-(6-methylbenzo[d]isoxazol-3-yl)phenyl]ethyl}-]-2-methylpropane-2-sulfinamide

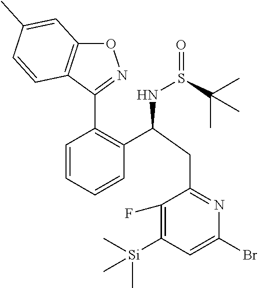 [SMILES: Cc1ccc2c(-c3ccccc3[C@H](Cc3nc(Br)cc([Si](C)(C)C)c3F)N[S@@](=O)C(C)(C)C)noc2c1]

Starting from (S,E)-2-methyl-N-[2-(6-methylbenzo[d]isoxazol-3-yl)benzylidene]propane-2-sulfinamide (Intermediate 40) and 6-Bromo-3-fluoro-2-methyl-4-(trimethylsilyl)pyridine (Intermediate 72A). LCMS (Method 3) RT 1.79 m/z 602/604 [MH⁺].

### Intermediate 6Z: (S)—N—{(S)-2-[6-Bromo-3-fluoro-4-(trimethylsilyl)pyridine-2-yl]-1-[2-(6-chlorobenzo[d]isoxazol-3-yl)phenyl]ethyl}-]-2-methylpropane-2-sulfinamide

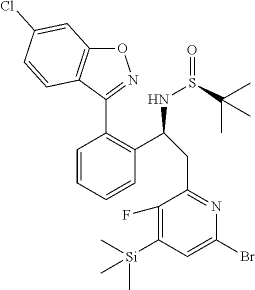 [SMILES: CC(C)(C)[S@](=O)N[C@@H](Cc1nc(Br)cc([Si](C)(C)C)c1F)c1ccccc1-c1noc2cc(Cl)ccc12]

Starting from (S,E)-N-[2-(6-chlorobenzo[d]isoxazol-3-yl)benzylidene]propane-2-sulfinamide (Intermediate 4P) and 6-bromo-3-fluoro-2-methyl-4-(trimethylsilyl)pyridine (Intermediate 72A). ¹H NMR (400 MHz, CDCl₃) 7.71-7.69 (1H, m), 7.61-7.56 (2H, m), 7.53-7.47 (2H, m), 7.46-7.40 (1H, m), 7.36 (1H, dd, J=1.7, 8.5 Hz), 7.20-1H, d, J=3.2 Hz), 5.12-5.05 (1H, m), 5.03-4.97 (1H, m), 3.29-3.21 (1H, m), 3.20-3.12 (1H, m), 1.07 (9H, s), 0.24 (9H, s).

### Intermediate 6AA: (S)—N—{(S)-2-[6-Bromo-3-fluoro-4-(trimethylsilyl)pyridine-2-yl]-1-[2-(6-fluorobenzo[d]isoxazol-3-yl)phenyl]ethyl}-2-methylpropane-2-sulfinamide

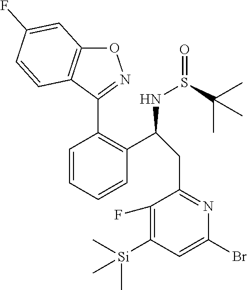 [SMILES: CC(C)(C)[S@](=O)N[C@@H](Cc1nc(Br)cc([Si](C)(C)C)c1F)c1ccccc1-c1noc2cc(F)ccc12]

Starting from (S,E)-N-[2-(6-fluorobenzo[d]isoxazol-3-yl)benzylidene]propane-2-sulfinamide (Intermediate 4Q) and 6-bromo-3-fluoro-2-methyl-4-(trimethylsilyl)pyridine (Intermediate 72A). LCMS (Method 3) RT 1.75 m/z 606/608 [MH⁺].

### Intermediate 6AB: (S)—N—{(S)-2-[6-Bromo-3-fluoro-4-(trimethylsilyl)pyridine-2-yl]-1-[2-(6-bromobenzo[d]isoxazol-3-yl)phenyl]ethyl}-2-methylpropane-2-sulfinamide

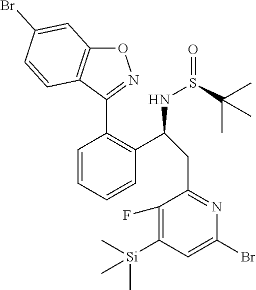 [SMILES: CC(C)(C)[S@](=O)N[C@@H](Cc1nc(Br)cc([Si](C)(C)C)c1F)c1ccccc1-c1noc2cc(Br)ccc12]

Starting from (S,E)-N-[2-(6-bromobenzo[d]isoxazol-3-yl)benzylidene]propane-2-sulfinamide (Intermediate 4A) and 6-bromo-3-fluoro-2-methyl-4-(trimethylsilyl)pyridine (Intermediate 72A). LCMS (Method 4) RT 2.28 m/z 666/668/670 [MH⁺].

### Intermediate 6AC: (S)—N—{(S)-2-[6-Bromo-3-fluoro-4-(trimethylsilyl)pyridine-2-yl]-1-[2-(6-cyanobenzo[d]isoxazol-3-yl)phenyl]ethyl}-2-methylpropane-2-sulfinamide

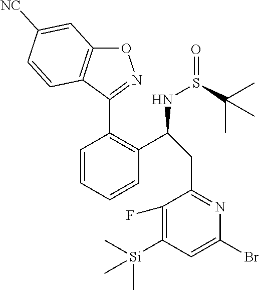 [SMILES: CC(C)(C)[S@](=O)N[C@@H](Cc1nc(Br)cc([Si](C)(C)C)c1F)c1ccccc1-c1noc2cc(C#N)ccc12]

Starting from (S,E)-N-[2-(6-cyanobenzo[d]isoxazol-3-yl)benzylidene]-2-methylpropane-2-sulfinamide (Intermediate 27Q) and 6-bromo-3-fluoro-2-methyl-4-(trimethylsilyl)pyridine (Intermediate 72A). ¹H NMR (400 MHz, CDCl₃) 8.04-8.02 (1H, m), 7.84-7.80 (1H, m), 7.66-7.63 (1H, m), 7.63-7.59 (1H, m), 7.57-7.52 (1H, m), 7.49-7.42 (2H, m), 7.21-7.19 (1H, m), 5.11-5.03 (1H, m), 4.82-4.77 (1H, m), 3.31-3.15 (2H, m), 1.06 (9H, s), 0.24 (9H, s).

### Intermediate 6AD: (S)—N—{(S)-2-[6-Bromo-3-methylpyridine-2-yl]-1-[2-(6-methylbenzo[d]isoxazol-3-yl)phenyl]ethyl}-2-methylpropane-2-sulfinamide

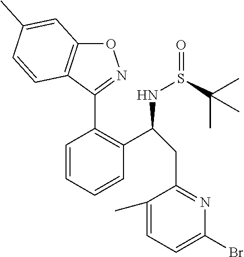 [SMILES: Cc1ccc2c(-c3ccccc3[C@H](Cc3nc(Br)ccc3C)N[S@@](=O)C(C)(C)C)noc2c1]

Starting from (S,E)-2-methyl-N-[2-(6-methylbenzo[d]isoxazol-3-yl)benzylidene]propane-2-sulfinamide (Intermediate 40) and 6-bromo-2,3-dimethylpyridine. LCMS (Method 3) RT 1.64 m/z 526/528 [MH⁺].

### Intermediate 6AE: (S)—N—{(S)-2-[6-Bromo-3-fluoro-4-(trimethylsilyl)pyridine-2-yl]-1-[2-(5-fluorbenzo[d]isoxazol-3-yl)phenyl]ethyl}-2-methylpropane-2-sulfinamide

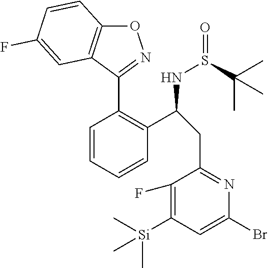 [SMILES: CC(C)(C)[S@](=O)N[C@@H](Cc1nc(Br)cc([Si](C)(C)C)c1F)c1ccccc1-c1noc2ccc(F)cc12]

Starting from (S,E)-N-[2-(5-fluorobenzo[d]isoxazol-3-yl)benzylidene]-2-methylpropane-2-sulfinamide (Intermediate 4R) and 6-bromo-3-fluoro-2-methyl-4-(trimethylsilyl)pyridine (Intermediate 72A). LCMS (Method 3) RT 1.83 m/z 628/630 [M+Na⁺].

### Intermediate 6AF: (S)—N—{(S)-2-[6-Bromo-3-methylpyridine-2-yl]-1-[2-(6-chlorobenzo[d]isoxazol-3-yl)phenyl]ethyl}-2-methylpropane-2-sulfinamide

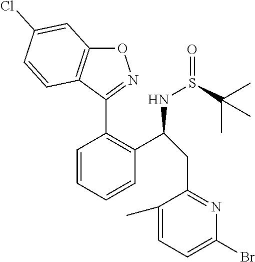 [SMILES: Cc1ccc(Br)nc1C[C@H](N[S@@](=O)C(C)(C)C)c1ccccc1-c1noc2cc(Cl)ccc12]

Starting from (S,E)-N-[2-(6-chlorobenzo[d]isoxazol-3-yl)benzylidene]-2-methylpropane-2-sulfinamide (Intermediate 4P) and 6-bromo-2,3-dimethylpyridine. LCMS (Method 3) RT 1.64 m/z 546/548 [MH⁺].

### Intermediate 6AG: (S)—N—{(S)-2-[6-Bromo-3-methylpyridine-2-yl]-1-[2-(6-fluorobenzo[d]isoxazol-3-yl)phenyl]ethyl}-2-methylpropane-2-sulfinamide

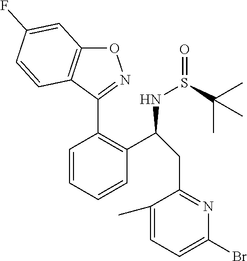 [SMILES: Cc1ccc(Br)nc1C[C@H](N[S@@](=O)C(C)(C)C)c1ccccc1-c1noc2cc(F)ccc12]

Starting from (S,E)-N-[2-(6-fluorobenzo[d]isoxazol-3-yl)benzylidene]-2-methylpropane-2-sulfinamide (Intermediate 4Q) and 6-bromo-2,3-dimethylpyridine. LCMS (Method 3) RT 1.56 m/z 530/532 [MH⁺].

### Intermediate 6AH: (S)—N—{(S)-2-[6-bromo-3-fluoro-4-(trimethylsilyl)pyridine-2-yl]-1-[2-(7-fluorbenzo[d]isoxazol-3-yl)phenyl]ethyl}-2-methylpropane-2-sulfinamide

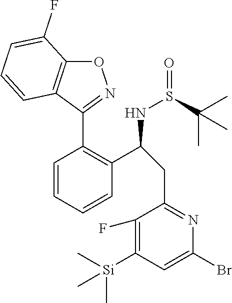 [SMILES: CC(C)(C)[S@](=O)N[C@@H](Cc1nc(Br)cc([Si](C)(C)C)c1F)c1ccccc1-c1noc2c(F)cccc12]

Starting from (S,E)-N-[2-(7-fluorobenzo[d]isoxazol-3-yl)benzylidene]-2-methylpropane-2-sulfinamide (Intermediate 4S) and 6-bromo-3-fluoro-2-methyl-4-(trimethylsilyl)pyridine (Intermediate 72A). ¹H NMR (400 MHz, CDCl₃) 7.62-7.59 (1H, m), 7.54-7.49 (2H, m), 7.46-7.43 (2H, m), 7.35-7.29 (2H, m), 7.20 (1H, d, J=3.2 Hz), 5.13-5.07 (1H, m), 5.06-5.01 (1H, m), 3.29-3.22 (1H, m), 3.21-3.14 (1H, m), 1.07 (9H, s), 0.24 (9H, s).

### Intermediate 6AI: (S)—N—{(S)-2-[6-Bromo-3-methylpyridine-2-yl]-1-[2-(6-bromobenzo[d]isoxazol-3-yl)phenyl]ethyl}-2-methylpropane-2-sulfinamide

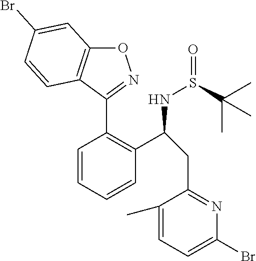 [SMILES: Cc1ccc(Br)nc1C[C@H](N[S@@](=O)C(C)(C)C)c1ccccc1-c1noc2cc(Br)ccc12]

Starting from (S,E)-N-[2-(6-bromobenzo[d]isoxazol-3-yl)benzylidene]-2-methylpropane-2-sulfinamide (Intermediate 4A) and 6-bromo-2,3-dimethylpyridine. LCMS (Method 3) RT 1.68 m/z 590/592/294) [MH⁺].

### Intermediate 7A: (S)—N—{(S)-1-[2-(6-Bromobenzo[d]isoxazol-3-yl)phenyl]-2-(5-cyanopyridine-2-yl)ethyl}-2-methylpropane-2-sulfinamide

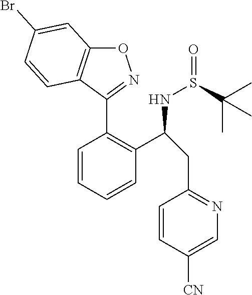 [SMILES: CC(C)(C)[S@](=O)N[C@@H](Cc1ccc(C#N)cn1)c1ccccc1-c1noc2cc(Br)ccc12]

A solution of Lithium bis(trimethylsilyl)amide (1M in THF, 2.2 mL) was added to a stirred, cooled solution of 2-methyl-5-cyanopyridine (0.236 g) in dry THF (3 mL) while maintaining the temperature below −60° C. The resultant mixture was stirred at −78° C. for 45 minutes. A solution of (S)—N-[2-(6-bromobenzo[d]isoxazol-3-yl)benzylidene]-2-methylpropane-2-sulfinamide (Intermediate 4A, 0.405 g) in dry THF (2 mL) was added dropwise and the mixture was stirred at −78° C. for a further 1 hour. The temperature was allowed to rise to approximately −20° C. and a saturated aqueous solution of ammonium chloride was added, followed by water. The mixture was extracted with ethyl acetate, dried (MgSO₄) and filtered. The filtrate was concentrated in vacuo and the residue was purified by FCC eluted with 20-100% ethyl acetate in pentane to give the title compound (0.328 g) as a clear gum which was used directly in the next stage.

By proceeding in a similar manner to Intermediate 7A, the following compounds were prepared:

### Intermediate 7B: (S)—N—{(S)-1-[2-(6-Bromobenzo[d]isoxazol-3-yl)phenyl]-2-(6-cyanopyridine-2-yl)ethyl}-2-methylpropane-2-sulfinamide

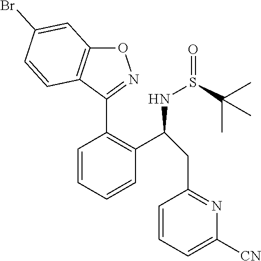 [SMILES: CC(C)(C)[S@](=O)N[C@@H](Cc1cccc(C#N)n1)c1ccccc1-c1noc2cc(Br)ccc12]

Starting from (S)—N-[2-(6-bromobenzo[d]isoxazol-3-yl)benzylidene]-2-methylpropane-2-sulfinamide (Intermediate 4A) and 2-methyl-6-cyanopyridine. LCMS (Method 7) RT 3.57 m/z 523/525 [MH⁺].

### Intermediate 8A: (S)-1-[2-(6-Bromobenzo[d]isoxazol-3-yl)phenyl]-2-(pyridine-2-yl)ethan-1-amine hydrochloride

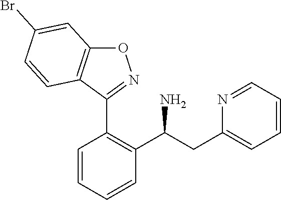 [SMILES: N[C@@H](Cc1ccccn1)c1ccccc1-c1noc2cc(Br)ccc12]

Hydrogen chloride (4M in dioxane, 10 mL) was added to a solution of (S)—N—{(S)-1-[2-(6-bromobenzo[d]isoxazol-3-yl)phenyl]-2-(pyridin-2-yl)ethyl}-2-methylpropane-2-sulfinamide (Intermediate 5A, 2.86 g) in methanol (15 mL) and the mixture was stirred at room temperature for 2 hours. The mixture was concentrated in vacuo and the residue was dissolved in a mixture of methanol and DCM and loaded onto a SCX-2 cartridge which was then washed with DCM and then methanol. The product was eluted using a solution of ammonia in methanol (2M) and then isolated as an orange gum (2.35 g). A portion of this (0.15 g) was purified by MDAP (basic) and the product was dissolved in acetonitrile (2 mL) and hydrochloric acid (1M, 4 mL) was added and the mixture was freeze dried to give the title compound as an off-white solid (0.12 g). LCMS Method (Method 1): RT 3.18 m/z 394, 396.

By proceeding in a similar manner to Intermediate 8A, the following compounds were prepared:

### Intermediate 8B: (S)-1-[2-(7-Bromobenzo[d]isoxazol-3-yl)phenyl]-2-(pyridine-2-yl)ethan-1-amine hydrochloride

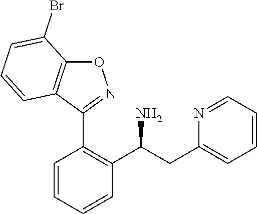 [SMILES: N[C@@H](Cc1ccccn1)c1ccccc1-c1noc2c(Br)cccc12]

Starting from (S)—N—{(S)-1-[2-(7-bromobenzo[d]isoxazol-3-yl)phenyl]-2-(pyridin-2-yl)ethyl}-2-methylpropane-2-sulfinamide (Intermediate 51) converting to the HCl salt by treatment with 0.1M hydrochloric acid in acetonitrile and freeze drying. LCMS (Method 1) RT 3.40 m/z 394/396 [MH⁺].

### Intermediate 9A: tert-Butyl (S)-{1-[2-(6-bromobenzo[d]isoxazol-3-yl)phenyl]-2-(pyridin-2-yl)ethyl}carbamate

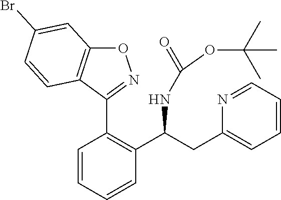 [SMILES: CC(C)(C)OC(=O)N[C@@H](Cc1ccccn1)c1ccccc1-c1noc2cc(Br)ccc12]

Di-tert-butyl carbonate (1.35 g) was added to a mixture of (S)-1-[2-(6-bromobenzo[d]isoxazol-3-yl)phenyl]-2-(pyridine-2-yl)ethan-1-amine (Intermediate 8A, 2.2 g) and sodium bicarbonate (0.521 g) in aqueous dioxane (1:1, 28 mL) and the mixture was stirred under an atmosphere of nitrogen overnight. The resultant mixture was added to water and extracted with ethyl acetate. The organic phase was washed with water, dried (MgSO₄) and filtered. The filtrate was concentrated in vacuo to give the title compound as an orange glass (2.43 g) which was used directly in the next stage.

By proceeding in a similar manner to Intermediate 9A, the following compounds were prepared:

### Intermediate 9B: tert-Butyl (S)-{1-[2-(benzo[d]isoxazol-3-yl)-5-bromophenyl]-2-(pyridin-2-yl)ethyl}carbamate

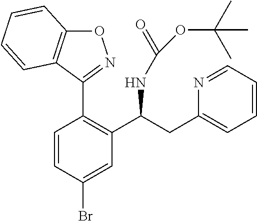 [SMILES: CC(C)(C)OC(=O)N[C@@H](Cc1ccccn1)c1cc(Br)ccc1-c1noc2ccccc12]

Starting from (S)-1-[2-(benzo[d]isoxazol-3-yl)-5-bromophenyl]-2-(pyridine-2-yl)ethan-1-amine (Example 83). LCMS (Method 5) RT 3.92 m/z 494/496 [MH⁺].

### Intermediate 9C: tert-Butyl (S)-{1-[2-(benzo[d]isoxazol-3-yl)-4-bromophenyl]-2-(pyridin-2-yl)ethyl}carbamate

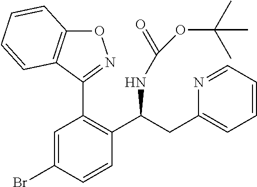 [SMILES: CC(C)(C)OC(=O)N[C@@H](Cc1ccccn1)c1ccc(Br)cc1-c1noc2ccccc12]

Starting from (S)-1-[2-(Benzo[d]isoxazol-3-yl)-4-bromophenyl]-2-(pyridine-2-yl)ethan-1-amine (Example 85). LCMS (Method 5) RT 3.87 m/z 494/496 [MH⁺].

### Intermediate 9D: tert-Butyl (S)-{1-[2-(benzo[d]isoxazol-3-yl)phenyl]-2-(6-bromopyridin-2-yl)ethyl}carbamate

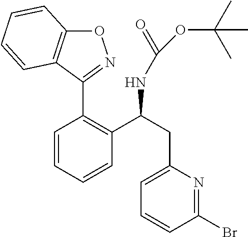 [SMILES: CC(C)(C)OC(=O)N[C@@H](Cc1cccc(Br)n1)c1ccccc1-c1noc2ccccc12]

Starting from (S)-1-[2-(benzo[d]isoxazol-3-yl)phenyl]-2-(6-bromopyridine-2-yl)ethan-1-amine (Example 44). LCMS (Method 5) RT 4.61 m/z 494/496 [MH⁺].

### Intermediate 10A: tert-Butyl (S)-{1-[2-(6-cyanobenzo[d]isoxazol-3-yl)phenyl]-2-(pyridine-2-yl)ethyl}carbamate

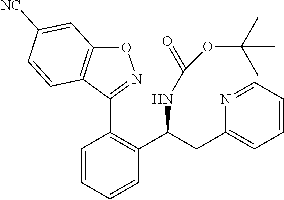 [SMILES: CC(C)(C)OC(=O)N[C@@H](Cc1ccccn1)c1ccccc1-c1noc2cc(C#N)ccc12]

Tetrakis(triphenylphosphine)palldium (0) (0.023 g) was added to a degassed solution of tert-butyl (S)-{1-[2-(6-bromobenzo[d]isoxazol-3-yl)phenyl]-2-(pyridin-2-yl)ethyl}carbamate (Intermediate 9A, 0.1 g) and zinc cyanide (0.356 g) in DMF (3 mL) and the resultant mixture was heated in a sealed vial at 150° C. for 25 minutes then allowed to cooled to room temperature and combined with a previous similar experiment. The combined mixtures were partitioned between water and ethyl acetate and the organic phase was washed with brine, dried (MgSO₄) and filtered. The filtrate was concentrated in vacuo and the residue was purified by FCC eluting with 0-60% diethyl ether in petroleum ether to give the title compound as a pale red solid (0.051 g). LCMS Method (Method 5) RT 2.94 minutes, m/z 441 [MH⁺] 341 [MH⁺−100] (loss of Boc group).

### Intermediate 11A: tert-Butyl (S)-{1-[2-(6-methylbenzo[d]isoxazol-3-yl)phenyl]-2-(pyridin-2-yl)ethyl}carbamate

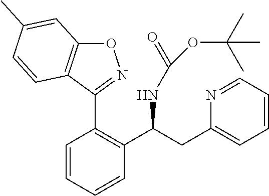 [SMILES: Cc1ccc2c(-c3ccccc3[C@H](Cc3ccccn3)NC(=O)OC(C)(C)C)noc2c1]

A mixture of tert-butyl (S)-{1-[2-(6-bromobenzo[d]isoxazol-3-yl)phenyl]-2-(pyridin-2-yl)ethyl}carbamate (Intermediate 9A, 0.05 g), trimethylboroxine (14.8 mL) and cesium carbonate (0.099 g) in dioxane (3 mL) and water (0.3 mL) was degassed and then treated with [1,1′-bis(diphenylphosphino)ferrocene]dichloropalladium (II) (0.008 g). The mixture was again degassed then stirred and heated at 100° C. overnight. After cooling to room temperature, the mixture was filtered through a pad of Celite® and the filtrate was concentrated in vacuo. The residue was purified by FCC eluting with 0-100% ethyl acetate in cyclohexane to give the title compound (0.03 g) as a white solid which was used directly in the next stage.

By proceeding in a similar manner to Intermediate 11A, the following compounds were prepared:

### Intermediate 11B: tert-Butyl (S)-{1-[2-(benzo[d]isoxazol-3-yl)-5-methylphenyl]-2-(pyridin-2-yl)ethyl}carbamate

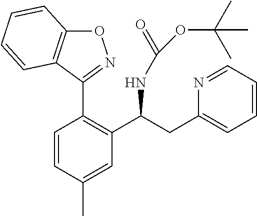 [SMILES: Cc1ccc(-c2noc3ccccc23)c([C@H](Cc2ccccn2)NC(=O)OC(C)(C)C)c1]

Starting from tert-butyl (S)-{1-[2-(benzo[d]isoxazol-3-yl)-5-bromophenyl]-2-(pyridin-2-yl)ethyl}carbamate (Intermediate 9B) and trimethyl boroxine and used directly in the next stage.

### Intermediate 11C: tert-Butyl (S)-{1-[2-(benzo[d]isoxazol-3-yl)-4-methylphenyl]-2-(pyridin-2-yl)ethyl}carbamate

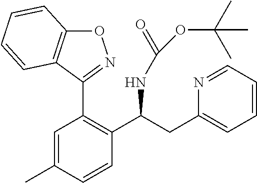 [SMILES: Cc1ccc([C@H](Cc2ccccn2)NC(=O)OC(C)(C)C)c(-c2noc3ccccc23)c1]

Starting from tert-butyl (S)-{1-[2-(benzo[d]isoxazol-3-yl)-4-bromophenyl]-2-(pyridine-2-yl)ethyl}carbamate (Intermediate 9C) and trimethyl boroxine. LCMS (Method 5) RT 3.45 m/z 430 [MH⁺].

### Intermediate 11D: tert-Butyl (S)-{1-[2-(benzo[d]isoxazol-3-yl)phenyl]-2-(6-methylpyridine-2-yl)ethyl}carbamate

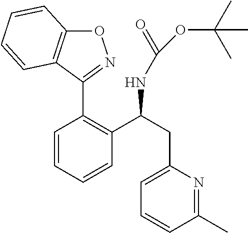 [SMILES: Cc1cccc(C[C@H](NC(=O)OC(C)(C)C)c2ccccc2-c2noc3ccccc23)n1]

Starting from tert-butyl (S)-{1-[2-(benzo[d]isoxazol-3-yl)phenyl]-2-(6-bromopyridin-2-yl)ethyl}carbamate (Intermediate 9D) and trimethyl boroxine. LCMS (Method 5) RT 2.93 m/z 430 [MH⁺].

### Intermediate 11E: (S)—N—{(S)-1-[2-(Benzo[d]isoxazol-3-yl)phenyl]-2-(5-methylpyridine-2-yl)ethyl}-2-methylpropane-2-sulfinamide

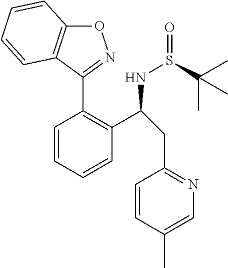 [SMILES: Cc1ccc(C[C@H](N[S@@](=O)C(C)(C)C)c2ccccc2-c2noc3ccccc23)nc1]

Starting from (S)—N—{(S)-1-[2-(benzo[d]isoxazol-3-yl)phenyl]-2-(5-bromopyridine-2-yl)ethyl}-2-methylpropane-2-sulfinamide (Intermediate 6D) and trimethyl boroxine. LCMS (Method 5) RT 2.74 m/z 434 [MH⁺].

### Intermediate 11F: (S)—N—{(S)-1-[2-(Benzo[d]isoxazol-3-yl)phenyl]-2-(3,6-dimethylpyridine-2-yl)ethyl}-2-methylpropane-2-sulfinamide

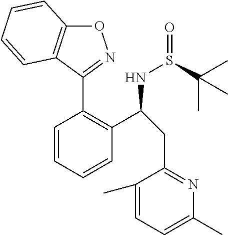 [SMILES: Cc1ccc(C)c(C[C@H](N[S@@](=O)C(C)(C)C)c2ccccc2-c2noc3ccccc23)n1]

Starting from (S)—N—{(S)-1-[2-(benzo[d]isoxazol-3-yl)phenyl]-2-(3-methyl-6-bromopyridine-2-yl)ethyl}-2-methylpropane-2-sulfinamide (Intermediate 61) and trimethyl boroxine. LCMS (Method 4) RT 1.08 m/z 448 [MH⁺].

### Intermediate 11G: (S)—N—{(S)-1-[2-(1-Isopropyl-1H-indazol-3-yl)phenyl]-2-[6-methylpyridin-2-yl]-ethyl}-2-methylpropane-2-sulfinamide

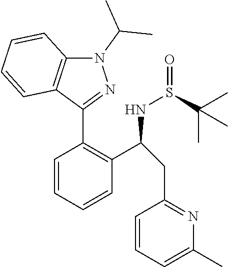 [SMILES: Cc1cccc(C[C@H](N[S@@](=O)C(C)(C)C)c2ccccc2-c2nn(C(C)C)c3ccccc23)n1]

Starting from (S)—N-{2-[6-bromopyridine-2-yl]-1-[2-(1-isopropyl-1H-indazol-3-yl)phenyl]ethyl}-2-methylpropane-2-sulfinamide (Intermediate 60) and trimethyl boroxine. LCMS (Method 4) RT 1.18 m/z 475 [MH⁺].

### Intermediate 11H: (S)—N—{(S)-1-[2-(Benzo[d]isoxazol-3-yl)phenyl]-2-[3-fluoro-6-methyl-4-(trimethylsilyl)pyridine-2-yl]ethyl}-2-methylpropane-2-sulfinamide

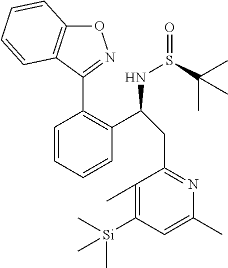 [SMILES: Cc1cc([Si](C)(C)C)c(C)c(C[C@H](N[S@@](=O)C(C)(C)C)c2ccccc2-c2noc3ccccc23)n1]

Starting from (S)—N—{(S)-1-[2-(benzo[d]isoxazol-3-yl)phenyl]-2-[6-bromo-3-fluoro-4-(trimethylsilyl)pyridine-2-yl]ethyl}-2-methylpropane-2-sulfinamide (Intermediate 6S) and trimethyl boroxine and heating at 90° C. for 2 hours. LCMS (Method 3) RT 1.74 m/z 524 [MH⁺].

### Intermediate 11I: 2-(6-Methylbenzo[d]isoxazol-3-yl)benzaldehyde

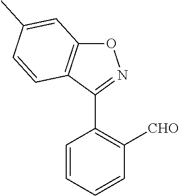 [SMILES: Cc1ccc2c(-c3ccccc3C=O)noc2c1]

Starting from 2-(6-bromobenzo[d]isoxazol-3-yl)benzaldehyde (Intermediate 3A) and trimethyl boroxine and heating at reflux for 4 hours. LCMS (Method 3) RT 1.43 m/z 238 [MH⁺].

### Intermediate 12A: tert-Butyl (S)-{1-[2-(6-cyclopropylbenzo[d]isoxazol-3-yl)phenyl]-2-(pyridine-2-yl)ethyl}carbamate

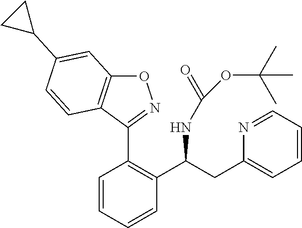 [SMILES: CC(C)(C)OC(=O)N[C@@H](Cc1ccccn1)c1ccccc1-c1noc2cc(C3CC3)ccc12]

A mixture of tert-butyl (S)-{1-[2-(6-bromobenzo[d]isoxazol-3-yl)phenyl]-2-(pyridine-2-yl)ethyl}carbamate (Intermediate 9A, 0.05 g), cyclopropyl boronic acid (0.011 g), tricyclohexylphosphine (0.003 g) and tripotassium phosphate (0.075 g) in toluene (2 mL) and water (0.1 mL) was degassed and treated with palladium acetate (0.001 g). The resultant mixture was again degassed and then stirred and heated at 100° C. overnight. After cooling, the mixture was filtered through Celite® and the filtrate was concentrated in vacuo. The residue was purified by FCC eluting with 0-100% ethyl acetate in petroleum ether to give the title compound which was used directly in the next stage.

### Intermediate 13A: tert-Butyl (S)-{1-[2-(6-vinylbenzo[d]isoxazol-3-yl)phenyl]-2-(pyridine-2-yl)ethyl}carbamate

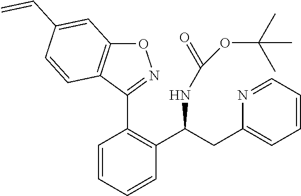 [SMILES: C=Cc1ccc2c(-c3ccccc3[C@H](Cc3ccccn3)NC(=O)OC(C)(C)C)noc2c1]

A mixture of tert-butyl (S)-{1-[2-(6-bromobenzo[d]isoxazol-3-yl)phenyl]-2-(pyridine-2-yl)ethyl}carbamate (Intermediate 9A, 1.0 g), 4,4,5,5-tetramethyl-2-vinyl-1,3,2-dioxaborolane (0.51 mL) and sodium bicarbonate (0.424 g) in dioxane (12 mL) and water (3 mL) was degassed and tetrakis-(triphenylphosphine)palladium (0.233 g) was added. The mixture was again degassed and the vial sealed then heated at 95° C. for 5 hours then at room temperature overnight. The mixture was filtered through Celite®, the filtrate was partitioned between ethyl acetate and water. The aqueous phase was further extracted with ethyl acetate and the combined organic phases were dried (Na₂SO₄) and filtered. The filtrate was concentrated in vacuo and the residue was purified by FCC eluting with 0-50% ether in petroleum ether to give the title compound (0.437 g) as an orange oil. LCMS (Method 5) RT 3.32 m/z 442.

By proceeding in a similar manner to Intermediate 13A, the following compounds were prepared:

### Intermediate 13B: (S)—N—{(S)-1-[2-(Benzo[d]isoxazol-3-yl)phenyl]-2-(6-vinylpyridin-2-yl)ethyl}-2-methylpropane-2-sulfinamide

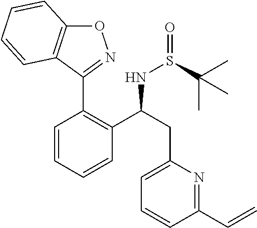 [SMILES: C=Cc1cccc(C[C@H](N[S@@](=O)C(C)(C)C)c2ccccc2-c2noc3ccccc23)n1]

Starting from (S)—N—{(S)-2-[6-bromopyridine-2-yl]-1-[2-(benzo[d]isoxazol-3-yl)phenyl]ethyl}propane-2-sulfinamide (Intermediate 6B) and 4,4,5,5-tetramethyl-2-vinyl-1,3,2-dioxaborolane. LCMS (Method 8) RT 3.18 m/z 446 [MH⁺].

### Intermediate 13C: (S)—N—{(S)-1-[2-(Benzo[d]isoxazol-3-yl)phenyl]-2-[3-fluoro-4-(trimethylsilyl)-6-vinylpyridin-2-yl]ethyl}-2-methylpropane-2-sulfinamide

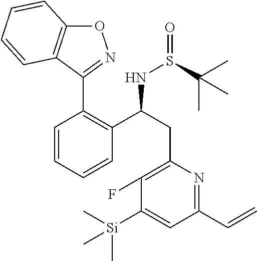 [SMILES: C=Cc1cc([Si](C)(C)C)c(F)c(C[C@H](N[S@@](=O)C(C)(C)C)c2ccccc2-c2noc3ccccc23)n1]

Starting from (S)—N—{(S)-1-[2-(benzo[d]isoxazol-3-yl)phenyl]-2-[6-bromo-3-fluoro-4-(trimethylsilyl)pyridin-2-yl]ethyl}-2-methylpropane-2-sulfinamide (Intermediate 6S) and 4,4,5,5-tetramethyl-2-vinyl-1,3,2-dioxaborolane. LCMS (Method 3) RT 1.80 m/z 536 [MH⁺].

### Intermediate 13D: (S)—N—{(S)-1-[2-(Benzo[d]isoxazol-3-yl)phenyl]-2-(3-methyl-6-vinylpyridin-2-yl)ethyl}-2-methylpropane-2-sulfinamide

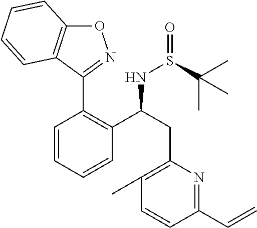 [SMILES: C=Cc1ccc(C)c(C[C@H](N[S@@](=O)C(C)(C)C)c2ccccc2-c2noc3ccccc23)n1]

Starting from (S)—N—{(S)-1-[2-(benzo[d]isoxazol-3-yl)phenyl]-2-(6-bromo-3-methylpyridin-2-yl)ethyl}-2-methylpropane-2-sulfinamide (Intermediate 61) and 4,4,5,5-tetramethyl-2-vinyl-1,3,2-dioxaborolane. LCMS (Method 4) RT 1.45 m/z 460 [MH⁺].

### Intermediate 14A: tert-Butyl (S)-{1-[2-(6-hydroxymethylbenzo[d]isoxazol-3-yl)phenyl]-2-(pyridine-2-yl)ethyl}carbamate

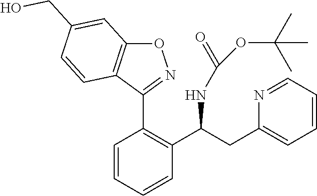 [SMILES: CC(C)(C)OC(=O)N[C@@H](Cc1ccccn1)c1ccccc1-c1noc2cc(CO)ccc12]

A solution of tert-butyl (S)-{1-[2-(6-vinylbenzo[d]isoxazol-3-yl)phenyl]-2-(pyridine-2-yl)ethyl}carbamate (Intermediate 13A, 0.302 g) in methanol (10 mL) and dioxane (2 mL) was cooled to −78° C. while nitrogen was bubbling through the solution. Once cooled, oxygen was bubbled through followed by ozone for 5-10 minutes followed again by oxygen to remove the excess ozone. The mixture was then placed under an atmosphere of nitrogen and sodium borohydride (0.076 g) was added and the mixture was stirred at −78° C. for 1 hour and then allowed to warm to room temperature and stirred overnight. A few drops of saturated aqueous sodium bicarbonate were added and the mixture was concentrated in vacuo, combined with an earlier experiment starting from 0.1 g of Intermediate 13A and purified by FCC eluting with 0-70% ethyl acetate in petroleum ether to give the title compound as a gum which was used directly in the next stage.

By proceeding in a similar manner to Intermediate 14A, the following compounds were prepared:

### Intermediate 14B: (S)—N—{(S)-1-[2-(Benzo[d]isoxazol-3-yl)phenyl]-2-(6-hydroxymethylpyridin-2-yl)ethyl}-2-methylpropane-2-sulfinamide

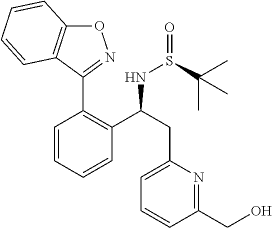 [SMILES: CC(C)(C)[S@](=O)N[C@@H](Cc1cccc(CO)n1)c1ccccc1-c1noc2ccccc12]

Starting from (S)—N—{(S)-1-[2-(benzo[d]isoxazol-3-yl)phenyl]-2-(6-vinylpyridin-2-yl)ethyl}-2-methylpropane-2-sulfinamide (Intermediate 13B). LCMS (Method 8) RT 2.40 m/z 450 [MH⁺].

### Intermediate 15A: tert-Butyl (S)-{1-[2-(6-[trimethylsilylethynyl]benzo[d]isoxazol-3-yl)phenyl]-2-(pyridine-2-yl)ethyl}carbamate

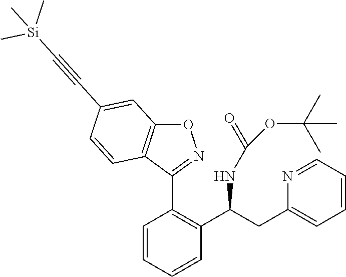 [SMILES: CC(C)(C)OC(=O)N[C@@H](Cc1ccccn1)c1ccccc1-c1noc2cc(C#C[Si](C)(C)C)ccc12]

A mixture of tert-butyl (S)-{1-[2-(6-bromobenzo[d]isoxazol-3-yl)phenyl]-2-(pyridine-2-yl)ethyl}carbamate (Intermediate 9A, 0.067 g), trimethylsilylacetylene (0.058 mL), copper (I) iodide (0.0026 g) and trimethylamine (0.057 mL) in DMF (2 mL) was degassed then treated with tetrakis(triphenylphosphine)palladium (0.016 g). The mixture was again degassed then stirred and heated at 95° C. overnight. After cooling, the mixture was partitioned between ethyl acetate and water. The aqueous phase was further extracted with ethyl acetate and the combined organic phases were dried (MgSO₄) and filtered. The filtrate was concentrated in vacuo and the residue was purified by FCC eluting with 0-30% ethyl acetate in petroleum ether to give the title compound as a gum (0.016 g) which was used directly in the next stage.

By proceeding in a similar manner to Intermediate 15A, the following compounds were prepared:

### Intermediate 15B: tert-Butyl (S)-{1-[2-(6-[3-hydroxyprop-1-yn-1-yl]benzo[d]isoxazol-3-yl)phenyl]-2-(pyridine-2-yl)ethyl}carbamate

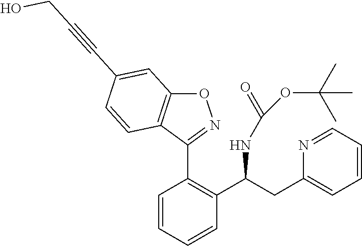 [SMILES: CC(C)(C)OC(=O)N[C@@H](Cc1ccccn1)c1ccccc1-c1noc2cc(C#CCO)ccc12]

Starting from tert-butyl (S)-{1-[2-(6-bromobenzo[d]isoxazol-3-yl)phenyl]-2-(pyridine-2-yl)ethyl}carbamate (Intermediate 9A) and prop-2-yn-1-ol. LCMS (Method 5) RT 2.73 m/z 470.

### Intermediate 16A: tert-Butyl (S)-{1-[2-(6-formylbenzo[d]isoxazol-3-yl)phenyl]-2-(pyridine-2-yl)ethyl}carbamate

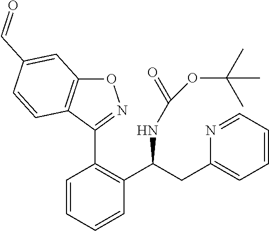 [SMILES: CC(C)(C)OC(=O)N[C@@H](Cc1ccccn1)c1ccccc1-c1noc2cc(C=O)ccc12]

Osmium tetraoxide (2.5% in tert-butanol, 3.3 mL) and sodium periodate (0.434 g) were added to a solution of tert-butyl (S)-{1-[2-(6-vinylbenzo[d]isoxazol-3-yl)phenyl]-2-(pyridine-2-yl)ethyl}carbamate (Intermediate 13A, 0.391 g) in dioxane (12 mL) and water (6 mL). The resultant mixture was stirred at room temperature for 72 hours. An aqueous solution of sodium sulphite was added and the phases were separated. The aqueous phase was extracted with ethyl acetate and the combined organic phases were dried (MgSO₄) and filtered. The filtrate was concentrated in vacuo to give the title compound (0.333 g) as a tan coloured solid. ¹H NMR (400 MHz, CDCl₃) 10.22 (1H, s), 8.53-8.49 (1H, m), 8.16 (1H, s), 8.03-7.99 (1H, m), 7.93-7.90 (1H, m), 7.57-7.35 (6H, m), 7.15-7.08 (1H, m), 6.45-6.42 (1H, m), 5.22-5.16 (1H, m), 3.36-3.29 (1H, m), 3.08-3.00 (1H, m), 1.31 (9H, s).

### Intermediate 17A: tert-Butyl (S)-{1-[2-(6-[1-hydroxyethyl]benzo[d]isoxazol-3-yl)phenyl]-2-(pyridine-2-yl)ethyl}carbamate

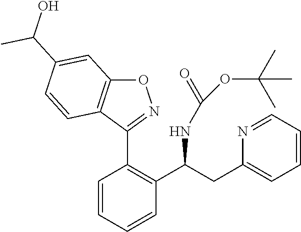 [SMILES: CC(O)c1ccc2c(-c3ccccc3[C@H](Cc3ccccn3)NC(=O)OC(C)(C)C)noc2c1]

A solution of methyl magnesium bromide (3M in ether, 0.38 mL) was added dropwise to a stirred, cooled solution of tert-butyl (S)-{1-[2-(6-formylbenzo[d]isoxazol-3-yl)phenyl]-2-(pyridine-2-yl)ethyl}carbamate (Intermediate 16A, 0.145 g) in dry THF (3 mL) at −78° C. On completion of the addition, the mixture was allowed to warm to room temperature and stirred for 72 hours. A saturated solution of ammonium chloride was added and the mixture was partitioned between ethyl acetate and water. The aqueous phase was further extracted with ethyl acetate and the combined organic phases were dried (MgSO₄) and filtered. The filtrate was concentrated in vacuo and purified by FCC eluting with 0-100% ethyl acetate in pentane to give the title compound (0.073 g). ¹H NMR (400 MHz, DMSO-d₆, 80° C.) 8.32 (1H, d, J=4.75 Hz), 7.72-7.67 (2H, m), 7.58-7.37 (6H, m), 7.09-7.04 (1H, m), 6.98 (1H, d, J=7.99 Hz), 6.83-6.75 (1H, br. s), 5.35-5.27 (1H, m), 5.07-5.027 (1H, m), 4.97-4.90 (1H, m), 3.11-3.02 (1H, m), 1.43 (3H, d, J=6.30 Hz), 1.15 (9H, s).

### Intermediate 18A: Methyl 2-(hydroxyimino)methylbenzoate

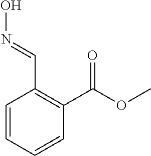

A solution of sodium acetate trihydrate (7.45 g) in water (74 mL) was added to a solution of methyl 2-formylbenzoate (5.0 g) in methanol (74 mL). Hydroxylamine hydrochloride was then added and the mixture was stirred at room temperature overnight. The mixture was concentrated in vacuo to remove the organic solvent and then extracted with ethyl acetate. The organic phase was washed with water, dried (Na₂SO₄) and filtered. The filtrate was concentrated in vacuo to give the title compound (5.6 g) as a sticky white solid. ¹H NMR (300 MHz, CDCl₃) 8.97 (1H, s), 8.01-7.95 (1H, m), 7.84-7.78 (1H, m), 7.59-7.42 (21H, m), 3.94 (3H, s);

By proceeding in a similar manner to Intermediate 18A, the following compounds were prepared:

### Intermediate 18B: Methyl 2-(hydroxyimino)methyl-5-methoxybenzoate

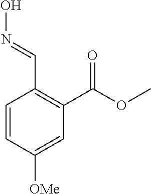

Starting from methyl 2-formyl-5-methoxybenzoate. LCMS (Method 5) RT 3.09 m/z 210 [MH⁺].

### Intermediate 18C: Methyl 2-(hydroxyimino)methyl-5-bromobenzoate

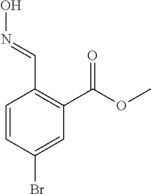

Starting from methyl 5-bromo-2-formylbenzoate. LCMS (Method 5) RT 3.56 m/z 258/260 [MH⁺]

### Intermediate 18D: Methyl 2-(hydroxyimino)methyl-4-bromobenzoate

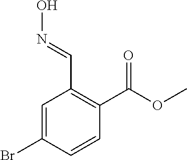

Starting from methyl 4-bromo-2-formylbenzoate. LCMS (Method 5) RT 3.54 m/z 258/260 [MH⁺]

### Intermediate 19A: Methyl 2-[chloro(hydroxyimino)methyl]benzoate

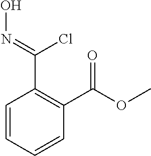

A solution of N-chlorosuccinimide (4.26 g) in DMF (2 mL) was added dropwise to a stirred, cooled solution of methyl 2-(hydroxyimino)methylbenzoate (Intermediate 18A, 5.8 g) in DMF (72 mL) at 0° C. The mixture was allowed to warm slowly to room temperature over ˜1 hour then stirred at that temperature for a further 1 hour. The resultant mixture was partitioned between ethyl acetate and water and the organic phase was washed with water, dried (Na₂SO₄) and filtered. The filtrate was concentrated in vacuo and the residue was purified by FCC eluting with 0-8% ethyl acetate in cyclohexane to give the title compound (2.38 g) as a colourless oil. ¹H NMR (300 MHz, CDCl₃) 7.92-7.87 (2H, m), 7.59-7.51 (2H, m), 3.91-3.90 (3H, s).

By proceeding in a similar manner to Intermediate 19A, the following compounds were prepared:

### Intermediate 19B: Methyl 2-[chloro(hydroxyimino)methyl]-5-methoxybenzoate

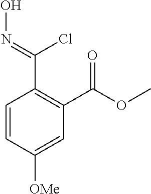 [SMILES: COC(=O)c1cc(OC)ccc1/C(Cl)=N/O]

Starting from methyl 2-(hydroxyimino)methyl-5-methoxybenzoate (Intermediate 18B). LCMS (Method 5) RT 3.45 m/z 208 [MH⁺-Cl].

### Intermediate 19C: Methyl 5-bromo-2-[chloro(hydroxyimino)methyl]benzoate

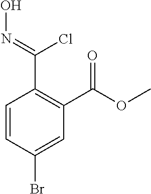

Starting from methyl 5-bromo-2-(hydroxyimino)methylbenzoate (Intermediate 18C). LCMS (Method 5) RT 3.45 m/z 256/258 [MH⁺-Cl].

### Intermediate 19D: Methyl 4-bromo-2-[chloro(hydroxyimino)methyl]benzoate

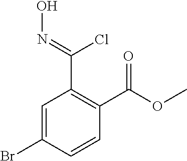

Starting from methyl 4-bromo-2-(hydroxyimino)methylbenzoate (Intermediate 18D). LCMS (Method 5) RT 3.85 m/z 256/258 [MH⁺-Cl].

### Intermediate 19E: Methyl 3-bromo-2-[chloro(hydroxyimino)methyl]benzoate

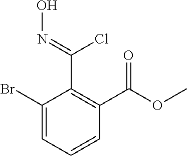

Starting from methyl 3-bromo-2-(hydroxyimino)methylbenzoate (Intermediate 37A). LCMS (Method 5) RT 3.73 m/z 256/258 [MH⁺-Cl].

### Intermediate 20A: (2,6-Dibromophenoxy)trimethylsilane

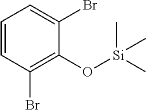

Bis(trimethylsilyl)amine (4.75 mL) was added dropwise to a solution of 2,6-dibromophenol (2.87 g) in dry THF (16 mL) under an atmosphere of argon. The resultant mixture was stirred and heated at reflux overnight. After cooling, the mixture was concentrate in vacuo to give the title compound (3.69 g) as a yellow oil. ¹H NMR (300 MHz, CDCl₃) 7.47 (2H, d, J=7.9 Hz), 6.71 (1H, t, J=8.1 Hz), 0.39 (9H, s).

### Intermediate 21A: 2-Bromo-6-trimethylsilylphenol

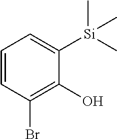

n-Butyllithium (2.5M in exanes was added dropwise to a stirred, cooled solution of (2,6-dibromophenoxy)trimethylsilane (Intermediate 20A, 2.87 g) in dry THF (108 mL) while maintaining the temperature below −65° C. The resultant mixture was allowed to warm to room temperature and stirred for 4 hours. A saturated aqueous solution of ammonium chloride was added and the mixture was extracted with diethyl ether. The combined organic phase was washed with water, dried (Na₂SO₄) and filtered. The filtrate was concentrated in vacuo and the residue was purified by FCC eluting with 0-7% ethyl acetate in cyclohexane to give the title compound (1.95 g) as a colourless oil. ¹H NMR (300 MHz, CDCl₃) 7.46 (1H, dd, J=1.5, 7.9 Hz), 7.29 (1H, dd, J=1.5, 7.4 Hz), 6.79 (1H, t, J=7.5 Hz), 5.70 (1H, s), 0.31 (9H, s);

### Intermediate 22A: 2-Bromo-6-(trimethylsilyl)phenyl trifluoromethansulfonate

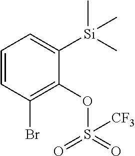 [SMILES: C[Si](C)(C)c1cccc(Br)c1OS(=O)(=O)C(F)(F)F]

Trifluoromethansulfonic anhydride (2.01 mL) was added dropwise to a stirred, cooled solution of 2-bromo-6-trimethylsilylphenol (Intermediate 21A, 1.95 g) and di-isopropylethylamine (2.1 mL) in dry DCM (84 mL) at 0° C. The mixture was stirred at 0° C. for 30 minutes then allowed to warm to room temperature and stirred overnight. It was diluted with DCM and washed with water, dried (MgSO₄) and filtered. The filtrate was concentrated in vacuo and the residue was purified by FCC eluting with 0-30% ethyl acetate in cyclohexane to give the title compound (2.09 g) as a yellow oil. ¹H NMR (300 MHz, CDCl₃) 7.67 (1H, dd, J=1.5, 7.8 Hz), 7.51 (1H, dd, J=1.5, 7.8 Hz), 7.23 (1H, t, J=7.8 Hz), 0.40 (9H, s).

### Intermediate 23A: Methyl 2-(4-bromobenzo[d]isoxazole-3-yl)benzoate

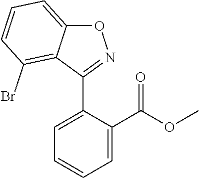 [SMILES: COC(=O)c1ccccc1-c1noc2cccc(Br)c12]

A solution of methyl 2-[chloro(hydroxyimino)methyl]benzoate (Intermediate 19A, 0.654 g) in acetonitrile (31 mL) was added dropwise to a solution of 2-bromo-6-(trimethylsilyl)phenyl trifluoromethansulfonate (Intermediate 22A, 2.08 g) and cesium fluoride (2.51 g) in acetonitrile (31 mL) under an atmosphere of argon. The mixture was stirred at room temperature overnight then filtered through Celite® and the filtrate was concentrated in vacuo. The residue was purified by FCC eluting with 0-30% ethyl acetate in cyclohexane to give the title compound (0.203 g) as a yellow oil. LCMS (Method 6) RT 3.77 m/z 332/334 [MH⁺].

By proceeding in a similar manner to Intermediate 23A, the following compounds were prepared:

### Intermediate 23B: Methyl 2-(benzo[d]isoxazole-3-yl)-5-methoxybenzoate

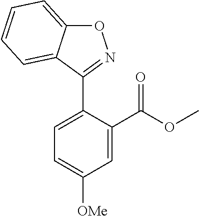 [SMILES: COC(=O)c1cc(OC)ccc1-c1noc2ccccc12]

Starting from methyl 2-[chloro(hydroxyimino)methyl]-5-methoxybenzoate (Intermediate 19B) and 2-(trimethylsilyl)phenyl trifluoromethansulfonate. LCMS (Method 5) RT 3.99 m/z 284.

### Intermediate 23C: Methyl 2-(benzo[d]isoxazole-3-yl)-5-bromobenzoate

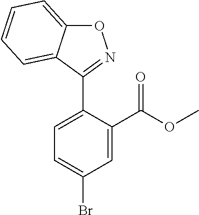 [SMILES: COC(=O)c1cc(Br)ccc1-c1noc2ccccc12]

Starting from methyl 5-bromo-2-[chloro(hydroxyimino)methyl]benzoate (Intermediate 19C) and 2-(trimethylsilyl)phenyl trifluoromethansulfonate. LCMS (Method 5) RT 4.33 m/z 332/334 [MH⁺].

### Intermediate 23D: Methyl 2-(benzo[d]isoxazole-3-yl)-4-bromobenzoate

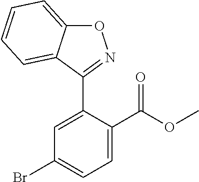 [SMILES: COC(=O)c1ccc(Br)cc1-c1noc2ccccc12]

Starting from methyl 4-bromo-2-[chloro(hydroxyimino)methyl]benzoate (Intermediate 19D) and 2-(trimethylsilyl)phenyl trifluoromethansulfonate. LCMS (Method 5) RT 4.31 m/z 332/334 [MH⁺].

### Intermediate 23E: Methyl 2-(benzo[d]isoxazole-3-yl)-3-bromobenzoate

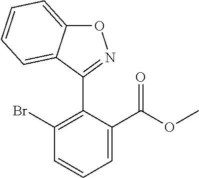 [SMILES: COC(=O)c1cccc(Br)c1-c1noc2ccccc12]

Starting from methyl 3-bromo-2-[chloro(hydroxyimino)methyl]benzoate (Intermediate 19E) and 2-(trimethylsilyl)phenyl trifluoromethansulfonate. LCMS (Method 5) RT 4.09 m/z 332/334 [MH⁺].

### Intermediate 24A: 2-(4-Bromobenzo[d]isoxazol-3-yl)phenylmethanol

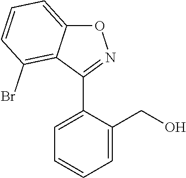 [SMILES: OCc1ccccc1-c1noc2cccc(Br)c12]

Lithium aluminium hydride (2M solution in THF, 0.318 mL) was added dropwise to a stirred, cooled solution of methyl 2-(4-bromobenzo[d]isoxazol-3-yl)benzoate (Intermediate 23A, 0.212 g) in dry THF (5 mL) at −20° C. The reaction mixture was allowed to slowly warm to 0° C. over 1 hour then an aqueous solution of sodium hydroxide (1M, 0.026 mL) was added, followed by water (0.076 mL). The resultant mixture was diluted with DCM and filtered through Celite®. The filtrate was concentrated in vacuo and the residue was purified by FCC eluting with 0-20% ethyl acetate in cyclohexane to give the title compound (0.122 g) as a yellow oil which was used directly in the next stage.

By proceeding in a similar manner to Intermediate 24A, the following compounds were prepared:

### Intermediate 24B: 2-(Benzo[d]isoxazol-3-yl)-5-methoxyphenylmethanol

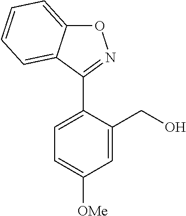 [SMILES: COc1ccc(-c2noc3ccccc23)c(CO)c1]

Starting from methyl 2-(benzo[d]isoxazole-3-yl)-5-methoxybenzoate (Intermediate 23B). LCMS (Method 5) RT 3.65 m/z 256 [MH⁺].

### Intermediate 24C: 2-(Benzo[d]isoxazol-3-yl)-5-bromophenylmethanol

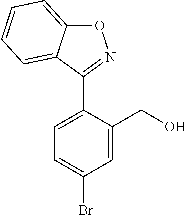 [SMILES: OCc1cc(Br)ccc1-c1noc2ccccc12]

Starting from methyl 2-(benzo[d]isoxazole-3-yl)-5-bromobenzoate (Intermediate 23C). LCMS (Method 5) RT 3.65 m/z 304/306 [MH⁺].

### Intermediate 24D: 2-(Benzo[d]isoxazol-3-yl)-4-bromophenylmethanol

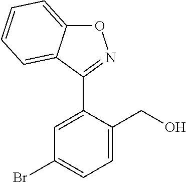 [SMILES: OCc1ccc(Br)cc1-c1noc2ccccc12]

Starting from methyl 2-(benzo[d]isoxazole-3-yl)-4-bromobenzoate (Intermediate 23D). LCMS (Method 5) RT 3.94 m/z 304/306 [MH⁺].

### Intermediate 24E: 2-(Benzo[d]isoxazol-3-yl)-3-bromophenylmethanol

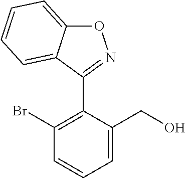 [SMILES: OCc1cccc(Br)c1-c1noc2ccccc12]

Starting from methyl 2-(benzo[d]isoxazole-3-yl)-3-bromobenzoate (Intermediate 23E). LCMS (Method 5) RT 3.68 m/z 304/306 [MH⁺].

### Intermediate 25A: 2-(4-Bromobenzo[d]isoxazol-3-yl)benzaldehyde

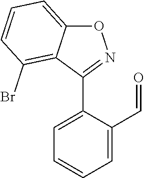 [SMILES: O=Cc1ccccc1-c1noc2cccc(Br)c12]

Activated manganese dioxide (0.351 g) was added to a solution of 2-(4-bromobenzo[d]isoxazol-3-yl)phenylmethanol (Intermediate 24A, 0.123 g) in DCM (9.5 mL) and the mixture was stirred and heated at 40° C. for 2.5 hours. After cooling, it was filtered through Celite®. The filtrate was concentrated in vacuo and the residue was purified by FCC eluting with 0-20% ethyl acetate in cyclohexane to give the title compound (0.084 g) as a yellow oil. ¹H NMR (300 MHz, CDCl₃) 9.96 (1H, s), 8.12-8.07 (1H, m), 7.81-7.70 (21H, m), 7.69-7.59 (2H, m), 7.49-7.45 (2H, m).

### Intermediate 26A: 2-(Benzo[d]isoxazol-3-yl)-5-methoxybenzaldehyde

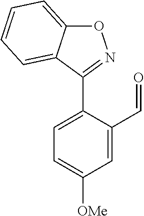 [SMILES: COc1ccc(-c2noc3ccccc23)c(C=O)c1]

DMP (0.732 g) was added to a solution of 2-(benzo[d]isoxazol-3-yl)-5-methoxyphenylmethanol (Intermediate 24B, 0.22 g) in DCM and the resultant suspension was stirred for 45 minutes. Aqueous sodium carbonate solution was added and the phases were separated. The aqueous phase was further extracted with DCM and the combined organic phases were filtered through a phase separator and the filtrate was concentrated in vacuo. The residue was purified by FCC eluting with 0-40% ethyl acetate in cyclohexane to give the title compound as an off-white solid (0.2 g). LCMS (Method 5) R/T 4.01 m/z 254 [MH⁺].

By proceeding in a similar manner to Intermediate 26A, the following compounds were prepared:

### Intermediate 26B: 2-(Benzo[d]isoxazol-3-yl)-5-bromobenzaldehyde

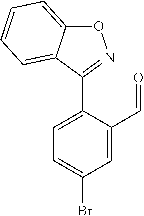 [SMILES: O=Cc1cc(Br)ccc1-c1noc2ccccc12]

Starting from 2-(benzo[d]isoxazol-3-yl)-5-bromophenylmethanol (Intermediate 24C). LCMS (Method 5) RT 4.28 m/z 302/304 [MH⁺].

### Intermediate 26C: 2-(Benzo[d]isoxazol-3-yl)-4-bromobenzaldehyde

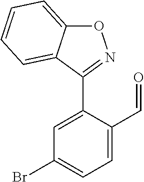 [SMILES: O=Cc1ccc(Br)cc1-c1noc2ccccc12]

Starting from 2-(benzo[d]isoxazol-3-yl)-4-bromophenylmethanol (Intermediate 24D). LCMS (Method 5) RT 4.25 m/z 302/304 [MH⁺].

### Intermediate 26D: 2-(Benzo[d]isoxazol-3-yl)-3-bromobenzaldehyde

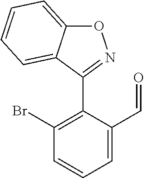 [SMILES: O=Cc1cccc(Br)c1-c1noc2ccccc12]

Starting from 2-(benzo[d]isoxazol-3-yl)-3-bromophenylmethanol (Intermediate 24E). LCMS (Method 5) RT 4.01 m/z 302/304 [MH⁺].

### Intermediate 27A: tert-Butyl (S)-{1-[2-(benzo[d]isoxazol-3-yl)-5-cyanophenyl]-2-(pyridine-2-yl)ethyl}carbamate

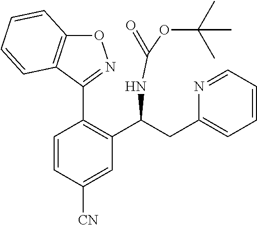 [SMILES: CC(C)(C)OC(=O)N[C@@H](Cc1ccccn1)c1cc(C#N)ccc1-c1noc2ccccc12]

Zinc cyanide (0.035 g) was added to a solution of tert-butyl (S)-{1-[2-(benzo[d]isoxazol-3-yl)-5-bromophenyl]-2-(pyridine-2-yl)ethyl}carbamate (Intermediate 9B, 0.097 g) in DMF (3 mL) and the solution was degassed. Tetrakis-(triphenylphosphine)palladium (0.023 g) was added and the mixture was again degassed then heated at 110° C. overnight. After cooling, the mixture was partitioned between ethyl acetate and water and the aqueous phase was extracted further with ethyl acetate. The combined organic phases were washed with brine, dried (Na₂SO₄) and filtered. The filtrate was concentrated in vacuo and the residue was purified by FCC eluting with 0-25% ethyl acetate in cyclohexane to give the title compound (0.055 g) as a gum. LCMS (Method 5) RT 3.54 m/z 441 [MH⁺].

By proceeding in a similar manner to Intermediate 27A, the following compounds were prepared:

### Intermediate 27B: tert-Butyl (S)-{1-[2-(benzo[d]isoxazol-3-yl)phenyl]-2-(6-cyanopyridine-2-yl)ethyl}carbamate

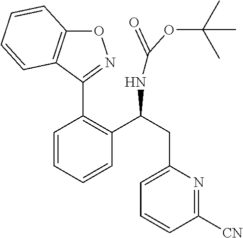 [SMILES: CC(C)(C)OC(=O)N[C@@H](Cc1cccc(C#N)n1)c1ccccc1-c1noc2ccccc12]

Starting from tert-butyl (S)-{1-[2-(benzo[d]isoxazol-3-yl)phenyl]-2-(6-bromopyridine-2-yl)ethyl}carbamate (Intermediate 9D). LCMS (Method 5) RT 4.32 m/z 463 [M+Na⁺].

### Intermediate 27C: (S)—N—{(S)-1-[2-(Benzo[d]isoxazol-3-yl)phenyl]-2-(5-cyanopyridine-2-yl)ethyl}-2-methylpropane-2-sulfinamide

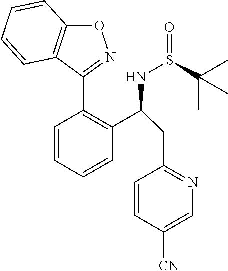 [SMILES: CC(C)(C)[S@](=O)N[C@@H](Cc1ccc(C#N)cn1)c1ccccc1-c1noc2ccccc12]

Starting from (S)—N—{(S)-1-[2-(benzo[d]isoxazol-3-yl)phenyl]-2-(5-bromopyridine-2-yl)ethyl}-2-methylpropane-2-sulfinamide (Intermediate 6D). LCMS (Method 5) RT 3.78 m/z 445 [MH⁺].

### Intermediate 27D: (S)—N—{(S)-1-[2-(6-Cyanobenzo[d]isoxazol-3-yl)phenyl]-2-(6-cyanopyridine-2-yl)ethyl}-2-methylpropane-2-sulfinamide

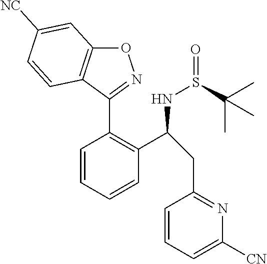 [SMILES: CC(C)(C)[S@](=O)N[C@@H](Cc1cccc(C#N)n1)c1ccccc1-c1noc2cc(C#N)ccc12]

Starting from (S)—N—{(S)-1-[2-(6-bromobenzo[d]isoxazol-3-yl)phenyl]-2-(6-bromopyridine-2-yl)ethyl}-2-methylpropane-2-sulfinamide (Intermediate 6E). LCMS (Method 7) RT 3.30 m/z 470 [MH⁺].

### Intermediate 27E: (S)—N—{(S)-1-[2-(6-Cyanobenzo[d]isoxazol-3-yl)phenyl]-2-(6-methylpyridine-2-yl)ethyl}-2-methylpropane-2-sulfinamide

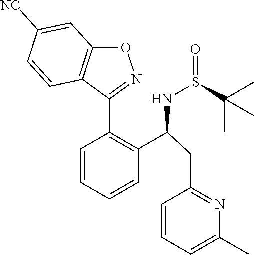 [SMILES: Cc1cccc(C[C@H](N[S@@](=O)C(C)(C)C)c2ccccc2-c2noc3cc(C#N)ccc23)n1]

Starting from (S)—N—{(S)-1-[2-(6-bromobenzo[d]isoxazol-3-yl)phenyl]-2-(6-methylpyridine-2-yl)ethyl}-2-methylpropane-2-sulfinamide (Intermediate 5L). LCMS (Method 7) RT 3.33 m/z 459 [MH⁺].

### Intermediate 27F: (S)—N—{(S)-1-[2-(6-Cyanobenzo[d]isoxazol-3-yl)phenyl]-2-(6-[methylamino]pyridine-2-yl)ethyl}-2-methylpropane-2-sulfinamide

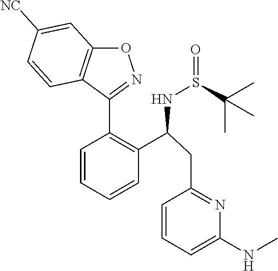 [SMILES: CNc1cccc(C[C@H](N[S@@](=O)C(C)(C)C)c2ccccc2-c2noc3cc(C#N)ccc23)n1]

Starting from (S)—N—{(S)-1-[2-(6-bromobenzo[d]isoxazol-3-yl)phenyl]-2-(6-[methylamino]pyridine-2-yl)ethyl}-2-methylpropane-2-sulfinamide (Intermediate 51A). LCMS (Method 6) RT 2.52 m/z 474 [MH⁺].

### Intermediate 27G: (S)—N-{(1S)-1-[2-(6-Cyanobenzo[d]isoxazol-3-yl)phenyl]-2-[6-{2-[(tetrahydro-2H-pyran-2-yl)oxy]ethoxy}pyridine-2-yl]ethyl}-2-methylpropane-2-sulfinamide

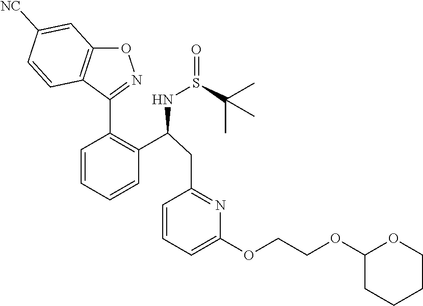 [SMILES: CC(C)(C)[S@](=O)N[C@@H](Cc1cccc(OCCOC2CCCCO2)n1)c1ccccc1-c1noc2cc(C#N)ccc12]

Starting from (S)—N-{(1S)-1-[2-(6-bromobenzo[d]isoxazol-3-yl)phenyl]-2-[6-{2-[(tetrahydro-2H-pyran-2-yl)oxy]ethoxy}pyridine-2-yl]ethyl}-2-methylpropane-2-sulfinamide (Intermediate 6G) but heating at 90° C. for 3 hours. LCMS (Method 6) RT 4.04 m/z 505 [MH⁺-84] (loss of THP group).

### Intermediate 27H: (S)—N—{(S)-1-[2-(Benzo[d]isoxazol-3-yl)phenyl]-2-(6-cyano-3-methylpyridine-2-yl)ethyl}-2-methylpropane-2-sulfinamide

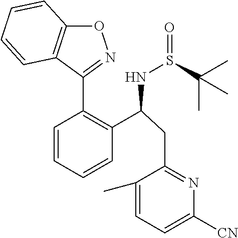 [SMILES: Cc1ccc(C#N)nc1C[C@H](N[S@@](=O)C(C)(C)C)c1ccccc1-c1noc2ccccc12]

Starting from (S)—N—{(S)-1-[2-(benzo[d]isoxazol-3-yl)phenyl]-2-(6-bromo-3-methylpyridin-2-yl)ethyl}-2-methylpropane-2-sulfinamide (Intermediate 61) but heating at 90° C. for 1 hour. LCMS (Method 5) RT 1.52 m/z 459 [MH⁺].

### Intermediate 271: (S)—N—{(S)-1-[2-(Benzo[d]isoxazol-3-yl)-6-fluorophenyl]-2-(6-cyanopyridine-2-yl)ethyl}-2-methylpropane-2-sulfinamide

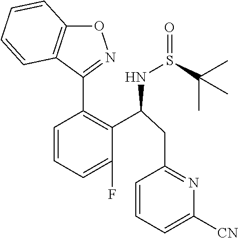 [SMILES: CC(C)(C)[S@](=O)N[C@@H](Cc1cccc(C#N)n1)c1c(F)cccc1-c1noc2ccccc12]

Starting from (S)—N—{(S)-1-[2-(benzo[d]isoxazol-3-yl)-6-fluorophenyl]-2-(6-bromopyridin-2-yl)ethyl}-2-methylpropane-2-sulfinamide (Intermediate 6J) but heating at 95° C. for 2 hours. LCMS (Method 4) RT 1.54 m/z 485 [M+Na⁺].

### Intermediate 27J: (S)—N—{(S)-2-[6-Cyanopyridine-2-yl]-1-[2-(1-isopropyl-1H-indazol-3-yl)phenyl]ethyl}-2-methylpropane-2-sulfinamide

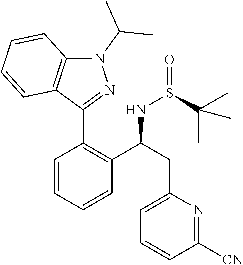 [SMILES: CC(C)n1nc(-c2ccccc2[C@H](Cc2cccc(C#N)n2)N[S@@](=O)C(C)(C)C)c2ccccc21]

Starting from (S)—N—{(S)-2-[6-bromopyridine-2-yl]-1-[2-(1-isopropyl-1H-indazol-3-yl)phenyl]ethyl}-2-methylpropane-2-sulfinamide (Intermediate 60) but heating at 90° C. overnight. LCMS (Method 4) RT 1.56 m/z 486 [MH⁺].

### Intermediate 27K: (S)—N—{(S)-1-[2-(Benzo[d]isoxazol-3-yl)phenyl]-2-[6-cyano-3-fluoro-4-(trimethylsilyl)pyridine-2-yl]ethyl}-2-methylpropane-2-sulfinamide

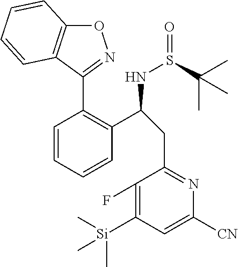 [SMILES: CC(C)(C)[S@](=O)N[C@@H](Cc1nc(C#N)cc([Si](C)(C)C)c1F)c1ccccc1-c1noc2ccccc12]

Starting from (S)—N—{(S)-1-[2-(benzo[d]isoxazol-3-yl)phenyl]-2-[6-bromo-3-fluoro-4-(trimethylsilyl)pyridine-2-yl]ethyl}-2-methylpropane-2-sulfinamide (Intermediate 6S). LCMS (Method 4) RT 1.70 m/z 535 [MH⁺].

### Intermediate 27L: (S)—N—{(S)-2-(3-Cyanopyridine-2-yl)-1-[2-(benzo[d]isoxazol-3-yl)phenyl]ethyl}-2-methylpropane-2-sulfinamide

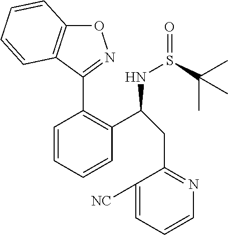 [SMILES: CC(C)(C)[S@](=O)N[C@@H](Cc1ncccc1C#N)c1ccccc1-c1noc2ccccc12]

Starting from (S)—N—{(S)-2-(3-bromopyridine-2-yl)-1-[2-(benzo[d]isoxazol-3-yl)phenyl]ethyl}-2-methylpropane-2-sulfinamide (Intermediate 6T). LCMS (Method 9) RT 3.63 m/z 445 [MH⁺].

### Intermediate 27M: (S)—N—{(S)-1-[2-(Benzo[d]isoxazol-3-yl)phenyl]ethyl}-2-(5-Cyano-3-methylpyridine-2-yl)-2-methylpropane-2-sulfinamide

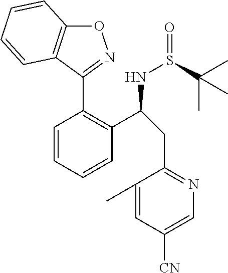 [SMILES: Cc1cc(C#N)cnc1C[C@H](N[S@@](=O)C(C)(C)C)c1ccccc1-c1noc2ccccc12]

Starting from (S)—N—{(S)-1-[2-(benzo[d]isoxazol-3-yl)phenyl]ethyl}-2-(5-bromo-3-methylpyridine-2-yl)-2-methylpropane-2-sulfinamide (Intermediate 6V) and used directly without further characterisation.

### Intermediate 27N: (S)—N—{(S)-1-[2-(Benzo[d]isoxazol-3-yl)phenyl]ethyl}-2-(6-cyano-5-fluoropyridine-2-yl)-2-methylpropane-2-sulfinamide

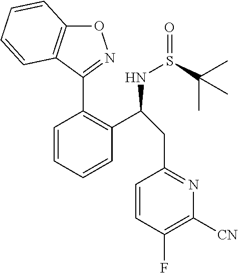 [SMILES: CC(C)(C)[S@](=O)N[C@@H](Cc1ccc(F)c(C#N)n1)c1ccccc1-c1noc2ccccc12]

Starting from (S)—N—{(S)-1-[2-(benzo[d]isoxazol-3-yl)phenyl]ethyl}-2-(6-bromo-5-fluoropyridine-2-yl)-2-methylpropane-2-sulfinamide (Intermediate 71H). LCMS (Method 3) RT 1.50 m/z 463 [MH⁺].

### Intermediate 270: (S)—N—{(S)-2-(6-cyano-3-fluoropyridine-2-yl)-1-[2-(6-methylbenzo[d]isoxazol-3-yl)phenyl]ethyl}-2-methylpropane-2-sulfinamide

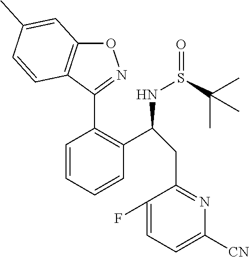 [SMILES: Cc1ccc2c(-c3ccccc3[C@H](Cc3nc(C#N)ccc3F)N[S@@](=O)C(C)(C)C)noc2c1]

Starting from (S)—N—{(S)-2-(6-bromo-3-fluoropyridine-2-yl)-1-[2-(6-methylbenzo[d]isoxazol-3-yl)phenyl]ethyl}-]-2-methylpropane-2-sulfinamide (Intermediate 71J) and heating at 90° C. for 1 hour. LCMS (Method 3) RT 1.42 m/z 477 [MH⁺].

### Intermediate 27P: (S)—N—{(S)-1-[2-(6-Chlorobenzo[d]isoxazol-3-yl)phenyl]-2-(6-cyano-3-fluoropyridine-2-yl)ethyl}-2-methylpropane-2-sulfinamide

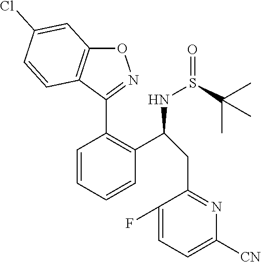 [SMILES: CC(C)(C)[S@](=O)N[C@@H](Cc1nc(C#N)ccc1F)c1ccccc1-c1noc2cc(Cl)ccc12]

Starting from (S)—N—{(S)-2-(6-bromo-3-fluoropyridine-2-yl)-1-[2-(6-chlorobenzo[d]isoxazol-3-yl)phenyl]ethyl}-2-methylpropane-2-sulfinamide (Intermediate 71K) and heating at 90° C. for 1 hour. LCMS (Method 3) RT 1.46 m/z 497 [MH⁺].

### Intermediate 27Q: (S,E)-N-[2-(6-Cyanobenzo[d]isoxazol-3-yl)benzylidene]-2-methylpropane-2-sulfinamide

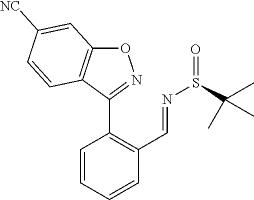 [SMILES: CC(C)(C)[S@](=O)/N=C/c1ccccc1-c1noc2cc(C#N)ccc12]

Starting from (S,E)-N-[2-(6-bromobenzo[d]isoxazol-3-yl)benzylidene]-2-methylpropane-2-sulfinamide (Intermediate 4A) and heating at 90° C. for 2 hours. LCMS (Method 3) RT 1.72 m/z 352 [MH⁺].

### Intermediate 27R: (S)—N—{(S)-2-(6-Cyano-3-methylpyridine-2-yl)-1-[2-(6-methylbenzo[d]isoxazol-3-yl)phenyl]ethyl}-2-methylpropane-2-sulfinamide

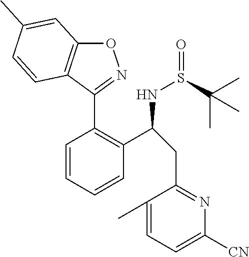 [SMILES: Cc1ccc2c(-c3ccccc3[C@H](Cc3nc(C#N)ccc3C)N[S@@](=O)C(C)(C)C)noc2c1]

Starting from (S)—N—{(S)-2-(6-bromo-3-methylpyridine-2-yl)-1-[2-(6-methylbenzo[d]isoxazol-3-yl)phenyl]ethyl}-]-2-methylpropane-2-sulfinamide (Intermediate 6AD) and heating at 90° C. for 2 hours. LCMS (Method 3) RT 1.50 m/z 473 [MH⁺].

### Intermediate 27S: (S)—N—{(S)-1-[2-(6-Bromobenzo[d]isoxazol-3-yl)phenyl]-2-(6-cyano-3-fluoropyridine-2-yl)ethyl}-2-methylpropane-2-sulfinamide

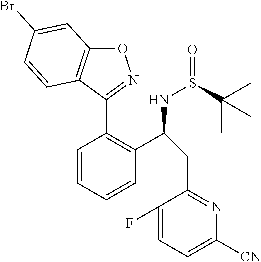 [SMILES: CC(C)(C)[S@](=O)N[C@@H](Cc1nc(C#N)ccc1F)c1ccccc1-c1noc2cc(Br)ccc12]

Starting from (S)—N—{(S)-2-(6-bromo-3-fluoropyridine-2-yl)-1-[2-(6-bromobenzo[d]isoxazol-3-yl)phenyl]ethyl}-2-methylpropane-2-sulfinamide (Intermediate 71M) but using potassium cyanide in place of zinc cyanide and heating at 70° C. for 2 hours. LCMS (Method 3) RT 1.52 m/z 541/543 [MH⁺].

### Intermediate 27T: (S)—N—{(S)-1-[2-(6-Bromobenzo[d]isoxazol-3-yl)phenyl]-2-(6-cyano-3-methylpyridine-2-yl)ethyl}-2-methylpropane-2-sulfinamide

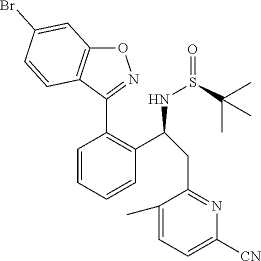 [SMILES: Cc1ccc(C#N)nc1C[C@H](N[S@@](=O)C(C)(C)C)c1ccccc1-c1noc2cc(Br)ccc12]

Starting from (S)—N—{(S)-2-[6-bromo-3-methylpyridine-2-yl]-1-[2-(6-bromobenzo[d]isoxazol-3-yl)phenyl]ethyl}-2-methylpropane-2-sulfinamide (Intermediate 6AI) but using potassium cyanide in place of zinc cyanide. LCMS (Method 3) RT 1.65 m/z 537/539 [MH⁺].

### Intermediate 28A: 2-(2-Bromo-6-methylphenyl)-1,3-dioxolane

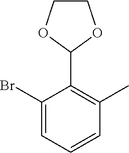

4-Methylbenzenesulfonic acid (0.19 g) and ethylene glycol (0.34 mL) were added to a solution of 2-bromo-6-methylbenzaldehyde (1.0 g) in toluene (10 mL) and the resultant mixture was stirred and heated at reflux under argon overnight. Further ethylene glycol (0.25 mL) and toluene (10 mL) were added and the mixture was stirred and heated at reflux with removal of water by means of a Dean and Stark apparatus for 24 hours. After cooling, the mixture was concentrated in vacuo and the residue was purified by FCC eluting with 0-30% ethyl acetate in cyclohexane to give the title compound (1.32 g) as a yellow oil. ¹H NMR (300 MHz, CDCl₃) 7.40 (1H, dd, J=1.8, 7.2 Hz), 7.11-7.07 (2H, m), 6.31 (1H, s), 4.26-4.17 (2H, m), 4.09-4.02 (2H, m), 2.46 (3H, s).

By proceeding in a similar manner to Intermediate 28A, the following compounds were prepared:

### Intermediate 28B: 2-(2,6-Dibromophenyl)-1,3-dioxolane

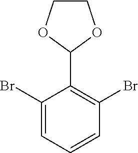 [SMILES: Brc1cccc(Br)c1C1OCCO1]

Starting from 2,6-dibromobenzaldehyde and ethylene glycol. LCMS (Method 5) RT 3.88 m/z 307/309/310 [MH⁺].

### Intermediate 28C: 2-(2-Bromo-6-fluorophenyl)-1,3-dioxolane

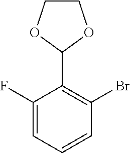 [SMILES: Fc1cccc(Br)c1C1OCCO1]

Starting from 2-bromo-6-fluorobenzaldehyde and ethylene glycol. ¹H NMR (400 MHz, CDCl₃) 7.36 (1H, td, J=1.1, 8.0 Hz), 7.19 (1H, dt, J=5.7, 8.1 Hz), 7.06-7.01 (1H, m), 6.33 (1H, d, J=1.2 Hz), 4.27-4.23 (2H, m), 4.09-4.03 (2H, m).

### Intermediates 29A, 29B and 29C: Diastereomers of (S)—N-{(1R)-1-[2-(benzo[d]isoxazol-3-yl)phenyl]-2-(pyridine-2-yl)propyl}-2-methylpropane-2-sulfinamide and (S)—N-{(1S)-1-[2-(benzo[d]isoxazol-3-yl)phenyl]-2-(pyridine-2-yl)propyl}-2-methylpropane-2-sulfinamide

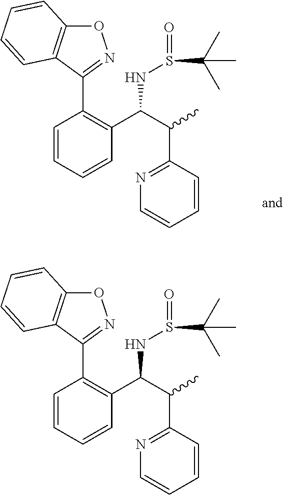

n-Butyllithium (1.6M in hexanes, 1.2 mL) was added to a stirred, cooled solution of 2-ethylpyridine (0.21 mL) in dry THF (3 mL) while maintaining the temperature below −60° C. The resultant mixture was stirred at −78° C. for 30 minutes then a solution of (S)—N-[2-(benzo[d]isoxazol-3-yl)benzylidene]-2-methylpropane-2-sulfinamide (Intermediate 4B, 0.3 g) in dry THF (2 mL) was added while maintaining the temperature below −60° C. The resultant solution was stirred at −78° C. for 2 hours then allowed to warm to room temperature and stirred for 2 hours. Water was added and the mixture was extracted with ethyl acetate, washed with water, dried (MgSO₄) and filtered. The filtrate was concentrated in vacuo and the residue was purified by FCC eluting with 50-100% ethyl acetate in pentane, followed by 0-2.5% methanol in ethyl acetate. Further purification of the fractions allowed the isolation of three of the four possible diastereomers.

Intermediate 29A: Diastereomer 1 (fastest running component). ¹H NMR (400 MHz, CDCl₃) 8.55 (1H, dd, J=1.0, 4.8 Hz), 7.71-7.60 (3H, m), 7.55-7.46 (2H, m), 7.40-7.31 (3H, m), 7.15-7.07 (2H, m), 6.84-6.81 (1H, m), 6.04 (1H, d, J=3.5 Hz), 4.96-4.92 (1H, m), 3.42-3.34 (1H, m), 1.36 (3H, d, J=6.2 Hz), 1.15-1.14 (9H, m).

Intermediate 29B: Diastereomer 2 (second fastest running component). ¹H NMR (400 MHz, CDCl₃) 8.38 (1H, dd, J=1.0, 4.8 Hz), 7.71-7.59 (4H, m), 7.49-7.32 (6H, m), 6.99 (1H, dd, J=5.1, 6.8 Hz), 5.67-5.62 (1H, m), 4.91-4.83 (1H, m), 3.33-3.24 (1H, m), 1.30 (3H, d, J=6.9 Hz), 1.08 (9H, s).

Intermediate 29C: Diastereomer 3 (Slowest running component). ¹H NMR (400 MHz, CDCl₃) 8.54 (1H, dd, J=0.9, 4.9 Hz), 7.73-7.60 (4H, m), 7.54-7.33 (5H, m), 7.13-7.07 (1H, m), 6.82-6.78 (1H, m), 6.04-5.98 (1H, m), 4.92-4.84 (1H, m), 3.30-3.27 (1H, m), 1.06 (3H, d, J=14.1 Hz), 1.02 (9H, s);

### Intermediate 30A: 1-[2-Benzo[d]isoxazol-3-yl)phenyl]ethan-1-ol

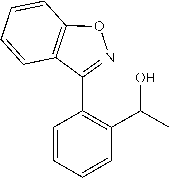 [SMILES: CC(O)c1ccccc1-c1noc2ccccc12]

A solution of methyl magnesium bromide (3M in ether, 2.4 mL) was added dropwise to a stirred, cooled solution of 2-(benzo[d]isoxazol-3-yl)benzaldehyde (Intermediate 3B, 0.78 g) in THF while maintaining the temperature below 5° C. The resultant mixture was stirred at <5° C. for 2 hours then 1M hydrochloric acid was added cautiously. The mixture was extracted with ethyl acetate, washed with water, dried (MgSO₄) and filtered. The filtrate was concentrated in vacuo to give the title compound (0.926 g) as a clear oil. ¹H NMR (400 MHz, CDCl₃) 7.79 (1H, dd, J=1.0, 7.8 Hz), 7.72-7.56 (5H, m), 7.50-7.45 (1H, m), 7.41-7.36 (1H, m), 5.02 (1H, q, J=6.5 Hz), 3.31 (1H, br s) 1.54 (3H, d, J=6.3 Hz).

### Intermediate 31A: 1-[2-(Benzo[d]isoxazol-3-yl)phenyl]ethan-1-one

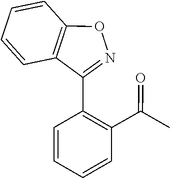 [SMILES: CC(=O)c1ccccc1-c1noc2ccccc12]

Pyridinium chlorochromate (1.06 g) was added to a mixture of 1-[2-benzo[d]isoxazol-3-yl)phenyl]ethan-1-ol (Intermediate 30A, 0.926 g) and Celite™ (0.73 g) in DCM at 0° C. On completion of the addition, the mixture was allowed to warm to room temperature and stirred for 3 days. The mixture was filtered through Celite™ and the filtrate was concentrated in vacuo. The residue was purified by FCC eluting with 10-30% ethyl acetate in pentane to give the title compound as a clear oil. ¹H NMR (400 MHz, CDCl₃) 7.82-7.80 (1H, m), 7.69-7.54 (6H, m), 7.36-7.32 (1H, m), 2.38 (3H, s).

### Intermediate 32A: (S)—N-{1-[2-(Benzo[d]isoxazol-3-yl)phenyl]ethylidene}-2-methylpropane-2-sulfinamide

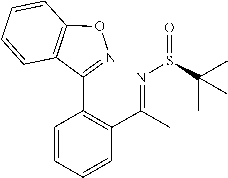 [SMILES: C/C(=N\[S@@](=O)C(C)(C)C)c1ccccc1-c1noc2ccccc12]

Titanium ethoxide (0.42 mL) was added to a solution of 1-[2-(benzo[d]isoxazol-3-yl)phenyl]ethan-1-one (Intermediate 31A, 0.237 g) and (S)-2-methylpropane-2-sulfinamide (0.11 g) in THF (2 mL). The vial was sealed and the mixture was stirred and heated at 60° C. for 24 hours. After cooling, the mixture was suspended in a mixture of ethyl acetate and water and filtered through Celite™. The layers were separated and the organic phase was washed with water, dried (MgSO₄) and filtered. The filtrate was concentrated in vacuo and the residue was purified by FCC eluting with 10-40% ethyl acetate in pentane to give the title compound (0.116 g) as a yellow gum. ¹H NMR (400 MHz, CDCl₃) 7.70-7.54 (7H, m), 7.36-7.31 (1H, m), 2.56 (3H, s), 1.02 (9H, s).

### Intermediate 33A: tert-Butyl (S)-{1-[2-(6-bromomethylbenzo[d]isoxazol-3-yl)phenyl}-2-(pyridine-2-yl)ethyl}carbamate

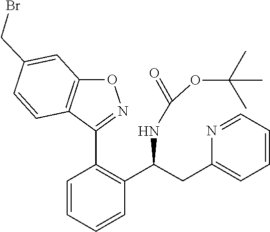 [SMILES: CC(C)(C)OC(=O)N[C@@H](Cc1ccccn1)c1ccccc1-c1noc2cc(CBr)ccc12]

Triphenylphosphine (0.019 g) was added to a cooled solution of tert-butyl (S)-{1-[2-(6-hydroxymethylbenzo[d]isoxazol-3-yl)phenyl]-2-(pyridine-2-yl)ethyl}carbamate (Intermediate 14A, 0.03 g) and carbon tetrabromide (0.025 g) in DCM (3 mL) at 0° C. On completion of the addition, the mixture was allowed to warm to room temperature and stirred for 2 hours. Further triphenylphosphine (0.01 g) and carbon tetrabromide (0.012 g) were added and stirring was continued for a further 2 hours. The mixture was treated with water and DCM and the layers were separated. The organic layer was dried (MgSO₄) and filtered and the filtrate was concentrated in vacuo. The residue was redissolved in DCM (3 mL) and triphenylphosphine (0.039 g) and carbon tetrabromide (0.049 g) were added. The resultant mixture was stirred at room temperature overnight. The mixture was partitioned between water and DCM. The aqueous layer was further extracted with DCM and the combined organic layers were washed with brine, dried (MgSO₄) and filtered. The filtrate was concentrated in vacuo and the residue was purified by FCC eluting with 0-60% diethyl ether in petrol to give the title compound (0.019 g) as a white solid. LCMS (Method 8) RT 3.13 m/z 508/510 [MH⁺].

### Intermediate 34A: tert-Butyl (S)-{1-[2-(6-methoxymethylbenzo[d]isoxazol-3-yl)phenyl}-2-(pyridine-2-yl)ethyl}carbamate

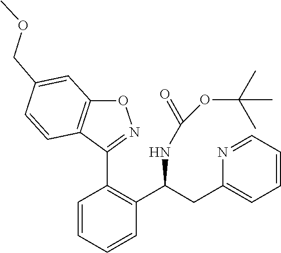 [SMILES: COCc1ccc2c(-c3ccccc3[C@H](Cc3ccccn3)NC(=O)OC(C)(C)C)noc2c1]

A solution of sodium methoxide (0.5M in methanol, 0.38 mL) was added to a solution of tert-butyl (S)-{1-[2-(6-bromomethylbenzo[d]isoxazol-3-yl)phenyl}-2-(pyridine-2-yl)ethyl}carbamate (Intermediate 33A, 0.064 g) in methanol (2 mL) and the mixture was stirred and heated at 55° C. overnight. After cooling, the mixture was concentrated in vacuo and the residue was purified by FCC eluting with 0-50% ethyl acetate in petrol to give the title compound (0.026 g). LCMS (Method 8) RT 2.83 m/z 460 [MH⁺].

### Intermediate 35A: tert-Butyl (S)-{1-[2-(6-[2-hydroxyethyl]benzo[d]isoxazol-3-yl)phenyl}-2-(pyridine-2-yl)ethyl}carbamate

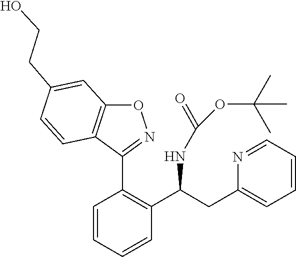 [SMILES: CC(C)(C)OC(=O)N[C@@H](Cc1ccccn1)c1ccccc1-c1noc2cc(CCO)ccc12]

Borane-THF (1M solution in THF, 3.15 mL) was added to a stirred, cooled solution of tert-butyl (S)-{1-[2-(6-vinylbenzo[d]isoxazol-3-yl)phenyl}-2-(pyridine-2-yl)ethyl}carbamate (Intermediate 13A, 0.2 g) in dry THF (6 mL) at 0° C. On completion of the addition, the mixture was allowed to warm to room temperature and stirred for 2 hours. Sodium hydroxide (3M aqueous solution, 2 mL) was added cautiously followed by hydrogen peroxide (30% w/w, 2 mL) and the resultant mixture was stirred at room temperature overnight. A 5% aqueous solution of potassium bisulfite was added and the mixture was extracted with ethyl acetate, washed with water, dried (MgSO₄) and filtered. The filtrate was concentrated in vacuo and the residue was purified by MDAP to give the title compound (0.044 g). LCMS (Method 5) RT 2.54 m/z 460 [MH⁺].

By proceeding in a similar manner to Intermediate 35A, the following compounds were prepared:

### Intermediate 35B: (S)—N—{(S)-1-[2-(Benzo[d]isoxazol-3-yl)phenyl]-2[6-(2-hydroxyethyl)pyridin-2-yl]ethyl}-2-methylpropane-2-sulfinamide

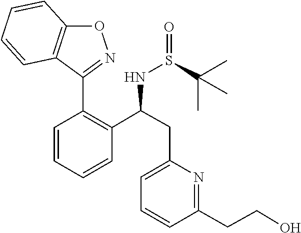 [SMILES: CC(C)(C)[S@](=O)N[C@@H](Cc1cccc(CCO)n1)c1ccccc1-c1noc2ccccc12]

Starting from (S)—N—{(S)-1-[2-(benzo[d]isoxazol-3-yl)phenyl]-2(6-vinylpyridin-2-yl)ethyl}-2-methylpropane-2-sulfinamide (Intermediate 13B). LCMS (Method 8) RT 2.20 m/z 464 [MH⁺].

### Intermediate 35C: (S)—N—{(S)-1-[2-(Benzo[d]isoxazol-3-yl)phenyl]-2-(6-ethylpyridin-2-yl)ethyl}-2-methylpropane-2-sulfinamide

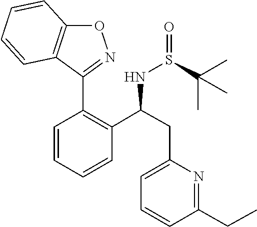 [SMILES: CCc1cccc(C[C@H](N[S@@](=O)C(C)(C)C)c2ccccc2-c2noc3ccccc23)n1]

Isolated as a bi-product from the formation of Intermediate 35B. LCMS (Method 8) RT 2.42 m/z 448 [MH⁺].

### Intermediate 35D: (S)—N—{(S)-1-[2-(Benzo[d]isoxazol-3-yl)phenyl]-2-[3-fluoro-6-(2-hydroxyethyl)-4-(trimethylsilyl)pyridin-2-yl]ethyl}-2-methylpropane-2-sulfinamide

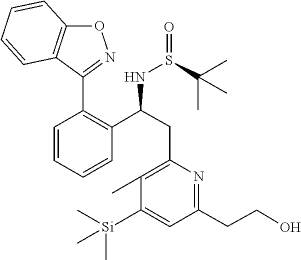 [SMILES: Cc1c([Si](C)(C)C)cc(CCO)nc1C[C@H](N[S@@](=O)C(C)(C)C)c1ccccc1-c1noc2ccccc12]

Starting from (S)—N—{(S)-1-[2-(benzo[d]isoxazol-3-yl)phenyl]-2-[3-fluoro-4-(trimethylsilyl)-6-vinylpyridin-2-yl]ethyl}-2-methylpropane-2-sulfinamide (Intermediate 13C). LCMS (Method 3) RT 1.60 m/z 554 [MH⁺].

### Intermediate 35E: (S)—N—{(S)-1-[2-(Benzo[d]isoxazol-3-yl)phenyl]-2-(6-ethyl-3-methylpyridin-2-yl)ethyl}-2-methylpropane-2-sulfinamide

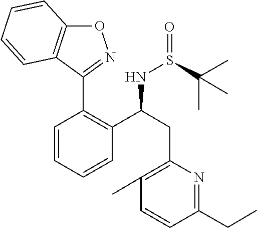 [SMILES: CCc1ccc(C)c(C[C@H](N[S@@](=O)C(C)(C)C)c2ccccc2-c2noc3ccccc23)n1]

Isolated as a by-product from the attempted formation of (S—N—{(S)-1-[2-(benzo[d]isoxazol-3-yl)phenyl]-2-[6-(2-hydroxyethyl)-5-methylpyridin-2-yl]ethyl}-2-methylpropane-2-sulfinamide from (S)—N—{(S)-1-[2-(benzo[d]isoxazol-3-yl)phenyl]-2-(3-methyl-6-vinylpyridin-2-yl)ethyl}-2-methylpropane-2-sulfinamide (Intermediate 13D) using 9-BBN in place of borane-THF. LCMS (Method 3) RT 1.17 m/z 462 [MH⁺].

### Intermediate 36A: Methyl 3-bromo-2-formylbenzoate

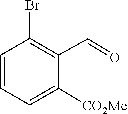 [SMILES: COC(=O)c1cccc(Br)c1C=O]

Dimethyl sulfate (1.16 mL) was added to a mixture of 3-bromo-2-formylbenzoic acid (2.33 g) and potassium carbonate (2.1 g) in acetone (40 mL) and the mixture was stirred for 2 hours. Iodomethane (0.7 mL) was added and the mixture was stirred for 3 days. Further potassium carbonate (2.1 g) and iodomethane (1.4 mL) were added and the mixture was stirred for 5 hours. The mixture was concentrated in vacuo and the residue was partitioned between ethyl acetate and water. The aqueous layer was further extracted with ethyl acetate and the combined organic layers were washed with water, dried (Na₂SO₄) and filtered. The filtrate was concentrated in vacuo and the residue was purified by FCC eluting with 0-75% DCM in cyclohexane to give the title compound (1.34 g) as a white solid which was used directly in the next stage.

### Intermediate 37A: Methyl 3-bromo-2-(hydroxyiminomethyl)benzoate

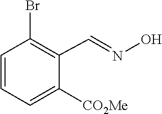 [SMILES: COC(=O)c1cccc(Br)c1/C=N/O]

Hydroxylamine hydrochloride (0.305 g) and pyridine (0.355 mL) were added to a solution of methyl 3-bromo-2-formylbenzoate (Intermediate 36A, 0.09 g) in THF (20 mL). The resultant mixture was stirred at room temperature for 2 days. The mixture was partitioned between ethyl acetate and water and the aqueous layer was extracted with ethyl acetate. The combined organic layers were washed with brine, dried (Na₂SO₄) and filtered. The filtrate was concentrated in vacuo and the residue was purified by FCC eluting with 0-50% ethyl acetate in cyclohexane to give the title compound (0.53 g). LCMS (Method 5) RT 3.26 m/z 258/260.

### Intermediate 38A: (3-Bromo-2-fluorophenyl)[2-(diethoxymethyl)phenyl]methanol

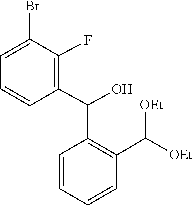 [SMILES: CCOC(OCC)c1ccccc1C(O)c1cccc(Br)c1F]

n-Butyllithium (1.6M solution in hexanes, 6.25 mL) was added dropwise to a cooled solution of 1-bromo-2-(diethoxymethyl)benzene (2.0 mL) in dry ether (20 mL) while maintaining the temperature below −60° C. The mixture was stirred for a further 1 hour at −78° C. then 3-bromo-2-fluorobenzaldehyde (2.03 g) in dry ether (8 mL) was added. The mixture was allowed to warm to room temperature and stirred overnight. Saturated aqueous ammonium chloride was added and the layers were separated. The aqueous layer was further extracted with ether and the combined organic layers were dried (MgSO₄) and filtered. The filtrate was concentrated in vacuo and the residue was purified by FCC eluting with 0-10% ethyl acetate in cyclohexane to give the title compound (2.93 g) as a yellow oil which was used directly in the next stage.

By proceeding in a similar manner to Intermediate 38A, the following compounds were prepared:

### Intermediate 38B: [3-Bromo-2-(1,3-dioxolan-2-yl)phenyl](2-fluorophenyl)methanol

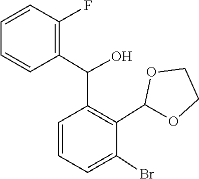 [SMILES: OC(c1ccccc1F)c1cccc(Br)c1C1OCCO1]

Starting from 2-(2,6-dibromophenyl)-1,3-dioxolane (Intermediate 28B) and 2-fluorobenzaldehyde. ¹H NMR (400 MHz, CDCl₃) 7.73-7.65 (1H, m), 7.52 (1H, dd, J=7.9, 1.4 Hz), 7.35-7.26 (1H, m), 7.22 (1H, dt, J=1.5, 7.6 Hz), 7.12 (1H, t, J=7.6 Hz), 7.05-6.96 (2H, m), 6.70 (1H, d, J=2.7 Hz), 6.42 (1H, s), 4.32-4.26 (2H, m), 4.13-4.07 (2H, m), 3.73 (1H, d, J=2.8 Hz).

### Intermediate 38C: (2-Fluoro-4-trifluoromethylphenyl)[2-(diethoxymethyl)phenyl]-methanol

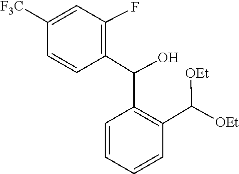 [SMILES: CCOC(OCC)c1ccccc1C(O)c1ccc(C(F)(F)F)cc1F]

Starting from 2-fluoro-4-trifluoromethylbenzaldehyde and 1-bromo-2-(diethoxymethyl)benzene and used directly in the next step.

### Intermediate 38D: (2,4-Difluorophenyl)[2-(diethoxymethyl)phenyl]methanol

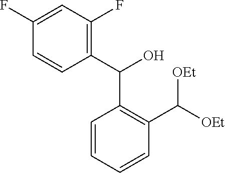 [SMILES: CCOC(OCC)c1ccccc1C(O)c1ccc(F)cc1F]

Starting from 2,4-difluorobenzaldehyde and 1-bromo-2-(diethoxymethyl)benzene.

¹H NMR (400 MHz, CDCl₃) 7.73-7.65 (1H, m), 7.52-7.48 (1H, m), 7.32-7.22 (2H, m), 7.02-6.93 (2H, m), 6.81-6.73 (1H, m), 6.61 (1H, d, J=2.9 Hz) 5.61 (1H, s), 3.92 (1H, d, J=3.2 Hz), 3.87-3.79 (1H, m), 3.66-3.47 (3H, m), 1.31-1.19 (6H, m)

### Intermediate 38E: [2-(Diethoxymethyl)phenyl](2,5-difluorophenyl)methanol

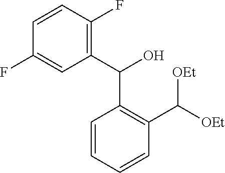 [SMILES: CCOC(OCC)c1ccccc1C(O)c1cc(F)ccc1F]

Starting from 2,5-difluorobenzaldehyde and 1-bromo-2-(diethoxymethyl)benzene. ¹H NMR (400 MHz, CDCl₃) 7.52-7.43 (2H, m), 7.34-7.21 (2H, m), 7.03-6.90 (3H, m), 6.62 (1H, d, J=3.1 Hz), 5.62 (1H, s), 3.96 (1H, d, J=3.2 Hz), 3.92-3.80 (1H, m), 3.70-3.45 (3H, m), 1.28 (3H, t, J=7.1 Hz), 1.23 (3H, t, J=7.1 Hz).

### Intermediate 38F: [2-(Diethoxymethyl)phenyl](2,3-difluorophenyl)methanol

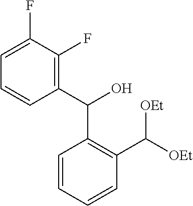 [SMILES: CCOC(OCC)c1ccccc1C(O)c1cccc(F)c1F]

Starting from 2,3-difluorobenzaldehyde and 1-bromo-2-(diethoxymethyl)benzene. ¹H NMR (400 MHz, CDCl₃) 7.52-7.47 (2H, m), 7.32-7.22 (2H, m), 7.20-7.08 (2H, m), 7.02-6.98 (1H, m), 6.68 (1H, d, J=3.1 Hz), 5.61 (1H, s), 4.00-3.97 (1H, m), 3.89-3.80 (1H, m), 3.67-3.57 (2H, m), 3.55-3.48 (1H, m), 1.28 (3H, t, J=7.1 Hz), 1.23 (3H, t, J=7.1 Hz).

### Intermediate 39A: (3-Bromo-2-fluorophenyl)[2-(3-diethoxymethyl)phenyl]methanone

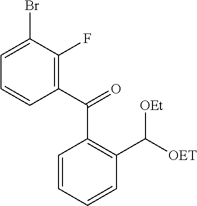

1,3-Dibromo-4,4-dimethylhydantoin (0.5 g) was added to a solution of (3-bromo-2-fluorophenyl)[2-(diethoxymethyl)phenyl]methanol (Intermediate 38A, 1.19 g) in tert-butanol (7.5 mL) and water (15 mL). This was followed by the addition of 2,2,6,6-tetramethylpiperidin-1-yl)oxy (TEMPO, 0.006 g) and sodium bicarbonate (0.564 g) and the resultant mixture was stirred at room temperature for 24 hours. Saturated aqueous sodium bicarbonate was added followed by a solution of sodium thiosulfate (0.1 g) in water (10 mL). The mixture was stirred for 10 minutes then extracted with ethyl acetate, dried (MgSO₄) and filtered. The filtrate was concentrated in vacuo and the residue was purified by FCC eluting with 0-20% ethyl acetate in cyclohexane to give the title compound as a colourless oil. LCMS (Method 5) RT 4.68 m/z 403/405 [M+Na⁺].

By proceeding in a similar manner to Intermediate 39A, the following compounds were prepared:

### Intermediate 39B: [3-Bromo-2-(1,3-dioxolan-2-yl)phenyl](2-fluorophenyl)methanone

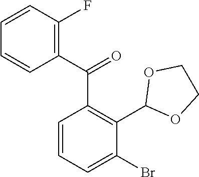 [SMILES: O=C(c1ccccc1F)c1cccc(Br)c1C1OCCO1]

Starting from: [3-bromo-2-(1,3-dioxolan-2-yl)phenyl](2-fluorophenyl)methanol (Intermediate 38B). LCMS (Method 5) RT 3.96 m/z 351/353 [MH⁺].

### Intermediate 39C: (2-Fluoro-4-trifluoromethylphenyl)[2-(3-diethoxymethyl)phenyl]methanone

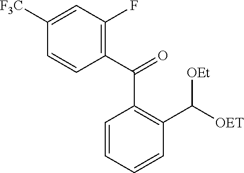

Starting from (2-fluoro-4-trifluoromethylphenyl)[2-(diethoxymethyl)phenyl]-methanol (Intermediate 38C). ¹H NMR (400 MHz, CDCl₃) 7.80 (1H, t, J=7.5 Hz), 7.73 (1H, d, J=7.7 Hz), 7.55-7.47 (2H, m), 7.42-7.36 (2H, m), 7.32-7.28 (1H, m), 5.76 (1H, s), 3.64-3.55 (2H, m), 3.51-3.41 (2H, m), 1.11 (6H, t, J=7.1 Hz)

### Intermediate 39D: [2-(Diethoxymethyl)phenyl](2,4-difluorophenyl)methanone

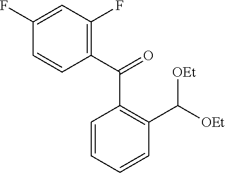 [SMILES: CCOC(OCC)c1ccccc1C(=O)c1ccc(F)cc1F]

Starting from [2-(diethoxymethyl)phenyl](2,4-difluorophenyl)methanol (Intermediate 38D). ¹H NMR (400 MHz, CDCl₃) 7.77-7.69 (2H, m), 7.49 (1H, dt, J=1.3, 7.5 Hz), 7.37 (1H, dt, J=1.3, 7.5 Hz), 7.30-7.26 (1H, m), 6.97-6.91 (1H, m), 6.87-6.81 (1H, m), 5.74 (1H, s), 3.64-3.55 (2H, m), 3.50-3.41 (2H, m), 1.10 (6H, t, J=7.1 Hz).

### Intermediate 39E: [2-(Diethoxymethyl)phenyl](2,5-difluorophenyl)methanone

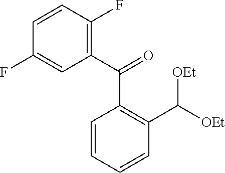 [SMILES: CCOC(OCC)c1ccccc1C(=O)c1cc(F)ccc1F]

Starting from [2-(diethoxymethyl)phenyl](2,5-difluorophenyl)methanol (Intermediate 38E). ¹H NMR (400 MHz, CDCl₃) 7.74-7.69 (1H, m), 7.51 (1H, dt, J=1.4, 7.5 Hz), 7.43-7.34 (2H, m), 7.33-7.28 (1H, m), 7.25-7.17 (1H, m), 7.12-7.03 (1H, m), 5.76 (1H, s), 3.65-3.53 (2H, m), 3.52-3.40 (2H, m), 1.11 (6H, t, J=7.1 Hz).

### Intermediate 39F: [2-(Diethoxymethyl)phenyl](2,3-difluorophenyl)methanone

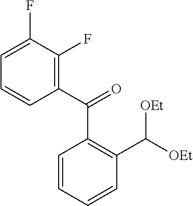 [SMILES: CCOC(OCC)c1ccccc1C(=O)c1cccc(F)c1F]

Starting from [2-(diethoxymethyl)phenyl](2,3-difluorophenyl)methanol (Intermediate 38F). ¹H NMR (400 MHz, CDCl₃) 7.75-7.71 (1H, m), 7.51 (1H, dt, J=1.5, 7.6 Hz), 7.45-7.29 (4H, m), 7.18-7.11 (1H, m), 5.77 (1H, s), 3.65-3.55 (2H, m), 3.51-3.42 (2H, m), 1.11 (6H, t, J=7.1 Hz).

### Intermediate 40A: tert-Butyl (S)-{1-[2-(benzo[d]isoxazol-3-yl)phenyl]-2-(6-methoxypyridine-2-yl)ethyl}carbamate

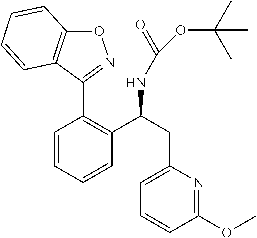 [SMILES: COc1cccc(C[C@H](NC(=O)OC(C)(C)C)c2ccccc2-c2noc3ccccc23)n1]

Sodium hydride (60% oil dispersion, 0.392 g) was added carefully to a solution of methanol (0.314 g) in DMF (3 mL). The resultant mixture was stirred for 20 minutes then a solution of tert-butyl (S)-{1-[2-(benzo[d]isoxazol-3-yl)phenyl]-2-(6-bromopyridine-2-yl)ethyl}carbamate (Intermediate 9D, 0.157 g) in DMF (1 mL) was added. The mixture was stirred for 2 hours then water was added and the mixture was extracted with ethyl acetate, washed with brine then filtered through a phase separator. The filtrate was concentrated in vacuo and the residue was purified by FCC eluting with 0-20% ethyl acetate in cyclohexane to give the title compound (0.042 g) as a yellow gum. LCMS (Method 5) RT 4.66 m/z 446 [MH⁺].

### Intermediate 41A: (S)—N—{(S)-1-[2-(Benzo[d]isoxazol-3-yl)phenyl]-2-(3-methylpyridin-2-yl)ethyl}-2-methylpropane-2-sulfinamide

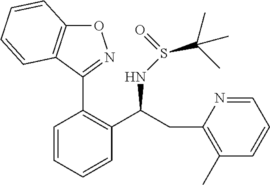 [SMILES: Cc1cccnc1C[C@H](N[S@@](=O)C(C)(C)C)c1ccccc1-c1noc2ccccc12]

Boron trifluoride etherate (0.285 mL) was added to a stirred, cooled solution of 2,3-dimethylpyridine (0.26 mL) in dry THF (3 mL) over 15 minutes while maintaining the temperature at 0° C. On completion of the addition, the mixture was cooled to −78° C. and n-butyllithium (1.6M in hexanes, 1.4 mL) was added at such a rate as to maintain the temperature below −70° C. The mixture was stirred at −78° C. for 45 minutes then a solution of (S)—N-[2-(benzo[d]isoxazol-yl)benzylidene]-2-methylpropane-2-sulfinamide (Intermediate 4B, 0.25 g) in THF (1.5 mL) was added while maintaining the temperature below −70° C. The mixture was stirred at −78° C. for 1 hour, then allowed to warm to room temperature and stirred overnight. Water was added and the mixture was extracted with DCM then filtered through a phase separator and the filtrate was concentrated in vacuo. The residue was purified by FCC eluting with 0-100% ethyl acetate in pentane, followed by 0-3.5% methanol in ethyl acetate to give the title compound (0.111 g) as a yellow gum. LCMS (Method 6) RT 2.48 m/z 434 [MH⁺].

### Intermediate 42A: (S)—N-{(1S)-1-[2-(Benzo[d]isoxazol-3-yl)phenyl]-3-methyl-2-(pyridin-2-yl)butyl}-2-methylpropane-2-sulfinamide

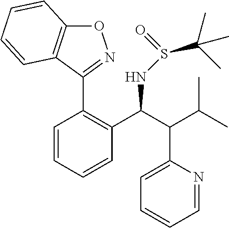 [SMILES: CC(C)C(c1ccccn1)[C@H](N[S@@](=O)C(C)(C)C)c1ccccc1-c1noc2ccccc12]

2-(2-Methylprop-1-yl)pyridine (0.384 mL) was added to potassium tert-butoxide (1M solution in THF, 2.3 mL) at −50° C. followed by the addition of a n-butyllithium (1.6M in hexanes, 1.44 mL). The resultant mixture was stirred at −50° C. for 2 hours then a solution of (S)—N-[2-(benzo[d]isoxazol-yl)benzylidene]-2-methylpropane-2-sulfinamide (Intermediate 4B, 0.25 g) in THF (1.5 mL) was added. The mixture was stirred at −50° C. for 30 minutes then allowed to warm to room temperature and stirred overnight. The mixture was re-cooled to −50° C. and a further amount of anion generated from 2-(2-methylprop-1-yl)pyridine (0.384 mL), potassium tert-butoxide (1M solution in THF, 2.3 mL) and n-butyllithium (1.6M in hexanes, 1.44 mL) was added and the mixture was stirred at −50° C. for 30 minutes and at room temperature for 2 hours. Water was added and the mixture was extracted with DCM, then filtered through a phase separator. The filtrate was concentrated in vacuo and the residue was purified by FCC eluting with 0-100% ethyl acetate in pentane to give the title compound (0.058 g) as a yellow gum. LCMS (Method 6) RT3.10 m/z 462 [MH⁺].

### Intermediate 43A: (S)—N—{(R)-1-[2-(benzo[d]isoxazol-3-yl)phenyl]-2-methyl-2-(pyridin-2-yl)propyl}-2-methylpropane-2-sulfinamide

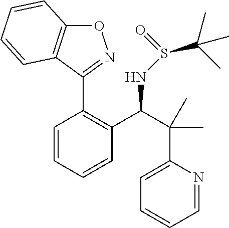 [SMILES: CC(C)(c1ccccn1)[C@H](N[S@@](=O)C(C)(C)C)c1ccccc1-c1noc2ccccc12]

n-Butyllithium (1.6M in hexanes, 0.96 mL) was added dropwise to a stirred, cooled mixture of potassium tert-butoxide (1M in THF, 1.5 mL) and di-isopropylamine (0.22 mL) while maintaining the temperature below −40° C. The temperature was raised to −25° C. and the mixture was stirred for 30 minutes then re-cooled to −50° C. A solution of 2-isopropylpyridine (0.13 mL) in THF (0.25 mL) was added and the mixture was stirred at −50° C. for 2 hours. A solution of (S)—N-[2-(benzo[d]isoxazol-yl)benzylidene]-2-methylpropane-2-sulfinamide (Intermediate 4B, 0.25 g) in THF (1.5 mL) was then added and the resultant mixture was stirred at −50° C. for 30 minutes then allowed to warm to room temperature and stirred overnight. It was diluted with saturated aqueous sodium bicarbonate solution and extracted with DCM. The organic phase was filtered through a phase separator and the filtrate was concentrated in vacuo. The residue was purified by FCC eluting with 0-100% ethyl acetate in hexane to give the title compound (0.4 g) as a yellow gum. LCMS (Method 6) RT 3.46 m/z 448 [MH⁺].

### Intermediate 44A: Methyl (S)-6-{2-[2-(benzo[d]isoxazol-3-yl)phenyl]-2-[(tert-butoxycarbonyl)amino]ethyl}pyridin-2-carboxylate

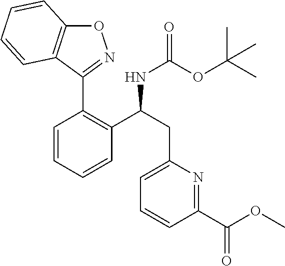 [SMILES: COC(=O)c1cccc(C[C@H](NC(=O)OC(C)(C)C)c2ccccc2-c2noc3ccccc23)n1]

A solution of tert-butyl (S)-{1-[2-(benzo[d]isoxazol-3-yl)phenyl]-2-(6-bromopyridine-2-yl)ethyl}carbamate (Intermediate 9D, 0.19 g) in triethylamine (4 mL) and methanol (0.3 g) was degassed and treated with palladium (II) acetate (0.003 g) and xantphos (0.013 g). The resultant mixture was again degassed then flushed with carbon monoxide gas. The resultant mixture was stirred and heated at 66° C. under an atmosphere of carbon monoxide overnight. After cooling, the mixture was concentrated in vacuo and the residue was partitioned between water and ethyl acetate. The aqueous phase was further extracted with ethyl acetate and the combined organic phases were dried by filtering through a phase separator and the filtrate was concentrated in vacuo. The residue was purified by FCC eluting with 0-50% ethyl acetate in cyclohexane. The main product was repurified by FCC eluting with 0-25% ethyl acetate in cyclohexane to give the title compound (0.063 g) as a white solid. LCMS (Method 5) RT 4.25 m/z 474 [MH⁺].

### Intermediate 45A: tert-Butyl (S)-{1-[2-(benzo[d]isoxazol-3-yl)phenyl]-2-[6-(dimethylcarbamoyl)pyridine-2-yl]ethyl}carbamate

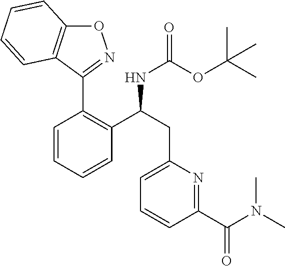 [SMILES: CN(C)C(=O)c1cccc(C[C@H](NC(=O)OC(C)(C)C)c2ccccc2-c2noc3ccccc23)n1]

A mixture of methyl (S)-6-{2-[2-(benzo[d]isoxazol-3-yl)phenyl]-2-[(tert-butoxycarbonyl)amino]ethyl}pyridin-2-carboxylate (Intermediate 44A, 0.063 g) and N,N-dimethylamine (2M in THF, 1 mL) was stirred and heated in a sealed vial at 100° C. overnight then heated at 120° C. for 48 hours. After cooling to room temperature, the mixture was concentrated in vacuo and the residue was purified by FCC eluting with 0-100% ethyl acetate in cyclohexane to give the title compound (0.013 g) as an off-white solid. LCMS (Method 5) RT 3.96 m/z 487 [MH⁺].

### Intermediate 46A: tert-Butyl (S)-{1-[2-(benzo[d]isoxazol-3-yl)phenyl]-2-[6-(1-methyl-1H-pyrazol-4-yl)pyridine-2-yl]ethyl}carbamate

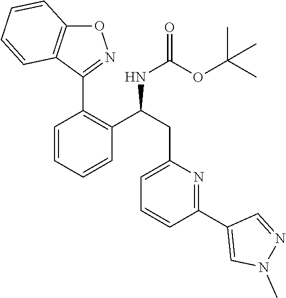 [SMILES: Cn1cc(-c2cccc(C[C@H](NC(=O)OC(C)(C)C)c3ccccc3-c3noc4ccccc34)n2)cn1]

Tetrakis(triphenylphosphine)palladium (0.023 g) was added to a degassed solution of tert-butyl (S)-{1-[2-(benzo[d]isoxazol-3-yl)phenyl]-2-(6-bromopyridine-2-yl)ethyl}carbamate (Intermediate 9D, 0.1 g), 1-methyl-1H-pyrazole-4-boronic acid (0.028 g) and sodium carbonate (0.043 g) in dioxane (2 mL) and water (0.92 mL) and the mixture was stirred and heated in a sealed vial at 90° C. overnight and then at 110° C. for 24 hours. Further 1-methyl-1H-pyrazole-4-boronic acid (0.064 g) and tetrakis(triphenylphosphine)palladium (0.028 g) were added and after degassing, the mixture was stirred and heated in a sealed vial at 110° C. overnight. After cooling, the mixture was treated with brine solution and extracted with ethyl acetate. The organic phase was filtered through a phase separator and the filtrate was concentrated in vacuo. The residue was purified by FCC eluting with 0-100% ethyl acetate in cyclohexane to give the title compound (0.022 g) as a colourless gum which was used directly in the next stage.

By proceeding in a similar manner to Intermediate 46A, the following compounds were prepared:

### Intermediate 46B: (S)—N—{(S)-1-[2-(Benzo[d]isoxazol-3-yl)phenyl]-2-[3-fluoro-6-(1H-pyrazol-4-yl)-4-(trimethylsilyl)pyridine-2-yl]ethyl}-2-methylpropane-2-sulfinamide

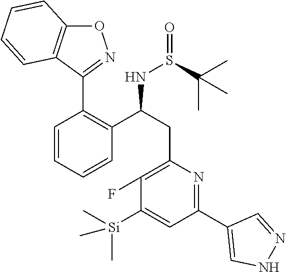 [SMILES: CC(C)(C)[S@](=O)N[C@@H](Cc1nc(-c2cn[nH]c2)cc([Si](C)(C)C)c1F)c1ccccc1-c1noc2ccccc12]

Starting from (S)—N—{(S)-1-[2-(benz[d]isoxazol-3-yl)phenyl-2-[6-bromo-3-fluoro-4(trimethylsilyl)pyridine-2-yl]ethyl}-2-methylpropane-2-sulfinamide (Intermediate 6S) and 4-(4,4,5,5-tetramethyl-1,3,2-dioxaborolan-2-yl)-1-H-pyrazole using potassium carbonate in place of sodium carbonate and heating at 130° C. for 2 hours. LCMS (Method 3) RT 1.60 m/z 576 [MH⁺] and RT 1.35 m/z 504 {MH⁺] for loss of the silyl group

### Intermediate 47A: (S)—N—{(S)-2-[6-(1H-Pyrazol-4-yl)pyridine-2-yl]-1-[2-(benzo[d]isoxazol-3-yl)phenyl]ethyl}-2-methylpropane-2-sulfinamide

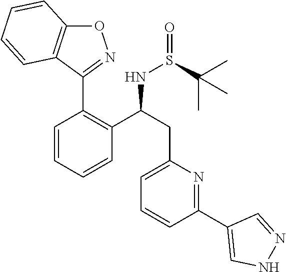 [SMILES: CC(C)(C)[S@](=O)N[C@@H](Cc1cccc(-c2cn[nH]c2)n1)c1ccccc1-c1noc2ccccc12]

A mixture of (S)—N—{(S)-2-[6-bromopyridine-2-yl]-1-[2-(benzo[d]isoxazol-3-yl)phenyl]ethyl}propane-2-sulfinamide (Intermediate 6B, 0.27 g), 4-(4,4,5,5-tetramethyl-1,3,2-dioxaborolan-2-yl)-1-H-pyrazole (0.126 g), tetrakis(triphenylphosphine)palladium (0.05 g) and potassium carbonate (0.22 g) in dimethoxyethane (12 mL) and water (0.5 mL) was degassed and then heated in the microwave at 110° C. for 45 minutes and then at 140° C. for a total of 1 hour. After cooling, the mixture was partitioned between ethyl acetate and water and the organic phase was dried (MgSO₄) and filtered. The filtrate was concentrated in vacuo and the residue was purified by FCC eluting with 0-10% methanol in ethyl acetate to give the title compound (0.079 g) as a white solid. LCMS (Method 6) RT 2.72 m/z 486 [MH⁺].

### Intermediate 48A: tert-Butyl (S)-{1-[2-(benzo[d]isoxazol-3-yl)phenyl]-2-[6-(oxazol-2-yl)pyridine-2-yl]ethyl}carbamate

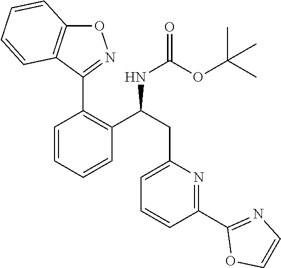 [SMILES: CC(C)(C)OC(=O)N[C@@H](Cc1cccc(-c2ncco2)n1)c1ccccc1-c1noc2ccccc12]

A mixture of tert-butyl (S)-{1-[2-(benzo[d]isoxazol-3-yl)phenyl]-2-(6-bromopyridine-2-yl)ethyl}carbamate (Intermediate 9D, 0.1 g) and 2-(tributylstannyl)oxazole (0.08 g) in dioxane (2 mL) was degassed and the mixture was then treated with tetrakis(triphenylphosphine)palladium (0.023 g). The mixture was then degassed again and then heated at 90° C. in a sealed tube overnight, then at 100° C. overnight. Further 2-(tributylstannyl)oxazole (0.08 g) and tetrakis(triphenylphosphine)palladium (0.028 g) were added and the mixture was stirred and heated in a sealed vial at 110° C. overnight. After cooling, the mixture was treated with brine solution and extracted with ethyl acetate. The organic phase was filtered through a phase separator and the filtrate was concentrated in vacuo to give the title compound (0.029 g) which was used directly in the next step.

### Intermediate 49A: tert-Butyl (S)-{1-[2-(benzo[d]isoxazol-3-yl)phenyl]-2-[6-(pyrrolidin-1-yl)pyridine-2-yl]ethyl}carbamate

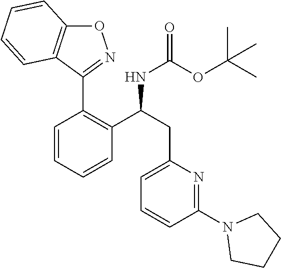 [SMILES: CC(C)(C)OC(=O)N[C@@H](Cc1cccc(N2CCCC2)n1)c1ccccc1-c1noc2ccccc12]

A solution of tert-butyl (S)-{1-[2-(benzo[d]isoxazol-3-yl)phenyl]-2-(6-bromopyridine-2-yl)ethyl}carbamate (Intermediate 9D, 0.05 g) and pyrrolidine (0.072 g) in 1-butanol (1 mL) was stirred and heated in a sealed vial at 90° C. for 48 hours. After cooling, the mixture was diluted with ethyl acetate and washed with water and the organic phase was filtered through a phase separator and the filtrate was concentrated in vacuo. The reaction was repeated using the same quantities and the combined residues were purified by MDAP (acidic) to give the title compound (0.049 g). LCMS (Method 5) RT 3.04 m/z 485 [MH⁺].

### Intermediate 50A: (S)—N—{(S)-1-[2-(Benzo[d]isoxazol-3-yl)phenyl]-2-[6-(3,5-dimethylisoxazol-4-yl)pyridine-2-yl]ethyl}-2-methylpropane-2-sulfinamide

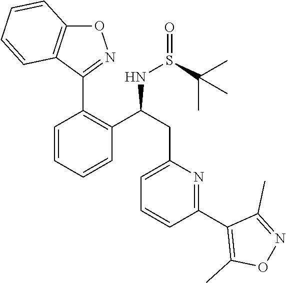 [SMILES: Cc1noc(C)c1-c1cccc(C[C@H](N[S@@](=O)C(C)(C)C)c2ccccc2-c2noc3ccccc23)n1]

A mixture of (S)—N—{(S)-1-[2-(benzo[d]isoxazol-3-yl)phenyl]-2-[6-bromopyridine-2-yl]ethyl}-2-methylpropane-2-sulfinamide (Intermediate 6B, 0.15 g), 3,5-dimethylisoxazol-4-ylboronic acid (0.051 g), tetrakis(triphenylphosphine)palladium (0.035 g) and cesium carbonate (0.293 g) in dioxane (1 mL) and water (0.5 mL) was degassed and then heated in the microwave at 140° C. for a total of 2 hours. After cooling, the mixture was partitioned between ethyl acetate and water. The organic phase was dried (MgSO₄) and filtered and the filtrate was concentrated in vacuo. The residue was purified by FCC eluting with 0-60% ethyl acetate in DCM to give the title compound (0.06 g) which was used directly in the next step.

### Intermediate 51A: (S)—N—{(S)-1-[2-(6-Bromobenzo[d]isoxazol-3-yl)phenyl]-2-(6-methylaminopyridine-2-yl)ethyl}-2-methylpropane-2-sulfinamide

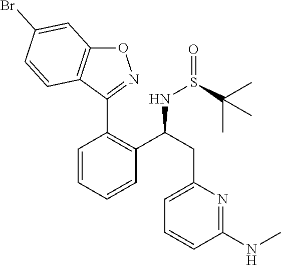 [SMILES: CNc1cccc(C[C@H](N[S@@](=O)C(C)(C)C)c2ccccc2-c2noc3cc(Br)ccc23)n1]

A mixture of (S)—N—{(S)-1-[2-(6-bromobenzo[d]isoxazol-3-yl)phenyl]-2-(6-fluoropyridine-2-yl)ethyl}-2-methylpropane-2-sulfinamide (Intermediate 6F, 0.169 g) and methylamine (2M in THF, 3 mL) was stirred and heated in a sealed vial at 90° C. for 48 hours. After cooling, the mixture was concentrated in vacuo and the residue was purified by FCC eluting with a 0-10% methanol in ethyl acetate to give the title compound (0.05 g) as an off white solid. LCMS (Method 6) RT 2.75 m/z 527/529 [MH⁺].

### Intermediate 52A: 2-Methyl-6-{2-[(tetrahydro-2H-pyran-2-yl)oxy]ethoxy}pyridine

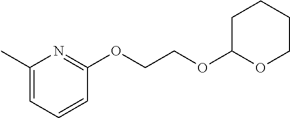 [SMILES: Cc1cccc(OCCOC2CCCCO2)n1]

Sodium hydride (60% dispersion in oil, 0.96 g) was added to a cooled solution of 2-[(tetrahydro-2H-pyran-2-yl)oxy]ethan-1-ol (3.49 g) in DMF (35 mL) at 0-5° C. The resultant mixture was stirred at 0° C. for 10 minutes then a solution of 2-fluoro-6-methylpyridine (2.55 g) in DMF (15 mL) was added. The mixture was stirred at 0° C. for 15 minutes then at room temperature for 1 hour. The mixture was concentrated in vacuo and the residue was partitioned between DCM and water. The organic phase was dried (MgSO₄ and filtered and the filtrate was concentrated in vacuo. The residue was purified by FCC eluting with 0-100% ethyl acetate in cyclohexane to give the title compound (4.27 g) as a colourless oil. LCMS (Method 6) RT 2.83 m/z 260 [M+Na⁺].

### Intermediate 53A: (S)—N—{(S)-1-[2-(Benzo[d]isoxazol-3-yl)phenyl]-2-[6-((R)-3-hydroxylpyrrolidin-1-yl)pyridine-2-yl]ethyl}-2-methylpropane-2-sulfinamide

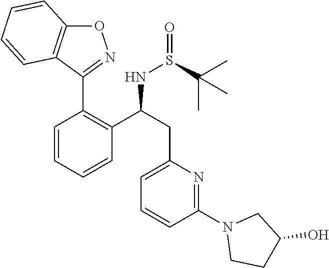 [SMILES: CC(C)(C)[S@](=O)N[C@@H](Cc1cccc(N2CC[C@@H](O)C2)n1)c1ccccc1-c1noc2ccccc12]

A mixture of (S)—N—{(S)-1-[2-(benzo[d]isoxazol-3-yl)phenyl]-2-(6-bromopyridine-2-yl)ethyl}-2-methylpropane-2-sulfinamide (Intermediate 6B, 0.15 g), (R)-3-hydroxypyrrolidine (0.05 mL), RuPhos pre-catalyst (0.049 g), RuPhos (0.028 g) and potassium phosphate tribasic (0.076 g) in tert-butanol (2.5 mL) was sealed in a vial and degassed, then heated at 100° C. overnight. After cooling, the mixture was diluted with water and extracted with ethyl acetate. The organic phase was washed with water, dried (Na₂SO₄) and filtered. The filtrate was concentrated in vacuo and the residue was purified by FCC eluting with 0-4% 2M ammonia in methanol in DCM to give the title compound (0.073 g) as an off white solid. LCMS (Method 6) RT 2.39 m/z 505 [MH⁺].

By proceeding in a similar manner to Intermediate 53A, the following compounds were prepared:

### Intermediate 53B: (S)—N—{(S)-1-[2-(Benzo[d]isoxazol-3-yl)phenyl]-2-[6-[2-methoxyethyl]methylamino)pyridine-2-yl]ethyl}-2-methylpropane-2-sulfinamide

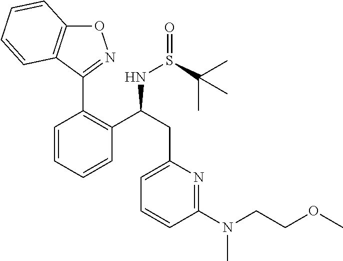 [SMILES: COCCN(C)c1cccc(C[C@H](N[S@@](=O)C(C)(C)C)c2ccccc2-c2noc3ccccc23)n1]

Staring from (S)—N—{(S)-1-[2-(benzo[d]isoxazol-3-yl)phenyl]-2-(6-bromopyridine-2-yl)ethyl}-2-methylpropane-2-sulfinamide (Intermediate 6B) and 2-methoxy-N-methylethan-1-amine and used without further characterisation.

### Intermediate 53C: (S)—N—{(S)-1-[2-(Benzo[d]isoxazol-3-yl)phenyl]-2-[6-(2-methoxyethyl amino)pyridine-2-yl]ethyl}-2-methylpropane-2-sulfinamide

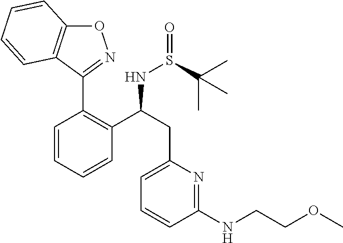 [SMILES: COCCNc1cccc(C[C@H](N[S@@](=O)C(C)(C)C)c2ccccc2-c2noc3ccccc23)n1]

Starting from (S)—N—{(S)-1-[2-(benzo[d]isoxazol-3-yl)phenyl]-2-(6-bromopyridine-2-yl)ethyl}-2-methylpropane-2-sulfinamide (Intermediate 6B) and 2-methoxyethan-1-amine and using BrettPhos and BrettPhos pre catalyst in place of RuPhos and RuPhos pre catalyst. LCMS (Method 6) RT 2.52 m/z 493 [MH⁺].

### Intermediate 53D: (S)—N—{(S)-1-[2-(Benzo[d]isoxazol-3-yl)phenyl]-2-[6-((S)-3-hydroxylpyrrolidin-1-yl)pyridine-2-yl]ethyl}-2-methylpropane-2-sulfinamide

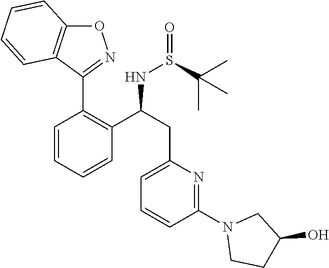 [SMILES: CC(C)(C)[S@](=O)N[C@@H](Cc1cccc(N2CC[C@H](O)C2)n1)c1ccccc1-c1noc2ccccc12]

Starting from (S)—N—{(S)-1-[2-(benzo[d]isoxazol-3-yl)phenyl]-2-(6-bromopyridine-2-yl)ethyl}-2-methylpropane-2-sulfinamide (Intermediate 6B) and (S)-3-hydroxypyrrolidine. LCMS (Method 6) RT 2.36 m/z 505 [MH⁺].

### Intermediate 53E: tert-Butyl (S)-(1-[2-(benz[d]isoxazol-3-yl)phenyl]-2-{6-[(2-hydroxyethyl)(methyl)amino]pyridin-2-yl}ethyl)carbamate

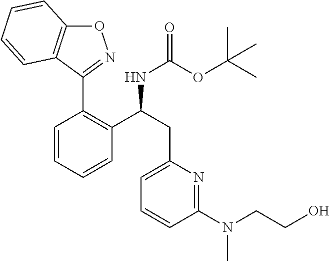 [SMILES: CN(CCO)c1cccc(C[C@H](NC(=O)OC(C)(C)C)c2ccccc2-c2noc3ccccc23)n1]

Starting from tert-butyl (S)-{1-[2-(6-bromobenzo[d]isoxazol-3-yl)phenyl]-2-(pyridine-2-yl)ethyl}carbamate (Intermediate 9D) and N-(2-hydroxyethyl)methylamine. LCMS (Method 6) RT 2.62 m/z 489 [MH⁺].

### Intermediate 54A: tert-Butyl (S)-{1-[2-(benz[d]isoxazol-3-yl)phenyl]-2-(6-vinylpyridin-2-yli)ethyl}carbamate

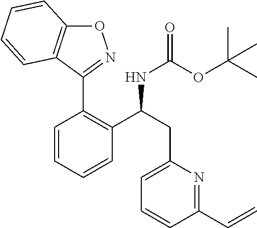 [SMILES: C=Cc1cccc(C[C@H](NC(=O)OC(C)(C)C)c2ccccc2-c2noc3ccccc23)n1]

A solution of di-tert-butyl carbonate (0.25 g) in methanol (2 mL) was added to a stirred mixture of (S)-1-[2-(benzo[d]isoxazol-3-yl)phenyl]-2(6-vinylpyridin-2-yl)ehtan-1-amine (Example 59, 0.332 g) and sodium carbonate (0.15 g) in methanol (5 mL). The mixture was stirred for 15 minutes then partitioned between DCM and water. The organic phase was dried (Na₂SO₄) and filtered and the residue was purified by FCC eluted with 0-50% ethyl acetate in cyclohexane to give the title compound (0.35 g) as a white solid. LCMS (Method 6) RT 3.63 m/z 442 [MH⁺].

### Intermediate 55A: tert-Butyl {(1S)-1-[2-(benz[d]isoxazol-3-yl)phenyl]-2-[6-(1,2-dihydroxyethyl)pyridin-2-yl]ethyl}carbamate

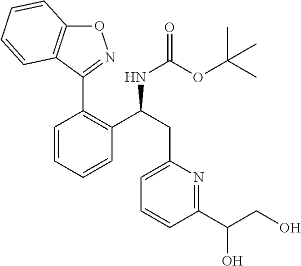 [SMILES: CC(C)(C)OC(=O)N[C@@H](Cc1cccc(C(O)CO)n1)c1ccccc1-c1noc2ccccc12]

Osmium tetraoxide (2.5% w/v in tert-butanol, 0.11 mL) was added to a stirred mixture of tert-butyl (S)-{1-[2-(benz[d]isoxazol-3-yl)phenyl]-2-(6-vinylpyridin-2-yl)ethyl}carbamate (Intermediate 54A, 0.338 g) and N-methylmorpholine N-oxide (0.31 g) in acetone (3 mL) and water (0.75 mL) and the resultant mixture was stirred for 1 hour. The mixture was partitioned between DCM and water and the organic phase was dried (Na₂SO₄) and filtered. The filtrate was concentrated in vacuo and the residue was purified by FCC eluting with 0-100% ethyl acetate in cyclohexane to give the title compound (0.364 g) as a white solid. LCMS (Method 6) RT 2.84 m/z 476 [MH⁺].

### Intermediate 56A: (S)—N—{(S)-1-[2-(Benzo[d]isoxazol-3-yl)phenyl]-2-[6-(methylsulfonyl)pyridine-2-yl]ethyl}propane-2-sulfinamide

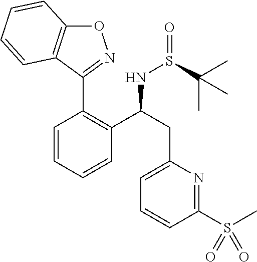 [SMILES: CC(C)(C)[S@](=O)N[C@@H](Cc1cccc(S(C)(=O)=O)n1)c1ccccc1-c1noc2ccccc12]

A mixture of (S)—N—{(S)-1-[2-(benzo[d]isoxazol-3-yl)phenyl]-2-(6-bromopyridine-2-yl)ethyl}propane-2-sulfinamide (Intermediate 6B, 0.163 g), sodium methanesulfinate (0.1 g) and copper (I) iodide (0.187 g) in DMF (4 mL) was stirred and heated in a sealed vial at 80° C. for 1 hour then at 90° C. overnight. After cooling, the mixture was concentrated in vacuo and the residue was partitioned between DCM and water. The organic phase was dried (Na₂SO₄) and filtered. The filtrate was concentrated in vacuo and the residue was purified by FCC eluting with 0-100% ethyl acetate in cyclohexane to give the title compound (0.053 g) as a foam. LCMS (Method 6) RT 2.37 m/z 520 [M+Na⁺].

### Intermediate 56B: (S)—N—{(S)-1-[2-(Benzo[d]isoxazol-3-yl)phenyl]-2-[3-fluoro-6-(methylsulfonyl)pyridine-2-yl]ethyl}-2-methylpropane-2-sulfinamide

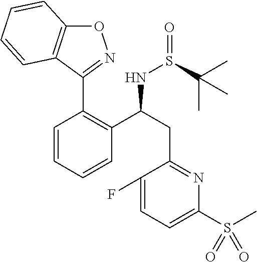 [SMILES: CC(C)(C)[S@](=O)N[C@@H](Cc1nc(S(C)(=O)=O)ccc1F)c1ccccc1-c1noc2ccccc12]

Starting from (S)—N—{(S)-1-[2-(benzo[d]isoxazol-3-yl)phenyl]-2-[6-bromo-3-fluoropyridine-2-yl]ethyl}-2-methylpropane-2-sulfinamide (Intermediate 71E). LCMS (Method 3) RT 1.43 m/z 516 [MH⁺].

### Intermediate 56C: (S)—N—{(S)-1-[2-(Benzo[d]isoxazol-3-yl)phenyl]-2-[5-fluoro-6-(methylsulfonyl)pyridine-2-yl]ethyl}-2-methylpropane-2-sulfinamide

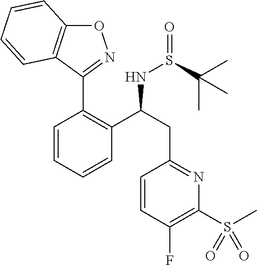 [SMILES: CC(C)(C)[S@](=O)N[C@@H](Cc1ccc(F)c(S(C)(=O)=O)n1)c1ccccc1-c1noc2ccccc12]

Starting from (S)—N—{(S)-1-[2-(benzo[d]isoxazol-3-yl)phenyl]-2-[6-bromo-5-fluoropyridine-2-yl]ethyl}-2-methylpropane-2-sulfinamide (Intermediate 71H). LCMS (Method 3) RT 1.51 m/z 516 [MH⁺]

### Intermediate 57A: Methyl 6-{(S)-2-[2-(benzo[d]isoxazol-3-yl)phenyl]-2-[((S)-tert-butylsulfinyl)amino]ethyl}pyridine-2-carboxylate

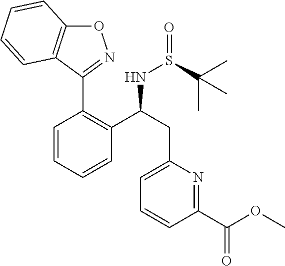 [SMILES: COC(=O)c1cccc(C[C@H](N[S@@](=O)C(C)(C)C)c2ccccc2-c2noc3ccccc23)n1]

Palladium acetate (0.006 g) and xantphos (0.029 g) were added to a degassed mixture of (S)—N—{(S)-1-[2-(benzo[d]isoxazol-3-yl)phenyl]-2-(6-bromopyridine-2-yl)ethyl}propane-2-sulfinamide (Intermediate 6B, 0.25 g) in trimethylamine (5 mL) and methanol (0.381 mL). Carbon monoxide was bubbled through the mixture and then the vial was sealed under an atmosphere of carbon monoxide. The resultant mixture was stirred and heated at 65° C. overnight. After cooling, the mixture was concentrated in vacuo and the residue was partitioned between ethyl acetate and water. The organic phase was dried (Na₂SO₄) and filtered. The filtrate was concentrated in vacuo and the residue was purified by FCC eluting with 0-3% methanol in ethyl acetate to give the title compound (0.146 g) which was used directly in the next stage.

### Intermediate 58A: 6-{(S)-2-[2-(Benzo[d]isoxazol-3-yl)phenyl]-2-[((S)-tert-butylsulfinyl)amino]ethyl}pyridine-2-carboxylic acid

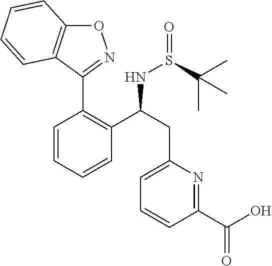 [SMILES: CC(C)(C)[S@](=O)N[C@@H](Cc1cccc(C(=O)O)n1)c1ccccc1-c1noc2ccccc12]

Lithium hydroxide (0.239 g) was added to a solution of methyl 6-{(S)-2-[2-(benzo[d]isoxazol-3-yl)phenyl]-2-[((S)-tert-butylsulfinyl)amino]ethyl}pyridine-2-carboxylate (Intermediate 57A, 0.272 g) in a mixture of dioxane (3 mL) and water (3 mL) and the resultant mixture was stirred at room temperature overnight. Aqueous potassium bisulphate solution (5% w/v) was added and the mixture was partitioned between ethyl acetate and water. The organic phase was dried (Na₂SO₄) and filtered. The filtrate was concentrated in vacuo to give the title compound (0.223 g) as a white solid. LCMS (Method 8) RT 2.89 m/z 464 [MH⁺].

### Intermediate 59A: 6-{(S)-2-[2-(Benzo[d]isoxazol-3-yl)phenyl]-2-[((S)-tert-butylsulfinyl)amino]ethyl}pyridine-2-carboxamide

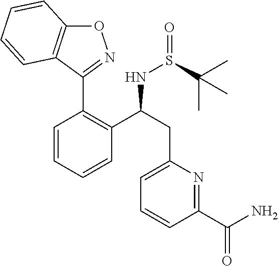 [SMILES: CC(C)(C)[S@](=O)N[C@@H](Cc1cccc(C(N)=O)n1)c1ccccc1-c1noc2ccccc12]

HATU (0.123 g) was added to a mixture of 6-{(S)-2-[2-(benzo[d]isoxazol-3-yl)phenyl]-2-[((S)-tert-butylsulfinyl)amino]ethyl}pyridine-2-carboxylic acid (Intermediate 58A, 0.075 g), di-isopropylethylamine (0.111 mL) and ammonium chloride (0.017 g) in DMF (1 mL) and the mixture was stirred for 3 days. The mixture was partitioned between ethyl acetate and water and the organic phase was dried (Na₂SO₄) and filtered. The filtrate was concentrated in vacuo to give the title compound (0.146 g) which was used directly in the next stage.

By proceeding in a similar manner to Intermediate 59A, the following compounds were prepared:

### Intermediate 59B: 6-{(S)-2-[2-(Benzo[d]isoxazol-3-yl)phenyl]-2-[((S)-tert-butylsulfinyl)amino]ethyl}-N-methylpyridine-2-carboxamide

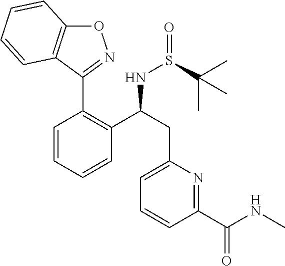 [SMILES: CNC(=O)c1cccc(C[C@H](N[S@@](=O)C(C)(C)C)c2ccccc2-c2noc3ccccc23)n1]

Starting from 6-{(S)-2-[2-(benzo[d]isoxazol-3-yl)phenyl]-2-[((S)-tert-butylsulfinyl)amino]ethyl}pyridine-2-carboxylic acid (Intermediate 58A) and methylamine solution in THF (2M) and used directly in the next stage.

### Intermediate 59C: (S)—N—{(S)-1-[2-(Benzo[d]isoxazol-3-yl)phenyl]-2-[6-(morpholine-4-carbonyl)pyridine-2-yl]ethyl}propane-2-sulfinamide

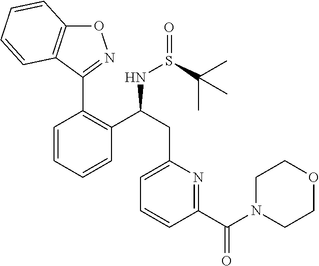 [SMILES: CC(C)(C)[S@](=O)N[C@@H](Cc1cccc(C(=O)N2CCOCC2)n1)c1ccccc1-c1noc2ccccc12]

Starting from 6-{(S)-2-[2-(benzo[d]isoxazol-3-yl)phenyl]-2-[((S)-tert-butylsulfinyl)amino]ethyl}pyridine-2-carboxylic acid (Intermediate 58A) and morpholine and used directly in the next stage.

### Intermediate 59D: 6-{(S)-2-[2-(Benzo[d]isoxazol-3-yl)phenyl]-2-[((S)-tert-butylsulfinyl)amino]ethyl}-N-(cyclopropylmethyl)pyridine-2-carboxamide

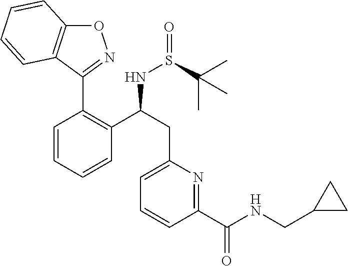 [SMILES: CC(C)(C)[S@](=O)N[C@@H](Cc1cccc(C(=O)NCC2CC2)n1)c1ccccc1-c1noc2ccccc12]

Starting from 6-{(S)-2-[2-(benzo[d]isoxazol-3-yl)phenyl]-2-[((S)-tert-butylsulfinyl)amino]ethyl}pyridine-2-carboxylic acid (Intermediate 58A) and cyclopropylemthylamine and used directly in the next stage.

### Intermediate 59E: 6-{(S)-2-[2-(Benzo[d]isoxazol-3-yl)phenyl]-2-[((S)-tert-butylsulfinyl)amino]ethyl}-N-(2-methoxyethyl)pyridine-2-carboxamide

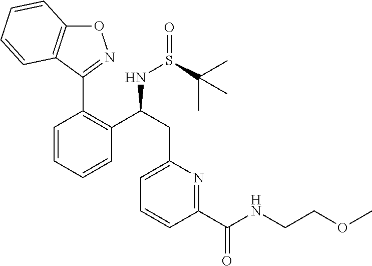 [SMILES: COCCNC(=O)c1cccc(C[C@H](N[S@@](=O)C(C)(C)C)c2ccccc2-c2noc3ccccc23)n1]

Starting from 6-{(S)-2-[2-(benzo[d]isoxazol-3-yl)phenyl]-2-[((S)-tert-butylsulfinyl)amino]ethyl}pyridine-2-carboxylic acid (Intermediate 58A) and 2-methoxyethylamine and used directly in the next stage.

### Intermediate 60A: (S)—N—{(S)-1-[2-(Benzo[d]isoxazol-3-yl)phenyl]-2-(6-morpholinopyridine-2-yl]ethyl}propane-2-sulfinamide

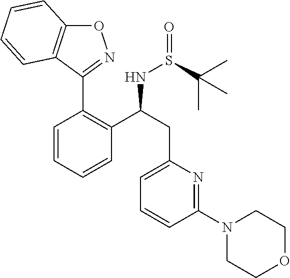 [SMILES: CC(C)(C)[S@](=O)N[C@@H](Cc1cccc(N2CCOCC2)n1)c1ccccc1-c1noc2ccccc12]

A mixture of (S)—N—{(S)-1-[2-(benzo[d]isoxazol-3-yl)phenyl]-2-(6-bromopyridine-2-yl)ethyl}propane-2-sulfinamide (Intermediate 6B, 0.075 g), morpholine (0.039 mL) and trimethylamine (0.07 mL) in 2-methoxyethanol (1 mL) was stirred and heated in a sealed vial at 100° C. for a total of 160 hours. After cooling, the mixture was diluted with saturated aqueous sodium bicarbonate solution and extracted with DCM. The organic phase was filtered through a phase separator and the filtrate was concentrated in vacuo. The residue was purified by FCC eluting with 0-4.5% 2M ammonia in methanol in DCM to give the title compound (0.143 g) as a brownish gum. LCMS (Method 8) RT 2.96 m/z 505 [MH⁺].

### Intermediate 61A: (S)—N—{(S)-1-[2-(Benzo[d]isoxazol-3-yl)phenyl]-2-(6-bromopyridine-2-yl]ethyl}-2,2,2-trifluoroacetamide

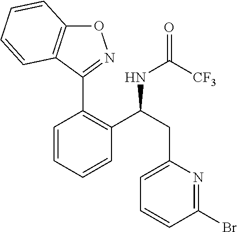 [SMILES: O=C(N[C@@H](Cc1cccc(Br)n1)c1ccccc1-c1noc2ccccc12)C(F)(F)F]

Trifluoroacetic anhydride (0.135 mL) was added slowly to a stirred, cooled solution of (S)-1-[2-(benzo[d]isoxazol-3-yl)phenyl]-2-(6-bromopyridine-2-yl)ethan-1-amine (Example 44, 0.349 g) and triethylamine (0.62 mL) in DCM (3 mL) while maintaining the temperature below 5° C. The mixture was allowed to come slowly to room temperature and stirred overnight then partitioned between DCM and 10% aqueous citric acid solution. The aqueous phase was further extracted with DCM and the combined organic phases were washed with 10% aqueous citric acid solution and sodium bicarbonate solution then dried (Na₂SO₄) and filtered. The filtrate was concentrated in vacuo to give the title compound (0.405 g). ¹H NMR (400 MHz, CDCl₃) 9.19 (1H, d, J=5.4 Hz), 7.83-7.81 (1H, m), 7.70-7.57 (3H, m), 7.44-7.35 (5H, m), 7.19 (1H, dd, J=1.6, 7.6 Hz), 6.99-6.96 (1H, m), 5.44-5.38 (1H, m), 3.35 (1H, dd, J=4.6, 14.1 Hz), 3.18 (1H, dd, J=7.3, 14.2 Hz).

### Intermediate 62A: tert-Butyl (S)-(6-{2-[2-(benzo[d]isoxazol-3-yl)phenyl]-2-(2,2,2-trifluoroacetamido)ethyl}pyridine-2-yl)carbamate

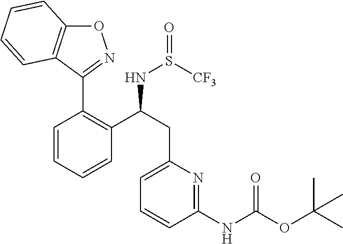 [SMILES: CC(C)(C)OC(=O)Nc1cccc(C[C@H](NS(=O)C(F)(F)F)c2ccccc2-c2noc3ccccc23)n1]

Tris(dibenzylideneacetone)dipalladium (0) (0.009 g) was added to a degassed suspension of (S)—N—{(S)-1-[2-(benzo[d]isoxazol-3-yl)phenyl]-2-(6-bromopyridine-2-yl]ethyl}-2,2,2-trifluoroacetamide (Intermediate 61A, 0.1 g), tert-butyl carbamate (0.029 g), cesium carbonate (0.134 g) and xantphos (0.012 g) in dioxane (1 mL). The resultant mixture was again degassed then heated in a sealed vial at 100° C. overnight. After cooling, the mixture was filtered through a pad of Celite™ and the filtrate was partitioned between ethyl acetate and water. The aqueous phase was further extracted with ethyl acetate and the combined organic phases were washed with brine, dried (Na₂SO₄) and filtered. The filtrate was concentrated in vacuo and the residue was purified by FCC eluting with 0-50% ethyl acetate in petroleum ether to give the title compound (0.046 g). LCMS (Method 8) RT 4.21 m/z 427 [MH⁺−100] loss of Boc.

### Intermediate 63A: (S)—N-{2-[6-Aminopyridine-2-yl]-1-[2-(benzo[d]isoxazol-3-yl)phenyl]ethyl}-2,2,2-trifluoroacetamide

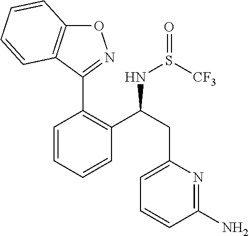 [SMILES: Nc1cccc(C[C@H](NS(=O)C(F)(F)F)c2ccccc2-c2noc3ccccc23)n1]

TFA (0.5 mL) was added to a solution of tert-butyl (S)-(6-{2-[2-(benzo[d]isoxazol-3-yl)phenyl]-2-(2,2,2-trifluoroacetamido)ethyl}pyridine-2-yl)carbamate (Intermediate 62A, 0.044 g) in DCM (1 mL) and the mixture allowed to stand at room temperature overnight then concentrated in vacuo. The residue was azeotroped with toluene to give the title compound (0.05 g) as a solid. LCMS (Method 8) RT 2.23 m/z 427 [MH⁺].

### Intermediate 64A: (S)—N-{1-[2-(Benzo[d]isoxazol-3-yl)phenyl]ethyl}-2-[6-(3-methylureido)pyridine-2-yl]-2,2,2-trifluoroacetamide

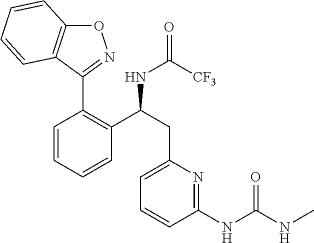 [SMILES: CNC(=O)Nc1cccc(C[C@H](NC(=O)C(F)(F)F)c2ccccc2-c2noc3ccccc23)n1]

Triphosgene (0.021 g) was added to a stirred, cooled solution of (S)—N-{2-[6-aminopyridine-2-yl]-1-[2-(benzo[d]isoxazol-3-yl)phenyl]ethyl}-2,2,2-trifluoroacetamide (Intermediate 63A, 0.06 g) and N,N-di-isopropylethylamine (0.049 mL) in dry THF (1 mL) while maintaining the temperature below 5° C. After stirring at 0° C. for a further 20 minutes methylamine (2M solution in THF, 0.14 mL) was added. The temperature was allowed to rise to room temperature and the mixture was stirred overnight. The mixture was partitioned between ethyl acetate and water and the aqueous phase was further extracted with ethyl acetate. The combined organic phases were dried (Na₂SO₄) and filtered. The filtrate was concentrated in vacuo and the residue was purified by FCC eluting with 0-70% ethyl acetate in petroleum ether to give the title compound (0.027 g). LCMS (Method 8) RT 3.14 m/z 484 [MH⁺].

By proceeding in a similar manner to Intermediate 64A, the following compounds were prepared:

### Intermediate 64B: Methyl (S)-(6-{2-[2-(benzo[d]isoxazol-3-yl)phenyl]-2,2,2-trifluoroacetamido}ethylpyridine-2-yl)carbamate

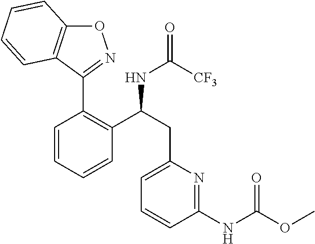 [SMILES: COC(=O)Nc1cccc(C[C@H](NC(=O)C(F)(F)F)c2ccccc2-c2noc3ccccc23)n1]

Starting from (S)—N-{2-[6-aminopyridine-2-yl]-1-[2-(benzo[d]isoxazol-3-yl)phenyl]ethyl}-2,2,2-trifluoroacetamide (Intermediate 63A) and replacing the methylamine solution by methanol. LCMS (Method 8) RT 3.63 m/z 485 [MH⁺]

### Intermediate 65A: (S)—N-{1-[2-(Benzo[d]isoxazol-3-yl)phenyl]-2-[6-(N-[methylsulfonyl]methylsulfonamido)pyridine-2-yl]ethyl}-2,2,2-trifluoroacetamide

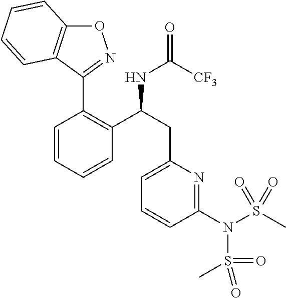 [SMILES: CS(=O)(=O)N(c1cccc(C[C@H](NC(=O)C(F)(F)F)c2ccccc2-c2noc3ccccc23)n1)S(C)(=O)=O]

Methanesulfonyl chloride (0.016 mL) was added to a stirred, cooled solution of (S)—N-{2-[6-aminopyridine-2-yl]-1-[2-(benzo[d]isoxazol-3-yl)phenyl]ethyl}-2,2,2-trifluoroacetamide (Intermediate 63A, 0.06 g) and triethylamine (0.059 mL) in DCM (1 mL) while maintaining the temperature below 5° C. The mixture was allowed to warm to room temperature and left to stand overnight. Pyridine (0.5 mL) was added followed by additional methanesulfonyl chloride (0.006 mL) and the mixture was stirred at room temperature for 1 hour. The mixture was concentrated in vacuo and the residue was partitioned between ethyl acetate and water. The aqueous phase was further extracted with ethyl acetate and the combined organic phases were washed with 10% aqueous citric acid solution, dried (Na₂SO₄) and filtered. The filtrate was concentrated in vacuo to give the title compound as a foam. LCMS (Method 8) RT 3.62 m/z 582 [MH⁺] plus a small amount of mono-sulfonamide RT 3.40 m/z 505 [MH⁺].

### Intermediate 66A: (E)-3-(6-{(S)-2-[2-(Benzo[d]isoxazol-3-yl)phenyl]-2-[((S)-tert-butylsulfinyl)amino]ethyl}pyridine-2-yl)-N,N-dimethylacrylamide

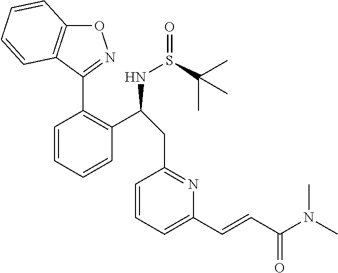 [SMILES: CN(C)C(=O)/C=C/c1cccc(C[C@H](N[S@@](=O)C(C)(C)C)c2ccccc2-c2noc3ccccc23)n1]

A mixture of (S)—N—{(S)-1-[2-(benzo[d]isoxazol-3-yl)phenyl]-2-(6-bromopyridine-2-yl)ethyl}-2-methylpropane-2-sulfinamide (Intermediate 6B, 0.29 g), N,N-dimethylacrylamide (0.087 g), tetrakis(triphenylphosphine) palladium (0.05 g) and potassium carbonate (0.16 g) in DMF (6 mL) was degassed then heated in the microwave at 100° C. for 30 minutes, then at 140° C. for 30 minutes and finally at 145° C. for 2 hours. After cooling, the mixture was concentrated in vacuo and the residue was partitioned between ethyl acetate and water. The organic phase was dried (Na₂SO₄) and filtered. The filtrate was concentrated in vacuo and the residue was purified by FCC eluting with 0-20% methanol in ethyl acetate to give the title compound (0.057 g). LCMS (Method 6) RT 3.18 m/z 517 [MH⁺]

### Intermediate 67A: 3-(6-{(S)-2-[2-(Benzo[d]isoxazol-3-yl)phenyl]-2-[((S)-tert-butylsulfinyl)amino]ethyl}pyridine-2-yl)-N,N-dimethylpropanamide

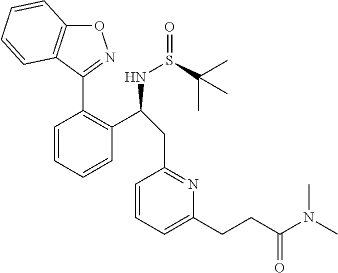 [SMILES: CN(C)C(=O)CCc1cccc(C[C@H](N[S@@](=O)C(C)(C)C)c2ccccc2-c2noc3ccccc23)n1]

A mixture of (E)-3-(6-{(S)-2-[2-(benzo[d]isoxazol-3-yl)phenyl]-2-[((S)-tert-butylsulfinyl)amino]ethyl}pyridine-2-yl)-N,N-dimethylacrylamide (Intermediate 66A, 0.057 g) and palladium on carbon (10%, 0.02 g) in ethanol (3 mL) was stirred under a balloon of hydrogen overnight. Further palladium on carbon (10%, 0.02 g) was added and the mixture was stirred under a balloon of hydrogen for 6 hours. After removal of the hydrogen atmosphere, the mixture was filtered through a pad of Celite™ and the filtrate was concentrated in vacuo. The residue was purified by FCC eluting with 0-20% methanol in ethyl acetate to give the title compound (0.024 g). LCMS (Method 6) RT 2.60 m/z 519 [MH⁺].

### Intermediate 68A: 2-(1-Methyl-1H-indazole-3-yl)benzaldehyde

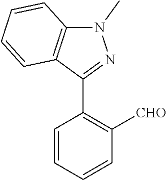 [SMILES: Cn1nc(-c2ccccc2C=O)c2ccccc21]

A solution of sodium carbonate (0.37 g) in water (5 mL) was added to a solution of 3-bromo-1-methyl-1H-indazole (0.317 g) and 2-formylphenylboronic acid (0.27 g) in DME (10 mL). After degassing, tetrakis(triphenylphosphine) palladium (0.087 g) was added and the resultant mixture was sealed in the vial and heated at 80° C. for 4 hours. The reaction was repeated on the same scale. After cooling, the two mixtures were combined and treated with water then extracted with ethyl acetate. The organic phases were washed with water, dried (MgSO₄) and filtered. The filtrate was concentrated in vacuo and the residue was purified by FCC eluting with 0-20% ethyl acetate in pentane to give the title compound (0.582 g) as an off-white solid. ¹H NMR (400 MHz, CDCl₃) 10.24 (1H, d, J=0.9 Hz), 8.11 (1H, dd, J=1.2, 7.8 Hz), 7.83-7.69 (3H, m), 7.57-7.47 (3H, m), 7.26 (1H, s), 4.17 (3H, s).

By proceeding in a similar manner to Intermediate 68A, the following compounds were prepared:

### Intermediate 68B: 2-(1-Isopropyl-1H-indazole-3-yl)benzaldehyde

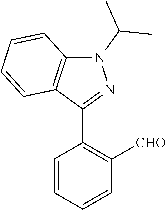 [SMILES: CC(C)n1nc(-c2ccccc2C=O)c2ccccc21]

Starting from 3-bromo-1-isopropyl-1H-indazole (Intermediate 69A) and 2-formylphenylboronic acid. ¹H NMR (400 MHz, CDCl₃) 10.24 (1H, s), 8.11 (1H, dd, J=1.2, 7.8 Hz), 7.87-7.81 (2H, m), 7.74-7.69 (1H, m), 7.56-7.52 (2H, m), 7.47-7.42 (1H, m), 7.26-7.21 (1H, m), 4.98-4.88 (1H, m), 1.65 (6H, d, J=6.5 Hz).

### Intermediate 69A: 3-Bromo-1-isopropylindazole

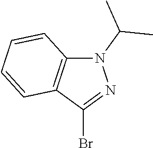

Cesium carbonate (3.91 g) was added to a solution of 3-bromoindazole (1.97 g) in DMF (30 mL) and the resultant solution was stirred at room temperature for 1 hour. A solution of 2-iodopropane (1.1 mL) in DMF (5 mL) was then added and the resultant mixture was stirred at room temperature overnight. The mixture was added to water and extracted with ethyl acetate, washed with water, dried and filtered. The filtrate was concentrated in vacuo and the residue was purified by FCC eluting with 0-10% ethyl acetate in pentane to give the title compound (1.88 g) as the main component isolated as a gum which solidified on standing. ¹H NMR (400 MHz, CDCl₃) 7.62-7.59 (1H, m), 7.42-7.40 (2H, m), 7.23-7.15 (1H, m), 4.87-4.76 (1H, m), 1.59 (6H, d, J=8.2 Hz).

### Intermediate 70A: (S)—N—{(S)-1-[2-(Benzo[d]isoxazol-3-yl)phenyl]-2-[5-fluoro-6-(trimethylsilyl)pyridine-2-yl]ethyl}-2-methylpropane-2-sulfinamide

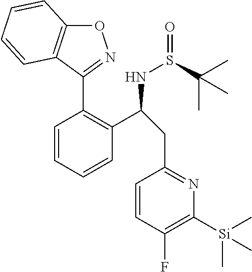 [SMILES: CC(C)(C)[S@](=O)N[C@@H](Cc1ccc(F)c([Si](C)(C)C)n1)c1ccccc1-c1noc2ccccc12]

A solution of LDA (2M in THF, heptane, ethylbenzene, 0.83 mL) was added to a stirred, cooled solution of 5-fluoro-2-methylpyridine (0.185 mL) in dry THF (8 mL) while maintaining the temperature below −70° C. After stirring the mixture at −75° C. for 1 hour, TMS chloride (0.21 mL) was added. The temperature was allowed to rise slowly to −5° C. and the mixture was stirred at that temperature for 45 minutes. The mixture was re-cooled to −60° C. and LDA (2M in THF, heptane, ethylbenzene, 0.83 mL) was added dropwise while maintaining the temperature below −70° C. The mixture was stirred for 1 hour at −75° C. then added by cannula to a cooled solution of (S)—N-[2-(benzo[d]isoxazol-yl)benzylidene]-2-methylpropane-2-sulfinamide (Intermediate 4B, 0.21 g) in dry THF (4 mL) while maintaining the temperature below −70° C. The mixture was stirred at −75° C. for 1 hour then allowed to warm to room temperature. The mixture was partitioned between ethyl acetate and saturated aqueous sodium bicarbonate solution. The organic phase was dried (MgSo₄) and filtered. The filtrate was concentrated in vacuo and the residue was purified by FCC eluting with 0-100% ethyl acetate in cyclohexane to give the title compound (0.16 g) as a yellow gummy solid. LCMS (Method 4) RT 2.13 m/z 510 [MH⁺].

### Intermediate 71A: (S)—N—{(S)-1-[2-(Benzo[d]isoxazol-3-yl)phenyl]-2-(5-fluoropyridine-2-yl)ethyl}-2-methylpropane-2-sulfinamide

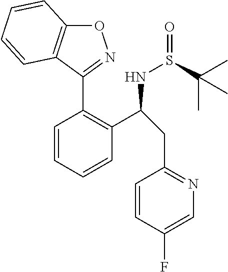 [SMILES: CC(C)(C)[S@](=O)N[C@@H](Cc1ccc(F)cn1)c1ccccc1-c1noc2ccccc12]

An aqueous solution of sodium hydroxide (2M, 12 mL) was added to a solution of (S)—N—{(S)-1-[2-(benzo[d]isoxazol-3-yl)phenyl]-2-[5-fluoro-6-(trimethylsilyl)pyridine-2-yl]ethyl}-2-methylpropane-2-sulfinamide (Intermediate 70A, 0.15 g) in IMS (60 mL) and the resultant mixture was stirred an heated at 85° C. for 50 minutes. After cooling, the mixture was combined with an earlier small scale reaction using (S)—N—{(S)-1-[2-(benzo[d]isoxazol-3-yl)phenyl]-2-[5-fluoro-6-(trimethylsilyl)pyridine-2-yl]ethyl}-2-methylpropane-2-sulfinamide (Intermediate 70A, 0.01 g) and concentrated in vacuo. The residue was diluted with water and extracted with DCM followed by ethyl acetate. The combined organic phases were dried (MgSo₄) and filtered. The filtrate was concentrated in vacuo to give the title compound (0.122 g) as a yellow gum. ¹H NMR (400 MHz, CDCl₃) 8.30 (1H, d, J=2.9 Hz), 7.70-7.61 (4H, m), 7.59-7.48 (2H, m), 7.46-7.35 (2H, m), 7.25-7.19 (1H, m), 7.05-6.99 (1H, m), 5.18 (1H, d, J=6.8 Hz), 5.10-5.04 (1H, m), 3.35 (1H, dd, J=4.8, 13.8 Hz), 3.19 (1H, dd, J=8.6, 13.9 Hz), 0.99 (9H, s).

By proceeding in a similar manner to Intermediate 71A, the following compounds were prepared:

### Intermediate 71B: (S)—N—{(S)-1-[2-(Benzo[d]isoxazol-3-yl)phenyl]-2-(6-cyano-3-fluoropyridine-2-yl)ethyl}-2-methylpropane-2-sulfinamide

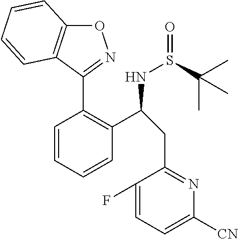 [SMILES: CC(C)(C)[S@](=O)N[C@@H](Cc1nc(C#N)ccc1F)c1ccccc1-c1noc2ccccc12]

Starting from (S)—N—{(S)-1-[2-(benzo[d]isoxazol-3-yl)phenyl]-2-[6-cyano-3-fluoro-4-(trimethylsilyl)pyridine-2-yl]ethyl}-2-methylpropane-2-sulfinamide (Intermediate 27K). LCMS (Method 4) m/z 463 [MH⁺].

### Intermediate 71C: (S)—N—{(S)-1-[2-(Benzo[d]isoxazol-3-yl)phenyl]-2-(3-fluoro-6-methylpyridine-2-yl)ethyl}-2-methylpropane-2-sulfinamide

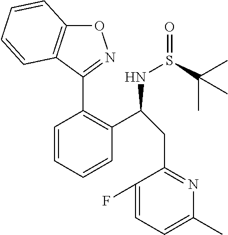 [SMILES: Cc1ccc(F)c(C[C@H](N[S@@](=O)C(C)(C)C)c2ccccc2-c2noc3ccccc23)n1]

Starting from (S)—N—{(S)-1-[2-(benzo[d]isoxazol-3-yl)phenyl]-2-[3-fluoro-6-methyl-4-(trimethylsilyl)pyridine-2-yl]ethyl}-2-methylpropane-2-sulfinamide (Intermediate 11H). LCMS (Method 4) RT 1.48 m/z 452 [MH⁺].

### Intermediate 71D: 6-{(S)-2-[2-(Benzo[d]isoxazol-3-yl)phenyl]-2-[((S)-tert-butylsulfinyl)amino]ethyl}-5-fluoro-N,N-dimethylpyridine-2-carboxamide

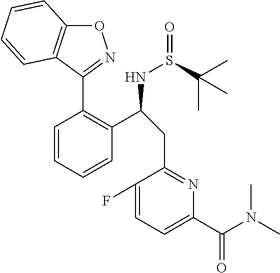 [SMILES: CN(C)C(=O)c1ccc(F)c(C[C@H](N[S@@](=O)C(C)(C)C)c2ccccc2-c2noc3ccccc23)n1]

Starting from 6-{(S)-2-[2-(benzo[d]isoxazol-3-yl)phenyl]-2-[((S)-tert-butylsulfinyl)amino]ethyl}-5-fluoro-N,N-dimethyl-4-(trimethylsilyl)pyridine-2-carboxamide (Intermediate 74A). LCMS (Method 8) RT 3.02 m/z 509 [MH⁺].

### Intermediate 71E: (S)—N—{(S)-1-[2-(Benzo[d]isoxazol-3-yl)phenyl]-2-(6-bromo-3-fluoropyridine-2-yl)ethyl}-2-methylpropane-2-sulfinamide

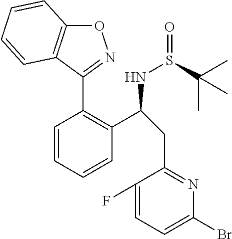 [SMILES: CC(C)(C)[S@](=O)N[C@@H](Cc1nc(Br)ccc1F)c1ccccc1-c1noc2ccccc12]

Starting from (S)—N—{(S)-1-[2-(benzo[d]isoxazol-3-yl)phenyl]-2-[6-bromo-3-fluoro-4-(trimethylsilyl)pyridine-2-yl]ethyl}-2-methylpropane-2-sulfinamide (Intermediate 6S). LCMS (Method 3) RT 1.61 m/z 516/518 [MH⁺].

### Intermediate 71F: (S)—N—{(S)-1-[2-(Benzo[d]isoxazol-3-yl)phenyl]-2-[3-fluoro-6-(2-hydroxyethyl)pyridine-2-yl)ethyl]-2-methylpropane-2-sulfinamide

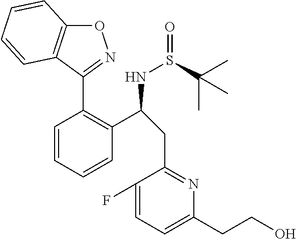 [SMILES: CC(C)(C)[S@](=O)N[C@@H](Cc1nc(CCO)ccc1F)c1ccccc1-c1noc2ccccc12]

Starting from (S)—N—{(S)-1-[2-(benzo[d]isoxazol-3-yl)phenyl]-2-[3-fluoro-6-(2-hydroxyethyl)-4-(trimethylsilyl)pyridine-2-yl)ethyl]-2-methylpropane-2-sulfinamide (Intermediate 35D) and used directly in the next stage.

### Intermediate 71G: (S)—N—{(S)-1-[2-(Benzo[d]isoxazol-3-yl)phenyl]-2-[3-fluoro-6-(1H-pyrazol-4-yl)pyridine-2-yl]ethyl}-2-methylpropane-2-sulfinamide

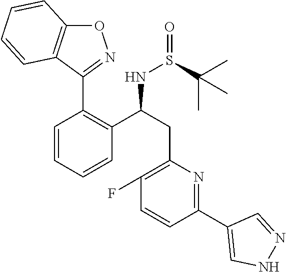 [SMILES: CC(C)(C)[S@](=O)N[C@@H](Cc1nc(-c2cn[nH]c2)ccc1F)c1ccccc1-c1noc2ccccc12]

Starting from (S)—N—{(S)-1-[2-(benzo[d]isoxazol-3-yl)phenyl]-2-[3-fluoro-6-(1H-pyrazol-4-yl)-4-(trimethylsilyl)pyridine-2-yl]ethyl}-2-methylpropane-2-sulfinamide (Intermediate 46B) and used directly in the next step

### Intermediate 71H: (S)—N—{(S)-1-[2-(Benzo[d]isoxazol-3-yl)phenyl]-2-(6-bromo-5-fluoropyridine-2-yl)ethyl}-2-methylpropane-2-sulfinamide

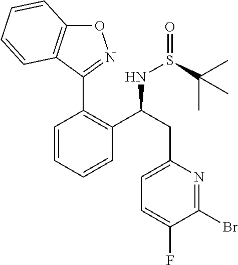 [SMILES: CC(C)(C)[S@](=O)N[C@@H](Cc1ccc(F)c(Br)n1)c1ccccc1-c1noc2ccccc12]

Starting from (S)—N—{(S)-1-[2-(benzo[d]isoxazol-3-yl)phenyl]ethyl}-2-[6-bromo-5-fluoro-4-(trimethylsilyl)pyridine-2-yl]-2-methylpropane-2-sulfinamide (Intermediate 6W). LCMS (Method 3) RT 1.66 m/z 516/518 [MH⁺].

### Intermediate 71I: (S)—N—{(S)-2-(6-Bromo-3-fluoropyridine-2-yl)-1-[2-(6-trifluoromethylbenzo[d]isoxazol-3-yl)phenyl]ethyl}-2-methylpropane-2-sulfinamide

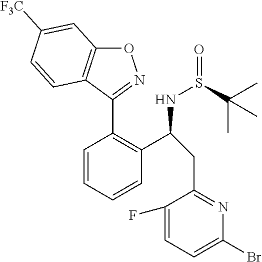 [SMILES: CC(C)(C)[S@](=O)N[C@@H](Cc1nc(Br)ccc1F)c1ccccc1-c1noc2cc(C(F)(F)F)ccc12]

Starting from (S)—N—{(S)-2-[6-bromo-3-fluoro-4-(trimethylsilyl)pyridine-2-yl]-1-[2-(6-trifluoromethylbenzo[d]isoxazol-3-yl)phenyl]ethyl}-2-methylpropane-2-sulfinamide (Intermediate 6X) but stirring at room temperature for 30 minutes. LCMS (Method 3) RT 1.59 m/z 584/586 [MH⁺].

### Intermediate 71J: (S)—N—{(S)-2-(6-Bromo-3-fluoropyridine-2-yl)-1-[2-(6-methylbenzo[d]isoxazol-3-yl)phenyl]ethyl}-2-methylpropane-2-sulfinamide

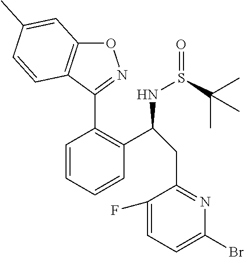 [SMILES: Cc1ccc2c(-c3ccccc3[C@H](Cc3nc(Br)ccc3F)N[S@@](=O)C(C)(C)C)noc2c1]

Starting from (S)—N—{(S)-2-[6-bromo-3-fluoro-4-(trimethylsilyl)pyridine-2-yl]-1-[2-(6-methylbenzo[d]isoxazol-3-yl)phenyl]ethyl}-2-methylpropane-2-sulfinamide (Intermediate 6Y) but using THF in place of IMS and stirring at room temperature for 1 hour. LCMS (Method 3) RT 1.54 m/z 530/532 [MH⁺].

### Intermediate 71K: (S)—N—{(S)-2-(6-bromo-3-fluoropyridine-2-yl)-1-[2-(6-chlorobenzo[d]isoxazol-3-yl)phenyl]ethyl}-2-methylpropane-2-sulfinamide

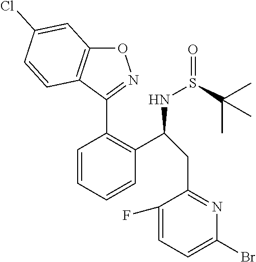 [SMILES: CC(C)(C)[S@](=O)N[C@@H](Cc1nc(Br)ccc1F)c1ccccc1-c1noc2cc(Cl)ccc12]

Starting from (S)—N—{(S)-2-[6-bromo-3-fluoro-4-(trimethylsilyl)pyridine-2-yl]-1-[2-(6-chlorobenzo[d]isoxazol-3-yl)phenyl]ethyl}-2-methylpropane-2-sulfinamide (Intermediate 6Z) but using THF in place of IMS and stirring at room temperature for 1 hour. LCMS (Method 4) RT 1.93 m/z 550/552 [MH⁺].

### Intermediate 71L: (S)—N—{(S)-2-(6-Bromo-3-fluoropyridine-2-yl)-1-[2-(6-fluorobenzo[d]isoxazol-3-yl)phenyl]ethyl}-2-methylpropane-2-sulfinamide

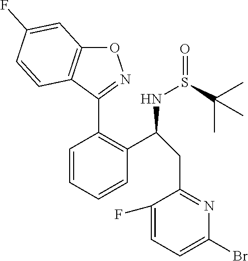 [SMILES: CC(C)(C)[S@](=O)N[C@@H](Cc1nc(Br)ccc1F)c1ccccc1-c1noc2cc(F)ccc12]

Starting from (S)—N—{(S)-2-[6-bromo-3-fluoro-4-(trimethylsilyl)pyridine-2-yl]-1-[2-(6-fluorobenzo[d]isoxazol-3-yl)phenyl]ethyl}-2-methylpropane-2-sulfinamide (Intermediate 6AA) but using THF in place of IMS and stirring at room temperature for 1 hour. LCMS (Method 3) RT 1.50 m/z 534/536 [MH⁺].

### Intermediate 71M: (S)—N—{(S)-2-(6-bromo-3-fluoropyridine-2-yl)-1-[2-(6-bromobenzo[d]isoxazol-3-yl)phenyl]ethyl}-2-methylpropane-2-sulfinamide

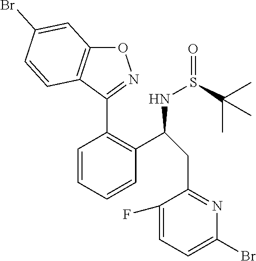 [SMILES: CC(C)(C)[S@](=O)N[C@@H](Cc1nc(Br)ccc1F)c1ccccc1-c1noc2cc(Br)ccc12]

Starting from (S)—N—{(S)-2-[6-bromo-3-fluoro-4-(trimethylsilyl)pyridine-2-yl]-1-[2-(6-bromobenzo[d]isoxazol-3-yl)phenyl]ethyl}-2-methylpropane-2-sulfinamide (Intermediate 6AB) but using THF in place of IMS and stirring at room temperature for 2 hours. ¹H NMR (400 MHz, CDCl₃) 7.88 (1H, d, J=1.0 Hz), 7.65 (1H, d, J=7.4 Hz), 7.57-7.44 (6H, m), 7.11 (1H, t, J=8.5 Hz), 5.14-5.09 (1H, m), 4.99-4.96 (1H, m), 3.27-3.23 (2H, m), 1.06-1.05 (9H, m).

### Intermediate 71N: (S)—N—{(S)-2-[6-Bromo-3-fluoro)pyridine-2-yl]-1-[2-(6-cyanobenzo[d]isoxazol-3-yl)phenyl]ethyl}-2-methylpropane-2-sulfinamide

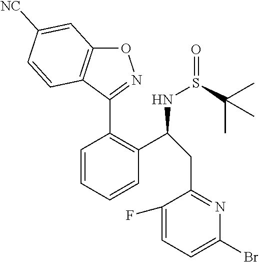 [SMILES: CC(C)(C)[S@](=O)N[C@@H](Cc1nc(Br)ccc1F)c1ccccc1-c1noc2cc(C#N)ccc12]

Starting from (S)—N—{(S)-2-[6-bromo-3-fluoro-4-(trimethylsilyl)pyridine-2-yl]-1-[2-(6-cyanobenzo[d]isoxazol-3-yl)phenyl]ethyl}-2-methylpropane-2-sulfinamide (Intermediate 6AC) but using THF in place of IMS and stirring at room temperature for 30 minutes. LCMS (Method 3) RT 1.44 m/z 541/543 [MH⁺].

### Intermediate 710: (S)—N—{(S)-2-[6-Bromo-3-fluoro)pyridine-2-yl]-1-[2-(5-fluorobenzo[d]isoxazol-3-yl)phenyl]ethyl}-2-methylpropane-2-sulfinamide

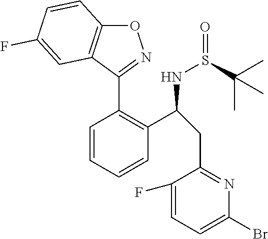 [SMILES: CC(C)(C)[S@](=O)N[C@@H](Cc1nc(Br)ccc1F)c1ccccc1-c1noc2ccc(F)cc12]

Starting from (S)—N—{(S)-2-[6-bromo-3-fluoro-4-(trimethylsilyl)pyridine-2-yl]-1-[2-(5-fluorobenzo[d]isoxazol-3-yl)phenyl]ethyl}-2-methylpropane-2-sulfinamide (Intermediate 6AE) but using THF in place of IMS and stirring at room temperature for 1 hour. LCMS (Method 3) RT 1.59 m/z 534/536 [MH⁺].

### Intermediate 71P: (S)—N—{(S)-2-[6-Bromo-3-fluoro)pyridine-2-yl]-1-[2-(7-fluorobenzo[d]isoxazol-3-yl)phenyl]ethyl}-2-methylpropane-2-sulfinamide

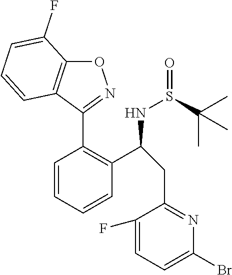 [SMILES: CC(C)(C)[S@](=O)N[C@@H](Cc1nc(Br)ccc1F)c1ccccc1-c1noc2c(F)cccc12]

Starting from (S)—N—{(S)-2-[6-bromo-3-fluoro-4-(trimethylsilyl)pyridine-2-yl]-1-[2-(7-fluorobenzo[d]isoxazol-3-yl)phenyl]ethyl}-2-methylpropane-2-sulfinamide (Intermediate 6AH) but using THF in place of IMS and stirring at room temperature for 30 minutes. ¹H NMR (400 MHz, CDCl3) 7.69-7.66 (1H, m), 7.58-7.51 (2H, m), 7.48-7.44 (2H, m), 7.38-7.30 (2H, m), 7.29-7.24 (1H, m), 7.12 (1H, t, J=8.5 Hz), 5.16-5.10 (1H, m), 5.05-5.00 (1H, m), 3.28-3.24 (2H, m), 1.06 (9H, s).

### Intermediate 72A: 6-Bromo-3-fluoro-2-methyl-4-(trimethylsilyl)pyridine

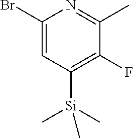

LDA (2M in THF, heptane, ethylbenzene, 5.7 mL) was added to a stirred, cooled solution of 6-bromo-3-fluoro-2-methylpyridine (2.16 g) in THF (20 mL) while maintaining the temperature below −70° C. The resultant mixture was stirred at −75° C. for 1 hour then trimethylsilyl chloride (1.49 mL) was added. The temperature was allowed to rise to −5° C. and the mixture was stirred for 1 hour before being allowed to come to room temperature. The mixture was partitioned between ethyl acetate and water and the organic phase was dried (Na₂SO₄) and filtered. The filtrate was concentrated in vacuo and the residue was purified by FCC eluting with 0-10% ethyl acetate in cyclohexane to give the title compound (2.1 g) as a white solid. LCMS (Method 4) RT 1.75 m/z 262/264 [MH⁺].

By proceeding in a similar manner to Intermediate 72A, the following compounds were prepared:

### Intermediate 72B: 2-Bromo-3-fluoro-6-methyl-4-(trimethylsilyl)pyridine

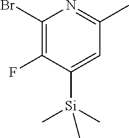

Starting from 2-bromo-3-fluoro-6-methylpyridine and trimethylsilyl chloride. LCMS (Method 3) RT 1.7 m/z 262/264 [MH⁺].

### Intermediate 73A: Ethyl 6-{(S)-2-[2-(benzo[d]isoxazol-3-yl)phenyl]-2-[((S)-tert-butylsulfinyl)amino]ethyl}-5-fluoro-4-(trimethylsilyl)pyridine-2-carboxylate

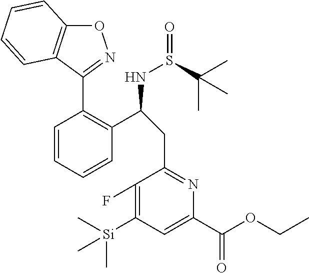 [SMILES: CCOC(=O)c1cc([Si](C)(C)C)c(F)c(C[C@H](N[S@@](=O)C(C)(C)C)c2ccccc2-c2noc3ccccc23)n1]

A mixture of (S)—N—{(S)-1-[2-(benzo[d]isoxazol-3-yl)phenyl]-2-[6-bromo-3-fluoro-4-(trimethylsilyl)pyridine-2-yl]ethyl}-2-methylpropane-2-sulfinamide (Intermediate 6S, 0.3 g), [1,1′-bis(diphenylphosphino)ferrocene]dichloropalladium (II) (0.04 g) and triethylamine (0.285 mL) in ethanol (15 mL) was sealed in a vial and carbon monoxide was bubbled through for 10 minutes. The resultant mixture was then stirred and heated at 80° C. under a balloon of carbon monoxide for 2 hours. After cooling, the mixture was filtered through Celite™ and the filtrate was concentrated in vacuo to give the crude title compound (0.37 g). LCMS (Method 4) RT 1.75 m/z 582 [MH⁺]

### Intermediate 74A: 6-{(S)-2-[2-(Benzo[d]isoxazol-3-yl)phenyl]-2-[((S)-tert-butylsulfinyl)amino]ethyl}-5-fluoro-N,N-dimethyl-4-(trimethylsilyl)pyridine-2-carboxamide

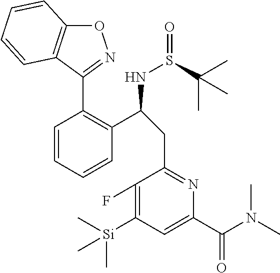 [SMILES: CN(C)C(=O)c1cc([Si](C)(C)C)c(F)c(C[C@H](N[S@@](=O)C(C)(C)C)c2ccccc2-c2noc3ccccc23)n1]

A mixture of ethyl 6-{(S)-2-[2-(benzo[d]isoxazol-3-yl)phenyl]-2-[((S)-tert-butylsulfinyl)amino]ethyl}-5-fluoro-4-(trimethylsilyl)pyridine-2-carboxylate (Intermediate 73A, 0.355 g), N,N-dimethylamine (2M solution in THF, 10 mL) and magnesium chloride (0.06 g) was stirred in a sealed vial at room temperature overnight. The resultant mixture was concentrated in vacuo and the residue was partitioned between DCM and water. The organic phase was dried (Na₂SO₄) and filtered and the filtrate was concentrated in vacuo to give the title compound (0.31 g). LCMS (Method 4) RT 1.54 m/z 581 [MH⁺]

### Intermediate 75A: N-(2-{(S)-2-[2-(Benzo[d]isoxazol-3-yl)phenyl]-2-[((S)-tert-butylsulfinyl)amino]ethyl}pyridine-3-yl)acetamide

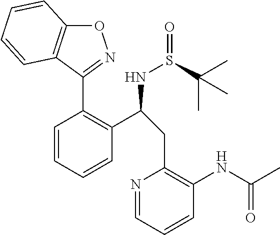 [SMILES: CC(=O)Nc1cccnc1C[C@H](N[S@@](=O)C(C)(C)C)c1ccccc1-c1noc2ccccc12]

Dioxane (2 mL) was added to a mixture of (S)—N—{(S)-2-(3-bromopyridine-2-yl)₁-[2-(benzo[d]isoxazol-3-yl)phenyl]ethyl}-2-methylpropane-2-sulfinamide (Intermediate 6T, 0.1 g), cesium carbonate (0.131 g), tris(dibenzylideneacetone)dipalladium(0) (0.009 g), acetamide (0.024 g) and xantphos (0.012 g) in a sealed vial under nitrogen and the mixture was then stirred and heated at 120° C. for 18 hours. After cooling, the mixture was filtered through Celite™ and the pad was washed with DCM. The filtrate was concentrated in vacuo and the residue was purified by MDAP to give the title compound (0.013 g). LCMS (Method 9) RT 2.98 m/z 477 [MH⁺].

### Intermediate 76A: 6-{(S)-2-[2-(Benzo[d]isoxazol-3-yl)phenyl]-2-[((S)-tert-butylsulfinyl)amino]ethyl}-N,N,5-trimethylpyridine-2-carboxamide

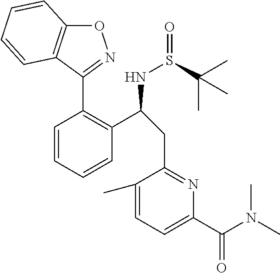 [SMILES: Cc1ccc(C(=O)N(C)C)nc1C[C@H](N[S@@](=O)C(C)(C)C)c1ccccc1-c1noc2ccccc12]

A vessel containing (S)—N—{(S)-1-[2-(benzo[d]isoxazol-3-yl)phenyl]-2-(6-bromo-3-methylpyridin-2-yl)ethyl}-2-methylpropane-2-sulfinamide (Intermediate 61, 0.127 g), palladium acetate (0.003 g), Xantphos (0.014 g) potassium phosphate (0.316 g) and dimethylamine hydrochloride (0.061 g) in toluene (1 mL) was sealed, evacuated and charged with carbon monoxide then heated at 80° C. under an atmosphere of carbon monoxide for 4 hours. After cooling, the mixture was diluted with water and extracted with ethyl acetate, washed with brine then saturated aqueous sodium bicarbonate, dried (Na₂SO₄) and filtered. The filtrate was concentrated in vacuo and the residue was purified by FCC eluting with 0-5% methanol in DCM to give the title compound (0.045 g) as a red solid. LCMS (Method 3) RT 1.40 m/z 505 [MH⁺].

### Intermediate 77A: (S)—N—{(S)-2-(3-Fluoro-6-methylsulfonylpyridine-2-yl)-1-[2-(6-trifluoromethylbenzo[d]isoxazol-3-yl)phenyl]ethyl}-2-methylpropane-2-sulfinamide

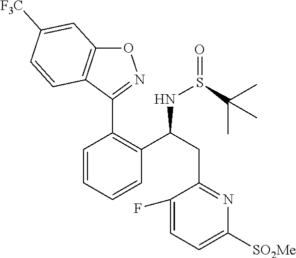 [SMILES: CC(C)(C)[S@](=O)N[C@@H](Cc1nc(S(C)(=O)=O)ccc1F)c1ccccc1-c1noc2cc(C(F)(F)F)ccc12]

A mixture of (S)—N—{(S)-2-(6-bromo-3-fluoropyridine-2-yl)-1-[2-(6-trifluoromethylbenzo[d]isoxazol-3-yl)phenyl]ethyl}-2-methylpropane-2-sulfinamide (Intermediate 71I, 0.15 g), potassium disulfite (0.11 g), palladium acetate (0.003 g), triphenylphosphine (0.01 g) sodium formate (0.038 g), 1,10-phenanthroline (0.007 g) and tetrabutylammonium bromide (0.091 g) was dissolved in degassed DMSO (1.5 mL) and sealed in a vial under argon then heated at 70° C. for 2 hours. After cooling, iodomethane (0.024 mL) was added and the mixture was stirred for 30 minutes. The mixture was diluted with ethyl acetate and washed with water, dried (MgSO₄) and filtered. The filtrate was concentrated in vacuo and the residue was purified by FCC eluting with 0-80% ethyl acetate in isohexane to give the title compound (0.089 g) as a pale yellow foam. ¹H NMR (400 MHz, CDCl₃) 7.98-7.92 (2H, m), 7.86-7.83 (1H, m), 7.73-7.58 (3H, m), 7.56-7.45 (3H, m), 5.33-5.27 (1H, m), 4.45-4.43 (1H, m), 3.53-3.45 (1H, m), 3.39-3.32 (1H, m), 3.07 (3H, s), 1.00-1.00 (9H, m).

By proceeding in a similar manner to Intermediate 77A, the following compounds were prepared:

### Intermediate 77B: (S)—N—{(S)-2-(3-Fluoro-6-methylsulfonylpyridine-2-yl)-1-[2-(6-methylbenzo[d]isoxazol-3-yl)phenyl]ethyl}-2-methylpropane-2-sulfinamide

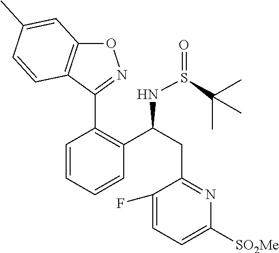 [SMILES: Cc1ccc2c(-c3ccccc3[C@H](Cc3nc(S(C)(=O)=O)ccc3F)N[S@@](=O)C(C)(C)C)noc2c1]

Starting from (S)—N—{(S)-2-(6-bromo-3-fluoropyridine-2-yl)-1-[2-(6-methylbenzo[d]isoxazol-3-yl)phenyl]ethyl}-2-methylpropane-2-sulfinamide (Intermediate 71J). LCMS (Method 3) RT 1.36 m/z 530 [MH⁺].

### Intermediate 77C: (S)—N—{(S)-1-[2-(6-chlorobenzo[d]isoxazol-3-yl)phenyl]-2-(3-fluoro-6-methylsulfonylpyridine-2-yl)ethyl}-2-methylpropane-2-sulfinamide

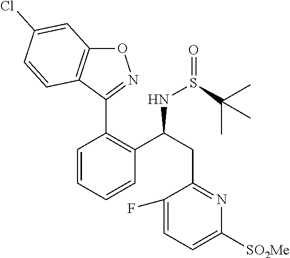 [SMILES: CC(C)(C)[S@](=O)N[C@@H](Cc1nc(S(C)(=O)=O)ccc1F)c1ccccc1-c1noc2cc(Cl)ccc12]

Starting from (S)—N—{(S)-2-(6-bromo-3-fluoropyridine-2-yl)-1-[2-(6-chlorobenzo[d]isoxazol-3-yl)phenyl]ethyl}-2-methylpropane-2-sulfinamide (Intermediate 71K). LCMS (Method 4) RT 1.69 m/z 550/552 [MH⁺].

### Intermediate 77D: (S)-2-Methyl-N—{(S)-2-(3-methyl-6-methylsulfonylpyridine-2-yl)-1-[2-(6-methylbenzo[d]isoxazol-3-yl)phenyl]ethyl}propane-2-sulfinamide

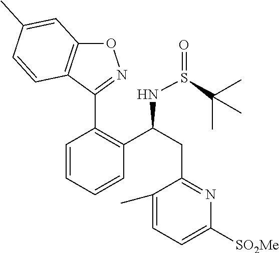 [SMILES: Cc1ccc2c(-c3ccccc3[C@H](Cc3nc(S(C)(=O)=O)ccc3C)N[S@@](=O)C(C)(C)C)noc2c1]

Starting from (S)—N—{(S)-2-(6-bromo-3-methylpyridine-2-yl)-1-[2-(6-methylbenzo[d]isoxazol-3-yl)phenyl]ethyl}-2-methylpropane-2-sulfinamide (Intermediate 6AD). LCMS (Method 3) RT 1.44 m/z 526 [MH⁺].

### Intermediate 77E: (S)—N—{(S)-2-(3-Fluoro-6-methylsulfonylpyridine-2-yl)-1-[2-(6-fluorobenzo[d]isoxazol-3-yl)phenyl]ethyl}-2-methylpropane-2-sulfinamide

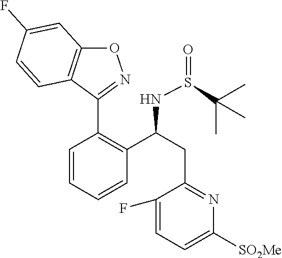 [SMILES: CC(C)(C)[S@](=O)N[C@@H](Cc1nc(S(C)(=O)=O)ccc1F)c1ccccc1-c1noc2cc(F)ccc12]

Starting from (S)—N—{(S)-2-(6-bromo-3-fluoropyridine-2-yl)-1-[2-(6-fluorobenzo[d]isoxazol-3-yl)phenyl]ethyl}-2-methylpropane-2-sulfinamide (Intermediate 71L). ¹H NMR (400 MHz, CDCl₃) 7.94-7.91 (1H, m), 7.70-7.62 (2H, m), 7.59-7.44 (4H, m), 7.37-7.34 (1H, m), 7.18-7.12 (1H, m), 5.29-5.24 (1H, m), 4.51-4.47 (1H, m), 3.52-3.45 (1H, m), 3.36-3.30 (1H, m), 3.08 (3H, s), 1.01 (9H, s).

### Intermediate 77F: (S)—N—{(S)-1-[2-(6-Bromobenzo[d]isoxazol-3-yl)phenyl]-2-(3-fluoro-6-methylsulfonylpyridine-2-yl)ethyl}-2-methylpropane-2-sulfinamide

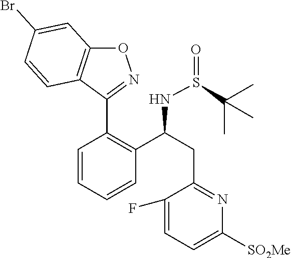 [SMILES: CC(C)(C)[S@](=O)N[C@@H](Cc1nc(S(C)(=O)=O)ccc1F)c1ccccc1-c1noc2cc(Br)ccc12]

Starting from (S)—N—{(S)-2-(6-bromo-3-fluoropyridine-2-yl)-1-[2-(6-bromobenzo[d]isoxazol-3-yl)phenyl]ethyl}-2-methylpropane-2-sulfinamide (Intermediate 71M). LCMS (Method 3) RT 1.40 m/z 594/596 [MH⁺].

### Intermediate 77G: (S)—N—{(S)-1-[2-(5-Fluorobenzo[d]isoxazol-3-yl)phenyl]-2-(3-fluoro-6-methylsulfonylpyridine-2-yl)ethyl}-2-methylpropane-2-sulfinamide

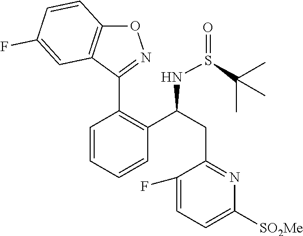 [SMILES: CC(C)(C)[S@](=O)N[C@@H](Cc1nc(S(C)(=O)=O)ccc1F)c1ccccc1-c1noc2ccc(F)cc12]

Starting from (S)—N—{(S)-2-[6-bromo-3-fluoro)pyridine-2-yl]-1-[2-(5-fluorobenzo[d]isoxazol-3-yl)phenyl]ethyl}-2-methylpropane-2-sulfinamide (Intermediate 710). ¹H NMR (300 MHz, CDCl₃) 7.93 (1H, dd, J=3.6, 8.5 Hz), 7.72-7.67 (1H, m), 7.67-7.43 (5H, m), 7.43-7.37 (1H, m), 7.37-7.30 (1H, m), 5.36-5.26 (1H, m), 4.59-4.53 (1H, m), 3.53-3.43 (1H, m), 3.39-3.29 (1H, m), 3.10 (3H, s), 1.01 (9H, s).

### Intermediate 77H: (S)—N—{(S)-1-[2-(6-Chlorobenzo[d]isoxazol-3-yl)phenyl]-2-(3-methyl-6-methylsulfonylpyridine-2-yl)ethyl}-2-methylpropane-2-sulfinamide

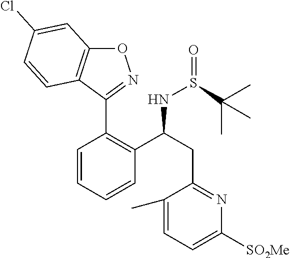 [SMILES: Cc1ccc(S(C)(=O)=O)nc1C[C@H](N[S@@](=O)C(C)(C)C)c1ccccc1-c1noc2cc(Cl)ccc12]

Starting from (S)—N—{(S)-2-[6-bromo-3-methylpyridine-2-yl]-1-[2-(6-chlorobenzo[d]isoxazol-3-yl)phenyl]ethyl}-]-2-methylpropane-2-sulfinamide (Intermediate 6AF). LCMS (Method 3) RT 1.47 m/z 546/548 [MH⁺].

### Intermediate 771: (S)—N—{(S)-1-[2-(6-Fluorobenzo[d]isoxazol-3-yl)phenyl]-2-(3-methyl-6-methylsulfonylpyridine-2-yl)ethyl}-2-methylpropane-2-sulfinamide

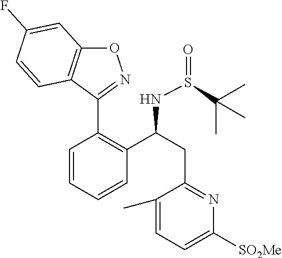 [SMILES: Cc1ccc(S(C)(=O)=O)nc1C[C@H](N[S@@](=O)C(C)(C)C)c1ccccc1-c1noc2cc(F)ccc12]

Starting from (S)—N—{(S)-2-[6-bromo-3-methylpyridine-2-yl]-1-[2-(6-fluorobenzo[d]isoxazol-3-yl)phenyl]ethyl}-]-2-methylpropane-2-sulfinamide (Intermediate 6AG). LCMS (Method 3) RT 1.42 m/z 530 [MH⁺].

### Intermediate 77J: (S)—N—{(S)-1-[2-(6-Chlorobenzo[d]isoxazol-3-yl)phenyl]-2-(3-fluoro-6-isopropylsulfonylpyridine-2-yl)ethyl}-2-methylpropane-2-sulfinamide

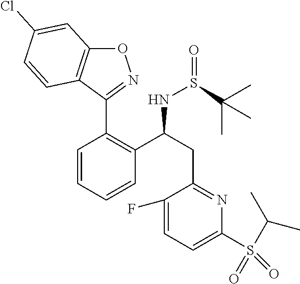 [SMILES: CC(C)S(=O)(=O)c1ccc(F)c(C[C@H](N[S@@](=O)C(C)(C)C)c2ccccc2-c2noc3cc(Cl)ccc23)n1]

Starting from (S)—N—{(S)-2-(6-bromo-3-fluoropyridine-2-yl)-1-[2-(6-chlorobenzo[d]isoxazol-3-yl)phenyl]ethyl}-2-methylpropane-2-sulfinamide (Intermediate 71K) and 2-iodopropane in place of iodomethane. LCMS (Method 3) RT 1.54 m/z 578 [MH⁺].

### Intermediate 77K: (S)—N—{(S)-1-[2-(7-Fluorobenzo[d]isoxazol-3-yl)phenyl]-2-(3-fluoro-6-methylsulfonylpyridine-2-yl)ethyl}-2-methylpropane-2-sulfinamide

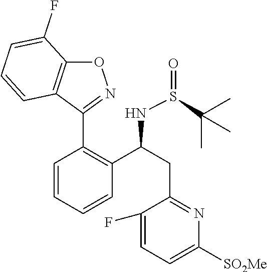 [SMILES: CC(C)(C)[S@](=O)N[C@@H](Cc1nc(S(C)(=O)=O)ccc1F)c1ccccc1-c1noc2c(F)cccc12]

Starting from (S)—N—{(S)-2-[6-bromo-3-fluoro)pyridine-2-yl]-1-[2-(7-fluorobenzo[d]isoxazol-3-yl)phenyl]ethyl}-2-methylpropane-2-sulfinamide (Intermediate 71P). LCMS (Method 3) RT 1.39 m/z 534 [MH⁺]

### Example 1: 1-[2-(Benzo[d]isoxazol-3-yl)phenyl]-2-(pyridin-2-yl)propan-1-amine hydrochloride (diastereomer 1)

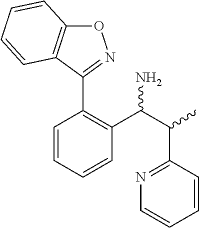 [SMILES: CC(c1ccccn1)C(N)c1ccccc1-c1noc2ccccc12]

A solution of hydrogen chloride in dioxane (4M, 1 mL) was added to a solution of diastereomer 1 of (S)—N-{1-[2-(benzo[d]isoxazol-3-yl)phenyl]-2-(pyridine-2-yl)propyl}-2-methylpropane-2-sulfinamide (Intermediate 29A, 0.031 g) in methanol (1 mL) and the resultant mixture was stirred for 2 hours. The mixture was concentrated in vacuo and the residue was dissolved in DCM and loaded onto an SCX-2 cartridge which was washed with DCM and then methanol. The product was isolated by elution with a solution of 2M ammonia in methanol. The resultant crude product was purified by FCC eluting with 2.5-5% of a mixture of 2M ammonia in methanol and DCM. After evaporation, the product was dissolved in acetonitrile and treated with 1M hydrochloric acid and the solution was freeze dried to give the title compound (0.012 g) as a white solid. ¹H NMR (400 MHz, DMSO-d₆) 8.54 (3H, br s), 8.48 (1H, d, J=4.88 Hz), 8.07 (1H, d, J=8.14 Hz), 7.9-7.86 (1H, m), 7.86-7.8 (1H, m), 7.79-7.63 (5H, m), 7.49-7.42 (2H, m), 7.33-7.27 (1H, m), 5.12-5.05 (1H, m), 3.69-3.61 (1H, m), 0.90 (3H, d, J=7.05 Hz). LCMS (Method 1) RT 2.79 m/z 330 [MH⁺].

By proceeding in a similar manner to Example 1, the following compounds were prepared.

### Example 2: 1-[2-(Benzo[d]isoxazol-3-yl)phenyl]-2-(pyridin-2-yl)propan-1-amine hydrochloride (diastereomer 2)

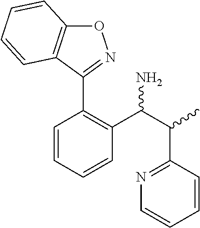 [SMILES: CC(c1ccccn1)C(N)c1ccccc1-c1noc2ccccc12]

Starting from diastereomer 2 of (S)—N-{1-[2-(benzo[d]isoxazol-3-yl)phenyl]-2-(pyridine-2-yl)propyl}-2-methylpropane-2-sulfinamide (Intermediate 29B). ¹H NMR (400 MHz, DMSO-d₆) 8.95 (3H, br s), 8.04 (1H, d, J=7.54), 7.85 (1H, d, J=8.55), 7.79-7.62 (4H, m), 7.53-7.35 (4H, m), 7.32 (1H, d, J=8.04), 6.99-6.84 (1H, m), 5.12-5.04 (1H, m), 3.39-3.32 (1H, m), 1.31 (3H, d, J=6.70). LCMS (Method 1) RT 2.79 m/z 330 [MH⁺].

### Example 3: 1-[2-(Benzo[d]isoxazol-3-yl)phenyl]-2-(pyridin-2-yl)propan-1-amine hydrochloride (diastereomer 3)

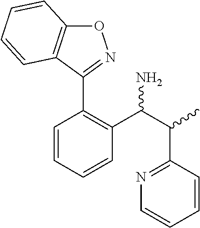 [SMILES: CC(c1ccccn1)C(N)c1ccccc1-c1noc2ccccc12]

Starting from diastereomer 3 of (S)—N-{1-[2-(benzo[d]isoxazol-3-yl)phenyl]-2-(pyridine-2-yl)propyl}-2-methylpropane-2-sulfinamide (intermediate 29C). ¹H NMR (400 MHz, DMSO-d₆) 8.69 (3H, br s), 8.54-8.50 (1H, m), 8.17 (1H, d, J=7.81), 7.98-7.90 (1H, m), 7.88 (1H, d, J=8.35), 7.80-7.63 (5H, m), 7.62-7.55 (1H, m), 7.48-7.32 (2H, m), 5.11-5.04 (1H, m), 3.81-3.73 (1H, m), 0.92 (3H, d, J=7.13 Hz). LCMS (Method 1) RT 2.82 m/z 330 [MH⁺].

### Example 4: 1-[2-(Benzo[d]isoxazol-3-yl)phenyl]-1-(pyridin-2-yl)propan-2-amine

hydrochloride

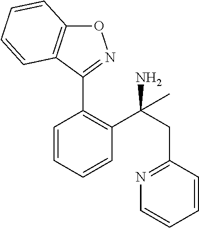 [SMILES: C[C@](N)(Cc1ccccn1)c1ccccc1-c1noc2ccccc12]

Starting from (S)—N-[2-(benzo[d]isoxazol-3-yl)phenyl]-1-(pyridin-2-yl)propan-2-yl]-2-methylpropan-2-sulfinamide (Intermediate 5G). ¹H NMR (400 MHz, DMSO-d₆) 9.09 (3H, br s), 8.57-8.52 (1H, m), 7.88 (1H, d, J=8.64 Hz), 7.81 (1H, t, J=7.34 Hz), 7.77-7.71 (2H, m), 7.61-7.56 (1H, m), 7.53-7.48 (1H, m), 7.46-7.36 (4H, m), 7.01 (1H, d, J=7.74), 3.65-3.54 (2H, m), 1.37 (3H, s). LCMS (Method 1) RT 3.07 m/z 330 [MH⁺].

### Example 5: (S)-1-[2-(Benzo[d]isoxazol-3-yl)phenyl]-2-[3-fluoro-6-(methylsulfonyl)pyridine-2-yl]ethan-1-amine hydrochloride

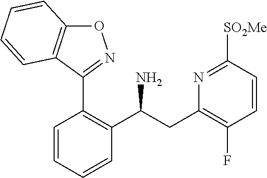 [SMILES: CS(=O)(=O)c1ccc(F)c(C[C@H](N)c2ccccc2-c2noc3ccccc23)n1]

Starting from (S)—N—{(S)-1-[2-(benzo[d]isoxazol-3-yl)phenyl]-2-[fluoro-6-(methyysulfonyl)pyridine-2-yl]ethyl}-2-methylpropane-2-sulfinamide (Intermediate 56B) and isolating the HCl salt by filtration from the reaction mixture and washing with ethyl acetate.

¹H NMR (400 MHz, DMSO-d₆) 8.98 (3H, br s), 8.26-8.23 (1H, m), 7.87-7.84 (1H, m), 7.82-7.70 (4H, m), 7.68-7.62 (3H, m), 7.46-7.42 (1H, m), 5.20-5.14 (1H, m), 3.67-3.56 (1H, m), 3.52-3.42 (1H, m), 2.92 (3H, s). LCMS (Method 1) RT 2.82 m/z 412 [MH⁺]

### Example 6: (S)-1-[2-(Benzo[d]isoxazol-3-yl)phenyl]-2-(6-cyano-5-fluoropyridine-2-yl)ethan-1-amine hydrochloride

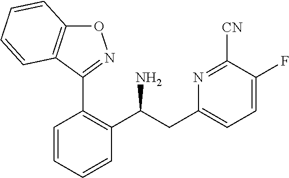 [SMILES: N#Cc1nc(C[C@H](N)c2ccccc2-c2noc3ccccc23)ccc1F]

Starting from (S)—N—{(S)-1-[2-(benzo[d]isoxazol-3-yl)phenyl]ethyl}-2-(6-cyano-5-fluoropyridine-2-yl)-2-methylpropane-2-sulfinamide (Intermediate 27N) isolated directly as the HCl salt by filtration. ¹H NMR (400 MHz, DMSO-d₆) 8.88 (3H, br s), 8.17-8.14 (1H, m), 7.89-7.85 (1H, m), 7.83-7.74 (3H, m), 7.67-7.58 (2H, m), 7.51-7.41 (2H, m), 7.33 (1H, dd, J=4.2, 8.9 Hz), 5.14 (1H, dd, J=5.9, 8.7 Hz), 3.47-3.35 (2H, m). LCMS (Method 1) RT 3.06 m/z 359 [MH⁺].

### Example 7: (S)-1-[2-(Benzo[d]isoxazol-3-yl)phenyl]-2-(5-fluoro-6-methylsulfonylpyridine-2-yl)ethan-1-amine hydrochloride

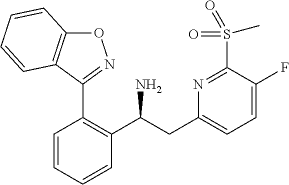 [SMILES: CS(=O)(=O)c1nc(C[C@H](N)c2ccccc2-c2noc3ccccc23)ccc1F]

Starting from (S)—N—{(S)-1-[2-(benzo[d]isoxazol-3-yl)phenyl]-2-[5luoro-6-(methylsulfonyl)pyridine-2-yl]ethyl}-2-methylpropane-2-sulfinamide (Intermediate 56C) and converting to the HCL salt by dissolving in dioxane and treating with 4M HCl in dioxane followed by concentration in vacuo. ¹H NMR (400 MHz, DMSO-d₆) 8.70 (3H, s), 8.15-8.12 (1H, m), 7.89-7.75 (4H, m), 7.74-7.64 (3H, m), 7.51-7.44 (2H, m), 5.15-5.11 (1H, m), 3.54 (1H, dd, J=7.8, 14.2 Hz), 3.41-3.35 (1H, m), 3.11 (3H, s). LCMS (Method 1) RT 2.89 m/z 412 [MH⁺].

### Example 8: (S)-6-{2-Amino-2-[2-(benzo[d]isoxazol-3-yl)phenyl]ethyl}-N,N,5-trimethylpyridine-2-carboxamide

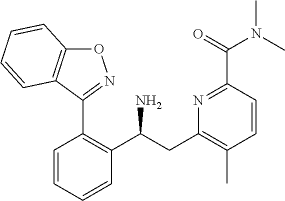 [SMILES: Cc1ccc(C(=O)N(C)C)nc1C[C@H](N)c1ccccc1-c1noc2ccccc12]

Starting from 6-{(S)-2-[2-(benzo[d]isoxazol-3-yl)phenyl]-2-[((S)-tert-butylsulfinyl)amino]ethyl}-N,N,5-trimethylpyridine-2-carboxamide (Intermediate 76A) and converting to the HCl salt by dissolving in dioxane and treating with 4M HCl in dioxane, dilution with water and freeze drying. ¹H NMR (400 MHz, DMSO-d₆) 8.68 (3H, br s), 8.16-8.12 (1H, m), 7.88-7.85 (1H, m), 7.80-7.61 (5H, m), 7.43 (2H, dd, J=7.7, 14.8 Hz), 7.20-7.17 (1H, m), 5.31-5.26 (1H, m), 3.30 (1H, dd, J=7.3, 15.1 Hz), 2.84 (3H, s), 2.52 (3H, s), 2.08 (3H, s) plus one proton hidden under the water. LCMS (Method 1) RT 3.02 m/z 401 [MH⁺].

### Example 9: (S)-1-[2-(Benzo[d]isoxazol-3-yl)phenyl]-2-(6-ethyl-3-methylpyridine-2-yl)ethan-1-amine hydrochloride

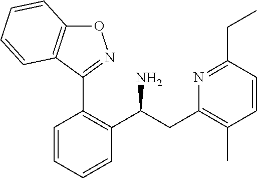 [SMILES: CCc1ccc(C)c(C[C@H](N)c2ccccc2-c2noc3ccccc23)n1]

Starting from (S)—N—{(S)-1-[2-(benzo[d]isoxazol-3-yl)phenyl]-2-(6-ethyl-3-methylpyridin-2-yl)ethyl}-2-methylpropane-2-sulfinamide (Intermediate 35E) and converting to the HCl salt by dissolving in dioxane and treating with 4M HCl in dioxane, dilution with water and freeze drying. ¹H NMR (400 MHz, DMSO-d₆+TFA-D) 8.27 (1H, d, J=8.0 Hz), 7.92 (1H, d, J=8.0 Hz), 7.90-7.84 (2H, m), 7.77 (1H, t, J=7.9 Hz), 7.69 (1H, t, J=8.1 Hz), 7.61 (1H, d, J=8.0 Hz), 7.53 (1H, d, J=8.1 Hz), 7.49-7.40 (2H, m), 5.32 (1H, m), 3.75 (1H, m), 3.65-3.55 (1H, m), 2.79-2.64 (2H, m), 2.00 (3H, s), 1.10 (3H, t, J=7.2 Hz). LCMS (Method 1) RT 2.72 m/z 358 [MH⁺].

### Example 10: (S)-2-(3-Fluoro-6-methylsulfonylpyridine-2-yl)-1-[2-(6-trifluoromethylbenzo[d]isoxazol-3-yl)phenyl]ethan-1-amine hydrochloride

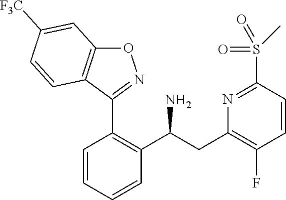 [SMILES: CS(=O)(=O)c1ccc(F)c(C[C@H](N)c2ccccc2-c2noc3cc(C(F)(F)F)ccc23)n1]

Starting from (S)—N—{(S)-2-(3-fluoro-6-methylsulfonylpyridine-2-yl)-1-[2-(6-trifluoromethylbenzo[d]isoxazol-3-yl)phenyl]ethyl}-2-methylpropane-2-sulfinamide (Intermediate 77A) and converting to the HCl salt by treating with 0.1M aqueous HCl and collecting the precipitated solid by filtration and drying in vacuo. ¹H NMR (400 MHz, DMSO-d₆) 8.70 (3H, br s), 8.40 (1H, s), 8.21-8.18 (1H, m), 7.90-7.75 (5H, m), 7.69-7.65 (2H, m), 5.17-5.11 (1H, m), 3.62-3.57 (1H, m), 3.52-3.44 (1H, m), 2.96-2.95 (3H, m). LCMS (Method 1) RT 3.29 m/z 480 [MH⁺].

### Example 11: (S)-2-(3-Fluoro-6-methylsulfonylpyridine-2-yl)-1-[2-(6-methylbenzo[d]isoxazol-3-yl)phenyl]ethan-1-amine hydrochloride

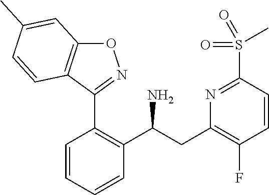

Starting from (S)—N—{(S)-2-(3-fluoro-6-methylsulfonylpyridine-2-yl)-1-[2-(6-methylbenzo[d]isoxazol-3-yl)phenyl]ethyl}-2-methylpropane-2-sulfinamide (Intermediate 77B) and converting to the HCl salt by dissolving in acetonitrile and treating with 0.1M hydrochloric acid and freeze drying. ¹H NMR (400 MHz, DMSO-d₆) 8.77-8.71 (3H, br s), 8.14-8.10 (1H, m), 7.84-7.73 (3H, m), 7.66-7.61 (3H, m), 7.53-7.49 (1H, m), 7.29-7.24 (1H, m), 5.21-5.16 (1H, m), 3.60-3.55 (1H, m), 3.49-3.41 (1H, m), 2.91 (3H, s), 2.53 (3H, s). LCMS (Method 1) RT 3.00 m/z 426 [MH⁺].

### Example 12: (S)-2-(6-Cyano-3-fluoropyridine-2-yl)-1-[2-(6-methylbenzo[d]isoxazol-3-yl)phenyl]ethan-1-amine hydrochloride

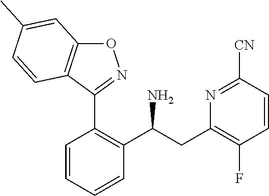 [SMILES: Cc1ccc2c(-c3ccccc3[C@@H](N)Cc3nc(C#N)ccc3F)noc2c1]

Starting from (S)—N—{(S)-2-(6-cyano-3-fluoropyridine-2-yl)-1-[2-(6-methylbenzo[d]isoxazol-3-yl)phenyl]ethyl}-2-methylpropane-2-sulfinamide (Intermediate 270) and converting to the HCl salt by dissolving in acetonitrile, treating with 0.1M aqueous HCl and freeze drying. ¹H NMR (400 MHz, DMSO-d₆) 8.74 (3H, br s), 8.11-8.07 (1H, m), 7.81-7.57 (6H, m), 7.40-7.37 (1H, m), 7.27-7.25 (1H, m), 5.21-5.15 (1H, m), 3.48-3.36 (2H, m), 2.54 (3H, s). LCMS (Method 1) RT 3.09 m/z 373 [MH⁺].

### Example 13: (S)-1-[2-(6-Chlorobenzo[d]isoxazol-3-yl)phenyl]-2-(3-fluoro-6-methylsulfonylpyridine-2-yl)ethan-1-amine hydrochloride

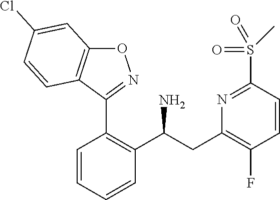 [SMILES: CS(=O)(=O)c1ccc(F)c(C[C@H](N)c2ccccc2-c2noc3cc(Cl)ccc23)n1]

Starting from (S)—N—{(S)-1-[2-(6-chlorobenzo[d]isoxazol-3-yl)phenyl]-2-(3-fluoro-6-methylsulfonylpyridine-2-yl)-ethyl}-2-methylpropane-2-sulfinamide (Intermediate 77C) and converting to the HCl salt by dissolving in acetonitrile and treating with 0.1M hydrochloric acid and freeze drying. ¹H NMR (400 MHz, DMSO-d₆) 8.77 (3H, br s), 8.19-8.14 (1H, m), 8.12-8.09 (1H, m), 7.85-7.77 (3H, m), 7.67-7.63 (3H, m), 7.49 (1H, dd, J=1.7, 8.5 Hz), 5.20-5.13 (1H, m), 3.64-3.56 (1H, m), 3.51-3.42 (1H, m), 2.95 (3H, s). LCMS (Method 1) RT 3.06 m/z 446 [MH⁺].

### Example 14: (S)-1-[2-(6-Chlorobenzo[d]isoxazol-3-yl)phenyl]-2-(6-cyano-3-fluoropyridine-2-yl) ethan-1-amine hydrochloride

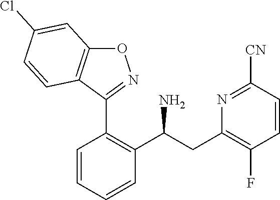 [SMILES: N#Cc1ccc(F)c(C[C@H](N)c2ccccc2-c2noc3cc(Cl)ccc23)n1]

Starting from (S)—N—{(S)-1-[2-(6-chlorobenzo[d]isoxazol-3-yl)phenyl]-2-(6-cyano-3-fluoropyridine-2-yl)ethyl}-2-methylpropane-2-sulfinamide (Intermediate 27P) and converting to the HCl salt by dissolving in acetonitrile and treating with 0.1M hydrochloric acid and freeze drying. ¹H NMR (400 MHz, DMSO-d₆) 8.82 (3H, br s), 8.18-8.13 (1H, m), 8.13-8.10 (1H, m), 7.84-7.71 (3H, m), 7.65-7.47 (4H, m), 5.21-5.15 (1H, m), 3.53-3.37 (2H, m). LCMS (Method 1) RT 3.19 m/z 393 [MH⁺].

### Example 15: (S)-2-(6-Bromo-3-fluoropyridine-2-yl)-1-[2-(6-fluorobenzo[d]isoxazol-3-yl)phenyl]-ethan-1-amine hydrochloride

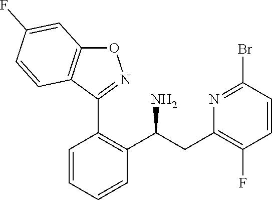 [SMILES: N[C@@H](Cc1nc(Br)ccc1F)c1ccccc1-c1noc2cc(F)ccc12]

Starting from (S)—N—{(S)-2-(6-bromo-3-fluoropyridine-2-yl)-1-[2-(6-fluorobenzo[d]isoxazol-3-yl)phenyl]ethyl}-2-methylpropane-2-sulfinamide (Intermediate 71L) and converting to the HCl salt by dissolving in ethyl acetate and adding HCl in ethyl acetate and evaporating. The product was then dissolved in water and freeze dried. ¹H NMR (400 MHz, DMSO-d₆) 8.69 (3H, br s), 8.15-8.11 (1H, m), 7.86 (1H, dd, J=2.1, 8.9 Hz), 7.80-7.74 (1H, m), 7.64-7.55 (2H, m), 7.52 (1H, dd, J=5.3, 8.9 Hz), 7.39 (1H, t, J=8.8 Hz), 7.32 (1H, dt, J=2.3, 9.3 Hz), 7.25 (1H, dd, J=3.6, 8.5 Hz), 5.17 (1H, m), 3.41-3.35 (1H, m), 3.30-3.25 (1H, m). LCMS (Method 1) RT 3.13 m/z 430/432 [MH⁺].

### Example 16: (S)-2-(6-Bromo-3-fluoropyridine-2-yl)-1-[2-(6-bromobenzo[d]isoxazol-3-yl)phenyl]-ethan-1-amine

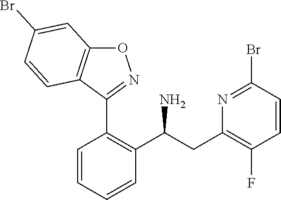 [SMILES: N[C@@H](Cc1nc(Br)ccc1F)c1ccccc1-c1noc2cc(Br)ccc12]

Starting from (S)—N—{(S)-2-(6-bromo-3-fluoropyridine-2-yl)-1-[2-(6-bromobenzo[d]isoxazol-3-yl)phenyl]ethyl}-2-methylpropane-2-sulfinamide (Intermediate 71M) but using HCl in ethyl acetate in place of HCl in dioxane. The product was isolated as the free base. ¹H NMR (400 MHz, CDCl₃) 7.87-7.85 (1H, m), 7.85-7.81 (1H, m), 7.60-7.55 (1H, m), 7.47 (1H, dd, J=1.6, 8.4 Hz), 7.44-7.38 (3H, m), 7.19 (1H, dd, J=3.5, 8.7 Hz), 7.08 (1H, t, J=8.5 Hz), 4.76-4.71 (1H, m), 3.27-3.20 (1H, m), 3.12-3.04 (1H, m). LCMS (Method 1) RT 3.43 m/z 490/492/494 [MH⁺].

### Example 17: (S)-2-(6-Bromo-3-fluoropyridine-2-yl)-1-[2-(6-cyanobenzo[d]isoxazol-3-yl)phenyl]-ethan-1-amine hydrochloride

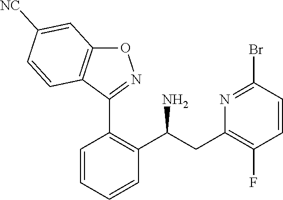 [SMILES: N#Cc1ccc2c(-c3ccccc3[C@@H](N)Cc3nc(Br)ccc3F)noc2c1]

Starting from (S)—N—{(S)-2-[6-bromo-3-fluoro)pyridine-2-yl]-1-[2-(6-cyanobenzo[d]isoxazol-3-yl)phenyl]ethyl}-2-methylpropane-2-sulfinamide (Intermediate 71N) and converting to the HCl salt by dissolving in acetonitrile and adding 0.1M HCl and freeze drying. ¹H NMR (400 MHz, DMSO-d₆) 8.99-8.89 (3H, br s), 8.61 (1H, s), 8.21-8.17 (1H, m), 7.85-7.77 (2H, m), 7.70-7.58 (3H, m), 7.41-7.36 (1H, m), 7.23 (1H, dd, J=3.5, 8.6 Hz), 5.20-5.14 (1H, m), 3.45-3.36 (1H, m), 3.30-3.26 (1H, m). LCMS (Method 1) RT 2.98 m/z 437/439 [MH⁺].

### Example 18: (S)-6-{2-Amino-2-[2-(6-methylbenzo[d]isoxazol-3-yl)phenyl]ethyl-2-cyano-5-methylpyridine hydrochloride

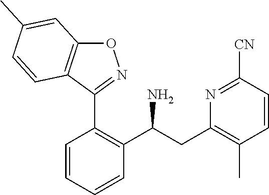 [SMILES: Cc1ccc2c(-c3ccccc3[C@@H](N)Cc3nc(C#N)ccc3C)noc2c1]

Starting from (S)—N—{(S)-2-(6-cyano-3-methylpyridine-2-yl)-1-[2-(6-methylbenzo[d]isoxazol-3-yl)phenyl]ethyl}-2-methylpropane-2-sulfinamide (Intermediate 27R) and converting to the HCl salt by dissolving in acetonitrile and treating with 0.1M HCl and freeze drying. ¹H NMR (400 MHz, DMSO-d₆) 8.72 (3H, s), 8.16-8.13 (1H, m), 7.80-7.74 (1H, m), 7.65-7.54 (4H, m), 7.49-7.40 (2H, m), 7.26-7.24 (1H, m), 5.31-5.25 (1H, m), 3.43-3.35 (2H, m), 2.54 (3H, s), 2.00 (3H, s). LCMS (Method 1) RT 3.47 m/z 369 [MH⁺].

### Example 19: (S)-2-(3-Methyl-6-methylsulfonylpyridin-2-yl)-1-[2-(6-methylbenzo[d]isoxazol-3-yl)phenyl]ethan-1-amine hydrochloride

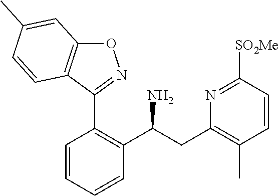 [SMILES: Cc1ccc2c(-c3ccccc3[C@@H](N)Cc3nc(S(C)(=O)=O)ccc3C)noc2c1]

Starting from (S)-2-methyl-N—{(S)-2-(3-methyl-6-methylsulfonylpyridine-2-yl)-1-[2-(6-methylbenzo[d]isoxazol-3-yl)phenyl]ethyl}propane-2-sulfinamide (Intermediate 77D) and converting to the HCl salt by dissolving in acetonitrile and treating with 0.1M HCl and freeze drying. ¹H NMR (400 MHz, DMSO-d₆) 8.71 (3H, br s), 8.18-8.14 (1H, m), 7.81-7.76 (1H, m), 7.67-7.61 (4H, m), 7.59-7.54 (1H, m), 7.52-7.48 (1H, m), 7.27-7.25 (1H, m), 5.32-5.26 (1H, m), 3.62-3.54 (1H, m), 3.38-3.34 (1H, m), 2.86 (3H, s), 2.53 (3H, s), 2.13 (3H, s). LCMS (Method 1) RT 3.34 m/z 422 [MH⁺].

### Example 20: (S)-2-(3-Fluoro-6-methylsulfonylpyridin-2-yl)-1-[2-(6-fluorobenzo[d]isoxazol-3-yl)phenyl]ethan-1-amine hydrochloride

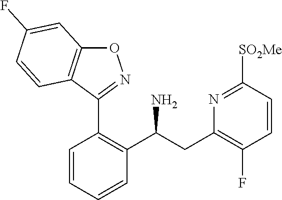 [SMILES: CS(=O)(=O)c1ccc(F)c(C[C@H](N)c2ccccc2-c2noc3cc(F)ccc23)n1]

Starting from (S)—N—{(S)-2-(3-fluoro-6-methylsulfonylpyridine-2-yl)-1-[2-(6-fluorobenzo[d]isoxazol-3-yl)phenyl]ethyl}-2-methylpropane-2-sulfinamide (Intermediate 77E) and converting to the HCl salt by dissolving in ethyl acetate and adding 1M HCl in ethyl acetate. The resultant solid was collected by filtration. ¹H NMR (400 MHz, DMSO-d₆) 8.78 (3H, s), 8.19-8.15 (1H, m), 7.86-7.75 (4H, m), 7.68-7.62 (3H, m), 7.36-7.30 (1H, m), 5.20-5.14 (1H, m), 3.62-3.56 (1H, m), 3.50-3.41 (1H, m), 2.95-2.94 (3H, m). LCMS (Method 1) RT 3.09 m/z 430 [MH⁺].

### Example 21: (S)-1-[2-(6-Bromobenzo[d]isoxazol-3-yl)phenyl]-2-(3-Fluoro-6-methylsulfonylpyridin-yl)ethan-1-amine hydrochloride

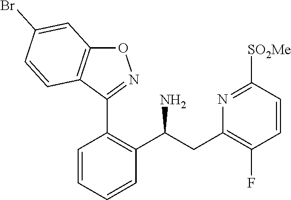 [SMILES: CS(=O)(=O)c1ccc(F)c(C[C@H](N)c2ccccc2-c2noc3cc(Br)ccc23)n1]

Starting from (S)—N—{(S)-1-[2-(6-bromobenzo[d]isoxazol-3-yl)phenyl]-2-(3-fluoro-6-methylsulfonylpyridine-2-yl)ethyl}-2-methylpropane-2-sulfinamide (Intermediate 77F) but using HCl in ethyl acetate in place of HCl in dioxane and converting to the HCl salt by dissolving in acetonitrile and adding HCl in ethyl acetate then collecting the product by filtration. ¹H NMR (400 MHz, DMSO-d₆) 8.73 (3H, br s), 8.25 (1H, s), 8.19-8.15 (1H, m), 7.84-7.77 (3H, m), 7.65-7.59 (4H, m), 5.18-5.12 (1H, m), 3.62-3.56 (1H, m), 3.50-3.39 (1H, m), 2.95 (3H, s). LCMS (Method 1) RT 3.32 m/z 490/492 [MH⁺].

### Example 22: (S)-1-[2-(5-Fluorobenzo[d]isoxazol-3-yl)phenyl]-2-(3-fluoro-6-methylsulfonylpyridin-2-yl)ethan-1-amine hydrochloride

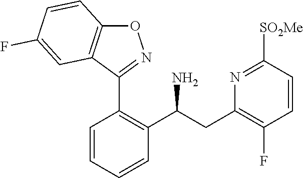 [SMILES: CS(=O)(=O)c1ccc(F)c(C[C@H](N)c2ccccc2-c2noc3ccc(F)cc23)n1]

Starting from (S)—N—{(S)-1-[2-(5-fluorobenzo[d]isoxazol-3-yl)phenyl]-2-(3-fluoro-6-methylsulfonylpyridine-2-yl)ethyl}-2-methylpropane-2-sulfinamide (Intermediate 77G) but using HCl in ethyl acetate in place of HCl in dioxane and collecting the product by filtration directly from the reaction mixture. ¹H NMR (400 MHz, DMSO-d₆) 8.73 (3H, br s), 8.15-8.12 (1H, m), 7.93 (1H, dd, J=3.8, 9.2 Hz), 7.86-7.76 (3H, m), 7.68-7.62 (3H, m), 7.48 (1H, dd, J=2.5, 8.0 Hz), 5.18-5.12 (1H, m), 3.60-3.55 (1H, m), 3.48-3.40 (1H, m), 2.94 (3H, s). LCMS (Method 1) RT 3.11 m/z 430 [MH⁺].

### Example 23: (S)-1-[2-(6-Chlorobenzo[d]isoxazol-3-yl)phenyl]-2-(3-methyl-6-methylsulfonylpyridin-2-yl)ethan-1-amine hydrochloride

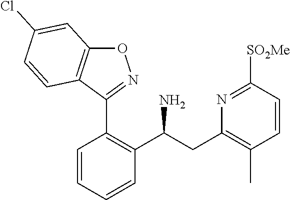 [SMILES: Cc1ccc(S(C)(=O)=O)nc1C[C@H](N)c1ccccc1-c1noc2cc(Cl)ccc12]

Starting from (S)—N—{(S)-1-[2-(6-chlorobenzo[d]isoxazol-3-yl)phenyl]-2-(3-methyl-6-methylsulfonylpyridine-2-yl)ethyl}-2-methylpropane-2-sulfinamide (Intermediate 77H) but using HCl in ethyl acetate in place of HCl in dioxane and collecting the product by filtration directly from the reaction mixture. ¹H NMR (400 MHz, DMSO-d₆) 8.72 (3H, br s), 8.18-8.15 (1H, m), 8.10 (1H, d, J=1.4 Hz), 7.84-7.78 (1H, m), 7.67-7.61 (4H, m), 7.58-7.55 (1H, m), 7.48 (1H, dd, J=1.7, 8.5 Hz), 5.31-5.26 (1H, m), 3.62-3.55 (1H, m), 3.39-3.36 (1H, m), 2.89 (3H, s), 2.14 (3H, s). LCMS (Method 1) RT 3.36 m/z 442 [MH⁺].

### Example 24: (S)-1-[2-(6-Fluorobenzo[d]isoxazol-3-yl)phenyl]-2-(3-methyl-6-methylsulfonylpyridin-2-yl)ethan-1-amine hydrochloride

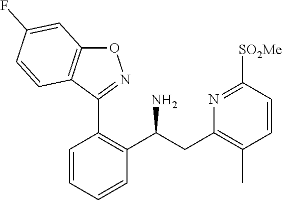 [SMILES: Cc1ccc(S(C)(=O)=O)nc1C[C@H](N)c1ccccc1-c1noc2cc(F)ccc12]

Starting from (S)—N—{(S)-1-[2-(6-fluorobenzo[d]isoxazol-3-yl)phenyl]-2-(3-methyl-6-methylsulfonylpyridine-2-yl)ethyl}-2-methylpropane-2-sulfinamide (Intermediate 771) but using HCl in ethyl acetate in place of HCl in dioxane and collecting the product by filtration directly from the reaction mixture. ¹H NMR (400 MHz, DMSO-d₆) 8.75 (3H, br s), 8.18-8.15 (1H, m), 7.86-7.78 (2H, m), 7.69-7.60 (4H, m), 7.58-7.54 (1H, m), 7.36-7.30 (1H, m), 5.30-5.29 (1H, m), 3.62-3.55 (1H, m), 3.39-3.35 (1H, m), 2.89 (3H, s), 2.13 (3H, s). LCMS (Method 1) RT 3.17 m/z 426 [MH⁺].

### Example 25: (S)-1-[2-(6-Chlorobenzo[d]isoxazol-3-yl)phenyl]-2-(3-fluoro-6-isopropyisulfonylpyridin-2-yl)ethan-1-amine hydrochloride

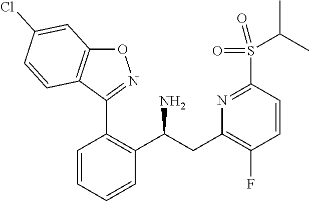 [SMILES: CC(C)S(=O)(=O)c1ccc(F)c(C[C@H](N)c2ccccc2-c2noc3cc(Cl)ccc23)n1]

Starting from (S)—N—{(S)-1-[2-(6-chlorobenzo[d]isoxazol-3-yl)phenyl]-2-(3-fluoro-6-isopropylsulfonylpyridine-2-yl)ethyl}-2-methylpropane-2-sulfinamide (Intermediate 77J) and converting to the HCl salt by dissolving in acetonitrile and adding 0.1M aqueous HCL then freeze drying. ¹H NMR (400 MHz, DMSO-d₆) 8.82 (3H, br s), 8.20-8.16 (1H, m), 8.14-8.11 (1H, m), 7.88-7.77 (3H, m), 7.68-7.63 (3H, m), 7.51 (1H, dd, J=1.7, 8.5 Hz), 5.12-5.12 (1H, m), 3.70-3.65 (1H, m), 3.53-3.44 (1H, m), 3.17-3.06 (1H, m), 1.00 (3H, d, J=6.9 Hz), 0.87 (3H, d, J=6.7 Hz). LCMS (Method 1) RT 3.51 m/z 474 [MH⁺].

### Example 26: (S)-1-[2-(7-Fluorobenzo[d]isoxazol-3-yl)phenyl]-2-(3-fluoro-6-methylsulfonylpyridin-2-yl)ethan-1-amine hydrochloride

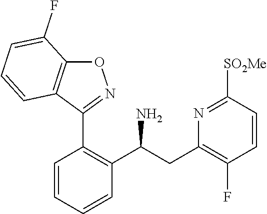 [SMILES: CS(=O)(=O)c1ccc(F)c(C[C@H](N)c2ccccc2-c2noc3c(F)cccc23)n1]

Starting from (S)—N—{(S)-1-[2-(7-fluorobenzo[d]isoxazol-3-yl)phenyl]-2-(3-fluoro-6-methylsulfonylpyridine-2-yl)ethyl}-2-methylpropane-2-sulfinamide (Intermediate 77K) but using HCl in ethyl acetate in place of HCl in dioxane and converting to the HCl salt by dissolving in acetonitrile and treating with 0.1M aqueous HCl and freeze drying. ¹H NMR (400 MHz, DMSO-d₆) 8.80 (3H, br s), 8.20-8.17 (1H, m), 7.81-7.78 (3H, m), 7.68-7.64 (3H, m), 7.47-7.42 (21H, m), 5.18-5.13 (1H, m), 3.61-3.56 (1H, m), 3.49-3.37 (1H, m), 2.95 (3H, s). LCMS (Method 1) RT 3.06 m/z 430 [MH⁺].

### Example 27: (S)-2-{2-Amino-2-[2-(6-bromobenzo[d]isoxazol-3-yl)phenyl]ethyl}-3-fluoropyridine-6-carbonitrile

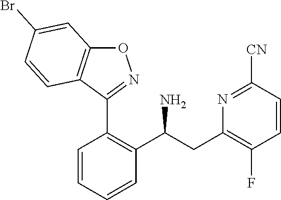 [SMILES: N#Cc1ccc(F)c(C[C@H](N)c2ccccc2-c2noc3cc(Br)ccc23)n1]

Starting from (S)—N—{(S)-1-[2-(6-bromobenzo[d]isoxazol-3-yl)phenyl]-2-(6-cyano-3-fluoropyridine-2-yl)ethyl}-2-methylpropane-2-sulfinamide (Intermediate 27S) but using HCl in ethyl acetate in place of HCl in dioxane and isolating the product as the free base. ¹H NMR (400 MHz, DMSO-d₆) 8.21 (1H, d, J=1.0 Hz), 7.99 (1H, d, J=7.5 Hz), 7.81 (1H, dd, J=3.7, 8.5 Hz), 7.71-7.52 (4H, m), 7.45-7.34 (2H, m), 4.56-4.50 (1H, m), 3.09-2.96 (2H, m), 2.19 (2H, br s). LCMS (Method 1) RT 3.45 m/z 437 [MH⁺].

### Example 28: (S)-2-{2-Amino-2-[2-(6-bromobenzo[d]isoxazol-3-yl)phenyl]ethyl}-3-methylpyridine-6-carbonitrile

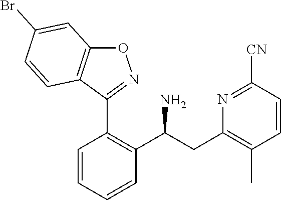 [SMILES: Cc1ccc(C#N)nc1C[C@H](N)c1ccccc1-c1noc2cc(Br)ccc12]

Starting from (S)—N—{(S)-1-[2-(6-bromobenzo[d]isoxazol-3-yl)phenyl]-2-(6-cyano-3-methylpyridine-2-yl)ethyl}-2-methylpropane-2-sulfinamide (Intermediate 27T) but using HCl in ethyl acetate in place of HCl in dioxane and isolating the product as the free base. ¹H NMR (400 MHz, DMSO-d₆) 8.17 (1H, d, J=1.1 Hz), 8.04 (1H, d, J=7.1 Hz), 7.64 (1H, dt, J=1.3, 7.6 Hz), 7.57-7.46 (4H, m), 7.42 (1H, dt, J=1.1, 7.5 Hz), 7.35 (1H, dd, J=1.5, 7.7 Hz), 4.65-4.60 (1H, m), 2.99-2.95 (2H, m), 2.13 (2H, br s), 1.92 (3H, s). LCMS (Method 2) RT 3.33 m/z 433 [MH⁺].

### Example 29: (S)-1-[2-(6-Cyanobenzo[d]isoxazol-3-yl)phenyl]-2-(pyridine-2-yl)ethan-1-amine

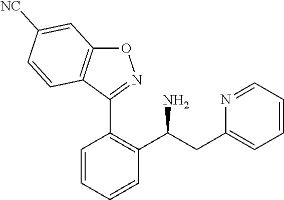 [SMILES: N#Cc1ccc2c(-c3ccccc3[C@@H](N)Cc3ccccn3)noc2c1]

A solution of tert-butyl (S)-{1-[2-(6-cyanobenzo[d]isoxazol-3-yl)phenyl]-2-(pyridine-2-yl)ethyl}carbamate (Intermediate 10A, 0.05 g) and TFA (1 mL) in DCM (2 mL) was stirred for 3 hours. The resultant mixture was concentrated in vacuo and the residue was redissolved in toluene and re-concentrated. The residue was dissolved in DCM, treated with MP-carbonate and stirred for 3 hours then filtered. The filtrate was concentrated in vacuo and the residue was purified by FCC eluting with 0-5% methanol in DCM. After evaporation, the residue was dissolved in a mixture of acetonitrile and water and the solution was freeze dried to give the title compound (0.016 g) as a tan coloured solid. ¹H NMR (400 MHz, DMSO-d₆) 8.51-8.49 (1H, m), 8.13-8.10 (1H, m), 7.92 (1H, d, J=7.87 Hz), 7.76 (1H, dd, J=1.30, 8.36 Hz), 7.73 (1H, dd, J=1.9, 8.36 Hz), 7.61-7.55 (1H, m), 7.47-7.40 (1H, m), 7.39-7.36 (2H, m), 6.98-6.94 (1H, m), 6.86-6.82 (1H, m), 4.43 (1H, t, J=7.21 Hz), 2.91-2.87 (2H, m), 1.99 (2H, br s). LCMS Method (Method 1): RT 2.62 m/z 341 [MH⁺].

By proceeding in a similar manner to Example 29, the following compounds were prepared:

### Example 30: (S)-1-[2-(6-Hyroxymethylbenzo[d]isoxazol-3-yl)phenyl]-2-(pyridine-2-yl)ethan-1-amine hydrochloride

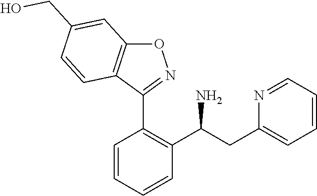 [SMILES: N[C@@H](Cc1ccccn1)c1ccccc1-c1noc2cc(CO)ccc12]

Starting from tert-Butyl (S)-{1-[2-(6-hydroxymethylbenzo[d]isoxazol-3-yl)phenyl]-2-(pyridine-2-yl)ethyl}carbamate (Intermediate 14A) and converting to the HCl salt by dissolving in acetonitrile, treating with 0.1M aqueous HCl and freeze drying. ¹H NMR (400 MHz, d₆-DMSO-d₆) 8.91-8.80 (3H br s), 8.20-8.15 (1H, m), 8.11 (1H, d, J=7.82 Hz), 7.76-7.67 (3H, m), 7.62-7.58 (2H, m), 7.48 (1H, d, J=8.40 Hz), 7.35 (1H, dd, J=1.0, 8.4 Hz), 7.22-7.12 (2H, m), 5.25-5.15 (1H, m), 4.70 (2H, s), 3.58-3.48 (1H, m), 3.43-3.33 (1H, m). LCMS (Method 1) RT 2.2, m/z 346 [MH⁺].

### Example 31: (S)-1-[2-(6-Ethynylbenzo[d]isoxazol-3-yl)phenyl]-2-(pyridine-2-yl)ethan-1-amine hydrochloride

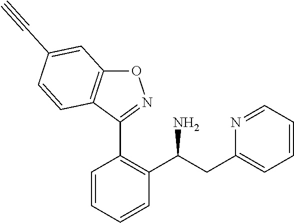 [SMILES: C#Cc1ccc2c(-c3ccccc3[C@@H](N)Cc3ccccn3)noc2c1]

Starting from tert-butyl (S)-{1-[2-(6-trimethylsilylethynylbenzo[d]isoxazol-3-yl)phenyl]-2-(pyridine-2-yl)ethyl}carbamate (Intermediate 15A) and converting to the HCl salt by treatment with 0.1M aqueous hydrochloric acid and freeze drying. ¹H NMR (400 MHz, DMSO-d₆) 8.84-8.76 (3H, br s), 8.16-8.07 (2H, m), 8.02 (1H, s), 7.77-7.71 (1H, m), 7.71-7.64 (1H, m), 7.62-7.58 (2H, m), 7.55-7.45 (2H, m), 7.20-7.09 (2H, m), 5.22-5.15 (1H, m), 4.50 (1H, s), 3.56-3.47 (1H, m), 3.41-3.33 (1H, m). LCMS (Method 1) RT 2.98 m/z 340

### Example 32: (S)-1-[2-(6-[3-Hydroxyprop-1-yn-1-yl]benzo[d]isoxazol-3-yl)phenyl]-2-(pyridine-2-yl)ethan-1-amine hydrochloride

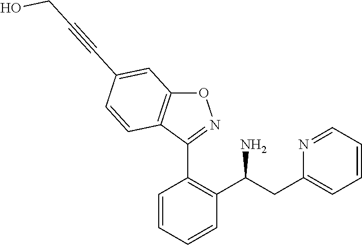 [SMILES: N[C@@H](Cc1ccccn1)c1ccccc1-c1noc2cc(C#CCO)ccc12]

Starting from tert-Butyl (S)-{1-[2-(6-[3-hydroxyprop-1-yn-1-yl]benzo[d]isoxazol-3-yl)phenyl]-2-(pyridine-2-yl)ethyl}carbamate (Intermediate 15B) and converting to the HCl salt by treatment with 0.1M aqueous hydrochloric acid and freeze drying. ¹H NMR (400 MHz, DMSO-d₆) 8.74 (3H, br s), 8.10-8.04 (2H, m), 7.93 (1H, s), 7.77-7.70 (1H, m), 7.66-7.56 (3H, m), 7.51-7.39 (2H, m), 7.14-7.04 (2H, m), 5.25-5.17 (1H, m), 4.35 (2H, s), 3.51-3.43 (1H, m), 3.38-3.29 (1H, m). LCMS (Method 1) RT 2.56 m/z 370

### Example 33: (S)-1-[2-(6-Methylbenzo[d]isoxazol-3-yl)phenyl]-2-(pyridine-2-yl)ethan-1-amine

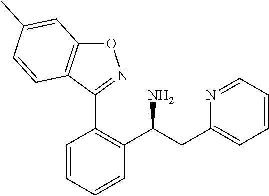 [SMILES: Cc1ccc2c(-c3ccccc3[C@@H](N)Cc3ccccn3)noc2c1]

A solution of crude tert-butyl (S)-{1-[2-(6-methylbenzo[d]isoxazol-3-yl)phenyl]-2-(pyridine-2-yl)ethyl}carbamate (Intermediate 11A, 0.03 g) and TFA (1 mL) in DCM (2 mL) was stirred for 3 hours. The resultant mixture was concentrated in vacuo and the residue was purified by FCC, eluting with 0-10% 2M ammonia/methanol in ethyl acetate to give the title compound (0.013 g) as a colourless oil. ¹H NMR (400 MHz, DMSO-d₆) 8.21-8.18 (1H, m), 7.88 (1H, d, J=7.95 Hz), 7.59 (1H, s), 7.57-7.52 (1H, m), 7.51-7.46 (1H, m), 7.43 (1H, d, J=7.95 Hz), 7.39-7.36 (2H, m), 7.20-7.17 (1H, m), 7.03-6.99 (1H, m), 6.90-6.86 (1H, m), 4.48-4.42 (1H, m), 2.94 (1H, dd, J=5.34, 13.48), 2.86 (1H, dd, J=8.37, 13.48), 2.47 (3H, s). LCMS (Method 1): RT 2.99 m/z 330.

By proceeding in a similar manner to Example 33, the following compounds were prepared:

### Example 34: (S)-1-[2-(6-Cyclopropylbenzo[d]isoxazol-3-yl)phenyl]-2-(pyridine-2-yl)ethan-1-amine

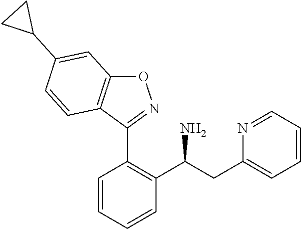 [SMILES: N[C@@H](Cc1ccccn1)c1ccccc1-c1noc2cc(C3CC3)ccc12]

Starting from tert-butyl (S)-{1-[2-(6-cyclopropylbenzo[d]isoxazol-3-yl)phenyl]-2-(pyridine-2-yl)ethyl}carbamate (Intermediate 12A). ¹H NMR (400 MHz, DMSO-d₆) 8.10-8.07 (1H, m), 7.89 (1H, d, J=7.72 Hz), 7.68-7.62 (1H, m), 7.57-7.47 (4H, m), 7.36 (1H, d J=8.30 Hz), 7.14-7.10 (1H, m), 7.03-6.98 (1H, m), 6.96 (1H, d, J=6.95 Hz), 5.01 (1H, t, J=7.14), 3.23-3.15 (2H, m), 2.16-2.08 (1H, m), 1.08-1.02 (2H, m), 0.84-0.78 (2H, m). LCMS (Method 1): RT 3.30 m/z 356.

### Example 35: (S)-1-[2-(6-[1-Hydroxyethyl]benzo[d]isoxazol-3-yl)phenyl]-2-(pyridine-2-yl)ethan-1-amine

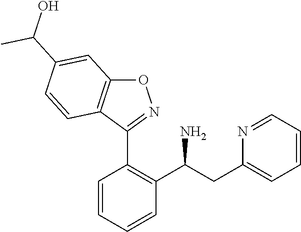 [SMILES: CC(O)c1ccc2c(-c3ccccc3[C@@H](N)Cc3ccccn3)noc2c1]

Starting from tert-butyl (S)-{1-[2-(6-[1-hydroxyethyl]benzo[d]isoxazol-3-yl)phenyl]-2-(pyridine-2-yl)ethyl}carbamate (Intermediate 17A). ¹H NMR (400 MHz, DMSO-d₆) 8.23-8.19 (1H, m), 7.90 (1H, d, J=7.89 Hz), 7.71 (1H, s), 7.60-7.54 (1H, m), 7.52-7.46 (2H, m), 7.41-7.38 (2H, m), 7.35 (1H, d, J=8.30 Hz), 7.05-6.99 (1H, m), 6.88 (1H, dt, J=0.99, 7.67 Hz), 5.43 (1H, br s), 4.94-4.86 (1H, m), 4.47-4.41 (1H, m), 2.98-2.82 (2H, m), 1.37 (3H, d, J=6.55 Hz). LCMS (Method 1) RT 2.41 m/z 360.

### Example 36: (S)-1-[2-(Benzo[d]isoxazol-3-yl)-5-methylphenyl]-2-(pyridine-2-yl)ethan-1-amine hydrochloride

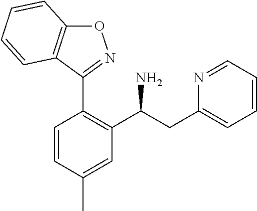 [SMILES: Cc1ccc(-c2noc3ccccc23)c([C@@H](N)Cc2ccccn2)c1]

Starting from tert-butyl (S)-{1-[2-(benzo[d]isoxazol-3-yl)-5-methylphenyl]-2-(pyridine-2-yl)ethyl}carbamate (Intermediate 11B) and converting to the HCl salt by dissolving in acetonitrile, treating with 0.1M hydrochloric acid and freeze drying. ¹H NMR (400 Mz, DMSO-d₆) 8.92-8.84 (3H, br s), 8.22-8.16 (1H, m), 8.01 (1H, s), 7.81 (1H, d, J=8.45 Hz), 7.79-7.73 (1H, m), 7.72-7.67 (1H, m), 7.55-7.47 (2H, m), 7.42-7.37 (2H, m), 7.17-7.19 (2H, m), 5.21-5.14 (1H, m), 3.63-3.55 (1H, m), 3.47-3.38 (1H, m), 2.45 (3H, s). LCMS (Method 1) RT 2.92 m/z 330 [MH⁺].

### Example 37: (S)-1-[2-(Benzo[d]isoxazol-3-yl)-5-cyanophenyl]-2-(pyridine-2-yl)ethan-1-amine hydrochloride

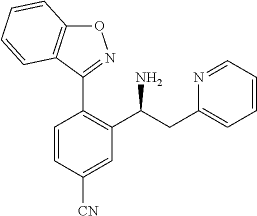 [SMILES: N#Cc1ccc(-c2noc3ccccc23)c([C@@H](N)Cc2ccccn2)c1]

Starting from tert-butyl (S)-{1-[2-(benzo[d]isoxazol-3-yl)-5-cyanophenyl]-2-(pyridine-2-yl)ethyl}carbamate (Intermediate 27A) and converting to the HCl salt by dissolving in acetonitrile, treating with 0.1M hydrochloric acid and freeze drying. ¹H NMR (400 MHz, DMSO-d₆) 9.03-8.95 (3H, br s), 8.67 (1H, d, J=1.76 Hz), 8.13-8.09 (1H, m), 8.07 (1H, dd, J=1.60, 7.92 Hz), 7.89-7.81 (2H, m), 7.77-7.68 (2H, m), 7.56 (1H, d, J=7.90 Hz), 7.46-7.41 (1H, m), 7.24-7.14 (2H, m), 5.28-5.20 (1H, m), 3.62-3.54 (1H, m), 3.49-3.41 (1H, m). LCMS (Method 1) RT 2.75 m/z 341 [MH⁺].

### Example 38: (S)-1-[2-(Benzo[d]isoxazol-3-yl)-4-methylphenyl]-2-(pyridine-2-yl)ethan-1-amine hydrochloride

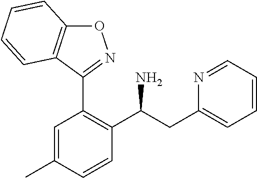 [SMILES: Cc1ccc([C@@H](N)Cc2ccccn2)c(-c2noc3ccccc23)c1]

Starting from tert-butyl (S)-{1-[2-(benzo[d]isoxazol-3-yl)-4-methylphenyl]-2-(pyridine-2-yl)ethyl}carbamate (Intermediate 11C) and converting to the hydrochloride salt by dissolving in acetonitrile, treating with 0.1M hydrochloric acid and freeze drying. ¹H NMR (400 MHz, DMSO-d₆) 8.79 (3H, br s), 8.19-8.13 (1H, m), 7.99 (1H, d, J=8.08 Hz), 7.85-7.81 (1H, m), 7.76-7.69 (2H, m), 7.58-7.51 (2H, m), 7.43-7.38 (2H, m), 7.23-7.14 (2H, m), 5.18-5.11 (1H, m), 3.57-3.49 (1H, m), 3.42-3.34 (1H, m), 2.38 (3H, S). LCMS (Method 1) RT 2.94 m/z 330 [MH⁺].

### Example 39: (S)-1-[2-(6-Methoxymethylbenzo[d]isoxazol-3-yl)phenyl]-2-(pyridine-2-yl)ethan-1-amine

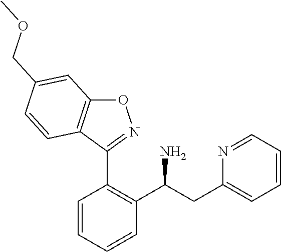 [SMILES: COCc1ccc2c(-c3ccccc3[C@@H](N)Cc3ccccn3)noc2c1]

Starting from (S)-{1-[2-(6-methyxymethylbenzo[d]isoxazol-3-yl)phenyl]-2-(pyridine-2-yl)ethyl}carbamate (Intermediate 34A). ¹H NMR (400 MHz, DMSO-d₆) 8.22-8.19 (1H, m), 7.91 (1H, d, J=8.08 Hz), 7.72 (1H, s), 7.61-7.52 (2H, m), 7.52-7.46 (1H, m), 7.42-7.38 (2H, m), 7.34-7.30 (1H, m), 7.04-6.99 (1H, m), 6.91-6.87 (1H, m), 4.60 (2H, s), 4.49-4.44 (1H, m), 3.34 (3H, s), 2.98-2.84 (2H, m). LCMS (Method 1) RT 2.74 m/z 360 [MH⁺].

### Example 40: (S)-1-[2-(6-[2-Hydroxyethyl]benzo[d]isoxazol-3-yl)phenyl]-2-(pyridine-2-yl)ethan-1-amine

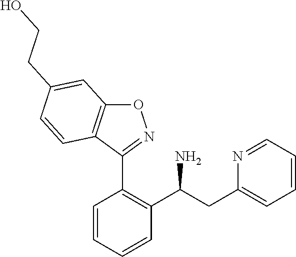 [SMILES: N[C@@H](Cc1ccccn1)c1ccccc1-c1noc2cc(CCO)ccc12]

Starting from (S)-{1-[2-(6-[2-hydroxyethyl]benzo[d]isoxazol-3-yl)phenyl]-2-(pyridine-2-yl)ethyl}carbamate (Intermediate 35A). ¹H NMR (400 MHz, DMSO-d₆) 8.23-8.20 (1H, m), 7.90 (1H, d, J=7.75 Hz), 7.63 (1H, s), 7.59-7.54 (1H, m), 7.52-7.44 (2H, m), 7.40-7.36 (2H, m), 7.26-7.22 (1H, m), 7.05-7.01 (1H, m), 6.91-6.87 (1H, m), 4.70 (1H, t, J=5.24 Hz), 4.45 (1H, dd, J=5.24, 8.20 Hz), 3.71-3.65 (2H, m), 2.98-2.83 (4H, m). LCMS (Method 1) RT 2.32 m/z 360 [MH⁺].

### Example 41: 1-(6-{(S)-2-Amino-2-[2-(benzo[d]isoxazol-3-yl)phenyl]ethyl}pyridine-2-yl)ethan-1,2-diol hydrochloride

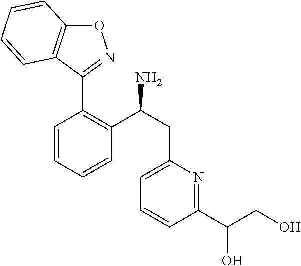 [SMILES: N[C@@H](Cc1cccc(C(O)CO)n1)c1ccccc1-c1noc2ccccc12]

Starting from tert-butyl {(1S)-1-[2-(benz[d]isoxazol-3-yl)phenyl]-2-[6-(1,2-dihydroxyethyl)pyridin-2-yl]ethyl}carbamate (Intermediate 55A) and converting to the HCl salt by treating with HCl in methanol and concentrating in vacuo. ¹H NMR (400 MHz, DMSO-d₆) 7.99 (1H, dd, J=4.0, 7.5 Hz), 7.86-7.84 (1H, m), 7.77-7.65 (3H, m), 7.63-7.51 (3H, m), 7.46-7.41 (1H, m), 7.22-7.19 (1H, m), 6.88-6.84 (1H, m), 6.60 (1H, br s), 5.22 (1H, dd, J=5.1, 12.4 Hz), 4.89-4.81 (1H, m), 4.59 (1H, br s), 4.42-4.36 (0.5H, m), 4.31-4.26 (0.5H, m), 3.48-3.39 (1H, m), 3.26-3.15 (3H, m). LCMS (Method 1) RT 2.67 m/z 376 [MH⁺].

### Example 42: (S)-1-[2-(Benzo[d]isoxazol-3-yl)phenyl]-2-(pyridine-2-yl)ethan-1-amine hydrochloride

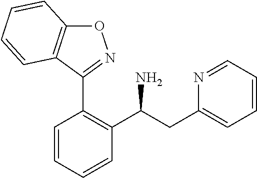 [SMILES: N[C@@H](Cc1ccccn1)c1ccccc1-c1noc2ccccc12]

A solution of (S)—N—{(S)-1-[2-(benzo[d]isoxazol-3-yl)phenyl]-2-(pyridine-2-yl)ethyl}-2-methylpropane-2-sulfinamide (Intermediate 6A, 0.1 g) in hydrogen chloride in methanol (1.25M, 4 mL) was stirred at room temperature for 2.5 hours. The resultant solution was concentrated in vacuo and the residue was purified by FCC eluting with 0-6% ammonia in methanol (2M) in DCM. After evaporation, the product was dissolved in a mixture of acetonitrile, water and aqueous hydrochloric acid (1M) to give the title compound as a pale yellow solid (0.06 g). ¹H NMR (400 MHz, DMSO-d₆) 8.97 (3H, br s), 8.31-8.20 (2H, m), 7.89-7.84 (2H, m), 7.80-7.73 (2H, m), 7.64-7.57 (3H, m), 7.46-7.42 (1H, m), 7.33-7.28 (2H, m), 5.23-5.17 (1H, m), 3.72-3.65 (1H, m), 3.54-3.44 (1H, m). LCMS (Method 1) RT2.79 m/z 316 [MH⁺].

By proceeding in a similar manner to Example 42, the following compounds were prepared:

### Example 43: (S)-1-[2-(4-Bromobenzo[d]isoxazol-3-yl)phenyl]-2-(pyridine-2-yl)ethan-1-amine hydrochloride

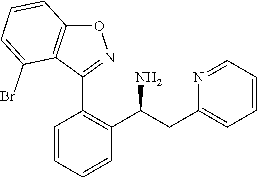 [SMILES: N[C@@H](Cc1ccccn1)c1ccccc1-c1noc2cccc(Br)c12]

Starting from of (S)—N—{(S)-1-[2-(4-bromobenzo[d]isoxazol-3-yl)phenyl]-2-(pyridine-2-yl)ethyl}-2-methylpropane-2-sulfinamide (Intermediate 5B) and converting to the hydrochloride salt by dissolving in acetonitrile, treating with 0.1M hydrochloric acid and freeze drying. ¹H NMR (400 MHz, DMSO-d₆) 8.77 (3H, brs), 8.05-7.95 (2H, m), 7.92 (1H, d, J=8.50 Hz), 7.76-7.50 (6H, m), 7.20-7.02 (2H, m), 4.99-4.75 (1H, m), plus 2 additional protons under the water peak. LCMS (Method 1) RT 3.03 m/z 394.

### Example 44: (S)-1-[2-(Benzo[d]isoxazol-3-yl)phenyl]-2-(6-bromopyridine-2-yl)ethan-1-amine hydrochloride

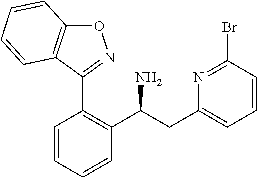 [SMILES: N[C@@H](Cc1cccc(Br)n1)c1ccccc1-c1noc2ccccc12]

Starting from (S)—N—{(S)-1-[2-(benzo[d]isoxazol-3-yl)phenyl]-2-(6-bromopyridine-2-yl)ethyl}-2-methylpropane-2-sulfinamide (Intermediate 6B) and converting to the hydrochloride salt by dissolving in acetonitrile, treating with 0.1M hydrochloric acid and freeze drying. ¹¹H NMR (400 MHz, DMSO-d₆) 8.75 (3H, br s), 8.10 (1H, d, J=8.0 Hz), 7.83 (1H, d, J=8.30 Hz), 7.77-7.68 (2H, m), 7.62-7.56 (2H, m), 7.49-7.46 (1H, m), 7.42-7.35 (2H, m), 7.12 (1H, d, J=8.0 Hz), 6.89 (1H, d, J=7.4 Hz), 5.18-5.13 (1H, m), 3.37-3.29 (1H, m), 3.27-3.19 (1H, m). LCMS (Method 1) RT 3.08 m/z 394/396 [MH⁺].

### Examples 45 & 46: (1S,2S)-1-[2-(Benzo[d]isoxazol-3-yl)phenyl]-2-(pyridin-2-yl)butan-1-amine hydrochloride and (1S,2R)-1-[2-(benzo[d]isoxazol-3-yl)phenyl]-2-(pyridin-2-yl)butan-1-amine hydrochloride

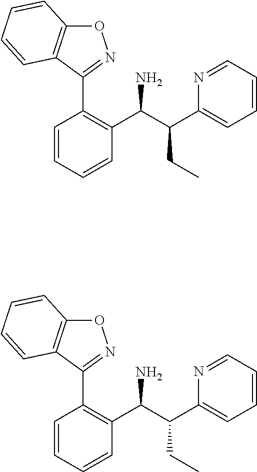 [SMILES: CC[C@H](c1ccccn1)[C@H](N)c1ccccc1-c1noc2ccccc12 ; CC[C@@H](c1ccccn1)[C@H](N)c1ccccc1-c1noc2ccccc12]

Starting from a diastereomeric mixture of (1S)—N—{(S)-1-[2-(benzo[d]isoxazol-3-yl)phenyl]-2-(pyridine-2-yl)butane}-2-methylpropane-2-sulfinamide (Intermediate 5K). The diastereomers were separated by MDAP and subsequently converted to the HCl salts by treatment with 1M hydrochloric acid in aqueous acetonitrile.

Example 45, Diastereomer 1: ¹H NMR (400 MHz, DMSO-d₆) 8.96 (3H, br s), 8.04 (1H, d, J=8.1 Hz), 7.86 (1H, d, J=8.3 Hz), 7.76-7.70 (1H, m), 7.67-7.61 (2H, m), 7.49-7.45 (1H, m), 7.44-7.37 (3H, m), 7.29-7.25 (1H, m), 7.69-7.87 (1H, m), 6.86-6.78 (1H, m), 5.13-5.05 (1H, m), 3.43-3.32 (2H, m), 2.00-1.90 (1H, m), 1.70-1.59 (1H, m), 0.49 (3H, t, J=7.4 Hz). LCMS (Method 1) RT 2.95 m/z 344 [MH⁺].

Example 46, Diastereomer 2: ¹H NMR (400 MHz, DMSO-d₆) 8.58 (3H, br s), 8.06 (1H, d, J=8.0 Hz), 7.94-7.86 (2H, m), 7.82-7.69 (5H, m), 7.69-7.63 (1H, m), 7.52-7.43 (2H, m), 7.43-7.36 (1H, m), 5.08-5.00 (1H, m), 3.49-3.39 (2H, m), 1.44-1.34 (1H, m), 1.31-1.19 (1H, m), 0.36 (3H, t, J=7.3 Hz). LCMS (Method 1) RT 3.31 m/z 344 [MH⁺].

### Example 47: (S)-1-[2-(Benzo[d]isoxazol-3-yl)phenyl]-2-(3-methylpyridine-2-yl)ethan-1-amine hydrochloride

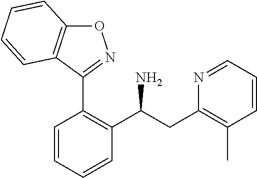 [SMILES: Cc1cccnc1C[C@H](N)c1ccccc1-c1noc2ccccc12]

Starting from (S)—N—{(S)-1-[2-(benzo[d]isoxazol-3-yl)phenyl]-(3-methylpyridin-2-yl)ethyl}-2-methylpropane-2-sulfinamide (Intermediate 41A) and converting to the HCl salt by treatment with 1M aqueous hydrochloric acid in acetonitrile and freeze drying. ¹H NMR (400 MHz, DMSO-d₆) 9.06 (3H, br s), 8.26 (1H, d, J=7.9 Hz), 83.11 (1H, br s), 7.82 (1H, d, J=8.55 Hz), 7.78-7.67 (3H, m), 7.64-7.51 (3H, m), 7.43-7.37 (1H, m), 7.25 (1H, m), 5.27-5.19 (1H, m), 3.69-3.61 (1H, m), 3.55-3.47 (1H, m), 2.04 (3H, s). LCMS (Method 1) RT 2.74 m/z 330 [MH⁺].

### Example 48: (S)-1-[2-(Benzo[d]isoxazol-3-yl)phenyl]-3-methyl-2-(pyridine-2-yl)butan-1-amine hydrochloride

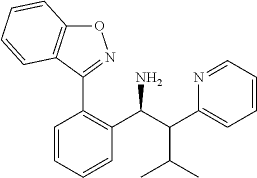 [SMILES: CC(C)C(c1ccccn1)[C@H](N)c1ccccc1-c1noc2ccccc12]

Starting from (S)—N—{(S)-1-[2-(benzo[d]isoxazol-3-yl)phenyl]-3-methyl-2-(pyridin-2-yl)ethyl}-2-methylpropane-2-sulfinamide (Intermediate 42A). ¹H NMR (400 MHz, DMSO-d₆) 8.75 (3H, br s), 7.93 (1H, d, J=7.7 Hz), 7.86-7.83 (1H, m), 7.77-7.72 (1H, m), 7.64 (1H, d, J=4.4 Hz), 7.55-7.33 (6H, m), 6.91-6.86 (1H, m), 6.82-6.78 (1H, m), 5.47 (1H, d, J=10.4 Hz), 3.63 (1H, dd, J=3.9, 10.4 Hz), 2.39-2.31 (1H, m), 0.77 (3H, d, J=6.8 Hz), 0.66 (3H, d, J=6.7 Hz). LCMS (Method 1) RT 3.17 m/z 358 [MH⁺].

### Example 49: (R)-1-[2-(Benzo[d]isoxazol-3-yl)phenyl]-2-methyl-2-(pyridine-2-yl)propane-1-amine hydrochloride

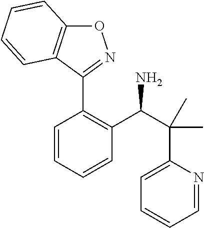 [SMILES: CC(C)(c1ccccn1)[C@H](N)c1ccccc1-c1noc2ccccc12]

Starting from (S)—N—{(R)-1-[2-(benzo[d]isoxazol-3-yl)phenyl]-2-methyl-2-(pyridin-2-yl)propyl}-2-methylpropane-2-sulfinamide (Intermediate 43A) and converting to the HCL salt by treatment with aqueous hydrochloric acid in acetonitrile and freeze drying. ¹H NMR (400 MHz, DMSO-d₆) 8.82 (3H, br s), 8.23-8.19 (1H, m), 7.83 (1H, d, J=8.3 Hz), 7.74-7.68 (1H, m), 7.65-7.54 (5H, m), 7.49-7.45 (1H, m), 7.43-7.38 (1H, m), 7.09 (1H, d, J=7.9 Hz), 7.05-7.00 (1H, m), 5.46-5.39 (1H, m), 1.24 (3H, s), 1.10 (3H, s). LCMS (Method 1) RT 3.26 m/z 344 [MH⁺].

### Example 50: (R)-1-(6-{(S)-2-Amino-2-[2-(benzo[d]isoxazol-3-yl)phenyl]ethyl}pyridine-2-yl)pyrrolidin-3-ol hydrochloride

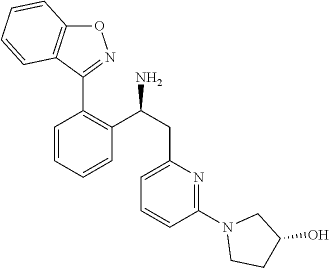 [SMILES: N[C@@H](Cc1cccc(N2CC[C@@H](O)C2)n1)c1ccccc1-c1noc2ccccc12]

Starting from (S)—N—{(S)-1-[2-(benzo[d]isoxazol-3-yl)phenyl]-2-[6-((R)-3-hydroxylpyrrolidin-1-yl)pyridine-2-yl]ethyl}propane-2-sulfinamide (Intermediate 53A) and converting to the HCl salt by treatment with aqueous hydrochloric acid in acetonitrile and freeze drying. ¹H NMR (400 MHz, DMSO-d₆ 80° C.) 8.79 (3H, br s), 8.12 (1H, d, J=8.1 Hz), 7.82-7.78 (1H, m), 7.76-7.69 (2H, m), 7.65-7.61 (2H, m), 7.57-7.55 (1H, m), 7.43-7.38 (1H, m), 7.36-7.30 (1H, m), 6.29 (1H, s), 6.21 (1H, d, J=7.1 Hz), 5.24-5.18 (1H, m), 4.37-4.36 (1H, m), 3.22-3.17 (5H, m), 2.00-1.83 (2H, m) plus one proton hidden under the water peak. LCMS (Method 2) RT 2.09 m/z 401 [MH⁺].

### Example 51: (S)-6-{2-Amino-2-[2-(benzo[d]isoxazol-3-yl)phenyl]ethyl}-N-(2-methoxyethyl)-N-methyl pyridin-2-amine hydrochloride

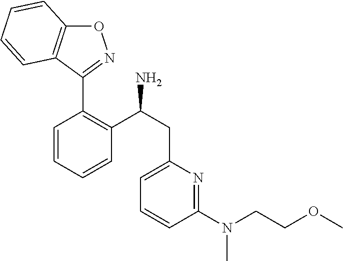 [SMILES: COCCN(C)c1cccc(C[C@H](N)c2ccccc2-c2noc3ccccc23)n1]

Starting from (S)—N—{(S)-1-[2-(benzo[d]isoxazol-3-yl)phenyl]-2-[6-[2-methoxyethyl]methylamino)pyridine-2-yl]ethyl}propane-2-sulfinamide (Intermediate 53B) and converting to the HCl salt by treating with 1M hydrochloric acid in acetonitrile and freeze drying. ¹H NMR (400 MHz, DMSO-d₆) 8.82 (3H, br s), 8.14 (1H, br s), 7.88-7.85 (1H, m), 7.79-7.72 (2H, m), 7.64-7.62 (2H, m), 7.56-7.51 (1H, m), 7.46-7.40 (2H, m), 7.32 (1H, br s), 6.21-6.15 (1H, br s), 5.25-5.20 (1H, m), 3.51-3.46 (2H, m), 3.31 (3H, s), 3.21-3.17 (4H, m), 2.82-2.66 (2H, m) plus one proton hidden under the water peak. LCMS (Method 1) RT 3.00 m/z 403 [MH⁺].

### Example 52: (S)-6-{2-Amino-2-[2-(benzo[d]isoxazol-3-yl)phenyl]ethyl}-N-(2-methoxyethyl)pyridin-2-amine hydrochloride

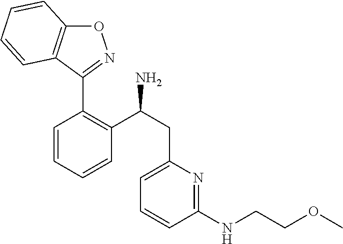 [SMILES: COCCNc1cccc(C[C@H](N)c2ccccc2-c2noc3ccccc23)n1]

Starting from (S)—N—{(S)-1-[2-(benzo[d]isoxazol-3-yl)phenyl]-2-[6-(2-methoxyethylamino)pyridine-2-yl]ethyl}propane-2-sulfinamide (Intermediate 53C) and converting to the HCl salt by treating with 1M hydrochloric acid in acetonitrile and freeze drying. ¹H NMR (400 MHz, DMSO-d₆) 8.68-8.67 (3H, br s), 8.06-8.04 (1H, m), 7.83-7.79 (1H, m), 7.76-7.70 (2H, m), 7.69-7.61 (3H, m), 7.45-7.41 (1H, m), 7.27 (1H, t, J=7.8 Hz), 6.42 (1H, d, J=4.7 Hz), 6.21 (1H, d, J=7.1 Hz), 5.14-5.11 (1H, m), 3.40-3.36 (4H, m), 3.26 (3H, s), 3.19-3.15 (2H, m). LCMS (Method 2) RT 2.29 m/z 389 [MH⁺].

### Example 53: (S)-1-(6-{(S)-2-Amino-2-[2-(benzo[d]isoxazol-3-yl)phenyl]ethyl}pyridine-2-yl)pyrrolidin-3-ol hydrochloride

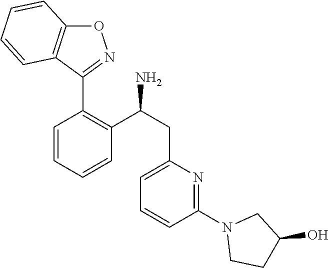 [SMILES: N[C@@H](Cc1cccc(N2CC[C@H](O)C2)n1)c1ccccc1-c1noc2ccccc12]

Starting from (S)—N—{(S)-1-[2-(benzo[d]isoxazol-3-yl)phenyl]-2-[6-((S)-3-hydroxylpyrrolidin-1-yl)pyridine-2-yl]ethyl}propane-2-sulfinamide (Intermediate 53D) and converting to the HCl salt by treatment with aqueous hydrochloric acid in acetonitrile and freeze drying. ¹H NMR (400 MHz, DMSO-d₆) 8.91 (3H, br s), 8.18 (1H, d, J=7.4 Hz), 7.85-7.83 (1H, m), 7.80-7.76 (1H, m), 7.75-7.71 (1H, m), 7.64-7.62 (2H, m), 7.54-7.51 (1H, m), 7.44-7.40 (2H, m), 6.57-6.34 (1H, m), 6.22-6.22 (1H, m), 5.27-5.26 (1H, m), 4.37-4.36 (1H, m), 3.25-3.17 (5H, m), 2.00-1.90 (2H, m) plus one proton hidden under the water peak. LCMS (Method 2) RT 2.10 m/z 401 [MH⁺].

### Example 54: (S)-1-[2-(Benzo[d]isoxazol-3-yl)phenyl]-2-(6-morpholinopyridine-2-yl)ethane-1-amine hydrochloride

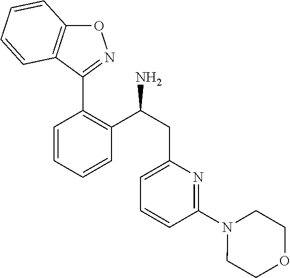 [SMILES: N[C@@H](Cc1cccc(N2CCOCC2)n1)c1ccccc1-c1noc2ccccc12]

Starting from (S)—N—{(S)-1-[2-(benzo[d]isoxazol-3-yl)phenyl]-2-(6-morpholinopyridine-2-yl]ethyl}propane-2-sulfinamide (Intermediate 60A) and converting to the HCl salt by treatment with aqueous hydrochloric acid in acetonitrile and freeze drying.

¹H NMR (400 MHz, DMSO-d₆) 8.74-8.73 (3H, br s), 8.13-8.09 (11H, m), 7.91-7.88 (11H, m), 7.79-7.74 (2H, m), 7.64-7.60 (2H, m), 7.59-7.56 (1H, m), 7.43 (1H, t, J=7.5 Hz), 7.30 (1H, t, J=7.8 Hz), 6.48-6.46 (1H, m), 6.29 (1H, d, J=7.2 Hz), 5.24-5.18 (1H, m), 3.54-3.47 (4H, m), 3.28-3.27 (1H, m), 3.15-2.94 (5H, m). LCMS (Method 2) RT 3.03 m/z 401 [MH⁺].

### Example 55: (S)-2-[(6-{(S)-2-Amino-2-[2-(benzo[d]isoxazol-3-yl)phenyl]ethyl}pyridine-2-yl)(methyl)amino]ethan-1-ol hydrochloride

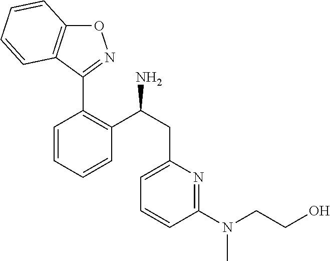 [SMILES: CN(CCO)c1cccc(C[C@H](N)c2ccccc2-c2noc3ccccc23)n1]

Starting from tert-butyl (S)-(1-[2-(benz[d]isoxazol-3-yl)phenyl]-2-{6-[(2-hydroxyethyl)(methyl)amino]pyridin-2-yl}}ethyl)carbamate (Intermediate 53E) and converting to the HCl salt by treatment with aqueous hydrochloric acid in acetonitrile and freeze drying. ¹H NMR (400 MHz, DMSO-d₆) 8.96-8.96 (3H, br s), 8.23-8.18 (1H, m), 7.89-7.86 (1H, m), 7.81-7.73 (2H, m), 7.64 (2H, d, J=4.1 Hz), 7.57-7.53 (1H, m), 7.44 (2H, t, J=7.2 Hz), 6.72-6.67 (1H, m), 6.29-6.25 (1H, m), 5.28-5.21 (1H, m), 3.54-3.47 (3H, m), 3.29-3.24 (1H, m), 2.94 (3H, s) plus 2 protons under the water peak. LCMS (Method 2) RT 2.36 m/z 389 [MH⁺]

### Example 56: (S)-1-[2-(Benzo[d]isoxazol-3-yl)phenyl]-2-(pyrimidine-2-yl)ethan-1-amine

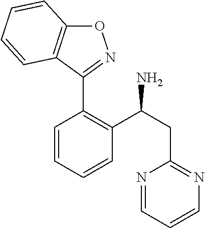 [SMILES: N[C@@H](Cc1ncccn1)c1ccccc1-c1noc2ccccc12]

Starting from (S)—N—{(S)-1-[2-(Benzo[d]isoxazol-3-yl)phenyl]-2-(pyrimidin-2-yl)ethyl}-2-methylpropane-2-sulfinamide (Intermediate 6H). ¹H NMR (400 MHz, DMSO-d₆) 8.47 (2H, d, J=4.9 Hz), 7.95-7.92 (1H, m), 7.85-7.82 (1H, m), 7.74-7.69 (1H, m), 7.66-7.58 (2H, m), 7.43-7.38 (3H, m), 7.16 (1H, t, J=4.9 Hz), 4.68 (1H, t, J=7.1 Hz), 3.12 (2H, d, J=7.1 Hz), 2.05 (2H, br s). LCMS (Method 1) RT 2.62 m/z 317 [MH⁺].

### Example 57: (S)-1-[2-(Benzo[d]isoxazol-3-yl)phenyl]-2-(6-bromo-3-methylpyridin-2-yl)ethan-1-amine hydrochloride

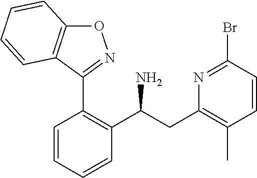 [SMILES: Cc1ccc(Br)nc1C[C@H](N)c1ccccc1-c1noc2ccccc12]

Starting from (S)—N—{(S)-1-[2-(benzo[d]isoxazol-3-yl)phenyl]-2-(6-bromo-3-methylpyridin-2-yl)ethyl}-2-methylpropane-2-sulfinamide (Intermediate 61) and converting to the HCl salt by treatment with aqueous hydrochloric acid in acetonitrile and freeze drying. ¹H NMR (400 MHz, DMSO-d₆) 8.89 (3H, br s), 8.19-8.15 (1H, m), 7.84 (1H, d, J=8.7 Hz), 7.80-7.70 (2H, m), 7.67-7.59 (2H, m), 7.52 (1H, d, J=7.9 Hz), 7.44-7.38 (1H, m), 7.17 (1H, d, J=7.9 Hz), 6.93 (1H, d, J=8.3 Hz), 5.35-5.28 (1H, m), 3.28 (2H, d, J=7.3 Hz), 1.82 (3H, s). LCMS (Method 2) rt 3.29 M/Z 408 [MH⁺].

### Example 58: (S)-1-[2-(Benzo[d]isoxazol-3-yl)phenyl]-2-(6-cyano-3-methylpyridin-2-yl)ethan-1-amine

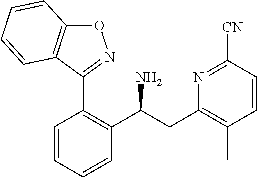 [SMILES: Cc1ccc(C#N)nc1C[C@H](N)c1ccccc1-c1noc2ccccc12]

Starting from (S)—N—{(S)-1-[2-(benzo[d]isoxazol-3-yl)phenyl]-2-(6-cyano-3-methylpyridine-2-yl)ethyl}-2-methylpropane-2-sulfinamide (intermediate 27H). ¹H NMR (400 MHz, DMSO-d₆) 8.04 (1H, d, J=7.2 Hz), 7.82-7.78 (1H, m), 7.73-7.67 (1H, m), 7.65-7.60 (1H, m), 7.59-7.55 (1H, m), 7.50 (1H, d, J=8.2 Hz), 7.46-7.35 (4H, m), 4.65 (1H, t, J=7.0 Hz), 2.98-2.94 (2H, m), 2.18 (2H, s), 1.90 (3H, s). LCMS (Method 1) RT 3.17 m/z 355 [MH⁺].

### Example 59: (S)-1-[2-(Benzo[d]isoxazol-3-yl)phenyl]-2-(3,6-dimethylpyridin-2-yl)ethan-1-amine

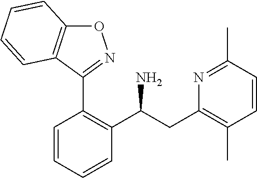 [SMILES: Cc1ccc(C)c(C[C@H](N)c2ccccc2-c2noc3ccccc23)n1]

Starting from (S)—N—{(S)-1-[2-(benzo[d]isoxazol-3-yl)phenyl]-2-(3,6-dimethylpyridine-2-yl)ethyl}-2-methylpropane-2-sulfinamide (Intermediate 11F). ¹H NMR (400 MHz, DMSO-d₆) 8.01 (1H, d, J=7.6 Hz), 7.82-7.79 (1H, m), 7.71-7.67 (1H, m), 7.62-7.54 (2H, m), 7.40-7.36 (3H, m), 7.15-7.12 (1H, m), 6.70-6.67 (1H, m), 4.65 (1H, t, J=6.8 Hz), 2.92-2.75 (2H, m), 2.23-2.15 (2H, m), 2.07-2.06 (3H, m), 1.78-1.77 (3H, m). LCMS (Method 2) RT 2.92 m/z 344 [MH⁺].

### Example 60: (S)-1-[2-(1-Methyl-1H-indazole-3-yl)phenyl]-2-(pyridine-2-yl)ethan-1-amine hydrochloride

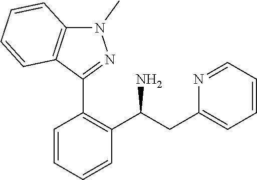 [SMILES: Cn1nc(-c2ccccc2[C@@H](N)Cc2ccccn2)c2ccccc21]

Starting from (S)-2-methyl-N-{1-[2-(1-methyl-1H-indazol-3-yl)phenyl]-2-[pyridine-2-yl]ethyl}propane-2-sulfinamide (Intermediate 5M) and converting to the HCl salt by treating with aqueous HCl in acetonitrile and freeze drying. ¹H NMR (400 MHz, DMSO-d₆) 8.91 (3H, br s), 8.50-8.45 (1H, m), 8.13-8.08 (1H, m), 7.99 (1H, t, J=7.2 Hz), 7.73-7.69 (1H, m), 7.62-7.54 (4H, m), 7.50-7.45 (2H, m), 7.36-7.30 (1H, m), 7.18 (1H, t, J=7.2 Hz), 5.45-5.45 (1H, m), 4.11 (3H, s), 3.58 (1H, dd, J=8.7, 13.9 Hz) plus one proton under the water peak. LCMS (Method 1) RT 3.01 m/z 329 [MH⁺].

### Example 61: (S)-1-[2-(1-Methyl-1H-indazole-3-yl)phenyl]-2-(6-methylpyridine-2-yl)ethan-1-amine hydrochloride

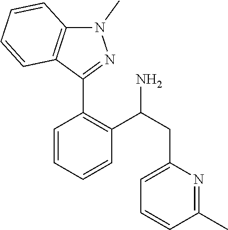 [SMILES: Cc1cccc(CC(N)c2ccccc2-c2nn(C)c3ccccc23)n1]

Starting from (S)-2-methyl-N-{1-[2-(1-methyl-1H-indazol-3-yl)phenyl]-2-[6-methylpyridine-2-yl]ethyl}propane-2-sulfinamide (Intermediate 5N) and converting to the HCl salt by treating with aqueous HCl in acetonitrile and freeze drying. ¹H NMR (400 MHz, DMSO-d₆) 8.98 (3H, br s), 8.14-8.09 (1H, m), 7.90-7.89 (1H, m), 7.73-7.70 (1H, m), 7.62-7.39 (5H, m), 7.27 (1H, s), 7.18-7.08 (2H, m), 5.65-5.65 (1H, m), 4.15-4.14 (3H, m), 3.73-3.73 (1H, m), 2.32-2.28 (3H, m) plus one proton under the water peak. LCMS (Method 1) RT 2.65 m/z 343 [MH⁺].

### Example 62: (S)-1-[2-(Benzo[d]isoxazol-3-yl)phenyl]-2-(6-trifluoromethylpyridin-2-yl)ethan-1-amine hydrochloride

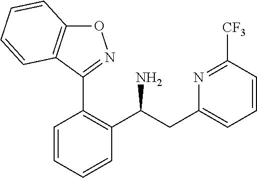 [SMILES: N[C@@H](Cc1cccc(C(F)(F)F)n1)c1ccccc1-c1noc2ccccc12]

Starting from (S)—N—{(S)-1-[2-(benzo[d]isoxazol-3-yl)phenyl]-2-(6-trifluoromethylpyridin-2-yl)ethyl}-2-methylpropane-2-sulfinamide (Intermediate 6M) and converting to the HCl salt by treating with aqueous HCl in acetonitrile and freeze drying. ¹H NMR (400 MHz, DMSO-d₆) 8.84 (3H, br s), 8.19-8.15 (1H, m), 7.84-7.69 (4H, m), 7.65-7.57 (2H, m), 7.49-7.44 (1H, m), 7.43-7.36 (2H, m), 7.17-7.13 (1H, m), 5.23-5.14 (1H, m), 3.50-3.43 (2H, m). LCMS (Method 1) RT 3.39 m/z 384 [MH⁺].

### Example 63: (S)-1-[2-(Benzo[d]isoxazol-3-yl)phenyl]-2-(3-fluoropyridin-2-yl)ethan-1-amine hydrochloride

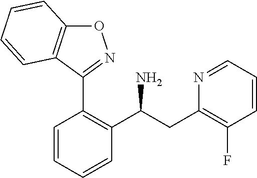 [SMILES: N[C@@H](Cc1ncccc1F)c1ccccc1-c1noc2ccccc12]

Starting from (S)—N—{(S)-1-[2-(benzo[d]isoxazol-3-yl)phenyl]-2-(3-fluoropyridin-2-yl)ethyl}-2-methylpropane-2-sulfinamide (Intermediate 6N) and converting to the HCl salt by treating with aqueous HCl in acetonitrile and freeze drying. ¹H NMR (400 MHz, DMSO-d₆) 8.96 (3H, br s), 8.17-8.14 (1H, m), 7.89-7.85 (1H, m), 7.77-7.71 (3H, m), 7.61 (2H, d, J=4.1 Hz), 7.55-7.51 (1H, m), 7.45-7.36 (2H, m), 7.08-7.03 (1H, m), 5.34-5.27 (1H, m), 3.53-3.37 (2H, m). LCMS (Method 1) RT 3.04 m/z 334 [MH⁺].

### Example 64: (S)-2-(6-Bromopyridine-2-yl)-1-[2-(1-isopropyl-1H-indazole-3-yl)phenyl]ethan-1-amine hydrochloride

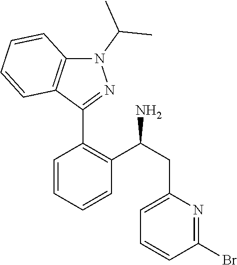 [SMILES: CC(C)n1nc(-c2ccccc2[C@@H](N)Cc2cccc(Br)n2)c2ccccc21]

Starting from (S)—N-{2-[6-bromopyridine-2-yl]-1-[2-(1-isopropyl-1H-indazol-3-yl)phenyl]ethyl}-2-methylpropane-2-sulfinamide (Intermediate 60) and converting to the HCl salt by treating with aqueous HCl in acetonitrile and freeze drying. ¹H NMR (400 MHz, DMSO-d₆) 8.61-8.53 (3H, br s), 8.01-7.97 (1H, m), 7.80-7.76 (1H, m), 7.69-7.64 (1H, m), 7.63-7.54 (3H, m), 7.51-7.43 (2H, m), 7.26-7.17 (2H, m), 7.08 (1H, d, J=7.3 Hz), 5.48-5.42 (1H, m), 5.13-5.02 (1H, m), 3.45-3.31 (2H, m), 1.58-1.49 (6H, m). LCMS (Method 1) RT 3.84 m/z 435 [MH⁺].

### Example 65: (S)-1-[2-(1-isopropyl-1H-indazole-3-yl)phenyl]-2-(pyridine-2-yl)ethan-1-amine hydrochloride

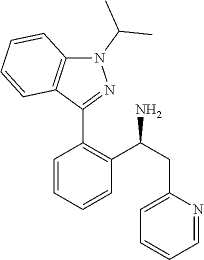 [SMILES: CC(C)n1nc(-c2ccccc2[C@@H](N)Cc2ccccn2)c2ccccc21]

Starting from (S)—N-{1-[2-(1-isopropyl-1H-indazol-3-yl)phenyl]-2-[pyridine-2-yl]ethyl}-2-methylpropane-2-sulfinamide (Intermediate 6P) and converting to the HCl salt by treating with aqueous HCl in acetonitrile and freeze drying. ¹H NMR (400 MHz, DMSO-d₆) 8.84 (3H, br s), 8.42 (1H, d, J=4.7 Hz), 8.06-8.02 (1H, m), 7.88 (1H, t, J=7.4 Hz), 7.79-7.76 (1H, m), 7.68-7.63 (1H, m), 7.61-7.54 (3H, m), 7.48-7.43 (1H, m), 7.38-7.30 (2H, m), 7.19 (1H, t, J=7.3 Hz), 5.53-5.46 (1H, m), 5.10-4.98 (1H, m), 3.75-3.65 (1H, m), 3.59-3.54 (1H, m), 1.53 (3H, d, J=6.6 Hz), 1.43 (3H, d, J=6.5 Hz). LCMS (Method 1) RT 3.47 m/z 357 [MH⁺].

### Example 66: (S)-1-[2-(1-Isopropyl-1H-indazole-3-yl)phenyl]-2-(6-methylpyridine-2-yl)ethan-1-amine hydrochloride

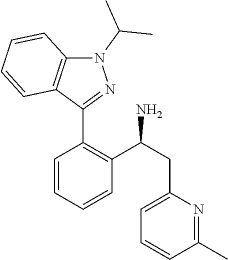 [SMILES: Cc1cccc(C[C@H](N)c2ccccc2-c2nn(C(C)C)c3ccccc23)n1]

Starting from (S)—N-{1-[2-(1-isopropyl-1H-indazol-3-yl)phenyl]-2-[6-methylpyridine-2-yl]ethyl}-2-methylpropane-2-sulfinamide (Intermediate 11G) and converting to the HCl salt by treating with aqueous HCl in acetonitrile and freeze drying. ¹H NMR (400 MHz, DMSO-d₆) 8.85 (3H, br s), 8.07-8.02 (1H, m), 7.80-7.76 (2H, m), 7.65-7.43 (5H, m), 7.23-7.12 (3H, m), 5.55-5.54 (1H, m), 5.11-5.00 (1H, m), 2.31 (3H, s), 1.60-1.55 (3H, m), 1.48-1.44 (3H, m) plus 2 protons hidden under the water peak. LCMS (Method 1) RT 3.30 m/z 371 [MH⁺].

### Example 67: (S)-2-(6-Cyanopyridine-2-yl)-1-[2-(1-isopropyl-1H-indazole-3-yl)phenyl]ethan-1-amine

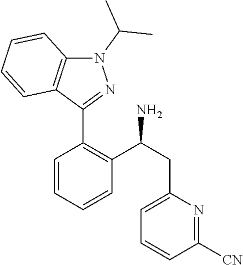 [SMILES: CC(C)n1nc(-c2ccccc2[C@@H](N)Cc2cccc(C#N)n2)c2ccccc21]

Starting from (S)—N-{2-[6-cyanopyridine-2-yl]-1-[2-(1-isopropyl-1H-indazol-3-yl)phenyl]ethyl}-2-methylpropane-2-sulfinamide (Intermediate 27J). ¹H NMR (400 MHz, CDCl₃) 7.74 (1H, dd, J=1.3, 7.8 Hz), 7.63-7.60 (1H, m), 7.58-7.52 (2H, m), 7.52-7.48 (1H, m), 7.48-7.34 (4H, m), 7.17-7.13 (2H, m), 4.97-4.87 (2H, m), 3.30 (1H, dd, J=5.0, 14.1 Hz), 3.15 (1H, dd, J=8.9, 14.1 Hz), 1.63 (3H, d, J=6.0 Hz), 1.60 (3H, d, J=6.6 Hz). LCMS (Method 1) RT 3.57 m/z 382 [MH⁺].

### Example 68: (S)-1-[2-(Benzo[d]isoxazol-3-yl)phenyl]-2-(6-fluoropyridin-2-yl)ethan-1-amine hydrochloride

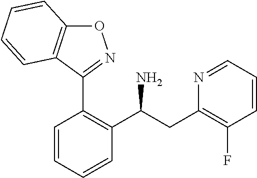 [SMILES: N[C@@H](Cc1ncccc1F)c1ccccc1-c1noc2ccccc12]

Starting from (S)—N—{(S)-1-[2-(benzo[d]isoxazol-3-yl)phenyl]-2-(6-fluoropyridine-2-yl)ethyl}-2-methylpropane-2-sulfinamide (Intermediate 6Q) and converting to the HCl salt by treating with aqueous HCl in acetonitrile and freeze drying. ¹H NMR (400 MHz, DMSO-d₆) 8.82 (3H, br s), 8.15-8.11 (1H, m), 7.88-7.85 (1H, m), 7.79-7.72 (2H, m), 7.70-7.60 (3H, m), 7.51-7.49 (1H, m), 7.44-7.39 (1H, m), 6.88 (1H, dd, J=2.3, 7.3 Hz), 6.70 (1H, dd, J=2.3, 8.2 Hz), 5.20-5.13 (1H, m), 3.39-3.22 (2H, m). LCMS (Method 1) RT 3.01 m/z 334 [MH⁺].

### Example 69: (S)-1-[2-(Benzo[d]isoxazol-3-yl)phenyl]-2-(5-fluoropyridin-2-yl)ethan-1-amine hydrochloride

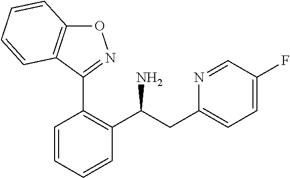 [SMILES: N[C@@H](Cc1ccc(F)cn1)c1ccccc1-c1noc2ccccc12]

Starting from (S)—N—{(S)-1-[2-(benzo[d]isoxazol-3-yl)phenyl]-2-(5-fluoropyridine-2-yl)ethyl}-2-methylpropane-2-sulfinamide (Intermediate 71A) and converting to the HCl salt by treating with aqueous HCl in acetonitrile and freeze drying. ¹H NMR (400 MHz, DMSO-d₆) 8.90 (3H, br s), 8.17-8.13 (1H, m), 7.89-7.84 (2H, m), 7.79-7.72 (2H, m), 7.62-7.60 (2H, m), 7.51-7.37 (3H, m), 7.02-6.97 (1H, m), 5.24-5.16 (1H, m), 3.43 (1H, dd, J=5.7, 14.1 Hz), 3.27 (1H, dd, J=8.6, 13.8 Hz). LCMS (Method 1) RT 3.01 m/z 334 [MH⁺].

### Example 70: (S)-1-[2-(Benzo[d]isoxazol-3-yl)phenyl]-2-(4-methoxypyridin-2-yl)ethan-1-amine

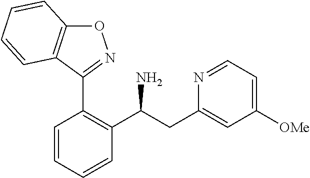 [SMILES: COc1ccnc(C[C@H](N)c2ccccc2-c2noc3ccccc23)c1]

Starting from (S)—N—{(S)-1-[2-(benzo[d]isoxazol-3-yl)phenyl]-2-(4-fluoropyridine-2-yl)ethyl}-2-methylpropane-2-sulfinamide (Intermediate 6R). Displacement of the fluorine by methoxy occurred during the reaction. ¹H NMR (400 MHz, MeOD) 7.96 (1H, d, J=5.9 Hz), 7.90-7.87 (1H, m), 7.78-7.64 (5H, m), 7.57-7.54 (1H, m), 7.45-7.40 (1H, m), 6.63-6.59 (1H, m), 6.59-6.55 (1H, m), 5.34-5.29 (1H, m), 3.72 (3H, s), 3.43-3.33 (21H, m). LCMS (Method 1) RT 2.28 m/z 346 [MH⁺].

### Example 71: (S)-1-[2-(Benzo[d]isoxazol-3-yl)phenyl]-2-(6-cyano-3-fluoropyridin-2-yl)ethan-1-amine formate

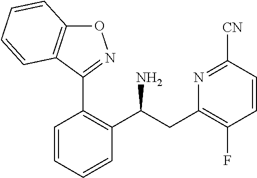 [SMILES: N#Cc1ccc(F)c(C[C@H](N)c2ccccc2-c2noc3ccccc23)n1]

Starting from (S)—N—{(S)-1-[2-(benzo[d]isoxazol-3-yl)phenyl]-2-(6-cyano-3-fluoropyridine-2-yl)ethyl}-2-methylpropane-2-sulfinamide (Intermediate 71B) and isolated from MDAP as a formate salt. ¹H NMR (400 MHz, CD₃CN) 8.07 (1H, s), 7.98-7.91 (1H, m), 7.75-7.60 (3H, m), 7.58-7.47 (4H, m), 7.44-7.36 (2H, m), 4.91-4.83 (1H, m), 3.26-3.19 (2H, m). LCMS (Method 1) RT 3.06 m/z 359 [MH⁺]

### Example 72: (S)-6-{2-Amino-2-[2-(benzo[d]isoxazol-3-yl)phenyl]ethyl}-5-fluoropyridine-2-carboxamide formate

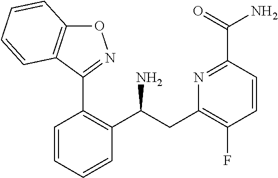 [SMILES: NC(=O)c1ccc(F)c(C[C@H](N)c2ccccc2-c2noc3ccccc23)n1]

Isolated as a formate salt from the formation of Example 71 as a by-product due to hydrolysis of the nitrile. ¹H NMR (400 MHz, CD₃CN) 8.10 (1H, s), 7.91 (1H, d, J=7.8 Hz), 7.81-7.77 (1H, m), 7.72-7.60 (3H, m), 7.53-7.43 (3H, m), 7.41-7.34 (2H, m), 7.20 (1H, br s), 5.89 (1H, br s), 4.80 (1H, t, J=7.1 Hz), 3.12 (2H, dd, J=2.3, 7.1 Hz). LCMS (Method 1) RT 2.73 m/z 377 [MH⁺].

### Example 73: (S)-1-[2-(Benzo[d]isoxazol-3-yl)phenyl]-2-(3-fluoro-6-methylpyridin-2-yl)ethan-1-amine hydrochloride

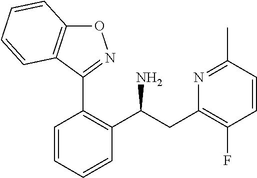 [SMILES: Cc1ccc(F)c(C[C@H](N)c2ccccc2-c2noc3ccccc23)n1]

Starting from (S)—N—{(S)-1-[2-(benzo[d]isoxazol-3-yl)phenyl]-2-(3-fluoro-6-methylpyridine-2-yl)ethyl}-2-methylpropane-2-sulfinamide (Intermediate 71C) and converting to the HCl salt by treating with aqueous HCl in acetonitrile and freeze drying. ¹H NMR (400 MHz, DMSO-d₆) 8.92 (3H, br s), 8.16-8.12 (1H, m), 7.89-7.86 (1H, m), 7.78-7.72 (2H, m), 7.63-7.55 (2H, m), 7.48-7.39 (2H, m), 7.27-7.21 (1H, m), 6.80 (1H, dd, J=3.8, 8.5 Hz), 5.34-5.27 (1H, m), 3.46-3.38 (1H, m), 3.26-3.21 (1H, m), 1.85 (3H, s). LCMS (Method 1) RT 3.11 m/z 348 [MH⁺].

### Example 74: (S)-6-{2-Amino-2-[2-(benzo[d]isoxazol-3-yl)phenyl]ethyl}-5-fluoro-N,N-dimethylpyridin-2-carboxamide hydrochloride

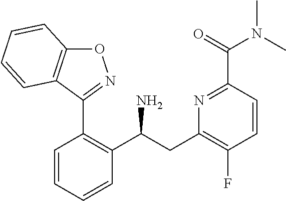 [SMILES: CN(C)C(=O)c1ccc(F)c(C[C@H](N)c2ccccc2-c2noc3ccccc23)n1]

Starting from 6-{(S)-2-[2-(benzo[d]isoxazol-3-yl)phenyl]-2-[((S)-tert-butylsulfinyl)amino]ethyl}-5-fluoro-N,N-dimethylpyridine-2-carboxamide (Intermediate 71D) and converting to the HCl salt by treating with aqueous HCl in acetonitrile and freeze drying. ¹H NMR (400 MHz, DMSO-d₆) 8.83 (3H, br s), 8.17-8.13 (1H, m), 7.89-7.86 (1H, m), 7.80-7.72 (2H, m), 7.67-7.59 (3H, m), 7.50-7.42 (2H, m), 7.39-7.35 (1H, m), 5.19 (1H, dd, J=5.9, 7.0 Hz), 3.42-3.35 (2H, m), 2.86 (3H, s), 2.48 (3H, s). LCMS (Method 1) RT 2.93 m/z 405 [MH⁺].

### Example 75: (S)-1-[2-(Benzo[d]isoxazol-3-yl)phenyl]-2-(3-cyanopyridin-2-yl)ethan-1-amine

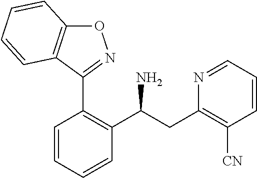 [SMILES: N#Cc1cccnc1C[C@H](N)c1ccccc1-c1noc2ccccc12]

Starting from (S)—N—{(S)-2-(3-cyanopyridine-2-yl)-1-[2-(benzo[d]isoxazol-3-yl)phenyl]ethyl}-2-methylpropane-2-sulfinamide (Intermediate 27L). ¹H NMR (400 MHz, DMSO-d₆) 8.50 (1H, d, J=4.5 Hz), 8.04 (1H, d, J=7.6 Hz), 7.95-7.85 (2H, m), 7.79-7.72 (2H, m), 7.70-7.65 (1H, m), 7.62-7.53 (2H, m), 7.54-7.48 (1H, m), 7.39 (1H, dd, J=4.9, 7.7 Hz), 6.28-6.28 (2H, m), 4.86-4.80 (1H, m), 3.04-2.98 (1H, m), 2.87-2.79 (1H, m). LCMS (Method 9) RT 2.70 m/z 341 [MH⁺].

### Example 76: (S)—N-(2-{2-Amino-2-[2-(benzo[d]isoxazol-3-yl)phenyl]ethyl}pyridin-3-yl)acetamide hydrochloride

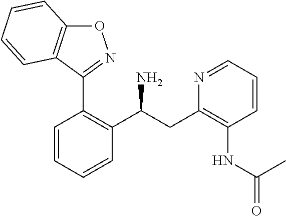 [SMILES: CC(=O)Nc1cccnc1C[C@H](N)c1ccccc1-c1noc2ccccc12]

Starting from N-(2-{(S)-2-[2-(benzo[d]isoxazol-3-yl)phenyl]-2-[((S)-tert-butylsulfinyl)amino]ethyl}pyridine-3-yl)acetamide (Intermediate 75A) and converting to the HCl salt by treating with aqueous HCl in acetonitrile and concentrating in vacuo. ¹H NMR (400 MHz, DMSO-d₆) 9.65-9.62 (1H, m), 8.71-8.68 (3H, br s), 8.10 (1H, d, J=7.6 Hz), 7.96-7.91 (2H, m), 7.83-7.74 (5H, m), 7.73-7.67 (1H, m), 7.55-7.50 (1H, m), 7.23-7.15 (1H, m), 5.41-5.36 (1H, m), 2.07-2.05 (3H, m) plus two protons hidden under the water peak. LCMS (Method 9) RT 2.45 m/z 373 [MH⁺].

### Example 77: (S)-2-(6-{2-Amino-2-(2-benzo[d]isoxazol-3-yl)phenyl]ethyl}-5-fluoropyridin-2-yl)ethan-1-ol hydrochloride

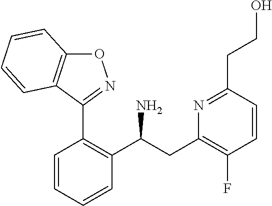 [SMILES: N[C@@H](Cc1nc(CCO)ccc1F)c1ccccc1-c1noc2ccccc12]

Starting from (S)—N—{(S)-1-[2-(benzo[d]isoxazol-3-yl)phenyl]-2-[3-fluoro-6-(2-hydroxyethyl)pyridine-2-yl)ethyl]-2-methylpropane-2-sulfinamide (Intermediate 71F) and converting to the HCl salt by treating with aqueous HCl in acetonitrile and concentrating in vacuo. ¹H NMR (400 MHz, DMSO-d₆) 8.87 (3H, br s), 8.13-8.09 (1H, m), 7.88-7.84 (1H, m), 7.78-7.71 (2H, m), 7.62-7.60 (2H, m), 7.54-7.50 (1H, m), 7.42 (1H, t, J=7.4 Hz), 7.29-7.24 (1H, m), 6.89 (1H, dd, J=3.8, 8.5 Hz), 5.32-5.25 (1H, m), 3.45-3.27 (4H, m), 2.44-2.36 (1H, m), 2.34-2.25 (1H, m). LCMS (Method 1) RT 2.90 m/z 378 [MH⁺].

### Example 78: (S)-1-[2-(Benzo[d]isoxazol-3-yl)phenyl]-2-[3-fluoro-6-(1H-pyrazol-4-yl)pyridin-2-yl)ethan-1-amine hydrochloride

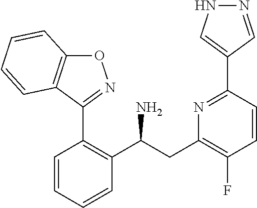 [SMILES: N[C@@H](Cc1nc(-c2cn[nH]c2)ccc1F)c1ccccc1-c1noc2ccccc12]

Starting from (S)—N—{(S)-1-[2-(benzo[d]isoxazol-3-yl)phenyl]-2-[3-fluoro-6-(1H-pyrazol-4-yl)pyridine-2-yl]ethyl}-2-methylpropane-2-sulfinamide (Intermediate 71G) and converting to the HCl salt by treating with aqueous HCl in acetonitrile and concentrating in vacuo. ¹H NMR (400 MHz, DMSO-d₆) 8.82 (3H, br s), 8.15-8.11 (1H, m), 7.80-7.75 (1H, m), 7.73 (2H, s), 7.67-7.59 (4H, m), 7.47-7.43 (1H, m), 7.41-7.27 (3H, m), 5.43-5.37 (1H, m), 3.40-3.34 (2H, m). LCMS (Method 1) RT 2.87 m/z 400 [MH⁺].

### Example 79: (S)-1-[2-(Benzo[d]isoxazol-3-yl)phenyl]-2-(3-cyclopropylpyridin-2-yl)ethan-1-amine formate

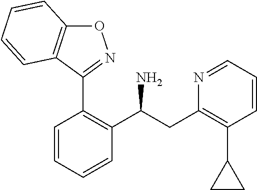 [SMILES: N[C@@H](Cc1ncccc1C1CC1)c1ccccc1-c1noc2ccccc12]

Starting from (S)—N—{(S)-1-[2-(benzo[d]isoxazol-3-yl)phenyl]ethyl}-2-(3-cyclopropylpyridine-2-yl)-2-methylpropane-2-sulfinamide (Intermediate 6U). Purification by MDAP provided the formate salt. ¹H NMR (400 MHz, DMSO-d₆) 8.10 (1H, s), 7.85-7.78 (2H, m), 7.65-7.62 (1H, m), 7.55-7.40 (3H, m), 7.31-7.25 (2H, m), 7.24-7.19 (1H, m), 6.91 (1H, dd, J=1.5, 7.8 Hz), 6.75 (1H, dd, J=4.8, 7.8 Hz), 4.66-4.61 (1H, m), 3.09-3.02 (1H, m), 2.97-2.89 (1H, m), 1.36-1.29 (1H, m), 0.51-0.37 (2H, m), 0.21-0.15 (2H, m). LCMS (Method 9) RT 2.78 m/z 356 [MH⁺].

### Example 80: (S)-1-[2-(Benzo[d]isoxazol-3-yl)phenyl]-2-(3-methyl-5-cyanopyridin-2-yl)ethan-1-amine

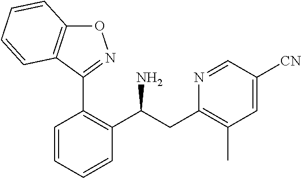 [SMILES: Cc1cc(C#N)cnc1C[C@H](N)c1ccccc1-c1noc2ccccc12]

Starting from (S)—N—{(S)-1-[2-(benzo[d]isoxazol-3-yl)phenyl]ethyl}-2-(5-cyano-2-methylpyridine-2-yl)-2-methylpropane-2-sulfinamide (Intermediate 27M). ¹H NMR (400 MHz, CDCl₃) 8.41-8.38 (1H, m), 7.92 (1H, d, J=7.6 Hz), 7.67-7.56 (3H, m), 7.52-7.43 (4H, m), 7.38-7.32 (1H, m), 4.99-4.93 (1H, m), 3.27 (1H, dd, J=5.1, 14.9 Hz), 3.16 (1H, dd, J=8.3, 14.9 Hz), 2.10 (3H, s). LCMS (Method 10) RT 3.28 m/z 355 [MH⁺].

### Example 81: (S)-1-[2-(6-Bromobenzo[d]isoxazol-3-yl)phenyl]-2-(5-cyanopyridine-2-yl)ethan-1-amine

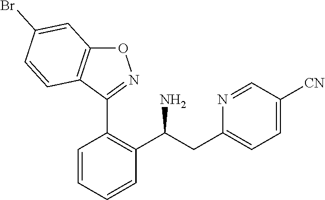 [SMILES: N#Cc1ccc(C[C@H](N)c2ccccc2-c2noc3cc(Br)ccc23)nc1]

Hydrogen chloride (4M in dioxane, 1 mL) was added to a solution of (S)—N—{(S)-1-[2-(6-bromobenzo[d]isoxazol-3-yl)phenyl]-2-(5-cyanopyridine-2-yl)ethyl}-2-methylpropane-2-sulfinamide (Intermediate 7A, 0.22 g) in methanol (2 mL) and the mixture was stirred at room temperature for 1 hour. The mixture was partitioned between DCM and water and the organic phase was dried (Na₂SO₄) and filtered. The filtrate was concentrated in vacuo and purified by FCC eluting with 0-8% ammonia in methanol (2M) in DCM to give the title compound (0.155 g) as a clear gum. ¹H NMR (400 MHz, CDCl₃) 8.65 (1H, d, J=1.5 Hz), 7.87-7.84 (2H, m), 7.76 (1H, dd, J=2.1, 8.2 Hz), 7.61-7.56 (1H, m), 7.50-7.39 (4H, m), 7.20 (1H, dd, J=0.7, 8.1 Hz), 4.75-4.70 (1H, m), 3.37-3.31 (1H, m), 3.18-3.11 (1H, m). LCMS (Method 1) RT 3.23 m/z 419/421 [MH⁺].

By proceeding in a similar manner to Example 81, the following compounds were prepared:

### Example 82: (S)-1-[2-(Benzo[d]isoxazol-3-yl)-5-methoxyphenyl]-2-(pyridine-2-yl)ethan-1-amine hydrochloride

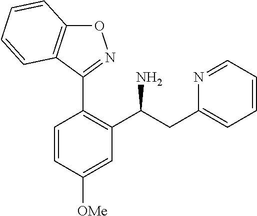 [SMILES: COc1ccc(-c2noc3ccccc23)c([C@@H](N)Cc2ccccn2)c1]

Starting from (S)—N—{(S)-1-[2-(benzo[d]isoxazol-3-yl)-5-methoxyphenyl]-2-(pyridine-2-yl)ethyl}-2-methylpropane-2-sulfinamide (Intermediate 5C) and converting to the HCl salt by treatment with 0.1M hydrochloric acid and freeze drying. ¹H NMR (400 MHz, DMSO-d₆ 1007038) 8.85 (3H, br s), 8.04 (1H, m), 7.80 (1H, d, J=8.38 Hz), 7.77-7.75 (1H, m), 7.71-7.66 (1H, m), 7.60-7.48 (3H, m), 7.41-7.35 (1H, m), 7.13 (1H, dd, J=8.38, 2.48 Hz), 7.08-6.99 (2H, m), 5.28-5.17 (1H, m), 3.90 (3H, s), 3.51-3.40 (1H, m), 3.37-3.27 (1H, m). LCMS (Method 1) RT 2.87 m/z 346 [MH⁺].

### Example 83: (S)-1-[2-(Benzo[d]isoxazol-3-yl)-5-bromophenyl]-2-(pyridine-2-yl)ethan-1-amine hydrochloride

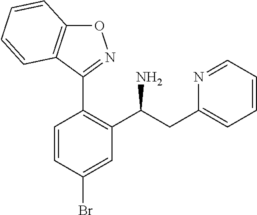 [SMILES: N[C@@H](Cc1ccccn1)c1cc(Br)ccc1-c1noc2ccccc12]

Starting from (S)—N—{(S)-1-[2-(benzo[d]isoxazol-3-yl)-5-bromophenyl]-2-(pyridine-2-yl)ethyl}-2-methylpropane-2-sulfinamide (Intermediate 5D) converting to the HCl salt by treatment with 0.1M hydrochloric acid and freeze drying. ¹H NMR (400 MHz, DMSO-d₆ 1007037) 8.93-8.83 (3H, br s), 8.38-8.36 (1H, m), 8.09-8.04 (1H, m), 7.84 (1H, d, J=8.35 Hz), 7.79 (1H, dd, J=8.35, 1.95 Hz), 7.74-7.69 (1H, m), 7.67-7.61 (1H, m), 7.58-7.52 (2H, m), 7.43-7.38 (1H, m), 7.16-7.07 (2H, m), 5.24-5.15 (1H, m), 3.54-3.45 (1H, m), 3.39-3.31 (1H, m). LCMS (Method 1) RT 3.069 m/z 394/396 [MH⁺].

### Example 84: (S)-1-[2-(Benzo[d]isoxazol-3-yl)-6-methylphenyl]-2-(pyridine-2-yl)ethan-1-amine hydrochloride

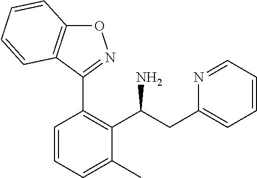 [SMILES: Cc1cccc(-c2noc3ccccc23)c1[C@@H](N)Cc1ccccn1]

Starting from (S)—N—{(S)-1-[2-(benzo[d]isoxazol-3-yl)-6-methylphenyl]-2-(pyridine-2-yl)ethyl}-2-methylpropane-2-sulfinamide (Intermediate 5E) converting to the HCl salt by treatment with 0.1M hydrochloric acid and freeze drying. ¹H NMR (400 MHz, DMSO-d₆, 80° C.) 8.20-8.15 (1H, br s), 7.87-7.81 (1H, m), 7.78-7.72 (1H, m), 7.60-7.38 (7H, m), 7.08-7.01 (1H, m), 6.84-6.78 (1H, m), 5.35-5.29 (1H, m), 3.46-3.37 (1H, m), 3.13-3.05 (1H, m), 2.59 (3H, s). LCMS (Method 1) RT 2.93 m/z 330 [MH⁺].

### Example 85: (S)-1-[2-(Benzo[d]isoxazol-3-yl)-4-bromophenyl]-2-(pyridine-2-yl)ethan-1-amine hydrochloride

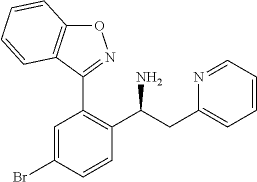 [SMILES: N[C@@H](Cc1ccccn1)c1ccc(Br)cc1-c1noc2ccccc12]

Starting from (S)—N—{(S)-1-[2-(benzo[d]isoxazol-3-yl)-4-bromophenyl]-2-(pyridine-2-yl)ethyl}-2-methylpropane-2-sulfinamide (Intermediate 5F) converting to the HCl salt by treatment with 0.1M hydrochloric acid and freeze drying. ¹H NMR (400 MHz, DMSO-d₆) 8.81 (3H, br s), 8.05-7.99 (2H, m), 7.96 (1H, dd, J=8.44, 2.30), 7.85 (1H, d, J=8.44), 7.78 (1H, d, J=2.30), 7.75-7.70 (1H, m), 7.62-7.55 (1H, m), 7.55-7.51 (1H, m), 7.44-7.39 (1H, m), 7.09-7.02 (2H, m), 5.13-5.05 (1H, m), 3.47-3.39 (1H, m), 3.34-3.27 (1H, m). LCMS (Method 1) RT 3.15 m/z 394/396 [MH⁺].

### Example 86: (S)-1-[2-(Benzo[d]isoxazol-3-yl)-3-bromophenyl]-2-(pyridine-2-yl)ethan-1-amine hydrochloride

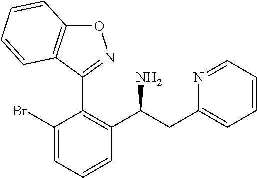 [SMILES: N[C@@H](Cc1ccccn1)c1cccc(Br)c1-c1noc2ccccc12]

Starting from (S)—N—{(S)-1-[2-(benzo[d]isoxazol-3-yl)-3-bromophenyl]-2-(pyridine-2-yl)ethyl}-2-methylpropane-2-sulfinamide (Intermediate 5H) and converting to the HCl salt by treatment with 0.1M hydrochloric acid and freeze drying. ¹H NMR (400 MHz, DMSO-d₆) 8.75 (1.8H, br s), 8.69 (1.2H, br s), 8.44-8.40 (0.4H, m), 8.09-8.03 (0.8H, m), 7.92-7.84 (2H, m), 7.82-7.61 (3.8H, m), 7.50-7.42 (1H, m), 7.34-7.27 (1H, m), 7.18-7.14 (0.4H, m), 7.01-6.92 (1H, m), 6.87-6.82 (0.6H, m), 4.61-4.50 (0.6H, m), 4.25-4.16 (0.4H, m), 3.32-3.22 (2H, m). LCMS (Method 1) RT 3.02 m/z 394/396 [MH⁺].

### Example 87: (S)-1-[2-(Benzo[d]isoxazol-3-yl)-6-bromophenyl]-2-(pyridine-2-yl)ethan-1-amine hydrochloride

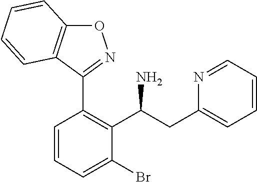 [SMILES: N[C@@H](Cc1ccccn1)c1c(Br)cccc1-c1noc2ccccc12]

Starting from (S)—N—{(S)-1-[2-(benzo[d]isoxazol-3-yl)-3-bromophenyl]-2-(pyridine-2-yl)ethyl}-2-methylpropane-2-sulfinamide (Intermediate 5J) and converting to the HCl salt by treatment with 0.1M HCl in acetonitrile and freeze drying. ¹H NMR (400 Mz, DMSO-d₆) 8.07 (1H, br s), 7.94-7.90 (1H, m), 7.83 (1H, d, J=8.3 Hz), 7.77-7.72 (1H, m), 7.61-7.57 (1H, m), 7.55-7.41 (4H, m), 7.05-7.01 (1H m), 6.91 (1H, d, J=7.7 Hz), 5.44 (1H, t, J=7.5 Hz), 3.50-3.36 (2H, m). LCMS (Method 1) RT 2.81 m/z 394/396 [MH⁺].

### Example 88: (S)-1-[2-(Benzo[d]isoxazol-3-yl)phenyl]-2-(5-bromopyridine-2-yl)ethan-1-amine hydrochloride

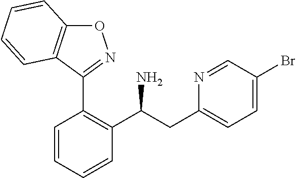 [SMILES: N[C@@H](Cc1ccc(Br)cn1)c1ccccc1-c1noc2ccccc12]

Starting from (S)—N—{(S)-1-[2-(benzo[d]isoxazol-3-yl)phenyl]-2-(5-bromopyridine-2-yl)ethyl}-2-methylpropane-2-sulfinamide (Intermediate 6D) and converting to the HCl salt by treatment with 0.1M HCl in acetonitrile and freeze drying. ¹H NMR (400 MHz, DMSO-d₆) 8.44 (3H, br s), 8.07 (1H, d, J=7.8 Hz), 7.96 (1H, d, J=2.3 Hz), 7.84 (1H, d, J=8.8 Hz), 7.74-7.67 (3H, m), 7.59-7.55 (2H, m), 7.46-7.38 (2H, m), 6.89 (1H, d, J=8.3 Hz), 5.15-5.09 (1H, m), 3.35-3.29 (1H, m), 3.24-3.17 (1H, m). LCMS (Method 1) RT 3.25 m/z 394/396 [MH⁺].

### Example 89: (S)-6-{2-Amino-2-[2-(Benzo[d]isoxazol-3-yl)phenyl]ethyl-N,N-dimethylpyridine-2-carboxamide hydrochloride

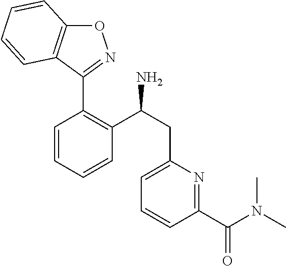 [SMILES: CN(C)C(=O)c1cccc(C[C@H](N)c2ccccc2-c2noc3ccccc23)n1]

Starting from tert-butyl (S)-{1-[2-(benzo[d]isoxazol-3-yl)phenyl]-2-[6-(dimethylcarbamoyl)pyridine-2-yl]ethyl}carbamate (Intermediate 44A) and converting to the HCl salt by treatment with 0.1M HCl in acetonitrile and freeze drying. ¹H NMR (400 MHz, DMSO-d₆) 8.65 (3H, br s), 8.09 (1H, d, J=7.9 Hz), 7.84 (1H, d, J=8.3 Hz), 7.77-7.69 (2H, m), 7.68-7.57 (4H, m), 7.44-7.38 (1H, m), 7.23 (1H, dd, J=0.9, 7.9 Hz), 7.09 (1H, dd, J=0.9, 7.6 Hz), 5.14-5.06 (1H, m), 3.47-3.42 (1H, m), 3.39-3.31 (1H, m), 2.84 (3H, s), 2.51 (3H, s). LCMS (Method 2) RT 2.75 m/z 387 [MH⁺]

### Example 90: (S)-1-[2-(Benzo[d]isoxazol-3-yl)phenyl]-2-[6-(1-methyl-1H-pyrazol-4-yl)pyridine-2-yl]ethan-1-amine hydrochloride

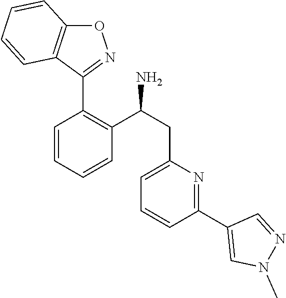 [SMILES: Cn1cc(-c2cccc(C[C@H](N)c3ccccc3-c3noc4ccccc34)n2)cn1]

Starting from tert-butyl (S)-{1-[2-(benzo[d]isoxazol-3-yl)phenyl]-2-[6-(1-methyl-1H-pyrazol-4-yl)pyridine-2-yl]ethyl}carbamate (Intermediate 46A) and converting to the HCl salt by treatment with 0.1M HCl in acetonitrile and freeze drying. ¹H NMR (400 MHz, DMSO-d₆) 8.77 (3H, br s), 8.12 (1H, d, J=7.9 Hz), 7.81-7.73 (2H, m), 7.72-7.68 (1H, m), 7.67-7.59 (4H, m), 7.49 (1H, t, J=7.8 Hz), 7.44 (1H, d, J=7.9 Hz), 7.36-7.30 (1H, m), 7.24 (1H, d, J=7.7 Hz), 6.76 (1H, d, J=7.7 Hz), 5.39-5.28 (1H, m), 3.84 (3H, s) 3.30-3.21 (1H, m), plus one proton under the water peak. LCMS (Method 2) RT 2.81 m/z 396 [MH⁺].

### Example 91: (S)-2-[6-(1H-Pyrazol-4-yl)pyridine-2-yl])-1-[2-(benzo[d]isoxazol-3-yl)phenyl]ethan-1-amine hydrochloride

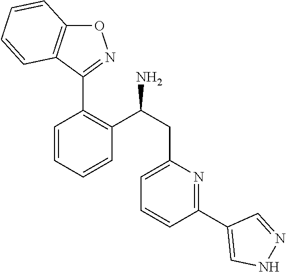 [SMILES: N[C@@H](Cc1cccc(-c2cn[nH]c2)n1)c1ccccc1-c1noc2ccccc12]

Starting from (S)—N—{(S)-2-[6-(1H-pyrazol-4-yl)pyridine-2-yl]-1-[2-(benzo[d]isoxazol-3-yl)phenyl]ethyl}propane-2-sulfinamide (Intermediate 47A) and converting to the HCl salt by dissolving in methanol and treating with 1.25M HCl in methanol and the concentrating in vacuo. ¹H NMR (400 MHz, DMSO-d₆) 12.94 (1H, br s), 8.07 (1H, d, J=8.0 Hz), 7.89 (1H, br s), 7.79-7.56 (7H, m), 7.55-7.45 (2H, m,), 7.40-7.27 (2H, m), 6.76 (1H, d, J=8.0 Hz), 5.19-5.09 (1H, m), 3.27-3.09 (2H, m). LCMS (Method 2) RT 2.75 m/z 382 [MH⁺]

### Example 92: (S)-1-[2-(Benzo[d]isoxazol-3-yl)phenyl]-2-(5-methylpyridine-2-yl)ethan-1-amine hydrochloride

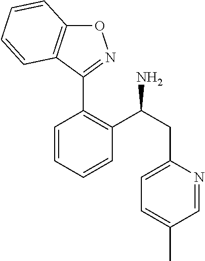 [SMILES: Cc1ccc(C[C@H](N)c2ccccc2-c2noc3ccccc23)nc1]

Starting from (S)—N—{(S)-1-[2-(benzo[d]isoxazol-3-yl)phenyl]-2-(5-methylpyridine-2-yl)ethyl}-2-methylpropane-2-sulfinamide (Intermediate 11E) and converting to the HCl salt by treatment with 0.1M aqueous HCl in acetonitrile and freeze drying. ¹H NMR (400 MHz, DMSO-d₆) 8.83 (3H, br s), 8.11 (1H, d, J=8.1 Hz), 7.97 (1H, br s), 7.88 (1H, d, J=8.6 Hz), 7.79-7.72 (2H, m), 7.65-7.60 (2H, m), 7.54 (1H, d, J=8.1 Hz), 7.47-7.42 (1H, m), 6.94 (2H, br s), 5.28-5.21 (1H, m), 2.13 (3H, s) plus 2 protons hidden under the water peak. LCMS (Method 2) RT 2.45 m/z 330 [MH⁺].

### Example 93: (S)-1-[2-(Benzo[d]isoxazol-3-yl)phenyl]-2-(5-cyanopyridine-2-yl)ethan-1-amine hydrochloride

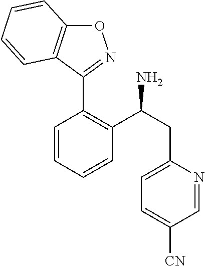 [SMILES: N#Cc1ccc(C[C@H](N)c2ccccc2-c2noc3ccccc23)nc1]

Starting from (S)—N—{(S)-1-[2-(benzo[d]isoxazol-3-yl)phenyl]-2-(5-cyanopyridine-2-yl)ethyl}-2-methylpropane-2-sulfinamide (Intermediate 27C) and converting to the HCl salt by treatment with 0.1M aqueous HCl in acetonitrile and freeze drying. ¹H NMR (400 MHz, DMSO-d₆) 8.83 (3H, br s), 8.22 (1H, dd, J=0.7, 5.1 Hz), 8.12 (1H, 7.9 Hz), 7.90-7.86 (1H, m), 7.81-7.73 (2H, m), 7.66-7.62 (2H, m), 7.56-7.53 (1H, m), 7.50-7.41 (3H, m), 5.31-5.19 (1H, m), 3.56-3.48 (1H, m), 3.42-3.34 (1H, m). LCMS (Method 2) RT 2.84 m/z 341 [MH⁺].

### Example 94: (S)-1-[2-(Benzo[d]isoxazol-3-yl)phenyl]-2-[6-(3,5-dimethylisoxazol-4-yl)pyridine-2-yl)-ethan-1-amine hydrochloride

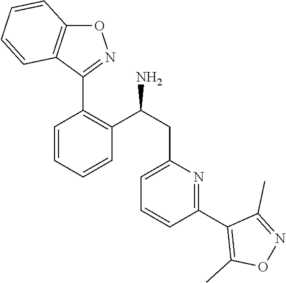 [SMILES: Cc1noc(C)c1-c1cccc(C[C@H](N)c2ccccc2-c2noc3ccccc23)n1]

Starting from (S)—N—{(S)-1-[2-(benzo[d]isoxazol-3-yl)phenyl]-2-[6-(3,5-dimethylisoxazol-4-yl)pyridine-2-yl]ethyl}propane-2-sulfinamide (Intermediate 50A) and converting to the HCl salt by dissolving in 1.25M HCl in methanol and concentrating in vacuo. ¹H NMR (400 MHz, DMSO-d₆) 8.07-8.03 (1H, m), 7.76 (1H, d, J=8.7 Hz), 7.74-7.59 (3H, m), 7.55-7.48 (3H, m), 7.38-7.33 (1H, m), 7.22-7.19 (1H, m), 6.92 (1H, d, J=7.3 Hz), 4.90 (1H, t, J=6.8 Hz), 3.22-3.08 (2H, m), 2.31 (3H, s), 2.09 (3H, s). LCMS (Method 1) RT 3.19 m/z 411 [MH⁺].

### Example 95: (S)-1-[2-(6-Cyanobenzo[d]isoxazol-3-yl)phenyl]-2-(6-cyanopyridine-2-yl)ethan-1-amine

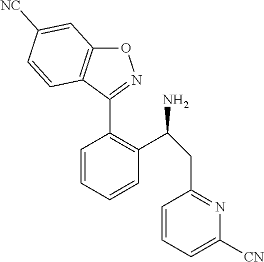 [SMILES: N#Cc1ccc2c(-c3ccccc3[C@@H](N)Cc3cccc(C#N)n3)noc2c1]

Starting from (S)—N—{(S)-1-[2-(6-Cyanobenzo[d]isoxazol-3-yl)phenyl]-2-(6-cyanopyridine-2-yl)ethyl}-2-methylpropane-2-sulfinamide (Intermediate 27D). ¹H NMR (400 MHz, DMSO-d₆) 8.56 (1H, s), 7.98 (1H, d, J=7.3 Hz), 7.81 (1H, dd, J=1.0, 8.2 Hz), 7.77-7.71 (2H, m), 7.68-7.63 (2H, m), 7.46-7.38 (2H, m), 7.16 (1H, dd, J=1.0, 7.8 Hz), 4.48 (1H, t, J=6.8 Hz), 3.03-2.97 (2H, m). LCMS (Method 1) RT 2.77 m/z 366 [MH⁺].

### Example 96: (S)-1-[2-(6-Cyanobenzo[d]isoxazol-3-yl)phenyl]-2-(6-methylpyridine-2-yl)ethan-1-amine

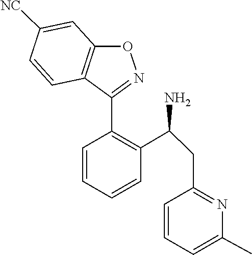 [SMILES: Cc1cccc(C[C@H](N)c2ccccc2-c2noc3cc(C#N)ccc23)n1]

Starting from (S)—N—{(S)-1-[2-(6-cyanobenzo[d]isoxazol-3-yl)phenyl]-2-(6-methylpyridine-2-yl)ethyl}-2-methylpropane-2-sulfinamide (Intermediate 27E). ¹H NMR (400 MHz, DMSO-d₆) 8.56 (1H, s), 8.00-7.96 (1H, m), 7.82-7.72 (2H, m), 7.66-7.61 (1H, m), 7.45-7.31 (3H, m), 6.82 (1H, d, J=7.4 Hz), 6.58 (1H, d, J=7.3 Hz), 4.49 (1H, t, J=7.1 Hz), 2.88-2.84 (2H, m), 2.09 (3H, s). LCMS (Method 1) RT 2.41 m/z 355 [MH⁺]

### Example 97: (S)-1-[2-(6-Cyanobenzo[d]isoxazol-3-yl)phenyl]-2-(6-methylaminopyridine-2-yl)ethan-1-amine dihydrochloride

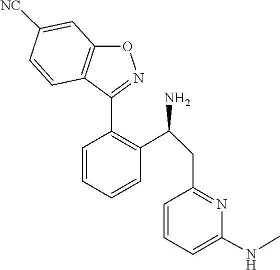 [SMILES: CNc1cccc(C[C@H](N)c2ccccc2-c2noc3cc(C#N)ccc23)n1]

Starting from (S)—N—{(S)-1-[2-(6-cyanobenzo[d]isoxazol-3-yl)phenyl]-2-(6-methylaminopyridine-2-yl)ethyl}-2-methylpropane-2-sulfinamide (Intermediate 27F) and converting to the HCl salt by dissolving in 1.25M HCl in methanol and concentrating in vacuo. ¹H NMR (400 MHz, DMSO-d₆) 8.55 (1H, s), 8.20 (2H, br s), 8.08 (1H, d, J=7.8 Hz), 7.82-7.73 (2H, m), 7.65 (1H, d, J=8.1 Hz), 7.60 (2H, d, J=3.9 Hz), 7.04 (1H, dd, J=7.3, 8.3 Hz), 6.18-6.12 (1H, m), 5.99-5.96 (2H, m), 5.07 (1H, t, J=7.1 Hz), 3.18-3.05 (1H, m), 2.93 (1H, dd, J=7.3, 13.7 Hz), 2.35 (3H, d, J=4.9 Hz). LCMS (Method 1) RT 2.07 m/z 370 [MH⁺].

### Example 98: (S)-1-[2-(Benzo[d]isoxazol-3-yl)phenyl]-2-(6-hydroxymethylpyridine-2-yl)ethan-1-amine hydrochloride

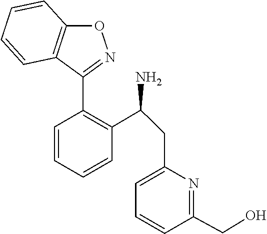 [SMILES: N[C@@H](Cc1cccc(CO)n1)c1ccccc1-c1noc2ccccc12]

Starting from (S)—N—{(S)-1-[2-(benzo[d]isoxazol-3-yl)phenyl]-2-(6-hydroxymethylpyridin-2-yl)ethyl}-2-methylpropane-2-sulfinamide (Intermediate 14B) and converting to the HCl salt by treating with HCl in methanol and concentrating in vacuo. ¹H NMR (400 MHz, DMSO-d₆) 8.00-7.97 (1H, m), 7.87-7.84 (1H, m), 7.76-7.65 (2H, m), 7.59-7.49 (4H, m), 7.44-7.39 (1H, m), 7.11 (1H, d, J=7.3 Hz), 6.80-6.76 (1H, m), 5.83 (2H, br s), 5.22-5.16 (1H, m), 4.79 (1H, t, J=6.8 Hz), 4.17 (2H, ddd, J=5.6, 14.7, 36.4 Hz), 3.10 (2H, d, J=6.8 Hz). LCMS (Method 1) RT 2.60 m/z 346 [MH⁺].

### Example 99: (S)-1-[2-(6-Cyanobenzo[d]isoxazol-3-yl)phenyl]-2-[6-(2-hydroxyethoxyl)pyridine-2-yl]ethan-1-amine hydrochloride

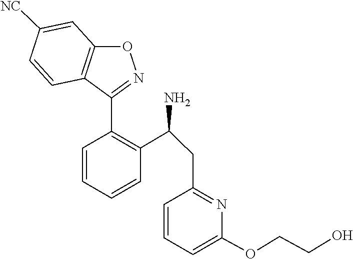 [SMILES: N#Cc1ccc2c(-c3ccccc3[C@@H](N)Cc3cccc(OCCO)n3)noc2c1]

Starting from (S)—N-{(1S)-1-[2-(6-cyanobenzo[d]isoxazol-3-yl)phenyl]-2-[6-{2-[(tetrahydro-2H-pyran-2-yl)oxy]ethoxy}pyridine-2-yl]ethyl}-2-methylpropane-2-sulfinamide (Intermediate 27G) and converting to the HCl salt by treating with HCl in methanol and concentrating in vacuo. ¹H NMR (400 MHz, DMSO-d₆) 8.56 (1H, s), 8.07-8.03 (1H, m), 7.81 (1H, dd, J=1.5, 8.3 Hz), 7.77-7.67 (2H, m), 7.57-7.53 (2H, m), 7.35 (1H, dd, J=7.1, 8.1 Hz), 6.82 (2H, s), 6.46 (1H, d, J=7.3 Hz), 6.35-6.32 (1H, m), 4.92 (1H, t, J=6.7 Hz), 4.75 (1H, s), 3.82-3.75 (1H, m), 3.60-3.48 (3H, m), 3.12-2.94 (2H, m). LCMS (Method 1) RT 2.82 m/z 401 [MH⁺].

### Example 100: (S)-1-[2-(Benzo[d]isoxazol-3-yl)phenyl]-2-[6-(2-hydroxyethyl)pyridine-2-yl]ethan-1-amine hydrochloride

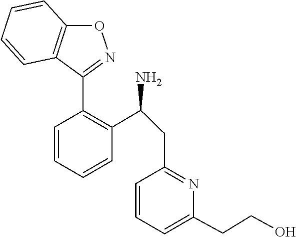 [SMILES: N[C@@H](Cc1cccc(CCO)n1)c1ccccc1-c1noc2ccccc12]

Starting from (S)—N—{(S)-1-[2-(benzo[d]isoxazol-3-yl)phenyl]-2-[6-(2-hydroxyethyl)pyridin-2-yl]ethyl}-2-methylpropane-2-sulfinamide (Intermediate 35B) and converting to the HCl salt by treating with HCl in methanol and concentrating in vacuo. ¹H NMR (400 MHz, DMSO-d₆) 8.01-7.97 (1H, m), 7.86-7.83 (1H, m), 7.75-7.70 (1H, m), 7.70-7.66 (1H, m), 7.57-7.51 (3H, m), 7.43-7.38 (2H, m), 6.89 (1H, d, J=7.5 Hz), 6.73-6.70 (1H, m), 5.94 (2H, br s), 4.83 (1H, t, J=6.8 Hz), 4.50 (1H, s), 3.48 (2H, t, J=6.8 Hz), 3.08 (2H, d, J=7.0 Hz), 2.59-2.52 (1H, m), 2.48-2.43 (1H, m). LCMS (Method 2) RT 2.44 m/z 360 [MH⁺].

### Example 101: (S)-1-[2-(Benzo[d]isoxazol-3-yl)phenyl]-2-(6-ethylpyridine-2-yl)ethan-1-amine hydrochloride

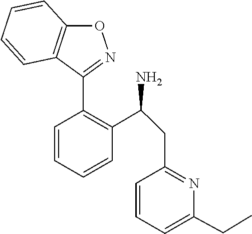 [SMILES: CCc1cccc(C[C@H](N)c2ccccc2-c2noc3ccccc23)n1]

Starting from (S)—N—{(S)-1-[2-(benzo[d]isoxazol-3-yl)phenyl]-2-(6-ethylpyridin-2-yl)ethyl}-2-methylpropane-2-sulfinamide (Intermediate 35C) and converting to the HCl salt by treating with HCl in methanol and concentrating in vacuo. ¹H NMR (400 MHz, DMSO-d₆) 8.02-7.99 (1H, m), 7.86-7.82 (1H, m), 7.75-7.71 (1H, m), 7.71-7.65 (1H, m), 7.55-7.50 (3H, m), 7.39 (2H, dd, J=7.5, 13.4 Hz), 6.84 (1H, d, J=7.6 Hz), 6.69-6.66 (1H, m), 5.94 (2H, s), 4.87 (1H, t, J=6.9 Hz), 3.08 (2H, d, J=7.0 Hz), 2.40-2.24 (2H, m), 0.92 (3H, t, J=7.6 Hz). LCMS (Method 2) RT 2.71 m/z 344 [MH⁺].

### Example 102: (S)-1-[2-(Benzo[d]isoxazol-3-yl)phenyl]-2-(6-vinylpyridin-2-yl)ethan-1-amine

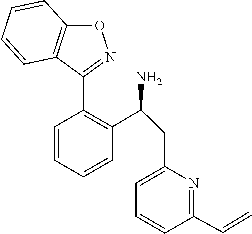 [SMILES: C=Cc1cccc(C[C@H](N)c2ccccc2-c2noc3ccccc23)n1]

Starting from (S)—N—{(S)-1-[2-(benzo[d]isoxazol-3-yl)phenyl]-2-(6-vinylpyridin-2-yl)ethyl}-2-methylpropane-2-sulfinamide (Intermediate 13B). ¹H NMR (400 MHz, CDCl₃) 7.91-7.87 (1H, m), 7.67-7.63 (1H, m), 7.61-7.52 (3H, m), 7.50-7.38 (3H, m), 7.34-7.29 (1H, m), 7.06 (1H, d, J=7.8 Hz), 6.86 (1H, d, J=7.6 Hz), 6.66 (1H, dd, J=10.7, 17.4 Hz), 6.02 (1H, dd, J=1.3, 17.5 Hz), 5.34 (1H, dd, J=1.2, 10.7 Hz), 4.71 (1H, dd, J=4.4, 9.0 Hz), 3.21 (1H, dd, J=4.4, 13.6 Hz), 3.03 (1H, dd, J=9.0, 13.5 Hz). LCMS (Method 6) RT 2.37 m/z 342 [MH⁺].

### Example 103: (S)-1-[2-(Benzo[d]isoxazol-3-yl)phenyl]-2-[6-(methylsulfonyl)pyridin-2-yl]ethan-1-amine

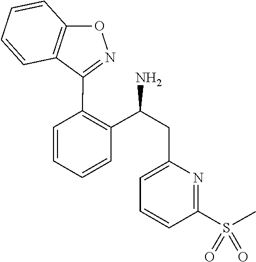 [SMILES: CS(=O)(=O)c1cccc(C[C@H](N)c2ccccc2-c2noc3ccccc23)n1]

Starting from (S)—N—{(S)-1-[2-(benzo[d]isoxazol-3-yl)phenyl]-2-[6-(methylsulfonyl)pyridine-2-yl]ethyl}propane-2-sulfinamide (Intermediate 56A). ¹H NMR (400 MHz, DMSO-d₆) 8.09-8.05 (1H, m), 7.92-7.83 (2H, m), 7.78-7.70 (3H, m), 7.68-7.64 (1H, m), 7.63-7.56 (2H, m), 7.45-7.41 (1H, m), 7.36-7.34 (1H, m), 7.04 (2H, br s), 4.93 (1H, t, J=7.0 Hz), 3.42-3.34 (2H, m), 2.95 (3H, s). LCMS (Method 1) RT 2.86 m/z 394 [MH⁺].

### Example 104: (S)-6-{2-Amino-2-[2-(benzo[d]isoxazol-3-yl)phenyl]ethyl}pyridine-2-carboxamide hydrochloride

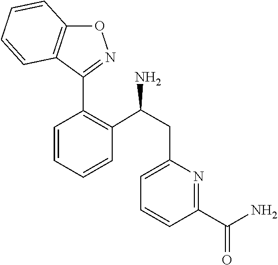 [SMILES: NC(=O)c1cccc(C[C@H](N)c2ccccc2-c2noc3ccccc23)n1]

Starting from 6-{(S)-2-[2-(benzo[d]isoxazol-3-yl)phenyl]-2-[((S)-tert-butylsulfinyl)amino]ethyl}pyridine-2-carboxamide (Intermediate 59A) and converting to the HCl salt by treating with HCl in methanol and concentrating in vacuo. ¹H NMR (400 MHz, DMSO-d₆) 8.01-7.97 (1H, m), 7.84-7.81 (1H, m), 7.76-7.69 (4H, m), 7.63-7.52 (4H, m), 7.47 (1H, s), 7.44-7.39 (1H, m), 7.23-7.20 (1H, m), 5.68 (2H, br s), 4.85 (1H, t, J=6.8 Hz), 3.20-3.16 (2H, m). LCMS (Method 1) RT 2.58 m/z 359 [MH⁺].

### Example 105: (S)-6-{2-Amino-2-[2-(benzo[d]isoxazol-3-yl)phenyl]ethyl}-N-methylpyridine-2-carboxamide hydrochloride

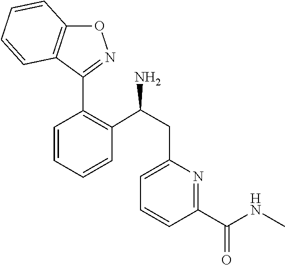 [SMILES: CNC(=O)c1cccc(C[C@H](N)c2ccccc2-c2noc3ccccc23)n1]

Starting from 6-{(S)-2-[2-(benzo[d]isoxazol-3-yl)phenyl]-2-[((S)-tert-butylsulfinyl)amino]ethyl}-N-methylpyridine-2-carboxamide (Intermediate 59B) and converting to the HCl salt by treating with HCl in methanol and concentrating in vacuo. ¹H NMR (400 MHz, DMSO-d₆) 8.03-7.99 (1H, m), 7.87-7.84 (1H, m), 7.76-7.68 (4H, m), 7.57-7.53 (3H, m), 7.40 (1H, t, J=7.2 Hz), 7.17 (1H, dd, J=1.3, 7.3 Hz), 5.97 (2H, br s), 4.93 (1H, t, J=6.9 Hz), 3.26-3.15 (3H, m), 2.76 (3H, d, J=5.0 Hz). LCMS (Method 1) RT 2.67 m/z 373 [MH⁺].

### Example 106: (S)-(6-{2-Amino-2-[2-(benzo[d]isoxazol-3-yl)phenyl]ethyl}pyridine-2-yl)(morpholino)methanone hydrochloride

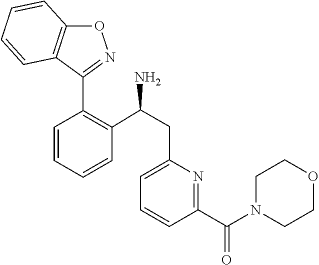 [SMILES: N[C@@H](Cc1cccc(C(=O)N2CCOCC2)n1)c1ccccc1-c1noc2ccccc12]

Starting from (S)—N—{(S)-1-[2-(benzo[d]isoxazol-3-yl)phenyl]-2-[6-(morpholine-4-carbonyl)pyridine-2-yl]ethyl}propane-2-sulfinamide (Intermediate 59C) and converting to the HCl salt by treating with HCl in methanol and concentrating in vacuo. ¹H NMR (400 MHz, DMSO-d₆) 8.02-7.99 (1H, m), 7.87-7.84 (1H, m), 7.76-7.63 (4H, m), 7.56-7.52 (2H, m), 7.43 (1H, t, J=7.2 Hz), 7.31 (1H, d, J=6.8 Hz), 7.08 (1H, d, J=7.0 Hz), 4.78-4.72 (1H, m), 3.61-3.52 (4H, m), 3.21-3.04 (4H, m) plus two protons under the water peak. LCMS (Method 1) RT 2.81 m/z 429 [MH⁺].

### Example 107: (S)-6-{2-Amino-2-[2-(benzo[d]isoxazol-3-yl)phenyl]ethyl}-N-(cyclopropylmethylpyridine-2-carboxamide hydrochloride

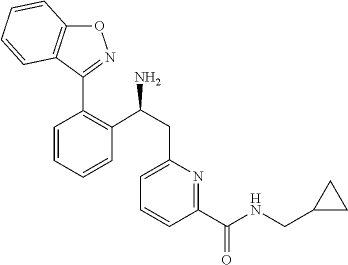 [SMILES: N[C@@H](Cc1cccc(C(=O)NCC2CC2)n1)c1ccccc1-c1noc2ccccc12]

Starting from 6-{(S)-2-[2-(benzo[d]isoxazol-3-yl)phenyl]-2-[((S)-tert-butylsulfinyl)amino]ethyl}-N-(cyclopropylmethyl)pyridine-2-carboxamide (Intermediate 59D) and converting to the HCl salt by treating with HCl in methanol and concentrating in vacuo.

¹H NMR (400 MHz, DMSO-d₆) 8.06-8.01 (1H, m), 7.96 (1H, d, J=8.2 Hz), 7.83-7.79 (1H, m), 7.73-7.67 (3H, m), 7.65-7.60 (1H, m), 7.57-7.53 (1H, m), 7.45-7.43 (2H, m), 7.41-7.36 (1H, m), 7.14-7.11 (1H, m), 4.59 (1H, t, J=6.9 Hz), 3.22-2.97 (4H, m), 1.05-0.95 (1H, m), 0.47-0.42 (2H, m), 0.25-0.21 (2H, m). LCMS (Method 2) RT 3.04 m/z 413 [MH⁺].

### Example 108: (S)-6-{2-[Amino-2-(benzo[d]isoxazol-3-yl)phenyl]ethyl}-N-(2-methoxyethyl)pyridine-2-carboxamide hydrochloride

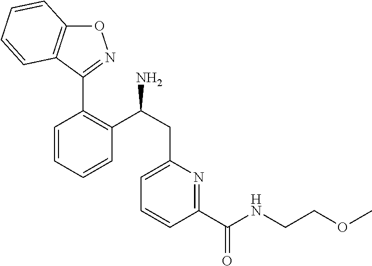 [SMILES: COCCNC(=O)c1cccc(C[C@H](N)c2ccccc2-c2noc3ccccc23)n1]

Starting from 6-{(S)-2-[2-(benzo[d]isoxazol-3-yl)phenyl]-2-[((S)-tert-butylsulfinyl)amino]ethyl}-N-(2-methoxyethyl)pyridine-2-carboxamide (Intermediate 59E) and converting to the HCl salt by treating with HCl in methanol and concentrating in vacuo.

¹H NMR (400 MHz, DMSO-d₆) 8.03-7.98 (1H, m), 7.98-7.94 (1H, m), 7.83-7.80 (1H, m), 7.73-7.66 (3H, m), 7.66-7.61 (1H, m), 7.55-7.52 (1H, m), 7.47-7.43 (2H, m), 7.40-7.36 (1H, m), 7.11 (1H, dd, J=2.3, 6.5 Hz), 4.60 (1H, t, J=6.9 Hz), 3.48-3.35 (4H, m), 3.27 (3H, s), 3.11-2.98 (2H, m). LCMS (Method 2) RT 2.75 m/z 417 [MH⁺].

### Example 109: (S)-3-(6-{2-Amino-2-[2-(benzo[d]isoxazol-3-yl)phenyl]ethyl}pyridine-2-yl)-N,N-dimethylpropanamide hydrochloride

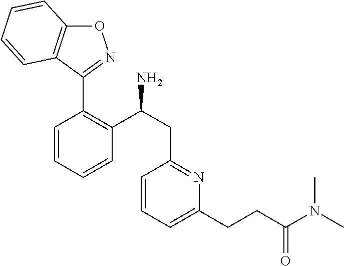 [SMILES: CN(C)C(=O)CCc1cccc(C[C@H](N)c2ccccc2-c2noc3ccccc23)n1]

Starting from 3-(6-{(S)-2-[2-(benzo[d]isoxazol-3-yl)phenyl]-2-[((S)-tert-butylsulfinyl)amino]ethyl}pyridine-2-yl)-N,N-dimethylpropanamide (Intermediate 67A) and converting to the HCl salt by treating with HCl in methanol and concentrating in vacuo. ¹H NMR (400 MHz, DMSO-d₆) 8.01-7.98 (1H, m), 7.86-7.82 (1H, m), 7.76-7.69 (2H, m), 7.64-7.54 (3H, m), 7.44-7.39 (2H, m), 6.91 (1H, d, J=7.6 Hz), 6.78-6.74 (1H, m), 5.07 (1H, t, J=6.8 Hz), 3.25-3.17 (2H, m), 2.88 (3H, s), 2.78 (3H, s), 2.69-2.54 (2H, m), 2.46-2.31 (2H, m). LCMS (Method 2) RT 2.69 m/z 415 [MH⁺].

### Example 110: (S)-6-{2-Amino-2-[2-(benzo[d]isoxazol-3-yl)-6-fluorophenyl]ethyl}-2-cyanopyridine hydrochloride

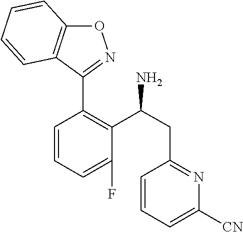 [SMILES: N#Cc1cccc(C[C@H](N)c2c(F)cccc2-c2noc3ccccc23)n1]

Starting from (S)—N—{(S)-1-[2-(benzo[d]isoxazol-3-yl)-6-fluorophenyl]-2-(6-cyanopyridine-2-yl)ethyl}-2-methylpropane-2-sulfinamide (Intermediate 271) and converting to the HCl salt by treating with HCl in methanol and concentrating in vacuo. ¹H NMR (400 MHz, DMSO-d₆) 8.44 (3H, br s), 7.89-7.86 (1H, m), 7.79-7.55 (5H, m), 7.50-7.39 (3H, m), 7.32-7.29 (1H, m), 5.11 (1H, t, J=7.4 Hz), 3.43-3.39 (2H, m). LCMS (Method 1) RT 2.94 m/z 359 [MH⁺].

### Example 111: (S)-1-[2-(Benzo[d]isoxazol-3-yl)-6-fluorophenyl]-2-(pyridin-2-yl)ethan-1-amine hydrochloride

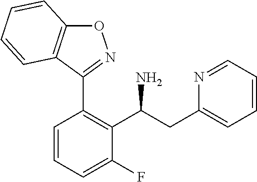 [SMILES: N[C@@H](Cc1ccccn1)c1c(F)cccc1-c1noc2ccccc12]

Starting from (S)—N—{(S)-1-[2-(benzo[d]isoxazol-3-yl)-6-fluorophenyl]-2-(pyridin-2-yl)ethyl}-2-methylpropane-2-sulfinamide (Intermediate 6K) and converting to the HCl salt by treating with HCl in methanol and concentrating in vacuo. ¹H NMR (400 MHz, DMSO-d₆) 7.90-7.87 (2H, m), 7.77-7.73 (1H, m), 7.57-7.38 (6H, m), 6.98-6.94 (2H, m), 5.08 (1H, t, J=7.8 Hz) plus two protons under the water peak. LCMS (Method 1) RT 2.75 m/z 334 [MH⁺].

### Example 112: (S)-1-[2-(Benzo[d]isoxazol-3-yl)-6-fluorophenyl]-2-(6-methylpyridin-2-yl)ethan-1-amine hydrochloride

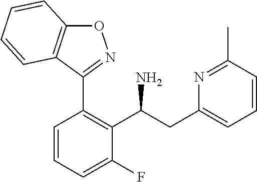 [SMILES: Cc1cccc(C[C@H](N)c2c(F)cccc2-c2noc3ccccc23)n1]

Starting from (S)—N—{(S)-1-[2-(benzo[d]isoxazol-3-yl)-6-fluorophenyl]-2-(6-methylpyridin-2-yl)ethyl}-2-methylpropane-2-sulfinamide (Intermediate 6L) and converting to the HCl salt by treating with HCl in methanol and concentrating in vacuo. ¹H NMR (400 MHz, DMSO-d₆) 8.02 (2H, s), 7.87 (1H, d, J=8.4 Hz), 7.78-7.73 (1H, m), 7.66-7.54 (2H, m), 7.42-7.28 (4H, m), 6.73 (1H, d, J=7.6 Hz), 6.66 (1H, d, J=7.6 Hz), 5.15 (1H, t, J=7.5 Hz), 3.27-3.23 (2H, m), 1.81 (3H, s). LCMS (Method 1) RT 2.57 m/z 348 [MH⁺].

### Example 113: (S)-1-[2-(5-Bromobenzo[d]isoxazol-3-yl)phenyl]-2-(pyridine-2-yl)ethan-1-amine hydrochloride

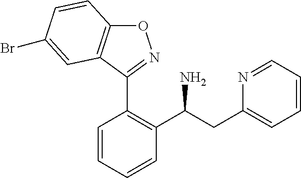 [SMILES: N[C@@H](Cc1ccccn1)c1ccccc1-c1noc2ccc(Br)cc12]

A solution of (S)—N—{(S)-1-[2-(benzo[d]isoxazol-3-yl)phenyl]-2-(pyridine-2-yl)ethyl}-2-methylpropane-2-sulfinamide (Intermediate 6A, 0.2 g) in acetonitrile (1.5 mL) was treated with 1,3-dibromo-5,5-dimethylhydantoin (0.096 g) and concentrated sulfuric acid (0.082 mL) and the mixture was stirred at room temperature for 2 hours. Further 1,3-dibromo-5,5-dimethylhydantoin (0.096 g) and concentrated sulfuric acid (0.026 mL) were added and the mixture was stirred for an additional 16 hours. Further 1,3-dibromo-5,5-dimethylhydantoin (0.24 g) was added in portions over the next 5 hours and the mixture was stirred for 72 hours. The mixture was loaded directly onto an SCX-2 cartridge which was washed with DCM and then a mixture of DCM and methanol (1:1). The product was eluted off the column with methanolic ammonia (2M) in DCM. The resultant material was purified by FCC using a C-18 cartridge and eluting with 10-30% acetonitrile in water containing 0.1% formic acid then by HPLC eluting with 10-30% acetonitrile in water containing 0.1% formic acid. The material was then converted to the hydrochloride salt by treatment with 0.1M aqueous hydrochloric acid and freeze drying. ¹H NMR (400 MHz, DMSO-d₆) 8.83 (3H, br s), 8.14-8.07 (2H, m), 7.88-7.82 (2H, m), 7.78-7.72 (1H, m), 7.71-7.64 (1H, m), 7.62-7.56 (3H, m), 7.18-7.08 (2H, m), 5.17-5.08 (1H, m), 2 additional peaks under the water. LCMS (Method 1) RT 3.19 m/z 394/396.

### Example 114: (S)-1-[2-(Benzo[d]isoxazol-3-yl)phenyl]-2-(6-methylpyridine-2-yl)ethan-1-amine hydrochloride

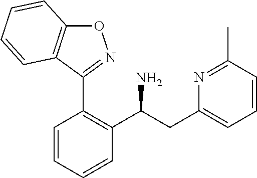 [SMILES: Cc1cccc(C[C@H](N)c2ccccc2-c2noc3ccccc23)n1]

tert-Butyl (S)-{1-[2-(benzo[d]isoxazol-3-yl)phenyl]-2-(6-methylpyridine-2-yl)ethyl}carbamate (Intermediate 11D, 0.035 g) was added to hydrogen chloride (4M in dioxane, 2 mL) and the resultant mixture was stirred at room temperature for 1 hour. Saturated aqueous sodium bicarbonate solution was added and the mixture was extracted with DCM and the organic layer was filtered through a hydrophobic frit. The filtrate was concentrated in vacuo and the residue was purified by FCC eluting with 0-5% methanol in DCM. After concentration of the appropriate fractions, the residue was dissolved in acetonitrile and treated with hydrochloric acid (0.1M) and the mixture was freeze dried to give the title compound (0.026 g) as a white solid. ¹H NMR (400 MHz, DMSO-d₆) 8.88 (3H, br s), 8.14 (1H, d, J=7.6 Hz), 7.87 (1H, d, J=8.5 Hz), 7.79-7.72 (2H, m), 7.64-7.56 (3H, m), 7.50-7.39 (3H, m), 6.96-6.76 (1H, br s), 5.29-5.21 (1H, br s), 2.03 (3H, br s), 2 additional peaks under the water. LCMS (Method 1) RT 3.08 m/z 330 [MH⁺].

By proceeding in a similar manner to Example 114, the following compounds were prepared:

### Example 115: (S)-1-[2-(Benzo[d]isoxazol-3-yl)phenyl]-2-(6-methoxypyridine-2-yl)ethan-1-amine hydrochloride

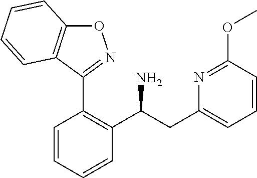 [SMILES: COc1cccc(C[C@H](N)c2ccccc2-c2noc3ccccc23)n1]

Starting from tert-butyl (S)-{1-[2-(benzo[d]isoxazol-3-yl)phenyl]-2-(6-methoxypyridine-2-yl)ethyl}carbamate (Intermediate 40A) and converting to the HCl salt by treatment with 0.1M HCl in acetonitrile and freeze drying. ¹H NMR (400 MHz, DMSO-d₆) 8.69 (3H, br s), 8.06 (1H, d, J=7.8 Hz), 7.83 (1H, d, J=8.5 Hz), 7.77-7.68 (2H, m), 7.61-7.57 (2H, m), 7.49-7.45 (1H, m), 7.41-7.32 (2H, m), 6.49 (1H, d, J=7.13 Hz), 6.33 (1H, d, J=8.5 Hz), 5.23-5.16 (1H, m), 3.27 (3H, s), 3.26-3.22 (1H, m), 3.16-3.08 (1H, m). LCMS (Method 1) RT 3.16 m/z 346 [MH⁺].

### Example 116: (S)-1-[2-(Benzo[d]isoxazol-3-yl)phenyl]-2-(6-cyanopyridine-2-yl)ethan-1-amine hydrochloride

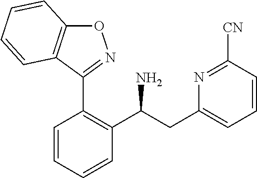 [SMILES: N#Cc1cccc(C[C@H](N)c2ccccc2-c2noc3ccccc23)n1]

Starting from tert-butyl (S)-{1-[2-(benzo[d]isoxazol-3-yl)phenyl]-2-(6-cyanopyridine-2-yl)ethyl}carbamate (Intermediate 6C) and converting to the HCl salt by treatment with 0.1M hydrochloric acid in acetonitrile and freeze drying. ¹H NMR (400 MHz, DMSO-d₆) 8.82 (3H, br s), 8.12 (1H, d, J=7.9 Hz), 7.83 (1H, d, J=8.3 Hz), 7.78-7.69 (3H, m), 7.63-7.54 (3H, m), 7.50-7.46 (1H, m), 7.43-7.37 (1H, m), 7.25-7.21 (1H, m), 5.18-5.12 (1H, m), 3.47-3.40 (1H, m), 3.37-3.29 (1H, m). LCMS (Method 1) RT 2.87 m/z 341 [MH⁺].

### Example 117: (S)-1-[2-(Benzo[d]isoxazol-3-yl)phenyl]-2-(4-bromopyridine-2-yl)ethan-1-amine hydrochloride)

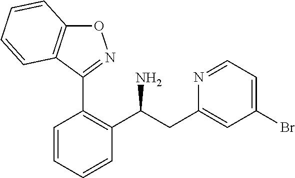 [SMILES: N[C@@H](Cc1cc(Br)ccn1)c1ccccc1-c1noc2ccccc12]

Starting from tert-butyl (S)-{1-[2-(benzo[d]isoxazol-3-yl)phenyl]-2-(4-bromopyridine-2-yl)ethyl}carbamate (Intermediate 27B) and converting to the HCl salt by treatment with 0.1M hydrochloric acid in acetonitrile and freeze drying. ¹H NMR (400 MHz, DMSO-d₆) 8.73 (3H, br s), 8.06 (1H, d, J=7.7 Hz), 7.86-7.84 (1H, m), 7.83 (1H, s), 7.76-7.69 (2H, m), 7.65-7.57 (2H, m), 7.55-7.51 (1H, m), 7.44-7.38 (1H, m), 7.28-7.26 (1H, m), 7.20 (1H, dd, J=2.1, 5.3 Hz), 5.24-5.16 (1H, m), 3.44-3.36 (1H, m), 3.30-3.23 (1H, m). LCMS (Method 1) RT 3.18 m/z 394/396 [MH⁺].

### Example 118: (S)-1-[2-(Benzo[d]isoxazol-3-yl)phenyl]-2-[6-(oxazol-2-yl)pyridine-2-yl]ethan-1-amine hydrochloride

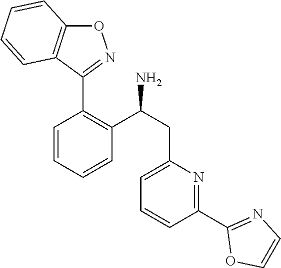 [SMILES: N[C@@H](Cc1cccc(-c2ncco2)n1)c1ccccc1-c1noc2ccccc12]

Starting from tert-Butyl (S)-{1-[2-(benzo[d]isoxazol-3-yl)phenyl]-2-[6-(oxazol-2-yl)pyridine-2-yl]ethyl}carbamate and converting to the HCl salt by treatment with aqueous HCl in acetonitrile and freeze drying. ¹H NMR (400 MHz, DMSO-d₆) 8.87 (3H, br s), 8.16 (1H, d, J=7.9 Hz), 8.08 (1H, d, J=0.6 Hz), 7.81-7.75 (1H, m), 7.66-7.52 (6H, m), 7.33 (1H, d, J=0.6 Hz), 7.32-7.24 (2H, m), 6.97 (1H, dd, J=1.8, 6.9 Hz), 5.34-5.27 (1H, m), 3.47-3.41 (1H, m), 3.35-3.30 (1H, m). LCMS (Method 2) RT 2.83 m/z 383 [MH⁺].

### Example 119: (S)-1-[2-(Benzo[d]isoxazol-3-yl)phenyl]-2-[6-(pyrrolidin-1-yl)pyridine-2-yl]ethan-1-amine hydrochloride

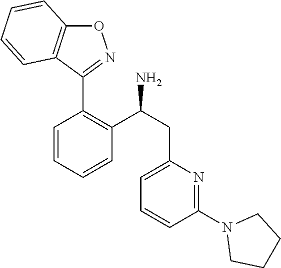 [SMILES: N[C@@H](Cc1cccc(N2CCCC2)n1)c1ccccc1-c1noc2ccccc12]

Starting from tert-butyl (S)-{1-[2-(benzo[d]isoxazol-3-yl)phenyl]-2-[6-(pyrrolidin-1-yl)pyridine-2-yl]ethyl}carbamate (Intermediate 49A) and converting to the HCl salt by treatment with 1M HCl in methanol and water and freeze drying. ¹H NMR (400 MHz, DMSO-d₆) 8.64 (3H, br s), 8.04 (1H, d, J=8.0 Hz), 7.83-7.78 (1H, m), 7.77-7.69 (2H, m), 7.64-7.59 (2H, m), 7.54-7.49 (1H, m), 7.43-7.38 (1H, m), 7.30-7.20 (1H, m), 6.21-6.10 (2H, m), 5.29-5.20 (1H, m), 3.15-2.96 (5H, m), 1.88-1.79 (4H, m), plus one proton hidden under the water peak. LCMS (Method 1) RT 2.33 m/z 385 [MH⁺].

### Example 120: (S)-{2-Amino-[2-(6-bromobenzo[d]isoxazol-3-yl)phenyl]ethyl}-6-cyanopyridine

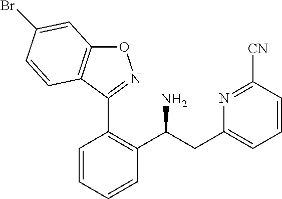 [SMILES: N#Cc1cccc(C[C@H](N)c2ccccc2-c2noc3cc(Br)ccc23)n1]

Hydrogen chloride (4M in dioxane, 0.75 mL) was added to a solution of (S)—N—{(S)-1-[2-(6-bromobenzo[d]isoxazol-3-yl)phenyl]-2-(6-cyanopyridine-2-yl)ethyl}-2-methylpropane-2-sulfinamide (Intermediate 7B, 0.053 g) in acetonitrile (3 mL) and the resultant mixture was stirred at room temperature for 2 hours. The mixture was concentrated in vacuo and the residue was purified by MDAP under basic conditions to give the title compound (0.031 g) as a white solid. ¹H NMR (400 MHz, DMSO-d₆) 8.18-8.16 (1H, m), 7.92 (1H, d, J=7.9 Hz), 7.71 (1H, t, J=7.7 Hz), 7.64-7.57 (2H, m), 7.55-7.52 (1H, m), 7.47 (1H, d, J=8.5 Hz), 7.42-7.33 (2H, m), 7.14 (1H, dd, J=1.1, 7.7 Hz), 4.47-4.42 (1H, m), 2.99-2.95 (2H, m). LCMS (Method 2) RT 3.32 m/z 419/421 [MH⁺].

### Example 121: (S)-6-{2-Amino-2-[2-(benzo[d]isoxazol-3-yl)phenyl]ethyl}pyridine-2-amine hydrochloride

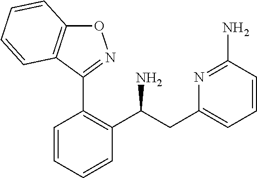 [SMILES: Nc1cccc(C[C@H](N)c2ccccc2-c2noc3ccccc23)n1]

A mixture of (S)—N—{(S)-2-[6-aminopyridine-2-yl]-1-[2-(benzo[d]isoxazol-3-yl)phenyl]ethyl}-2,2,2-trifluoroacetamide (Intermediate 63A, 0.046 g) and potassium carbonate (0.090 g) in methanol (3 mL) and water (1 mL) was stirred and heated at 55° C. for 3 days. After cooling, the mixture was concentrated in vacuo and the residue was suspended in a mixture of methanol and DCM and the solid was removed by filtration. The filtrate was passed down an SCX-2 cartridge which was washed with methanol before eluting the product off using 2M ammonia in methanol. After concentration of the basic fractions, the residue was treated with 1.25M HCl in methanol and concentrated in vacuo. The residue was freeze dried from acetonitrile and water to give the title compound (0.025 g) as a pale orange solid. ¹H NMR (400 MHz, DMSO-d₆) 7.95-7.92 (1H, m), 7.87-7.84 (1H, m), 7.76-7.64 (3H, m), 7.62-7.52 (2H, m), 7.45-7.42 (1H, m), 7.16 (1H, t, J=8.0 Hz), 6.21-6.18 (1H, m), 6.10 (1H, d, J=7.1 Hz), 5.77 (2H, s), 4.65-4.59 (1H, m), 2.91-2.85 (2H, m). LCMS (Method 2) RT 1.90 m/z 331 [MH⁺].

By proceeding in a similar manner to Example 121, the following compounds were prepared:

### Example 122: (S)-1-(6-{2-Amino-2-[2-(benzo[d]isoxazol-3-yl)phenyl]ethyl}pyridine-2-yl)-3-methylurea hydrochloride

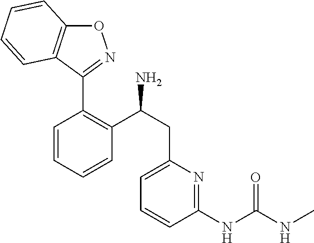 [SMILES: CNC(=O)Nc1cccc(C[C@H](N)c2ccccc2-c2noc3ccccc23)n1]

Starting from (S)—N-{1-[2-(benzo[d]isoxazol-3-yl)phenyl]ethyl}-2-[6-(3-methylureido)pyridine-2-yl]-2,2,2-trifluoroacetamide (Intermediate 64A) and converting to the HCl salt by treating with HCl in methanol and concentrating in vacuo followed by freeze drying from acetonitrile and water. ¹H NMR (400 MHz, DMSO-d₆) 8.98 (1H, s), 8.01-7.97 (1H, m), 7.88 (1H, br s), 7.81 (1H, d, J=8.5 Hz), 7.73-7.67 (2H, m), 7.55-7.49 (3H, m), 7.40-7.33 (2H, m), 6.85-6.81 (1H, m), 6.48-6.45 (1H, m), 4.85-4.78 (1H, m), 3.12-2.97 (2H, m), 2.61-2.58 (3H, m). LCMS (Method 2) RT 2.54 m/z 388.

### Example 123: (S)—N-(6-{2-Amino-2-[2-(benzo[d]isoxazol-3-yl)phenyl]ethyl}pyridine-2-yl)methanesulfonamide hydrochloride

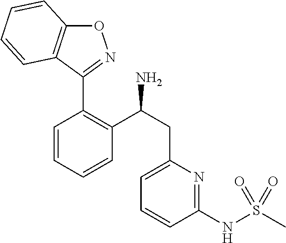 [SMILES: CS(=O)(=O)Nc1cccc(C[C@H](N)c2ccccc2-c2noc3ccccc23)n1]

Starting from (S)—N-{1-[2-(benzo[d]isoxazol-3-yl)phenyl]-2-[6-(N-[methylsulfonyl]methylsulfonamido)pyridine-2-yl]ethyl}-2,2,2-trifluoroacetamide (Intermediate 65A) and converting to the HCl salt by treating with HCl in methanol and concentrating in vacuo. ¹H NMR (400 MHz, DMSO-d₆) 7.93 (1H, d, J=7.8 Hz), 7.87-7.83 (1H, m), 7.76-7.61 (4H, m), 7.60-7.54 (1H, m), 7.52-7.43 (2H, m), 6.68-6.57 (2H, m), 4.83-4.77 (1H, m), 3.16-3.07 (2H, m), 3.02 (3H, s). LCMS (Method 2) RT 2.97 m/z 409 [MH⁺]

### Example 124: Methyl (S)-(6-{2-Amino-2-[2-(benzo[d]isoxazol-3-yl)phenyl]ethyl}pyridine-2-yl)carbamate hydrochloride

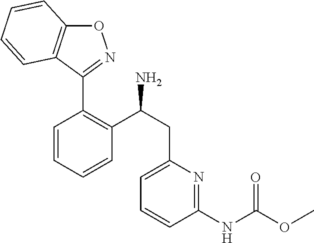 [SMILES: COC(=O)Nc1cccc(C[C@H](N)c2ccccc2-c2noc3ccccc23)n1]

Starting from methyl (S)-(6-{2-[2-(benzo[d]isoxazol-3-yl)phenyl]-2,2,2-trifluoroacetamido}ethylpyridine-2-yl)carbamate (Intermediate 64B) and converting to the HCl salt by treating with HCl in methanol and concentrating in vacuo. ¹H NMR (400 MHz, DMSO-d₆) 10.18 (1H, s), 8.83 (3H, br s), 8.02-7.99 (1H, m), 7.85-7.82 (1H, m), 7.77-7.72 (3H, m), 7.72-7.62 (3H, m), 7.53 (1H, d, J=8.5 Hz), 7.47-7.42 (1H, m), 6.79 (1H, d, J=7.3 Hz), 5.05-4.99 (1H, m), 3.69 (3H, s), 3.41 (1H, dd, J=8.9, 16.0 Hz), 3.26 (1H, dd, J=4.1, 16.0 Hz). LCMS (Method 2) RT 3.13 m/z 389 [MH⁺].

### Example 125: (S)-1-[2-(Benzo[d]isoxazol-3-yl)phenyl]-2-[3-methyl-6-(methylsulfonyl)pyridine-2-yl]ethan-1-amine hydrochloride

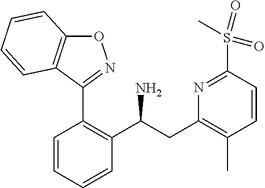 [SMILES: Cc1ccc(S(C)(=O)=O)nc1C[C@H](N)c1ccccc1-c1noc2ccccc12]

A mixture of (S)—N—{(S)-1-[2-(benzo[d]isoxazol-3-yl)phenyl]-2-(3-methyl-6-bromopyridine-2-yl)ethyl}-2-methylpropane-2-sulfinamide (Intermediate 61, 0.127 g), sodium methylsulfinate (0.04 g), (1S,2S)-cyclohexanediamine (0.011 g) and copper (I) trifluoromethanesulfonate benzene complex (0.019 g) in DMSO (2.5 mL) was stirred and heated in a sealed vial at 110° C. under argon overnight. After cooling, the mixture was diluted with methanol and passed through an SCX-2 column which was then washed with methanol. The product was eluted off the column with 2M ammonia in methanol and the product was purified by MDAP (basic). The resultant gum was dissolved in dioxane (2 mL) and treated with HCl (1M solution in ether, 0.5 mL), and the mixture was stirred for 1.5 hours. The mixture was concentrated in vacuo then stirred at reflux in ethyl acetate for 1 hour. After cooling, the solid was collected by filtration to give the title compound (0.027 g) as a white solid.

¹H NMR (400 MHz, DMSO-d₆) 8.77 (3H, br s), 8.21-8.17 (1H, m), 7.87-7.83 (1H, m), 7.83-7.77 (1H, m), 7.76-7.71 (1H, m), 7.69-7.59 (4H, m), 7.56-7.52 (1H, m), 7.45-7.41 (1H, m), 5.30 (1H, t, J=7.2 Hz), 3.60 (1H, dd, J=6.6, 14.4 Hz), 3.38-3.33 (1H, m), 2.87 (3H, s), 2.12 (3H, s). LCMS (Method 1) RT 2.92 m/z 408 [MH⁺].

### Biological Assays

The biological effects of the compounds may be assessed using one of more of the assays described herein.

### Example 126: Assay for HCN1 HCN2 and HCN4

### Example 126A, Using PatchXpress 7000A

Solutions for recording HCN currents were:

External Recording Solution

| | | |
|---|---|---|
| NaCl | 110 | mM |
| KCl | 30 | mM |
| MgCl2 | 1 | mM |
| CaCl2 | 1.8 | mM |
| HEPES | 10 | mM |
| Glucose | 5 | mM |

pH 7.4 (titrated with NaOH)

Internal Recording Solution

| | | |
|---|---|---|
| KCl | 60 | mM |
| KF | 70 | mM |
| NaCl | 10 | mM |
| HEPES | 10 | mM |
| EGTA | 11 | mM |
| MgATP | 2 | mM |

pH 7.35 (titrated with KOH)

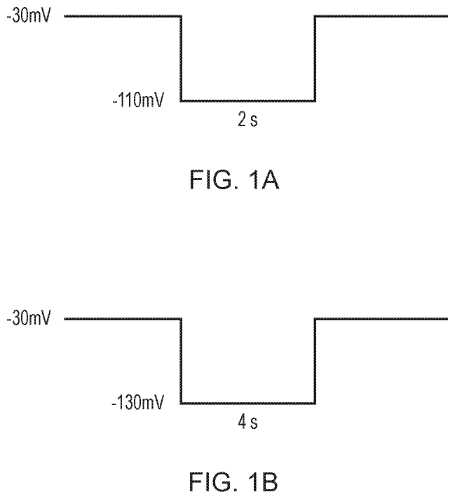

For HCN1 and HCN2, the pulse protocol involved stepping from a holding potential of −30 mV to −110 mV (see FIG. 1A) for 2 seconds to evoke the current. The membrane voltage was then stepped back to −30 mV for a further 8 seconds. This sequence was evoked repeatedly every 10 seconds throughout the experiment, starting prior to drug (Control A) and during cumulative additions of 5 increasing compound concentrations, then finally a 100% inhibiting concentration of cesium chloride (CsCl, 3 mM).

For HCN4, the pulse protocol involved stepping from a holding potential of −30 mV to −130 mV (see FIG. 1B) for 4 seconds to evoke the current. The membrane voltage was then stepped back to −30 mV, the voltage protocol had a start-to-start interval of 14 seconds, starting prior to drug (Control A) and during cumulative additions of increasing compound concentrations, then finally a 100% inhibiting concentration of cesium chloride (CsCl, 3 mM).

The peak inward current measured at the end of the pulse to −110 mV (HCN1 and HCN2) or −130 mV (HCN4) was measured and any leak current subtracted to calculate the HCN current. The HCN current amplitude was measured after each control or compound addition and normalized to the control amplitude (Control A).

All experiments were performed at room temperature (approximately 22° C.).

Each test compound concentration was applied to the cell for seven (7) minutes, at which point the next cumulative concentration was applied. 3 mM CsCl was applied to each cell for 2 minutes at the end of each experiment (Control B) to determine 100% inhibition level of the HCN current.

### Example 126B, Using the Sophion Qube

Solutions for recording HCN currents were:

External Recording Solution

| | | |
|---|---|---|
| NaCl | 110 | mM |
| KCl | 30 | mM |
| MgCl2 | 1 | mM |
| CaCl2 | 1.8 | mM |
| HEPES | 10 | mM |
| Glucose | 10 | mM |

pH 7.4 (titrated with NaOH)

Internal Recording Solution

| | | |
|---|---|---|
| KCl | 20 | mM |
| KF | 110 | mM |
| NaCl | 10 | mM |
| HEPES | 10 | mM |
| EGTA | 11 | mM |
| MgATP | 2 | mM |

pH 7.2 (titrated with KOH)
Osmolality adjusted to 290 mOsm

For HCN1 and HCN2 the cells were held at −30 mV and then stepped to −110 mV for 2 seconds before stepping back to −30 mV, this represents 1 experimental sweep. This voltage protocol was applied every 20 seconds for the duration of the experiment. Both the vehicle (0.3% DMSO) and full block (3 mM CsCl) addition periods were applied for 10 experimental sweeps each. The compound addition period was applied for 30 sweeps.

For HCN4, the cells were held at −30 mV and then stepped to −130 mV for 4 seconds before stepping back to −30 mV, this represents 1 experimental sweep. This voltage protocol was applied every 20 seconds for the duration of the experiment. Both the vehicle (0.3% DMSO) and full block (3 mM CsCl) addition periods were applied for 10 experimental sweeps each. The compound addition period was applied for 30 sweeps.

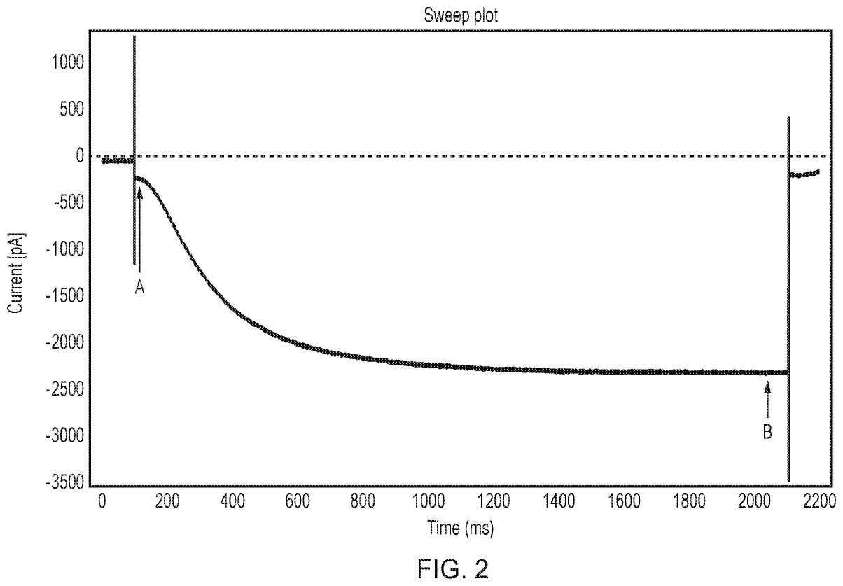

The currents evoked by the step to −110 mV (HCN1 and HCN2) or −130 mV (HCN4) were measured for the analysis of the percentage inhibition by test compounds. The current amplitudes were measured by subtracting metric A from metric B (see FIG. 2) with inhibition calculated by normalising to the vehicle addition (0.3% DMSO) and full inhibition by 3 mM CsCl in the same well.

The potency (IC₅₀) of test compound to inhibit the HCN ion channel was determined from a concentration-response curve generated from up to 8 test compound concentrations with up to 8 replicates per concentration. In total, compound was applied to the well for 600 seconds.

### Example 127: Assay for hERG

### Example 127A, Using IonWorks Quattro

Solutions for recording hERG currents were:

External Recording Solution

| | | |
|---|---|---|
| NaCl | 138 | mM |
| KCl | 2.7 | mM |
| MgCl2 | 0.5 | mM |
| CaCl2 | 0.9 | mM |
| Na₂HPO₄ | 8 | mM |
| KH₂PO₄ | 1.5 | mM |

pH 7.3 (titrated with NaOH)

Internal Recording Solution

| | | |
|---|---|---|
| KCl | 140 | mM |
| MgCl₂ | 1 | mM |
| HEPES | 20 | mM |
| EGTA | 1 | mM |

pH 7.3 (titrated with KOH)

Electrophysiological recordings were made from a Human Embryonic Kidney (HEK) cell line stably expressing the full length hERG channel. Single cell ionic currents were measured in the perforated patch clamp configuration (100 μg ml⁻¹ amphotericin) at room temperature (approx. 22° C.) using an IonWorks Quattro from Molecular Devices.

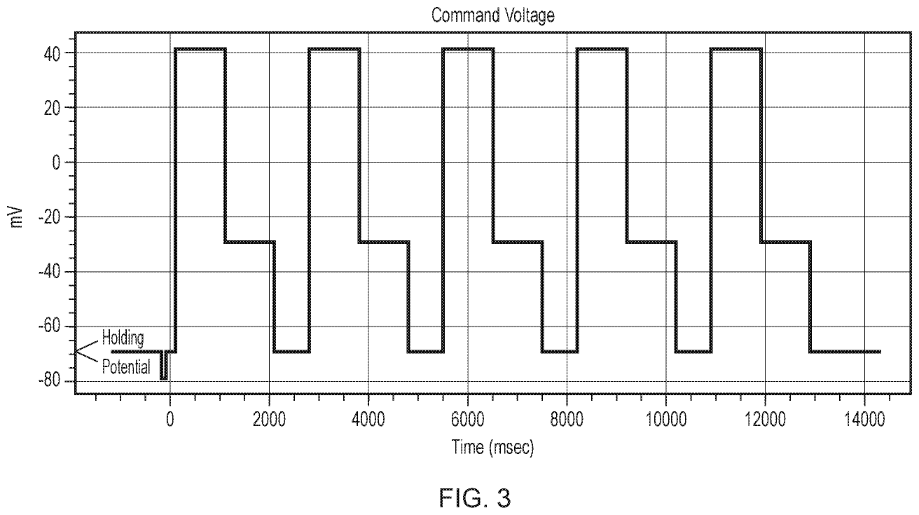

Cells were clamped at a holding potential of −70 mV for 30 s and then stepped to +40 mV for 1 s. This was followed by a hyperpolarising step of is to −30 mV to evoke the hERG tail current. This sequence was repeated 5 times at a frequency of 0.25 Hz (see FIG. 3). Currents were measured from the tail step at the 5th pulse and referenced to the holding current. Compounds were then incubated for 6-7 minutes prior to a second measurement of the hERG signal using an identical pulse train.

The potency (IC50) of test compounds to inhibit the hERG channel were determined from a concentration-response curve generated from up to 8 test compound concentrations with up to 4 replicates per concentration.

### Example 127B, Using Sophion Qube

Solutions for recording hERG currents were:

External Recording Solution

| | | |
|---|---|---|
| NaCl | 145 | mM |
| KCl | 4 | mM |
| MgCl2 | 1 | mM |
| CaCl2 | 2 | mM |
| HEPES | 10 | mM |
| Glucose | 10 | mM |

pH 7.4 (titrated with NaOH)

Internal Recording Solution

| | | |
|---|---|---|
| KCl | 20 | mM |
| KF | 120 | mM |
| HEPES | 10 | mM |
| EGTA | 10 | mM |

pH 7.2 (titrated with KOH)

The cells were held at a voltage of −80 mV and then stepped to +40 mV for 2 seconds before stepping to −40 mV for a further 2 seconds, this represents 1 experimental sweep. This voltage protocol was applied every 15 seconds for the duration of the experiment. Both the vehicle and 1st compound addition periods were applied for 10 sweeps. The 2nd compound addition period was applied for 20 sweeps. The compound concentration was added to the test well twice to assure complete exchange of the external buffer with the test compound. In total, compound was applied to the well for 450 seconds.

The peak tail currents evoked by the step to −40 mV were measured for the analysis of the percentage inhibition by test compounds. The peak tail currents were first normalised to the vehicle addition (0.3% DMSO) in the same well.

The potency (IC50) of test compounds to inhibit the hERG channel were determined from a concentration-response curve generated from up to 8 test compound concentrations with up to 4 replicates per concentration.

### Example 128: Assay for hNaᵥ1.5

### Example 128A, Using IonWorks Quattro

Solutions for recording Naᵥ1.5 currents were:

External Recording Solution

| | | |
|---|---|---|
| NaCl | 137 | mM |
| KCl | 4 | mM |
| MgCl2 | 1 | mM |
| CaCl2 | 1.8 | mM |
| HEPES | 10 | mM |

pH 7.3 (titrated with NaOH)

Internal Recording Solution

| | | |
|---|---|---|
| K-gluconate | 90 | mM |
| KCl | 40 | mM |
| NaCl | 10 | mM |
| MgCl₂ | 3.2 | mM |
| HEPES | 5 | mM |
| EGTA | 3.2 | mM |

pH 7.3 (titrated with KOH)

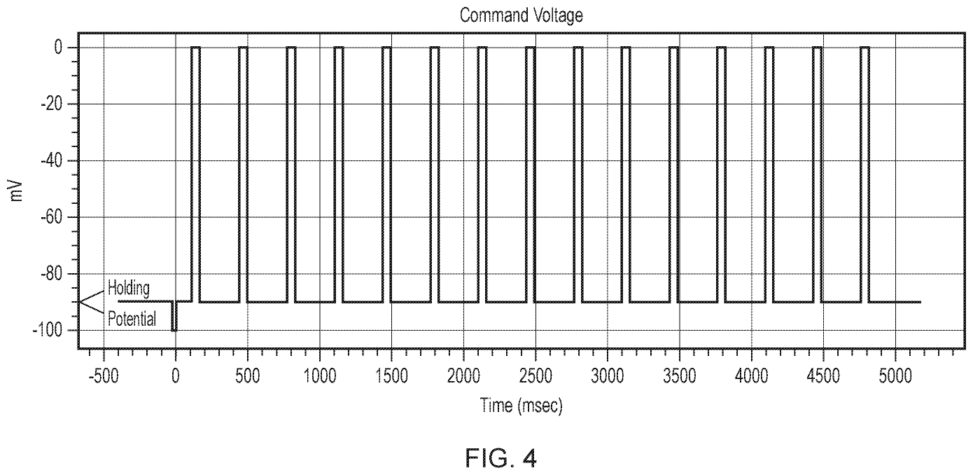

Electrophysiological recordings were made from a human embryonic kidney (HEK) cell line stably expressing the full length hNaᵥ1.5. Population patch clamp measurements were made in the perforated patch clamp configuration (100 μg ml⁻¹ amphotericin) at room temperature (approx. 22° C.) using an IonWorks Quattro from Molecular Devices. The voltage protocol is illustrated in FIG. 4. Currents were first measured under control (pre-compound addition) conditions. Compounds were then incubated for 5-7 minutes prior to a second measurement of the hNaᵥ1.5 signal using an identical pulse train. Currents were measured from the depolarising step at the 15^{th} pulse and referenced to the holding current.

### Example 128B, Using Sophion Qube

The cells were held at −100 mV followed by a depolarising step to −10 mV for 100 milliseconds before stepping back to −100 mV, this represents 1 experimental sweep. This voltage protocol was applied at 0.1 Hz and 4 Hz, to evaluate both tonic block, and the potential for use-dependent block of the hNaᵥ1.5 channel. The vehicle and compound addition periods were applied for 20 sweeps at 0.1 Hz to assess tonic block, and as a train of 20 depolarisations at a frequency of 4 Hz to test for use-dependent block.

For tonic block (0.1 Hz), the peak currents evoked by the step to −10 mV were measured for the analysis of the percentage inhibition by test compounds. For use-dependent block (4 Hz), the peak current evoked at the 20^{th} depolarising step to −10 mV was measured for the analysis of the percentage inhibition by test compounds. Peak currents were first normalised to the vehicle addition (0.3% DMSO) in the same well. The potency (IC₅₀) of test compounds to inhibit the hNay1.5 channel were determined from a concentration-response curve generated from up to 8 test compound concentrations with up to 4 replicates per concentration and are quoted for the use dependent block.

### Example 129: MDCK Assay

The bi-directional MDCK permeability assay in MDCK-MDR1 cells was performed using MDCK-MDR1 cells (Solvo Biotechnology) seeded onto 24-well Transwell plates at 2.35×105 cells per well and used in confluent monolayers after a 3 day culture at 37° C. under 5% CO₂. Test compounds were added (10 μM, 0.1% DMSO final, n=2) to donor compartments of the Transwell plate assembly in assay buffer (Hanks balanced salt solution supplemented with 25 mM HEPES, adjusted to pH 7.4) for both apical to basolateral (A>B) and basolateral to apical (B>A) measurements. A parallel series of incubations were performed in the presence of the transporter inhibitor elacridar (5 μM) which was added to both compartments in the transwell plate. Incubations were performed at 37° C., with samples removed from both donor and acceptor chambers at T=0 and 1 hour for recovery assessment and compound analysed by mass spectrometry (LC-MS/MS), including an analytical internal standard.

Apparent permeability (Papp) values were determined from the relationship:

Papp=[CompoundAcceptor T=end]×VAcceptor/([CompoundDonor T=0]×VDonor)/incubation time×VDonor/Area×60×10−6 cm/s

Where V is the volume of each Transwell compartment (apical 125 μL, basolateral 600 μL), and concentrations are the relative MS responses for compound (normalized to internal standard) in the donor chamber before incubation and acceptor chamber at the end of the incubation.

Area=area of cells exposed for drug transfer (0.33 cm2).

Efflux ratios (Papp B>A/Papp A>B) were calculated for each compound from the mean Papp values in each direction. The MDCK-MDR1 cell line has been engineered to over-express the efflux transporter, MDR1 (P-glycoprotein), and a finding of good permeability B>A, but poor permeability A>B, suggests that a compound is a substrate for this transporter. The efflux ratios were also calculated in the same way from the runs carried out in the presence of the inhibitor. The net flux is the ratio of the efflux in the absence of inhibitor to that in the presence of inhibitor. A net flux value>5 (i.e. efflux ratio without inhibitor divided by efflux ratio plus inhibitor) is indicative of compounds being substrates for the transporter P-gp and would therefore have a greater likelihood of being restricted from the CNS (i.e. peripherally restricted).

Lucifer Yellow (LY) was added to the apical buffer in all wells to assess viability of the cell layer. As LY cannot freely permeate lipophilic barriers, a high degree of LY transport indicates poor integrity of the cell layer and wells with a LY Papp>10×10⁻⁶ cm/s were rejected. Note that an integrity failure in one well does not affect the validity of other wells on the plate.

Compound recovery from the wells was determined from MS responses (normalized to internal standard) in donor and acceptor chambers at the end of incubation compared to response in the donor chamber pre-incubation. Recoveries<50% suggest compound solubility, stability or binding issues in the assay which may reduce the reliability of a result.

### Biological Data

Table 2 shows the IC₅₀ values in μM for HCN2, HCN4, HCN1 using the PatchXpress protocol (PX) and hERG and Naᵥ1.5 using the ionWorks (IW) protocol for the compounds of the invention.

TABLE 2

| | PX HCN2 | PX HCN4 | PX HCN1 | IW hERG | IW Naᵥ1.5 |
|---|---|---|---|---|---|
| Compound | IC₅₀ | IC₅₀ | IC₅₀ | IC₅₀ | IC₅₀ |
| No | [μM] | [μM] | [μM] | [μM] | [μM] |
| 1 | 7.4 | — | — | — | — |
| 2 | 0.96 | >30 | — | 6.1 | 5.7 |
| 3 | 2.3 | >30 | — | 2.5 | 4.6 |
| 4 | 17.0 | — | — | — | — |
| 5 | 0.31 | 19.5 | 8.7 | 10.8 | 29.6 |
| 6 | 0.39 | 8.1 | 1.8 | — | — |
| 7 | 5.7 | — | — | — | — |
| 8 | 2.5 | — | — | — | — |
| 9 | 0.26 | 1.7 | 0.37 | — | — |
| 29 | 4.3 | >30 | 8.9 | 2.2 | >33 |
| 30 | 3.0 | 12.8 | 3.0 | 3.3 | >33 |
| 31 | 1.6 | 6.7 | — | — | — |
| 32 | 8.5 | — | — | — | — |
| 33 | 1.7 | >30 | — | 4.4 | 8.8 |
| 34 | 16.7 | — | — | — | — |
| 35 | 8.9 | — | — | — | — |
| 36 | 1.2 | >30 | — | 5.0 | 5.6 |
| 37 | 14.0 | — | — | — | — |
| 38 | 13.0 | — | — | — | — |
| 39 | 4.0 | >30 | — | 7.6 | 27.3 |
| 40 | 15.6 | — | — | 7.8 | >33 |
| 41 | 14.1 | >30 | — | — | — |
| 42 | 1.1 | >30 | — | 3.8 | 6.9 |
| 43 | 16.0 | — | — | — | — |
| 44 | 1.2 | 7.2 | — | 12.8 | 3.7 |
| 45 | 22.1 | — | — | — | — |
| 46 | 3.0 | >30 | — | 8.2 | 1.3 |
| 47 | 0.6 | >30 | — | 2.8 | 5.8 |
| 48 | 5.4 | >30 | — | 16.0 | 5.3 |
| 49 | 6.5 | 22.6 | — | 5.2 | 3.8 |
| 50 | 5.6 | 4.9 | — | — | — |
| 51 | 1.7 | 3.4 | — | — | — |
| 52 | 1.6 | 6.0 | — | 1.2 | 1.6 |
| 53 | 6.2 | — | — | — | — |
| 54 | 1.0 | 2.1 | — | — | — |
| 55 | 0.8 | 2.0 | — | — | — |
| 56 | 1.2 | 30 | — | 14.4 | 7.0 |
| 57 | 0.6 | 2.4 | — | — | — |
| 58 | 0.5 | 7.2 | — | 6.6 | 7.6 |
| 59 | 0.9 | 6.8 | — | — | — |
| 60 | 3.1 | >30 | >30 | 0.32 | 9.2 |
| 61 | 3.5 | >30 | — | 0.46 | 6.7 |
| 62 | 0.7 | 19.2 | — | 3.2 | 3.8 |
| 63 | 0.5 | >30 | 8 | 6.1 | 7.3 |
| 64 | 1.3 | 6.4 | — | — | — |
| 65 | 1.1 | 5.1 | — | — | — |
| 66 | 0.9 | 6.8 | — | — | — |
| 67 | 1.3 | 19.5 | — | 3.7 | 9.5 |
| 68 | 1.0 | 10.5 | 2.0 | 29.4 | 10.9 |
| 69 | 0.5 | 8.2 | 1.4 | 10.4 | 19.6 |
| 70 | 1.0 | 19.1 | 3.8 | 13.4 | 3.2 |
| 71 | 0.5 | 3.5 | 4.4 | 9.7 | 22.6 |
| 72 | 1.2 | 10.3 | 1.0 | 20.9 | 18.6 |
| 73 | 0.4 | 2.8 | 0.5 | 23.8 | 12.0 |
| 74 | 1.8 | 20.9 | 4.6 | 9.9 | 11.4 |
| 75 | 1.2 | 6.6 | — | 1.5 | 6.6 |
| 76 | 8.7 | — | — | — | — |
| 77 | 0.9 | 10.4 | 2.2 | >33 | 6.5 |
| 78 | 1.0 | 8.3 | — | 7.6 | 6.4 |
| 79 | 4.9 | — | — | — | — |
| 80 | 1.9 | — | — | — | — |
| 81 | 10.8 | >30 | — | 0.93 | 25.4 |
| 82 | 26.6 | — | — | — | — |
| 83 | 1.8 | >30 | — | — | — |
| 84 | 0.9 | 27.0 | — | 3.0 | 5.7 |
| 85 | 13.0 | — | — | — | — |
| 86 | 17.4 | — | — | — | — |
| 87 | 1.7 | 18.8 | — | — | — |
| 88 | 2.7 | >30 | — | — | — |
| 89 | 3.1 | >30 | — | 19.1 | 5.8 |
| 90 | 2.9 | 7.1 | — | 4.1 | 10.6 |
| 91 | 1.0 | 9.9 | — | 1.8 | 3.4 |
| 92 | 1.9 | >30 | — | 6.4 | 1.4 |
| 93 | 1.8 | >30 | — | 7.5 | 2.2 |
| 94 | 1.2 | 4.7 | — | — | — |
| 95 | 1.4 | >30 | — | 2.9 | >33 |
| 96 | 2.9 | >30 | — | — | — |
| 97 | 2.6 | 13.3 | — | 0.54 | 8.4 |
| 98 | 2.0 | 13.2 | — | — | — |
| 99 | 2.4 | >30 | — | 2.1 | 18.5 |
| 100 | 2.3 | >30 | — | 12.8 | 2.8 |
| 101 | 0.7 | 13.1 | — | 2.8 | 1.6 |
| 103 | 3.0 | 22.4 | — | 3.6 | 3.9 |
| 104 | 2.6 | 10.4 | — | 1.0 | 6.8 |
| 105 | 4.2 | 6.2 | — | 4.8 | 5.8 |
| 106 | 8.5 | >30 | — | — | — |
| 107 | 1.4 | 3.2 | — | — | — |
| 108 | 3.5 | 5.1 | — | — | — |
| 109 | 2.4 | >30 | — | 4.3 | 3.2 |
| 110 | 2.6 | 15.1 | — | 4..4 | >33 |
| 111 | 1.8 | >30 | — | 7.4 | 27.9 |
| 112 | 2.0 | 4.2 | — | 10.2 | 24.2 |
| 113 | 22.9 | — | — | — | — |
| 114 | 0.62 | 9 | 4.5 | 17.3 | 6.3 |
| 115 | 0.69 | 8.0 | — | 7.7 | 2.2 |
| 116 | 0.7 | 13.5 | 3.4 | 10.3 | 11.1 |
| 117 | 1.4 | 12.2 | — | 2.2 | 2.7 |
| 118 | 1.7 | 5.2 | — | 3.1 | 7.8 |
| 119 | 0.8 | 2.1 | — | 1.3 | 3.3 |
| 120 | 1.6 | 2.5 | — | — | — |
| 121 | 1.3 | >30 | — | 8.6 | 1.6 |
| 122 | 9.5 | 28.1 | — | — | — |
| 123 | 5.9 | >30 | — | — | — |
| 124 | 2.1 | >30 | — | 2.3 | 1.3 |
| 125 | 0.24 | 1.2 | 0.4 | — | — |

Table 3 shows the IC₅₀ values in μM for HCN2, HCN4, HCN1, hERG and Na_{y}1.5 using the Sophion Qube protocol (SQ) for the compounds of the invention

TABLE 3

| | SQ HCN2 | SQ HCN4 | SQ HCN1 | SQ hERG | SQ Naᵥ1.5 |
|---|---|---|---|---|---|
| Compound | IC₅₀ | IC₅₀ | IC₅₀ | IC₅₀ | IC₅₀ |
| No | [μM] | [μM] | [μM] | [μM] | [μM] |
| 5 | 0.7 | 20.2 | 2.9 | 9.2 | — |
| 10 | 17.2 | — | — | — | — |
| 11 | 3.2 | 30 | — | 4.7 | 21.4 |
| 12 | 1.8 | — | 2.8 | — | — |
| 13 | 2.6 | >30 | 3.2 | 1.4 | 15.1 |
| 14 | 1.3 | 2.7 | 2.4 | 4.1 | 14.8 |
| 15 | 1.2 | — | 5.1 | — | — |
| 16 | 2.0 | — | 3.0 | — | — |
| 17 | 5.8 | — | — | 3.1 | — |
| 18 | 0.7 | 14.6 | 7.6 | 5.1 | 10.3 |
| 19 | 1.4 | 13.1 | 2.9 | 1.3 | 11.5 |
| 20 | 3.1 | >30 | 16.9 | 2.9 | 28.1 |
| 21 | 2.5 | — | 5.3 | — | — |
| 22 | 0.8 | 20.8 | 27.4 | 5.7 | 24.1 |
| 23 | 1.7 | — | — | 1.2 | 13.9 |
| 24 | 1.3 | 7.8 | 4.2 | 3.4 | 22.4 |
| 25 | >30 | — | — | — | — |
| 26 | 1.8 | >30 | 25.7 | 15.1 | 19.2 |
| 27 | 1.4 | — | — | — | — |
| 28 | 1.0 | — | — | — | — |

Table 4 shows the efflux ratios and the net flux values for the compounds tested in the MOCK assay.

| | Efflux | Efflux | |
|---|---|---|---|
| | ratio | ratio in | |
| Compound | without | presence of | Net |
| no | inhibitor | inhibitor | flux |
| 2 | 1.1 | 0.8 | 1.4 |
| 5 | 18.9 | 1.4 | 13.5 |
| 11 | 5.2 | 0.6 | 8.7 |
| 12 | 1.1 | 1.1 | 1.0 |
| 13 | 6.2 | 0.9 | 6.9 |
| 14 | 1.5 | 0.9 | 1.7 |
| 18 | 1.1 | 0.6 | 1.8 |
| 19 | 12.4 | 0.7 | 17.7 |
| 20 | 6.2 | 0.6 | 10.3 |
| 22 | 4.5 | 0.6 | 7.5 |
| 24 | 10.4 | 0.7 | 14.8 |
| 26 | 4.2 | 0.7 | 6.0 |
| 29 | 1.4 | 0.7 | 2.0 |
| 30 | 9.6 | 1.1 | 8.7 |
| 34 | 1.0 | 0.5 | 2.0 |
| 36 | 0.8 | 0.6 | 1.3 |
| 39 | 2.7 | 1.7 | 1.6 |
| 42 | 1.5 | 0.8 | 1.9 |
| 56 | 2.5 | — | — |
| 58 | 1.4 | — | — |
| 63 | 1.0 | — | — |
| 65 | 5.6 | — | — |
| 68 | 0.8 | 0.7 | 1.1 |
| 69 | 0.8 | 0.7 | 1.1 |
| 70 | 1.6 | 0.7 | 2.3 |
| 71 | 1.1 | 0.6 | 1.8 |
| 72 | 11.3 | 1.1 | 10.3 |
| 73 | 1.0 | 0.6 | 1.7 |
| 74 | 16.7 | 0.8 | 20.9 |
| 81 | 1.2 | 0.9 | 1.3 |
| 84 | 0.8 | 0.7 | 1.1 |
| 89 | 24.6 | 0.7 | 35.1 |
| 91 | 23.1 | 1.0 | 23.1 |
| 95 | 4.5 | 0.8 | 5.6 |
| 97 | 12.2 | 0.6 | 20.3 |
| 99 | 78.5 | 1.3 | 60.4 |
| 100 | 8.7 | — | — |
| 103 | 45.5 | 1.1 | 41.4 |
| 104 | 31.3 | 1.0 | 31.3 |
| 105 | 33.8 | 1.9 | 17.8 |
| 111 | 1.0 | — | — |
| 114 | 1.0 | 0.7 | 1.4 |
| 115 | 0.8 | 0.8 | 1.0 |
| 116 | 1.1 | 0.7 | 1.6 |

### Example 130: Effect on Tinnitus by Pharmacological Block of HCN2 Ion Channels

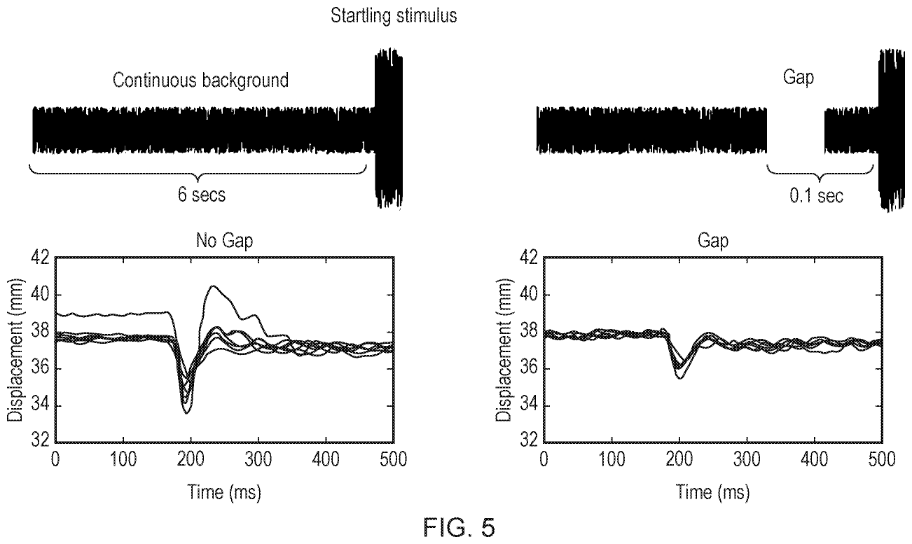

Tinnitus in guinea pigs was monitored using the gap induced inhibition of the acoustic startle (GPIAS) test (see FIG. 5). GPIAS is reduced when tinnitus was present; see Berger, J. I. et al. Effects of the cannabinoid CB1 agonist ACEA on salicylate ototoxicity, hyperacusis and tinnitus in guinea pigs. Hearing research, (2017), and Coomber, B. et al. Neural changes accompanying tinnitus following unilateral acoustic trauma in the guinea pig. Eur J Neurosci 40, 2427-2441, (2014).

In FIG. 5, sound stimulus (above) and corresponding pinna reflex (below) are shown in freely moving guinea pigs. Stimuli with no gap and gap are presented in a randomised order. Traces contaminated by movement (the upper trace in “no gap”) were removed before analysis.

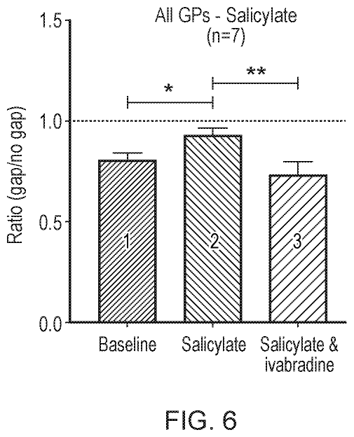

Tinnitus was induced within 1-2 hours in humans by high doses of salicylate. A similar short-term tinnitus model was implemented in guinea pigs by i.p. injection of salicylate. In all animals, salicylate caused behavioural inhibition of GPIAS (see bar 2 in FIG. 6). Block of HCN ion channels by the non-selective inhibitor ivabradine (which blocks HCN1-4 equally) reversed GPIAS (see bar 3 in FIG. 6). Thus, it was found that HCN ion channel block reverses behavioural signs of tinnitus in this short-term (salicylate) model.

Salicylate (350 mg/kg, i.p.) impairs behavioural gap detection 2 h after salicylate administration (see bar 2 in of FIG. 6). Gap detection was restored by blocking HCN channels with ivabradine (5 mg/kg, s.c.).

Mild unilateral noise exposure has been found to reduce GPIAS in around 40% of guinea pigs, an observation that resembles the effect of noise in humans, where noise exposure causes tinnitus in some but not all subjects. The noise-exposure model is more clinically relevant than the salicylate model, as it parallels a common cause of tinnitus in humans. A second important point is that it is long-term, while tinnitus induced by salicylate is rapidly reversed following salicylate exposure.

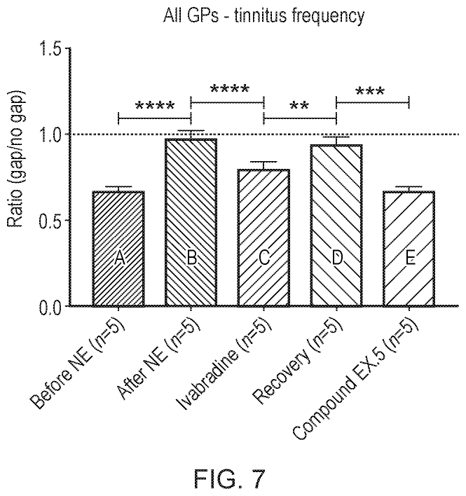

The reduced GPIAS seen following noise exposure (see bar B in FIG. 7) was found to be rapidly and completely reversed by HCN ion channel block with ivabradine, which blocks all four HCN ion channel isoforms equally (see bar C in FIG. 7). GPIAS returns following drug wash-out (see bar D in FIG. 7). Thus, it was found that block of HCN2 ion channels abolishes behavioural signs of tinnitus.

An HCN2-selective compound in accordance with present invention, Example 5 (“compound 476” in FIG. 7), chemically unrelated to ivabradine, also caused a complete reversal of “tinnitus” behaviour (see bar E in FIG. 7). In control experiments on noise-exposed guinea pigs showing no behavioural evidence of tinnitus, ivabradine was without effect on GPIAS (n=3, results not shown). The similar results obtained in the short-term salicylate model and in the long-term noise-exposure model suggest that tinnitus is both initiated and maintained by activity of HCN2 ion channels.
Bar A: naïve guinea pigs showed a large reduction in the acoustic startle response following a brief gap in continuous noise.
Bar B: following unilateral noise exposure (NE, 110 dB, 1 h, 8 weeks prior to testing) around 40% of guinea pigs developed impaired GPIAS.
Bar C: non-selective HCN inhibitor ivabradine (5 mg/kg, s.c.) fully restores GPIAS. Dark grey bar: reduced GPIAS returns following drug washout (1-2 d).
Bar E: Example 5, a compound with high selectivity for HCN2 over HCN1 (28×) and HCN4 (63×) fully restored GPIAS at the same dose that achieves full block of neuropathic pain (0.5 mg/kg, s.c.).

In control experiments on noise-exposed guinea pigs showing no behavioural tinnitus, ivabradine was without effect on gap detection (not shown).

### Example 131: Evaluation of CNS Penetration of HCN Blocker Ivabradine

Ivabradine in guinea pig plasma, brain (somatosensory cortex) and auditory nerve were assayed at 30 min after injection, the time used in Example 130.

Ratios of total concentrations in preliminary experiments for plasma: brain: auditory nerve were 1:0.12:0.57 (n=2). The small amount (12% of plasma level) detected in brain is largely accounted for by the presence of ivabradine within the vascular supply of the brain. As in other species, therefore, ivabradine is strongly excluded from guinea pig brain because of its hydrophilicity and Pgp substrate activity; see Young, G. T., Emery, E. C., Mooney, E. R., Tsantoulas, C. & McNaughton, P. A. Inflammatory and neuropathic pain are rapidly suppressed by peripheral block of hyperpolarisation-activated cyclic nucleotide-gated ion channels. Pain 155, 1708-1719, (2014).

The ratio of 0.57 between auditory nerve and plasma total concentrations shows that ivabradine is not excluded from auditory nerve, which is therefore accessible to plasma concentrations of ivabradine. The difference from a value of 1 may be accounted for by differences in binding to proteins in plasma and auditory nerve. Thus, it was found that the HCN blocker ivabradine penetrates the auditory nerve but not the CNS.

### Example 132: Effect of Genetic Deletion or Pharmacological Block of HCN2 on Hearing Thresholds

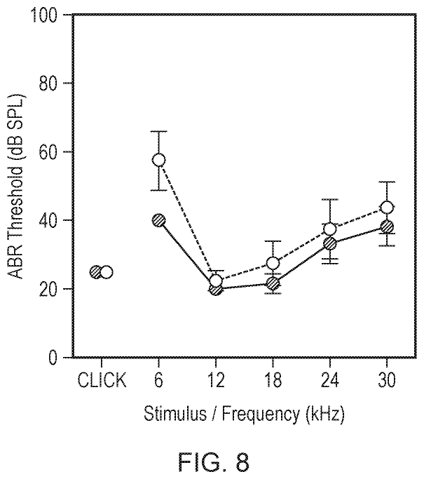

It was tested whether genetic deletion or pharmacological block of HCN2 affects auditory brainstem response (ABR) thresholds to tone pulses, with frequencies from 3 kHz to 42 kHz. Results are shown in FIG. 8, in which WT mice and sox10-Cre^{+/-}/fHCN2 litter mates (auditory-targeted HCN2 deletion, see above) show no significant difference in ABR threshold or latency. Similar results were obtained in adult mice treated with the non-selective HCN blocker ivabradine and with Example 29, an HCN2-selective blocker in accordance with the invention (20 mg/kg ip). No significant difference in hearing in mice with a global genetic deletion of HCN2 was found, but in this case the hearing was compared with WT littermates at age 2 weeks as the HCN2^{-/-} mice die by 3-4 weeks. It was concluded that HCN2 does not participate in normal hearing but is only activated in pathological circumstances, such as following noise exposure.

Mice carrying an auditory-targeted HCN2 deletion (upper line of unfilled dots in FIG. 8) and WT littermates (lower line of filled dots in FIG. 8) show no significant difference in either threshold (FIG. 8) or response latency (data not shown, latency of P1 and N1 waves measured with click and at 12 KHz and 18 kHz). Deletion of HCN2 expressed in spiral ganglion neurons therefore does not affect normal hearing thresholds or response latencies. Bars show SD (n=6). Thus, it was found that genetic deletion or pharmacological block of HCN2 does not affect normal hearing thresholds.

### Example 133: Mechanical Analgesic Effect of Compound of Example 5 in a Mouse Neuropathic Pain Model Tested Using a Von Frey Filament

The compound of Example 5 was tested in a mouse neuropathic pain model using WT Black6 strain mice. The model used was analogous to the model described in Seltzer Z, Dubner R, & Shir Y (1990), A novel behavioural model of neuropathic pain disorders produced in rats by partial sciatic nerve injury, Pain 43: 205-218). Further details of the methods used are described in Young G T et al. (2014), Inflammatory and neuropathic pain are rapidly suppressed by peripheral block of hyperpolarisation-activated cyclic nucleotide-gated ion channels; Pain 155: 1708-1719; and Tsantoulas C et al., (2017), Hyperpolarization-activated cyclic nucleotide-gated 2 (HCN2) ion channels drive pain in mouse models of diabetic neuropathy. Sci Transl Med 9: eaam6072.

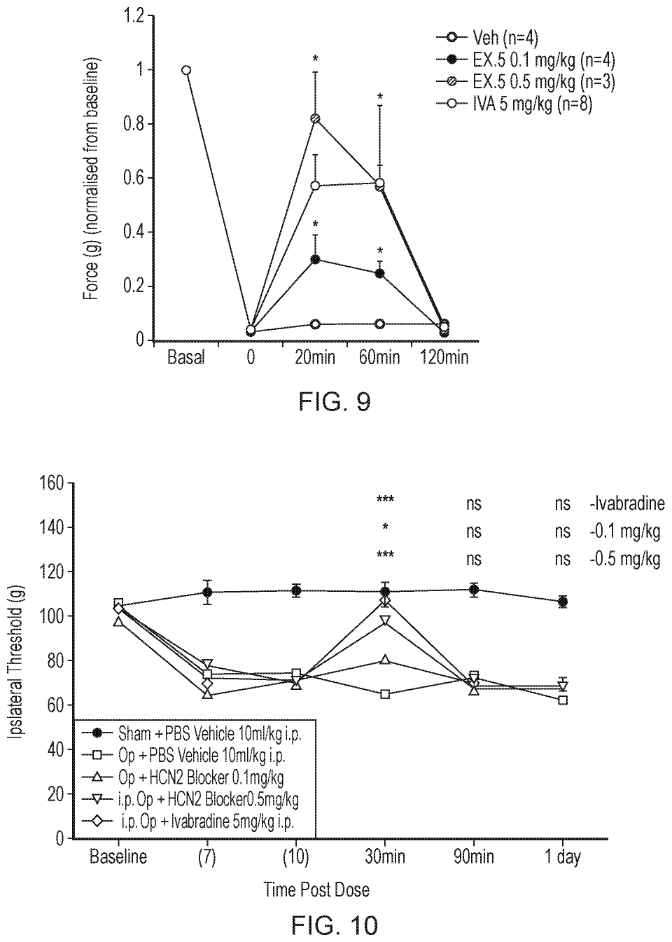

The compound of Example 5 was administered i.p. to the mice on day 5 following partial sciatic nerve ligation surgery, average data from 3-4 mice. The mechanical pain threshold was measured by manual von Frey filament applied to hind paw on the operated side, using the “up-down” method. Example 5 was compared to ivabradine at 5 mg/kg i.p. and i.p. injection of vehicle. The compound of Example 5 delivered full analgesia at 0.5 mg/kg i.p. and partial analgesia at 0.1 mg/kg i.p. (see FIG. 9 (significance over vehicle injection shown (*, p<0.05))). No hyperalgesia observed in the contralateral (unoperated) hind paw of the mice.

### Example 134: Analgesic Effect of Compound of Example 5 in a Mouse Neuropathic Pain Model Tested with Paw Pressure (Randall Sellito Test)

The compound of Example 5 was tested in the mouse model described in Example 133, but by assessing pain from mechanical paw pressure using the Randall Sellito test. The compound of Example 5 delivers full analgesia at 0.5 mg/kg i.p. and partial analgesia at 0.1 mg/kg i.p. The compound was compared with analgesia delivered by ivabradine at 5 mg/kg i.p. and with injection of vehicle only (see FIG. 10).

### Example 135: Thermal Analgesic Effect of Compound of Example 116 in a Mouse Neuropathic Pain Model

The Compound of Example 116 was tested in the mouse model described in Example 133, but pain threshold was assessed by thermal withdrawal latency (seconds) measured using infrared stimulus as described in Emery E C et al., (2011), HCN2 ion channels play a central role in inflammatory and neuropathic pain, Science 333: 1462-1466.

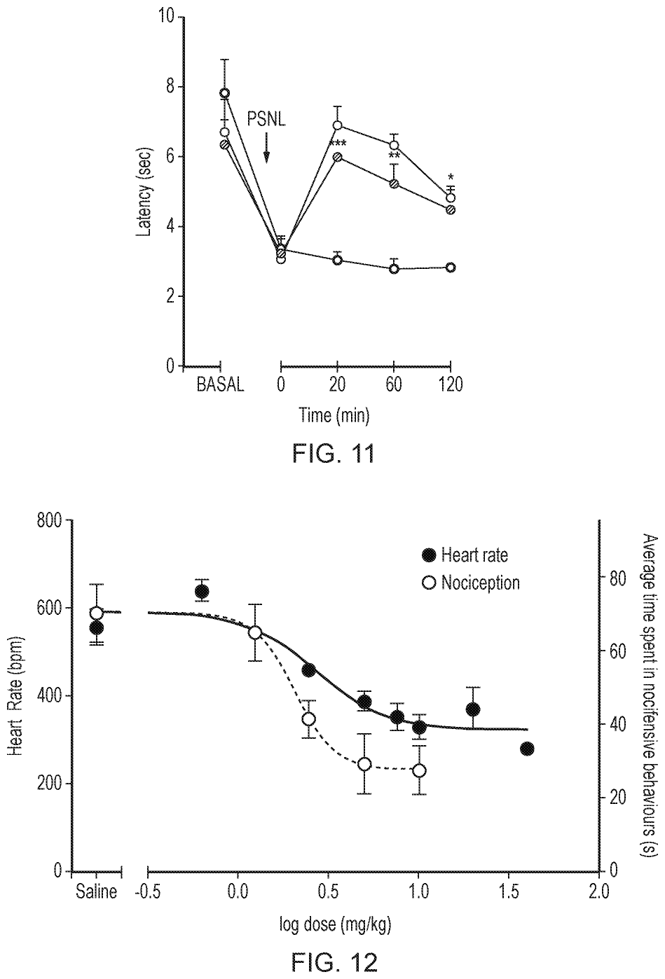

The compound of Example 116 delivered full analgesia in the thermal test at a dose of 1.5 mg/kg i.p. The compound was compared with analgesia delivered by ivabradine at 5 mg/kg i.p. and with injection of vehicle only (see FIG. 11 (significance is relative to vehicle)).

### Example 136: Selective Block of HCN2 Over HCN4 Delivers Effective Analgesia with Minimal Bradycardia in Mice

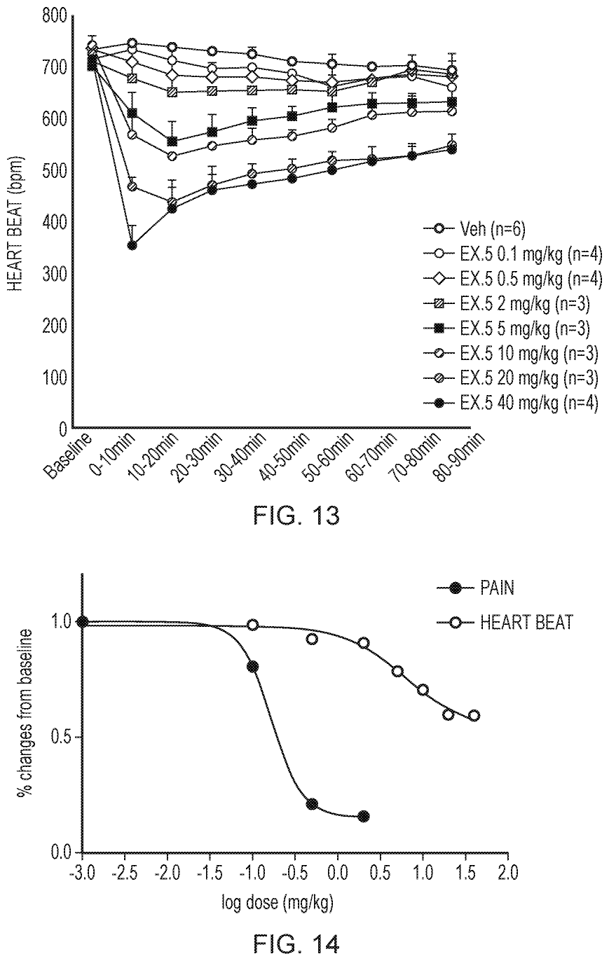

The compound of Example 5 was administered i.p. at doses of from 0.1 mg/kg to 40 mg/kg to awake, behaving Black6 strain mice together with a vehicle only control arm. Heart rate in the mice were measured with MouseOx pulse oximeter. The effect on heart rate with dose is shown in FIG. 13.

The compound of Example 5 gave full analgesia at an i.p. dose of 0.5 mg/kg (see Example 133), and at this dose the compound produced minimal bradycardia. The compound of Example 5 is approximately 63× selective for HCN2 over HCN4 measured using the PatchXpress 7000A automated patch clamp system described in Example 126.

FIG. 14 illustrates collected bradycardia data measured 10-20 min following injection of the compound of Example 5 in experiments similar to those above in mice, compared with maximal analgesia in experiments similar to those described in Example 133 above. Each point is mean from n=3-4 mice of Black6 strain. Analgesic ED₅₀=0.17 mg/kg i.p., giving a 37× therapeutic window for analgesia over bradycardia in mice (ED₅₀ for bradycardia=6.13 mg/kg i.p.). No neurological effects were observed in the mice until doses of 40 mg/kg i.p. when mild tremor was observed. This provides a 235× window for analgesia over neurological effects in the mice tested.

In comparison FIG. 12 shows that ivabradine blocks pain but because ivabradine does not discriminate between HCN2 and HCN4, there is little therapeutic window between analgesia and bradycardia in Black6 mice. This data is based on inflammatory pain measured in a formalin model implemented in Black6 mice. Heart rate was measured using MouseOx pulse oximeter in awake, behaving, mice (published data from Young G T et al. (2014), Pain 155: 1708-1719).

### Example 137: Repeated Subthreshold Injections of the Compound of Example 5 Confers Gradual Build Up of Analgesia

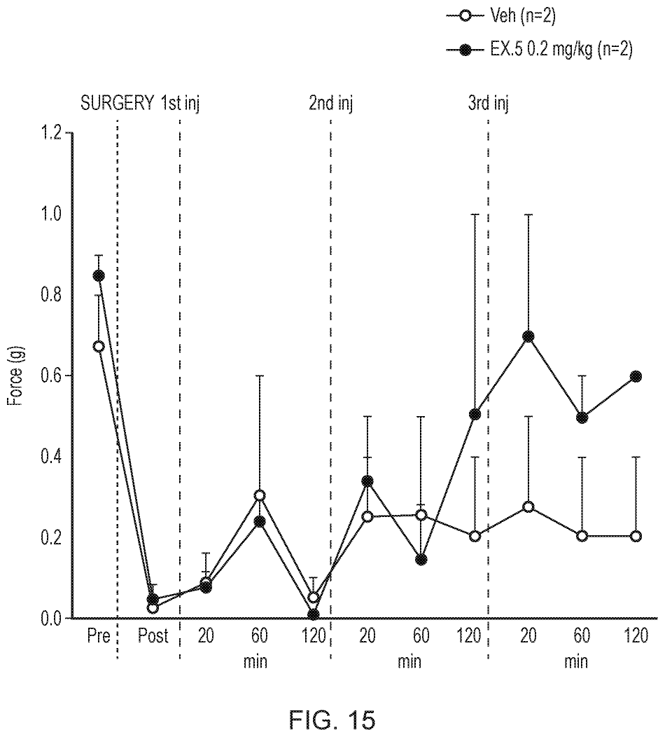

The compound of Example 5 was tested in the mouse neuropathic pain model described in Example 133. Mechanical pain threshold was measured as in Example 133 1 day before and 8 days after partial sciatic nerve ligation (PSNL) neuropathic pain induction. The test compound was administered at a low dose of 0.2 mg/kg every two hours with a control arm of vehicle only injections. FIG. 15 shows the analgesic effect over time as the successive doses of test compound were administered. Consistent with the data in Example 133 and FIG. 9, the first dose of 0.2 mg/kg resulted in only partial analgesia. However FIG. 15 shows that subsequent doses of the test compound resulted in an increase in analgesia towards full analgesia.

### Example 138: Regular Dosing of the Compound of Example 5 Provides a Cumulative Analgesic Effect

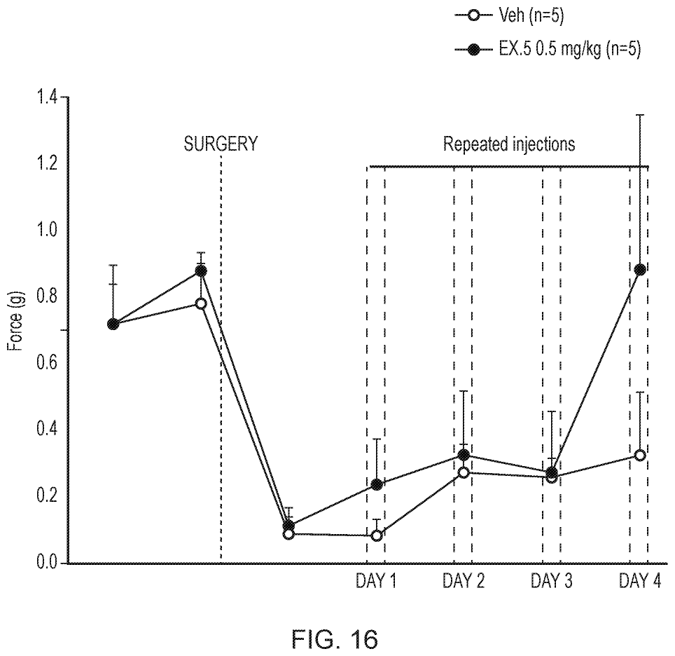

The compound of Example 5 was tested in the mouse neuropathic pain model described in Example 133. Mechanical pain threshold was measured as in Example 133 1 day before and 5-8 days after partial sciatic nerve ligation (PSNL) neuropathic pain induction. The test compound was administered at a dose of 0.5 mg/kg twice-daily at 8 hour intervals with a control arm of vehicle only injections, and mechanical pain threshold was measured 4 hours after the first injection. FIG. 16 shows the analgesic effect over time as the successive doses of test compound were administered.

The compound of Example 5 resulted in little analgesia when measured 4 hours after first injection (consistent with FIG. 9) but with successive doses injected twice-daily over 4 days, a build up to full and sustained analgesia was observed.

1. A compound of the formula (I), or a pharmaceutically acceptable salt thereof: 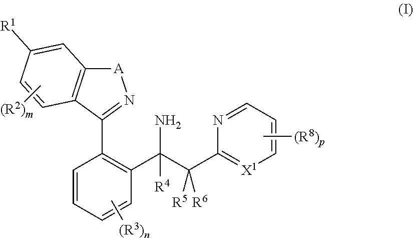
wherein
R¹ is selected from: H, halo, —CN, C₁₋₆ alkyl, C₁₋₆ haloalkyl, —OR^{B1}, C₂₋₆ alkenyl, C₂₋₆ alkynyl, C₃₋₆ cycloalkyl and C₃₋₆ cycloalkyl-C₁₋₆ alkyl-, and wherein any alkyl, alkenyl, alkynyl or cycloalkyl group in R¹ is optionally substituted with 1 to 4 substituents independently selected from halo, C₁₋₄ alkyl, C₁₋₄ haloalkyl and —OR^{B2};
R² is independently at each occurrence selected from: halo, C₁₋₆ alkyl and C₁₋₆ haloalkyl;
A is O or NR⁹;
R⁹ is selected from H, C₁₋₆ alkyl, C₃₋₆ cycloalkyl and C₃₋₆ cycloalkyl-C₁₋₆ alkyl-;
R³ is independently at each occurrence selected from: halo, —CN, C₁₋₆ alkyl, C₁₋₆ haloalkyl, —NR^{A3}R^{A3} and —OR^{B3};
R⁴, R⁵ and R⁶ are each independently selected from: H and C₁₋₄alkyl,
or R⁵ and R⁶ together with the carbon atom to which they are attached form a C₃₋₆ cycloalkyl;
X¹ is N or CR⁷;
R⁷ is selected from: H, halo, —CN, C₁₋₆ alkyl, C₁₋₆ haloalkyl, C₃₋₆ cycloalkyl, —C(O)NR^{A4}R^{A4}, —N(R^{A4})C(O)R^{B4}, —C(O)R^{B4} and —S(O)XR^{B4};
R⁸ is independently at each occurrence selected from: halo, —CN, nitro, C₁₋₆ alkyl, C₁₋₆ haloalkyl, C₂₋₆alkenyl, C₂₋₆ alkynyl, —OR¹⁰, —NR¹⁰R¹¹, —S(O)ₓR¹⁰, —C(O)R¹⁰, —OC(O)R¹⁰, —C(O)OR^{10A}, —C(O)NR¹⁰R¹¹, —N(R¹¹)C(O)R¹⁰, —N(R¹¹)C(O)NR¹⁰R¹¹, —N(R¹¹)C(O)OR¹⁰, —N(R¹¹) SO₂R¹⁰, —SO₂NR¹⁰R¹¹, C₃₋₆ cycloalkyl, 3 to 7 membered heterocyclyl, phenyl and 5 or 6 membered heteroaryl; wherein said alkyl, alkenyl, alkynyl, cycloalkyl, or heterocyclyl group is optionally substituted with from 1 to 4 R¹² groups, and said phenyl or heteroaryl group is optionally substituted with from 1 to 4 R¹³ groups;
R¹⁰ is independently at each occurrence selected from: H, C₁₋₆ alkyl, C₁₋₆ haloalkyl and C₃₋₆ cycloalkyl; wherein said alkyl or cycloalkyl group is optionally substituted with from 1 to 4 R¹⁴ groups;
R^{10A} is selected from: C₁₋₆ alkyl, C₁₋₆ haloalkyl and C₃₋₆ cycloalkyl; wherein said alkyl or cycloalkyl group is optionally substituted with from 1 to 4 R¹⁴ groups;
R¹¹ is independently at each occurrence selected from: H and C₁₋₆ alkyl;
or R¹⁰ and R¹¹ together with the nitrogen to which they are attached form a 4 to 7 membered heterocyclyl, wherein said heterocyclyl is optionally substituted with 1 or 2 substituents selected from halo, ═O, C₁₋₄ alkyl, C₁₋₄ haloalkyl and —OR^{B7};
R¹² and R¹⁴ are each independently at each occurrence selected from: halo, ═O, —CN, nitro, C₁₋₄ alkyl, C₁₋₄ haloalkyl, C₃₋₆ cycloalkyl, —OR^{B5}, —NR^{A5}R^{A5}, —S(O)ₓR^{B5}, —C(O)R^{B5}, —NR^{A5}C(O)R^{B5}, —C(O)NR^{A5}R^{A5}, —NR^{A5}SO₂R^{B5} and —SO₂NR^{A5}R^{A5};
R¹³ is independently at each occurrence selected from: halo, —CN, nitro, C₁₋₄ alkyl, C₁₋₄ haloalkyl, C₃₋₆ cycloalkyl, —OR^{B6}, —NR^{A6}R^{A6}, —S(O)ₓR^{B6}, —C(O)R^{A6}, —NR^{A6}C(O)R^{B6}, —C(O)NR^{A6}R^{A6}, —NR^{A6}S02R^{B6}, —SO₂NR^{A6}R^{A6};
R^{B1} and R^{B3} are independently at each occurrence selected from: H, C₁₋₆ alkyl and C₁₋₆ haloalkyl;
R^{B2}, R^{B4}, R^{B5}, R^{B6} and R^{B7} are independently at each occurrence selected from: H, C₁₋₄ alkyl and C₁₋₄ haloalkyl;
R^{A3}, R^{A4}, R^{A5} and R^{A6} are independently at each occurrence selected from H and C₁₋₄ alkyl;
m and p are each independently an integer selected from: 0, 1, 2 and 3;
n is an integer selected from: 0, 1, 2, 3 and 4; and
x is independently at each occurrence an integer selected from 0, 1, 2 and 3;
with the proviso that the compound is not a compound of the formula (A) or formula (B):

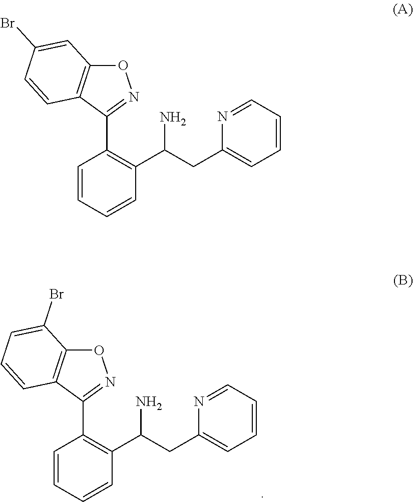 [SMILES: NC(Cc1ccccn1)c1ccccc1-c1noc2cc(Br)ccc12 ; NC(Cc1ccccn1)c1ccccc1-c1noc2c(Br)cccc12]

2. The compound of claim 1, wherein m is 0.

3. The compound of claim 1 or claim 2, wherein R¹ is selected from: H, —CN, C₁₋₄ alkyl, C₂₋₄ alkenyl, C₂₋₄ alkynyl, C₃₋₅ cycloalkyl, and wherein said alkyl, alkenyl, alkynyl or cycloalkyl group is optionally substituted with 1 to 4 substituents independently selected from C₁₋₄ alkyl and —OR^{B2}.

4. The compound of claim 1 or claim 2, wherein R¹ is selected from H, —CN and C₁₋₃ alkyl.

5. The compound of claim 1 or claim 2, wherein R¹ is H.

6. The compound of any of claims 1 to 5, wherein A is O.

7. The compound of any of claims 1 to 5, wherein A is NR⁹ and R⁹ is selected from: H and C₁₋₄ alkyl, for example R⁹ is methyl, ethyl or isopropyl.

8. The compound of any of claims 1 to 7, wherein R³ is selected from halo, —CN, C₁₋₄ alkyl, C₁₋₄ haloalkyl, —OC₁₋₄ alkyl and —NR^{A3}R^{A3}, for example wherein R³ is selected from:
halo, —CN and C₁₋₃ alkyl.

9. The compound of any of claims 1 to 8, wherein n is 0 or 1.

10. The compound of any of claims 1 to 9, wherein R⁴ and R⁵ are H and R⁶ is H or C₁₋₃ alkyl, for example wherein R⁴, R⁵ and R⁶ are H.

11. The compound of any of claims 1 to 10, wherein X¹ is CR⁷.

12. The compound of any of claims 1 to 11, wherein R⁷ is selected from H, halo, —CN, C₁₋₄ alkyl, C₁₋₄ haloalkyl, C₃₋₅ cycloalkyl, —N(R^{A4})C(O)R^{B4} and C(O)R^{B4}.

13. The compound of any of claims 1 to 11, wherein R⁷ is selected from halo (e.g. F), and C₁₋₄ alkyl (e.g. methyl).

14. The compound of any of claims 1 to 13, wherein p is 0 or 1.

15. The compound of any of claims 1 to 14, wherein the group of the formula: 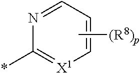
is

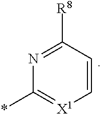

16. The compound of any of claims 1 to 15, wherein R³ is independently at each occurrence selected from: halo, —CN, C₁₋₆ alkyl, C₁₋₆ haloalkyl, —OR¹⁰, —NR¹⁰R¹¹, —S(O)ₓR¹⁰ (wherein x is 0, 1 or 2, preferably 1 or 2), —C(O)NR¹⁰R¹¹, —N(R¹¹)C(O)R¹⁰, —N(R¹¹)C(O)NR¹⁰R¹¹, —N(R¹¹)C(O)OR¹⁰, —N(R¹¹) SO₂R¹⁰, C₃₋₆ cycloalkyl, 4 to 6 membered heterocyclyl and 5 or 6 membered heteroaryl containing 1 or 2 ring nitrogen atoms;
wherein said alkyl, cycloalkyl, or heterocyclyl group is optionally substituted with from 1 to 4 R¹² groups, and said heteroaryl group is optionally substituted with from 1 to 4 R¹³ groups.

17. The compound of any of claims 1 to 15, wherein R³ is independently at each occurrence selected from: —CN, —OR¹⁰, —NR¹⁰R¹¹, —S(O)₂R¹⁰, —S(O)R¹⁰, —C(O)NR¹⁰R¹¹, —N(R¹¹)C(O)R¹⁰, —N(R¹¹)C(O)NR¹⁰R¹¹, —N(R¹¹)C(O)OR¹⁰, —SO₂NR¹⁰R¹¹, —N(R¹¹) SO₂R¹⁰, —C₁₋₄ alkyl-CN, —C₁₋₄ alkyl-OR^{B5}, —C₁₋₄ alkyl-NR^{A5}R^{A5}, —C₁₋₄ alkyl-S(O)₂R^{B5}, —C₁₋₄ alkyl-NR^{A5}C(O)R^{B5}, —C₁₋₄alkyl-C(O)NR^{A5}R^{A5}, —C₁₋₄alkyl-NR^{A5}SO₂R^{B5}, —C₁₄alkyl-SO₂NR^{A5}R^{A5}.

18. The compound of any of claims 1 to 15, wherein R³ is independently at each occurrence selected from: halo (e.g. F or Br), —CN, methyl, ethyl, 2-hydroxyethyl, methoxy, 2-hydroxyethoxy, —CF₃, —NH₂, —S(O)₂Me, —C(O)NH₂, —C(O)N(H)Me, —C(O)N(Me)₂, —(CH₂)₂C(O)NH₂, —(CH₂)₂C(O)N(H)Me, —(CH₂)₂C(O)N(Me)₂, —NHC(O)OMe, and pyrazolyl.

19. The compound of any of claims 1 to 15, wherein R³ is —S(O)₂C₁₋₄alkyl (e.g. —S(O)₂Me).

20. The compound of any of claims 1 to 10, wherein the group of the formula 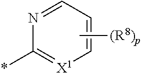
is selected from

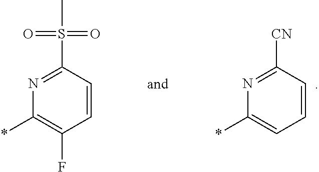 [SMILES: *c1nc(S(C)(=O)=O)ccc1F ; *c1cccc(C#N)n1]

21. The compound of any of claims 1 to 13, wherein the compound of the formula (I) is a compound of the formula (IV): 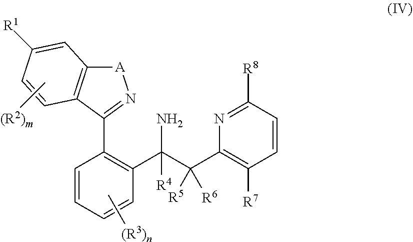
wherein R⁷ is not H.

22. The compound of claim 21, wherein
R⁷ is selected from: halo, —CN, C₁₋₄ alkyl and C₁₋₄ haloalkyl; and
R⁸ is selected from: halo, —CN, C₁₋₄ alkyl, C₁₋₄ haloalkyl, —C₁₋₄ alkyl-OH, —C₁₋₄ alkyl-OMe, —C₁₋₄ alkyl-C(O)NH₂, —C₁₋₄ alkyl-C(O)N(H)—C₁₋₄ alkyl, —C₁₋₄ alkyl-C(O)N(C₁₋₄ alkyl)₂, —OC₁₋₄ alkyl, —OC₂₋₄ alkyl-OH, —OC₂₋₄ alkyl-O—C₁₋₄ alkyl, —NH₂, —NH(C₁₋₄ alkyl), —N(C₁₋₄ alkyl)₂, —C(O)NH₂, —C(O)N(H)—C₁₋₄ alkyl, —C(O)N(C₁₋₄ alkyl)₂, —SO₂C₁₋₄ alkyl and pyrazolyl.

23. The compound of claim 21 or claim 22, wherein R⁷ is selected from F and methyl.

24. The compound of any of claims 1 to 23, wherein group 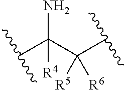
is of the formula 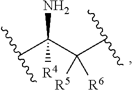
optionally wherein the group 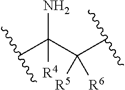
is:

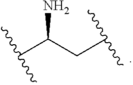 [SMILES: *C[C@@H](*)N]

25. A compound of claim 1 selected from a compound shown in Table 1 in the description, or a pharmaceutically acceptable salt thereof.

26. A pharmaceutical composition comprising a compound of any of claims 1 to 25, except the compounds of the formulae (A) and (B) are not excluded, or a pharmaceutically acceptable salt thereof, and a pharmaceutically acceptable excipient.

27. A compound of any of claims 1 to 25, except the compounds of the formulae (A) and (B) are not excluded, or a pharmaceutically acceptable salt thereof, for use as a medicament.

28. A compound of any of claims 1 to 25, except the compounds of the formulae (A) and (B) are not excluded, or a pharmaceutically acceptable salt thereof, for use in the treatment of a disease or medical condition mediated by hyperpolarisation activated cyclic-nucleotide modulated ion channel 2 (HCN2).

29. A method of treating a disease or medical condition mediated by HCN2 in a subject in need thereof, the method comprising administering to the subject an effective amount of: a compound of any of claims 1 to 25, except the compounds of the formulae (A) and (B) are not excluded, or a pharmaceutically acceptable salt thereof.

30. The compound for the use of claim 28, or the method of treatment of claim 29, wherein the disease or medical condition mediated by HCN2 is pain, for example neuropathic pain or inflammatory pain.

31. A HCN2 inhibitor for use in the treatment of tinnitus or a related condition.

32. The HCN2 inhibitor for the use of claim 31, wherein the HCN2 inhibitor is a compound of any of claims 1 to 25, except the compounds of the formulae (A) and (B) are not excluded, or a pharmaceutically acceptable salt thereof.

33. A HCN2 inhibitor for use in the treatment of migraine.

34. The HCN2 inhibitor for the use of claim 33, wherein the HCN2 inhibitor is a compound of any of claims 1 to 25, except the compounds of the formulae (A) and (B) are not excluded, or a pharmaceutically acceptable salt thereof.
